(19) United States
(12) Patent Application Publication
(10) Pub. No.: US 2024/0175034 A1
(43) Pub. Date: May 30, 2024

(54) FN3 DOMAIN-SIRNA CONJUGATES AND USES THEREOF

(71) Applicant: ARO BIOTHERAPEUTICS COMPANY, Philadelphia, PA (US)

(72) Inventors: Swapnil Kulkarni, Philadelphia, PA (US); Thomas Zengeya, Philadelphia, PA (US); Sukumar Sakamuri, Philadelphia, PA (US); Jacqueline Lee, Philadelphia, PA (US); Steven G. Nadler, Philadelphia, PA (US); Meera Ramanujam, Philadelphia, PA (US); Karyn T. O’Neil, Philadelphia, PA (US); Stephen Anderson, Philadelphia, PA (US)

(21) Appl. No.: 18/490,450

(22) Filed: Oct. 19, 2023

Related U.S. Application Data
(60) Provisional application No. 63/380,112, filed on Oct. 19, 2022.
(60) Provisional application No. 63/505,898, filed on Jun. 2, 2023.

(51) Int. Cl. C12N 15/113; A61P 37/06

(52) U.S. Cl. CPC C12N 15/1138; A61P 37/06; C12N 2310/14; C12N 2310/312; C12N 2310/315; C12N 2310/321; C12N 2310/322; C12N 2310/3513

(57) ABSTRACT

The present disclosure relates to compositions, such as siRNA molecules and FN3 domains conjugated to the same, as well as methods of making and using the molecules.

### CROSS-REFERENCE TO RELATED APPLICATIONS

The present application claims priority to U.S. Provisional Application No. 63/380,112, filed Oct. 19, 2022, and U.S. Provisional Application No. 63/505,898, filed Jun. 2, 2023, each of which is hereby incorporated by reference in its entirety.

### REFERENCE TO AN ELECTRONIC SEQUENCE LISTING

This application contains a Sequence Listing which has been submitted electronically in XML format and is hereby incorporated by reference in its entirety. The Sequence Listing is named “ROO-032US_SL.xml”, was created on Jan. 4, 2024, and is 4,104,653 bytes in size.

### FIELD

The present embodiments relate to siRNA molecules that can be conjugated to fibronectin type III domains (FN3) and methods of making and using the molecules.

### BACKGROUND

Therapeutic nucleic acids include, e.g., small interfering RNA (siRNA), micro RNA (miRNA), antisense oligonucleotides, ribozymes, plasmids, immune stimulating nucleic acids, antisense, antagomir, antimir, microRNA mimic, supermir, U1 adaptor, and aptamer. In the case of siRNA or miRNA, these nucleic acids can downregulate intracellular levels of specific proteins through a process termed RNA interference (RNAi). The therapeutic applications of RNAi are extremely broad, since siRNA and miRNA constructs can be synthesized with any nucleotide sequence directed against a transcript of any target protein. To date, siRNA constructs have shown the ability to specifically downregulate target proteins in both in vitro and in vivo models. In addition, siRNA constructs are currently being evaluated in clinical studies and have been approved for a variety of diseases.

However, two problems currently faced by siRNA constructs are, first, their susceptibility to nuclease digestion in plasma and, second, their limited ability to gain access to the intracellular compartment where they can bind the RISC (RNA-induced Silencing Complex) when administered systemically as the free siRNA or miRNA. Certain delivery systems, such as lipid nanoparticles formed from cationic lipids with other lipid components, such as cholesterol and PEG lipids, carbohydrates (such as GalNAc trimers) have been used to facilitate the cellular uptake of the oligonucleotides. However, these have not been shown to be successful in efficiently and effectively delivering siRNA to its intended target in tissues other than the liver.

CD40 and its ligand CD40L (or CD154) are transmembrane proteins expressed by immune cells, including B cells, T cells, and dendritic cells. CD40 functions to amplify the immune response by stimulating T cells and activation and maturation of B cells. In autoimmune diseases, the role of CD40 in the activation of the immune system also includes the production of autoimmune antibodies. For example, studies have shown a role for CD40 in rheumatoid arthritis, autoimmune thyroid disease, type I diabetes, neuroinflammatory diseases such as multiple sclerosis, psoriasis, inflammatory bowel disease, systemic lupus erythematosus, and lupus nephritis (see, e.g., Zheng et al., Arthritis Res. & Therapy, 2010, 12:R13; Peters et al., Semin Immunol., 2009, 21(5):293-300; and Ripoll et al., PLoS One, 2013, 8(6):e65068).

What is needed are compositions and methods for delivering therapeutic nucleic acids, such as small interfering RNA (siRNA), to intended cellular targets to downregulate the production and expression of CD40 in subjects suffering from autoimmune diseases. Further, what is needed is a FN3 domain with optimized properties for clinical use that can specifically bind to CD71, and methods of using such molecules for novel therapeutics that enable intracellular access via receptor-mediated internalization of CD71. The present embodiments fulfills these needs as well as others.

### SUMMARY

Provided herein are compositions comprising siRNA molecules comprising a sense strand and an antisense strand, such as those provided herein. In some embodiments, the siRNA molecule targets the CD40 gene. In some embodiments, the siRNA further comprises a linker covalently attached to the sense strand or the antisense strand of the siRNA. In some embodiments, the linker is attached to a 5′ end or a 3′ end of the sense strand or the antisense strand. In some embodiments, the siRNA molecule further comprises a vinyl phosphonate modification on the sense strand or on the antisense strand. In some embodiments, the vinyl phosphonate modification is on a 5′ end or a 3′ end of the sense strand or the antisense strand. In some embodiments, the sense strand comprises a nucleic acid sequence selected from the group consisting of SEQ ID NOs: 1890, 1893, 1941, 1942, 1944, 46-178, 312-331, 1850, 1851, 1891, 1892, 1894-1928, 1932-1940, 1943, 1945-1959, 2298, 2302, 2304, 352-356, 673-805, 939-958, 2070, 2071, 2110-2148, 2152-2179, 2300, 2306, and 2308. In some embodiments, the antisense strand comprises a nucleic acid sequence selected from the group consisting of SEQ ID NOs: 2290, 2293, 2051, 2052, 2054, 179-311, 332-351, 1960, 1961, 2000-2038, 2042-2050, 2053, 2055-2069, 2291, 2292, 2294-2297, 2299, 2303, 2305, 356-359, 806-938, 959-978, 2180, 2181, 2220-2258, 2262-2289, 2301, 2307, and 2309. In some embodiments, the siRNA molecule comprises an siRNA pair as set forth in Table 3A, Table 3B, Table 4A, Table 4B, Table 5A, or Table 5B.

In some embodiments, the composition further comprises one or more FN3 domains conjugated to the siRNA molecule. In some embodiments, the one or more FN3 domains comprises an FN3 domain that binds to CD71. In some embodiments, the FN3 domain comprises an amino acid sequence that is at least 87%%, 88%, 89%, 90%, 91%, 92%, 93%, 94%, 95%, 96%, 97%, 98%, 99% identical to, or is identical to, a sequence selected from any one of SEQ ID NOs: 570, 672, 1848, 1773, 1849, 1767, 360-569, 571-644, 663-67, 1395-1772, 1774-1766, and 1768-1847.

In some embodiments, the one or more FN3 domains comprises at least two FN3 domains linked by a peptide linker. In some embodiments, the linker comprises an amino acid sequence selected from the group consisting of SEQ ID NOs: 645-661.

Also provided herein are compositions having a formula of:

(X₁)ₙ—(X₂)_{q}—(X₃)_{y}-L-X₄;

C—(X₁)ₙ—(X₂)_{q}-L-X₄—(X₃)_{y};

(X₁)ₙ—(X₂)_{q}-L-X₄—(X₃)_{y}—C;

C—(X₁)ₙ—(X₂)_{q}-L-X₄-L-(X₃)_{y};

or (X₁)ₙ—(X₂)_{q}-L-X₄-L-(X₃)_{y}—C,

wherein: X₁ is a first FN3 domain; X₂ is a second FN3 domain; X₃ is a third FN3 domain or half-life extender molecule; L is a linker; and X₄ is a nucleic acid molecule, such as an siRNA that targets CD40, such as those provided herein. C is a polymer, such as PEG, an albumin binding protein, or an aliphatic chain that bind to serum proteins, wherein n, q, and y are each independently 0 or 1. In some embodiments, X₁, X₂, and X₃ bind to the same or different target proteins.

Also provided herein are compositions having a formula A₁-B₁, wherein A₁ has a formula of (C)ₙ-(L₁)ₜ-X_{S} and B₁ has a formula of X_{AS}-(L₂)_{q}-(F₁)_{y} or A₁ has a formula of (F₁)ₙ-(L₁)ₜ-X_{S} and B₁ has a formula of X_{AS}-(L₂)_{q}-(C)_{y}, wherein: C is a polymer, such as PEG, albumin binding protein, or an aliphatic chain that binds to serum proteins;
L₁ and L₂ are each, independently, a linker;
X_{S} is a 5′ to 3′ oligonucleotide sense strand of a double stranded siRNA molecule;
X_{AS} is a 3′ to 5′ oligonucleotide antisense strand of a double stranded siRNA molecule;
F₁ is a polypeptide comprising at least one FN3 domain;
wherein n, t, q, and y are each independently 0 or 1;
wherein X_{S} and X_{AS} form a double stranded oligonucleotide molecule to form the composition/complex that targets CD40.

In some embodiments, a method of treating an immunological disease in a subject in need thereof is provided herein, the method comprising administering to the subject a composition, such as any composition provided herein. In some embodiments, a method of reducing mRNA expression of a target gene in a cell, such as an immune cell, is provided herein, the method comprising contacting the immune cell with a composition of any composition as provided herein. In some embodiments, a method of delivering an siRNA molecule to a cell, such as an immune cell, in a subject is provided herein, the method comprising administering to the subject a pharmaceutical composition comprising any composition provided herein. In some embodiments, a method of delivering an siRNA molecule targeting CD40 to a CD71 expressing immune cell in a subject is provided herein, the method comprising administering to the subject a pharmaceutical composition comprising any composition provided herein, wherein the siRNA molecule downregulates mRNA expression of CD40 in the CD71 expressing immune cell.

Further provided herein is a method of reducing one or more serum cytokines, the method comprising administering a CD40 targeting siRNA molecule to one or more CD71 expressing immune cells. In some embodiments, the one or more serum cytokines comprises IFN-γ, IL-6, TNF-α, IL-12, IP-10, RANTES, or any combination thereof. In some embodiments, the one or more CD71 expressing immune cells comprises a B cell, a T cell, or a combination thereof.

Further provided herein is a method of selectively reducing a population of CD71 expressing immune cells, the method comprising administering a CD40 targeting siRNA molecule to a population of CD71 expressing immune cells. In some embodiments, the population of CD71 expressing immune cells comprises a B cell, a T cell, or a combination thereof.

### DESCRIPTION OF THE DRAWINGS

FIG. 1 depicts a flow chart of steps and assessed properties for in silico screening of CD40 siRNA.

FIG. 2 depicts titration curves for exemplary CD40 siRNAs in Raji cells (FIG. 2, Panel A) and A20 cells (FIG. 2, Panel B).

FIG. 3A depicts in vitro relative CD40 mRNA expression in donated human dendritic cells that have been activated and exposed to CD40 ligand, with or without treatment with an exemplary CD71 binding FN3 domain and CD40 targeting siRNA conjugate.

FIG. 3B depicts in vitro IL-12 production in donated human dendritic cells, activated or not activated, exposed to CD40 ligand or not exposed to CD40 ligand, and treated or not treated with an exemplary CD71 binding FN3 domain and CD40 targeting siRNA conjugate.

FIG. 4 depicts in vitro relative CD40 mRNA expression in donated human dendritic cells activated and treated with increasing concentrations of exemplary CD71 binding FN3 domain and CD40 targeting siRNA conjugates. CD40 mRNA expression in treated cells is normalized to mRNA expression in activated, untreated dendritic cells. As concentration (in nM) of conjugate increases, relative CD40 mRNA expression decreases across all donors.

FIG. 5 depicts in vitro relative CD40 protein expression over time in donated human dendritic cells activated and treated with exemplary CD71 binding FN3 domain and CD40 targeting siRNA conjugates, or activated and not treated (“Activation Only”). CD40 protein expression in treated cells is normalized to protein expression in activated, untreated dendritic cells.

FIG. 6 depicts in vitro cytokine production in donated dendritic cells activated and treated with exemplary CD71 binding FN3 domain and CD40 targeting siRNA conjugates, in dendritic cells activated and treated with negative control, and in dendritic cells activated and not treated.

FIG. 7 depicts in vivo serum cytokine levels in mice activated and treated with exemplary CD71 binding FN3 domain and CD40 targeting siRNA conjugates, in mice activated and treated with negative control, in mice activated and treated only with CD71 binding FN3 domain, in mice activated and treated with vehicle, and in naïve mice neither activated nor treated.

FIG. 8 depicts in vivo serum cytokine levels in mice induced with EAE, an animal disease model, and activated and treated with exemplary CD71 binding FN3 domain and CD40 targeting siRNA conjugates. Further depicted are EAE mice activated and treated with negative control, EAE mice activated and treated only with CD71 binding FN3 domain, EAE mice activated and treated with vehicle, and healthy, naïve mice neither activated nor treated.

FIG. 9 depicts in vivo frequency of B cells in draining lymph node tissue and spinal cord tissue collected from mice induced with EAE, an animal disease model, and activated and treated with exemplary CD71 binding FN3 domain and CD40 targeting siRNA conjugates. Further depicted are EAE mice activated and treated with positive control, EAE mice activated and treated with vehicle, and healthy, naïve mice neither activated nor treated.

FIG. 10 depicts in vivo frequency of dendritic cells, CD8 T cells, and CD4 T cells in spinal cord tissue collected from mice induced with EAE, an animal disease model, and activated and treated with exemplary CD71 binding FN3 domain and CD40 targeting siRNA conjugates. Further depicted are EAE mice activated and treated with positive control and EAE mice activated and treated with vehicle.

FIG. 11 depicts in vivo frequency of lymphocytes, monocytes, and macrophages in spinal cord tissue collected from mice induced with EAE, an animal disease model, and activated and treated with exemplary CD71 binding FN3 domain and CD40 targeting siRNA conjugates. Further depicted are EAE mice activated and treated with positive control and EAE mice activated and treated with vehicle.

### DETAILED DESCRIPTION

As used in this specification and the appended claims, the singular forms “a,” “an,” and “the” include plural referents unless the content clearly dictates otherwise. Thus, for example, reference to “a cell” includes a combination of two or more cells, and the like.

“Fibronectin type III domain” or “FN3 domain” refers to a polypeptide sequence occurring frequently in proteins including fibronectins, tenascin, intracellular cytoskeletal proteins, cytokine receptors and prokaryotic enzymes (Bork and Doolittle, Proc Nat Acad Sci USA 89:8990-8994, 1992; Meinke et al., J Bacteriol 175:1910-1918, 1993; Watanabe et al., J Biol Chem 265:15659-15665, 1990). Exemplary FN3 domains are the 15 different FN3 domains present in human tenascin C, the 15 different FN3 domains present in human fibronectin (FN), and non-natural synthetic FN3 domains as described for example in U.S. Pat. No. 8,278,419. Individual FN3 domains are referred to by domain number and protein name, e.g., the 3^{rd} FN3 domain of tenascin (TN3), or the 10^{th} FN3 domain of fibronectin (FN10). As used throughout, “Centyrin” also refers to an FN3 domain. Further, FN3 domains as described herein are not antibodies as they do not have the structure of a variable heavy (V_{H}) and/or light (V_{L}) chain.

“Autoimmune disease” refers to disease conditions and states wherein the immune response of an individual is directed against the individual's own constituents, resulting in an undesirable and often debilitating condition. As used herein, “autoimmune disease” is intended to further include autoimmune conditions, syndromes, and the like. Autoimmune diseases include, but are not limited to, Addison's disease, allergy, allergic rhinitis, ankylosing spondylitis, asthma, atherosclerosis, autoimmune diseases of the ear, autoimmune diseases of the eye, autoimmune atrophic gastritis, autoimmune hepatitis, autoimmune hemolytic anemia, autoimmune parotitis, autoimmune uveitis, celiac disease, primary biliary cirrhosis, benign lymphocytic angiitis, COPD, colitis, coronary heart disease, Crohn's disease, diabetes (Type I), depression, diabetes, including Type 1 and/or Type 2 diabetes, epididymitis, glomerulonephritis, Goodpasture's syndrome, Graves' disease, Guillain-Barre syndrome, Hashimoto's disease, hemolytic anemia, idiopathic thrombocytopenic purpura, inflammatory bowel disease (IBD), immune response to recombinant drug products, e.g., factor VII in hemophilia, juvenile idiopathic arthritis, systemic lupus erythematosus, lupus nephritis, male infertility, mixed connective tissue disease, multiple sclerosis, myasthenia gravis, oncology, osteoarthritis, pain, primary myxedema, pemphigus, pernicious anemia, polymyositis, psoriasis, psoriatic arthritis, reactive arthritis, rheumatic fever, rheumatoid arthritis, sarcoidosis, scleroderma, Sjögren's syndrome, spondyloarthropathies, sympathetic ophthalmia, T-cell lymphoma, T-cell acute lymphoblastic leukemia, testicular angiocentric T-cell lymphoma, thyroiditis, transplant rejection, ulcerative colitis, autoimmune uveitis, and vasculitis. Autoimmune diseases include, but are not limited to, conditions in which the tissue affected is the primary target, and in some cases, the secondary target. Such conditions include, but are not limited to, AIDS, atopic allergy, bronchial asthma, eczema, leprosy, schizophrenia, inherited depression, transplantation of tissues and organs, chronic fatigue syndrome, Alzheimer's disease, Parkinson's disease, myocardial infarction, stroke, autism, epilepsy, Arthus' phenomenon, anaphylaxis, and alcohol and drug addiction.

“Capture agent” refers to substances that bind to a particular type of cell and enable the isolation of that cell from other cells. Exemplary capture agents are magnetic beads, ferrofluids, encapsulating reagents, molecules that bind the particular cell type, and the like.

“Sample” refers to a collection of similar fluids, cells, or tissues isolated from a subject, as well as fluids, cells, or tissues present within a subject. Exemplary samples are tissue biopsies, fine needle aspirations, surgically resected tissue, organ cultures, cell cultures and biological fluids such as blood, serum and serosal fluids, plasma, lymph, urine, saliva, cystic fluid, tear drops, feces, sputum, mucosal secretions of the secretory tissues and organs, vaginal secretions, ascites fluids, fluids of the pleural, pericardial, peritoneal, abdominal and other body cavities, fluids collected by bronchial lavage, synovial fluid, liquid solutions contacted with a subject or biological source, for example, cell and organ culture medium including cell or organ conditioned medium and lavage fluids, and the like.

“Substituting,” “substituted,” “mutating,” or “mutated” refers to altering, deleting or inserting one or more amino acids or nucleotides in a polypeptide or polynucleotide sequence to generate a variant of that sequence.

“Variant” refers to a polypeptide or a polynucleotide that differs from a reference polypeptide or a reference polynucleotide by one or more modifications, for example, substitutions, insertions, or deletions.

“Specifically binds” or “specific binding” refers to the ability of an FN3 domain to bind to its target, such as CD71, with a dissociation constant (K_{D}) of about 1×10⁻⁶ M or less, for example, about 1×10⁻⁷ M or less, about 1×10⁻⁸ M or less, about 1×10⁻⁹ M or less, about 1×10⁻¹⁰ M or less, about 1×10⁻¹¹ M or less, about 1×10⁻¹² M or less, or about 1×10⁻¹³ M or less. Alternatively, “specific binding” refers to the ability of an FN3 domain to bind to its target (e.g., CD71) at least 5-fold above a negative control in standard solution ELISA assay.

Specific binding can also be demonstrated using the proteome array as described herein. In some embodiments, a negative control is an FN3 domain that does not bind CD71. In some embodiments, an FN3 domain that specifically binds CD71 may have cross-reactivity to other related antigens, for example, to the same predetermined antigen from other species (homologs), such as Macaca Fascicularis (cynomolgus monkey, cyno) or Pan troglodytes (chimpanzee).

“Library” refers to a collection of variants. The library may be composed of polypeptide or polynucleotide variants.

“Stability” refers to the ability of a molecule to maintain a folded state under physiological conditions such that it retains at least one of its normal functional activities, for example, binding to a predetermined antigen such as CD71.

“CD71” refers to human CD71 protein having the amino acid sequence of SEQ ID NOs: 3 or 4. In some embodiments, SEQ ID NO: 3 is full length human CD71 protein. In some embodiments, SEQ ID NO: 4 is the extracellular domain of human CD71.

“Tencon” refers to the synthetic fibronectin type III (FN3) domain having the consensus sequence:

(SEQ ID NO: 1)
LPAPKNLVVSEVTEDSLRLSWTAPDAAFDSFLIQYQESEKVGEAINLTV
PGSERSYDLTGLKPGTEYTVSIYGVKGGHRSNPLSAEFTT

and described in U.S. Pat. Pub. No. 2010/0216708.

“Immune cell” refers to cells of the immune system categorized as lymphocytes (T-cells, B-cells and NK cells), neutrophils, or monocytes/macrophages. Immune cells also include dendritic cells. A “dendritic cell” refers to a type of antigen-presenting cell (APC) that forms an important role in the adaptive immune system. The main function of dendritic cells is to present antigens to T lymphocytes, and to secrete cytokines that may further modulate the immune response directly or indirectly. Dendritic cells have the capacity to induce a primary immune response in inactive or resting naïve T lymphocytes.

“Vector” refers to a polynucleotide capable of being duplicated within a biological system or that can be moved between such systems. Vector polynucleotides typically contain elements, such as origins of replication, polyadenylation signal or selection markers that function to facilitate the duplication or maintenance of these polynucleotides in a biological system. Examples of such biological systems may include a cell, virus, animal, plant, and reconstituted biological systems utilizing biological components capable of duplicating a vector. The polynucleotide comprising a vector may be DNA or RNA molecules, or a hybrid of these.

“Expression vector” refers to a vector that can be utilized in a biological system or in a reconstituted biological system to direct the translation of a polypeptide encoded by a polynucleotide sequence present in the expression vector.

“Polynucleotide” refers to a synthetic molecule comprising a chain of nucleotides covalently linked by a sugar-phosphate backbone or other equivalent covalent chemistry. cDNA is a typical example of a polynucleotide.

“Polypeptide” or “protein” refers to a molecule that comprises at least two amino acid residues linked by a peptide bond to form a polypeptide. Small polypeptides of less than about 50 amino acids may be referred to as “peptides”.

“Valent” refers to the presence of a specified number of binding sites specific for an antigen in a molecule. As such, the terms “monovalent”, “bivalent”, “tetravalent”, and “hexavalent” refer to the presence of one, two, four and six binding sites, respectively, specific for an antigen in a molecule.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. Except when noted, the terms “patient” or “subject” are used interchangeably.

“Isolated” refers to a homogenous population of molecules (such as synthetic polynucleotides or polypeptides such as FN3 domains) which have been substantially separated and/or purified away from other components of the system the molecules are produced in, such as a recombinant cell, as well as a protein that has been subjected to at least one purification or isolation step. “Isolated FN3 domain” refers to an FN3 domain that is substantially free of other cellular material and/or chemicals and encompasses FN3 domains that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Migration,” when used in reference to the movement of cells, means that the cells are moving from one location to another. For example, a cell (e.g., a white blood cell or an immune cell) moving from a blood vessel to a tissue can be said to be migrating from the blood vessel to the tissue. Migration can also include the process of “margination,” which refers to a cell moving from the interior of a blood vessel towards the blood vessel wall. Migration can also include adhesion of the cell to the blood vessel wall, as well as transmigration across the blood vessel wall to enter a tissue.

### Compositions

In some embodiments, a composition comprising a polypeptide, such as a polypeptide comprising an FN3 domain, linked to an oligonucleotide molecule are provided. The oligonucleotide molecule can be, for example, an siRNA molecule. In some embodiments, the FN3 domain is a CD71-binding FN3 domain as provided for herein. In some embodiments, the oligonucleotide is a CD40 siRNA that binds to a CD40 RNA, such as mRNA as provided for herein. In some embodiments, the composition further comprises a polymer as provided for herein.

In some embodiments, the siRNA molecule is a double-stranded RNAi (dsRNA) agent capable of inhibiting the expression of a target gene. The dsRNA agent comprises a sense strand (passenger strand) and an antisense strand (guide strand). In some embodiments, each strand of the dsRNA agent can range from 12-40 nucleotides in length. For example, each strand can be from 14-40 nucleotides in length, 17-37 nucleotides in length, 25-37 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length.

In some embodiments, the sense strand and antisense strand typically form a duplex dsRNA. The duplex region of a dsRNA agent may be from 12-40 nucleotide pairs in length. For example, the duplex region can be from 14-40 nucleotide pairs in length, 17-30 nucleotide pairs in length, 25-35 nucleotides in length, 27-35 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotide pairs in length.

In some embodiments, the dsRNA comprises one or more overhang regions and/or capping groups of dsRNA agent at the 3′ end, or 5′ end, or both ends of a strand. The overhang can be 1-10 nucleotides in length, 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. The overhangs can be the result of one strand being longer than the other, or the result of two strands of the same length being staggered. The overhang can form a mismatch with the target mRNA, or it can be complementary to the gene sequences being targeted, or it can be another sequence. The first and second strands can also be joined, e.g., by additional bases to form a hairpin, or by other non-base linkers.

In some embodiments, the nucleotides in the overhang region of the dsRNA agent can each independently be a modified or unmodified nucleotide including, but not limited to, 2′-sugar modified, such as 2-F, 2′-O-methyl, 2′-O-(2-methoxyethyl), 2′-O-(2-methoxyethyl), 2′-O-(2-methoxyethyl), and any combinations thereof. For example, TT (UU) can be an overhang sequence for either end on either strand. The overhang can form a mismatch with the target mRNA, or it can be complementary to the gene sequences being targeted, or it can be another sequence.

The 5′- or 3′-overhangs at the sense strand, antisense strand, or both strands of the dsRNA agent may be phosphorylated. In some embodiments, the overhang region contains two nucleotides having a phosphorothioate, phosphorodithioate, phosphonate, phosphoramidate, or mesyl phosphoramidate between the two nucleotides, where the two nucleotides can be the same or different. In one embodiment, the overhang is present at the 3′-end of the sense strand, antisense strand, or both strands. In one embodiment, this 3′-overhang is present in the antisense strand. In one embodiment, this 3′-overhang is present in the sense strand.

The dsRNA agent may comprise only a single overhang, which can strengthen the interference activity of the dsRNA, without affecting its overall stability. For example, the single-stranded overhang is located at the 3′-terminal end of the sense strand or, alternatively, at the 3′-terminal end of the antisense strand. The dsRNA may also have a blunt end, located at the 5′ end of the antisense strand (or the 3′ end of the sense strand) or vice versa. Generally, the antisense strand of the dsRNA has a nucleotide overhang at the 3′ end, and the 5′ end is blunt. While not bound by theory, the asymmetric blunt end at the 5′ end of the antisense strand and 3′ end overhang of the antisense strand favor the guide strand loading into the RNA induced silencing complex (RISC). For example, the single overhang comprises at least two, three, four, five, six, seven, eight, nine, or ten nucleotides in length.

In some embodiments, the dsRNA agent may also have two blunt ends, at both ends of the dsRNA duplex.

In some embodiments, every nucleotide in the sense strand and antisense strand of the dsRNA agent may be modified. Each nucleotide may be modified with the same or different modification, which can include one or more alterations of one or both of the non-linking phosphate oxygens and/or of one or more of the linking phosphate oxygens; alteration of a constituent of the ribose sugar, e.g., of the 2 hydroxyl on the ribose sugar; wholesale replacement of the phosphate moiety with “dephospho” linkers; modification or replacement of a naturally occurring base; and replacement or modification of the ribose-phosphate backbone.

In some embodiments, all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl (2′-OMe) modified instead of the ribosugar of the nucleobase, and modifications in the phosphate group, e.g., phosphorothioate, phosphorodithoate, phosphonate, phosphoramidate, or mesyl phosphoramidate modifications. Overhangs need not be homologous with the target sequence.

In some embodiments, each residue of the sense strand and antisense strand is independently modified with LNA, HNA, CeNA, 2′-methoxyethyl, 2′-O-methyl, 2′-O-allyl, 2′-C-allyl, 2′-deoxy, or 2′-fluoro. The strands can contain more than one modification. In one embodiment, each residue of the sense strand and antisense strand is independently modified with 2′-O-methyl or 2′-fluoro.

In some embodiments, at least two different modifications are typically present on the sense strand and antisense strand. Those two modifications may be the 2′-deoxy, 2′-O-methyl or 2′-fluoro modifications, acyclic nucleotides, or others.

In one embodiment, the sense strand and antisense strand each comprises two differently modified nucleotides selected from 2′-fluoro, 2′-O-methyl, or 2′-deoxy.

The dsRNA agent may further comprise at least one phosphorothioate, phosphorodithoate, phosphonate, phosphoramidate, mesyl phosphoramidate, or methylphosphonate internucleotide linkage. The phosphorothioate, phosphorodithoate, phosphonate, phosphoramidate, mesyl phosphoramidate, or methylphosphonate internucleotide linkage modification may occur on any nucleotide of the sense strand or antisense strand or both in any position of the strand. For instance, the internucleotide linkage modification may occur on every nucleotide on the sense strand and/or antisense strand; each internucleotide linkage modification may occur in an alternating pattern on the sense strand or antisense strand; or the sense strand or antisense strand comprises both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In some embodiments, the dsRNA agent comprises the phosphorothioate, phosphorodithoate, phosphonate, phosphoramidate, mesyl phosphoramidate, or methylphosphonate internucleotide linkage modification in the overhang region. For example, the overhang region comprises two nucleotides having a phosphorothioate, phosphorodithoate, phosphonate, phosphoramidate, mesyl phosphoramidate, or methylphosphonate internucleotide linkage between the two nucleotides. Internucleotide linkage modifications also may be made to link the overhang nucleotides with the terminal paired nucleotides within the duplex region. For example, at least 2, 3, 4, or all the overhang nucleotides may be linked through phosphorothioate, phosphorodithoate, phosphonate, phosphoramidate, mesyl phosphoramidate, or methylphosphonate internucleotide linkage, and optionally, there may be additional phosphorothioate, phosphorodithoate, phosphonate, phosphoramidate, mesyl phosphoramidate, or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. In some embodiments, these terminal three nucleotides may be at the 3′ end of the antisense strand.

In some embodiments, the dsRNA composition is linked by a modified base or nucleoside analogue as described in U.S. Pat. No. 7,427,672, which is incorporated herein by reference. In some embodiments, the modified base or nucleoside analogue is referred to as the linker or L in formulas described herein.

In some embodiments, the modified base or nucleoside analogue has the structure as shown in Chemical Formula I and a salt thereof:

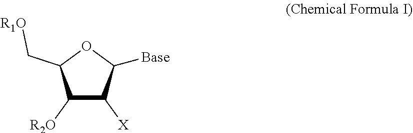

where Base represents an aromatic heterocyclic group or aromatic hydrocarbon ring group optionally having a substituent, R₁ and R₂ are identical or different, and each represent a hydrogen atom, a protective group for a hydroxyl group for nucleic acid synthesis, an alkyl group, an alkenyl group, a cycloalkyl group, an aryl group, an aralkyl group, an acyl group, a sulfonyl group, a silyl group, a phosphate group, a phosphate group protected with a protective group for nucleic acid synthesis, or —P(R₄)R₅ where R₄ and R₅ are identical or different, and each represent a hydroxyl group, a hydroxyl group protected with a protective group for nucleic acid synthesis, a mercapto group, a mercapto group protected with a protective group for nucleic acid synthesis, an amino group, an alkoxy group having 1 to 5 carbon atoms, an alkylthio group having 1 to 5 carbon atoms, a cyanoalkoxy group having 1 to 6 carbon atoms, or an amino group substituted by an alky group having 1 to 5 carbon atoms, and X denotes OMe or F.

In some embodiments, the modified base or nucleoside analogue has the structure as shown in Chemical Formula I and salts thereof, wherein R₁ is a hydrogen atom, an aliphatic acyl group, an aromatic acyl group, an aliphatic or aromatic sulfonyl group, a methyl group substituted by one to three aryl groups, a methyl group substituted by one to three aryl groups having an aryl ring substituted by a lower alkyl, lower alkoxy, halogen, or cyano group, or a silyl group.

In some embodiments, the modified base or nucleoside analogue has the structure as shown in Chemical Formula I and salts thereof, wherein R₁ is a hydrogen atom, an acetyl group, a benzoyl group, a methanesulfonyl group, a p-toluenesulfonyl group, a benzyl group, a p-methoxybenzyl group, a trityl group, a dimethoxytrityl group, a monomethoxytrityl group, or a tert-butyldiphenylsilyl group.

In some embodiments, the modified base or nucleoside analogue has the structure as shown in Chemical Formula I and salts thereof, wherein R₂ is a hydrogen atom, an aliphatic acyl group, an aromatic acyl group, an aliphatic or aromatic sulfonyl group, a methyl group substituted by one to three aryl groups, a methyl group substituted by one to three aryl groups having an aryl ring substituted by a lower alkyl, lower alkoxy, halogen, or cyano group, a silyl group, a phosphoroamidite group, a phosphonyl group, a phosphate group, or a phosphate group protected with a protective group for nucleic acid synthesis.

In some embodiments, the modified base or nucleoside analogue has the structure as shown in Chemical Formula I and salts thereof, wherein R₂ is a hydrogen atom, an acetyl group, a benzoyl group, a methanesulfonyl group, a p-toluenesulfonyl group, a benzyl group, a p-methoxybenzyl group, a tert-butyldiphenylsilyl group, —P(OC₂H₄CN)(N(i-Pr)₂), —P(OCH₃)(N(i-Pr)₂), a phosphonyl group, or a 2-chlorophenyl- or 4-chlorophenylphosphate group.

In some embodiments, the modified base or nucleoside analogue has the structure as shown in Chemical Formula I and salts thereof, wherein Base is a purin-9-yl group, a 2-oxopyrimidin-1-yl group, or a purin-9-yl group or a 2-oxopyrimidin-1-yl group having a substituent selected from the following a group: A hydroxyl group, a hydroxyl group protected with a protective group for nucleic acid synthesis, an alkoxy group having 1 to 5 carbon atoms, a mercapto group, a mercapto group protected with a protective group for nucleic acid synthesis, an alkylthio group having 1 to 5 carbon atoms, an amino group, an amino group protected with a protective group for nucleic acid synthesis, an amino group substituted by an alkyl group having 1 to 5 carbon atoms, an alkyl group having 1 to 5 carbon atoms, and a halogen atom.

In some embodiments, the modified base or nucleoside analogue has the structure as shown in Chemical Formula I and salts thereof, wherein Base is 6-aminopurin-9-yl (i.e., adeninyl), 6-aminopurin-9-yl having the amino group protected with a protective group for nucleic acid synthesis, 2,6-diaminopurin-9-yl, 2-amino-6-chloropurin-9-yl, 2-amino-6-chloropurin-9-yl having the amino group protected with a protective group for nucleic acid synthesis, 2-amino-6-fluoropurin-9-yl, 2-amino-6-fluoropurin-9-yl having the amino group protected with a protective group for nucleic acid synthesis, 2-amino-6-bromopurin-9-yl, 2-amino-6-bromopurin-9-yl having the amino group protected with a protective group for nucleic acid synthesis, 2-amino-6-hydroxypurin-9-yl (i.e., guaninyl), 2-amino-6-hydroxypurin-9-yl having the amino group protected with a protective group for nucleic acid synthesis, 6-amino-2-methoxypurin-9-yl, 6-amino-2-chloropurin-9-yl, 6-amino-2-fluoropurin-9-yl, 2,6-dimethoxypurin-9-yl, 2,6-dichloropurin-9-yl, 6-mercaptopurin-9-yl, 2-oxo-4-amino-1,2-dihydropyrimidin-1-yl (i.e., cytosinyl), 2-oxo-4-amino-1,2-dihydropyrimidin-1-yl having the amino group protected with a protective group for nucleic acid synthesis, 2-oxo-4-amino-5-fluoro-1,2-dihydropyrimidin-1-yl, 2-oxo-4-amino-5-fluoro-1,2-dihydropyrimidin-1-yl having the amino group protected with a protective group for nucleic acid synthesis, 4-amino-2-oxo-5-chloro-1,2-dihydropyrimidin-1-yl, 2-oxo-4-methoxy-1,2-dihydropyrimidin-1-yl, 2-oxo-4-mercapto-1,2-dihydropyrimidin-1-yl, 2-oxo-4-hydroxy-1,2-dihydropyrimidin-1-yl (i.e., uracinyl), 2-oxo-4-hydroxy-5-methyl-1,2-dihydropyrimidin-1-yl (i.e., thyminyl), 4-amino-5-methyl-2-oxo-1,2-dihydropyrimidin-1-yl (i.e., 5-methylcytosinyl), or 4-amino-5-methyl-2-oxo-1,2-dihydropyrimidin-1-yl having the amino group protected with a protective group for nucleic acid synthesis.

In some embodiments, the modified base or nucleoside analogue has the structure as shown in Chemical Formula IB and a salt thereof:

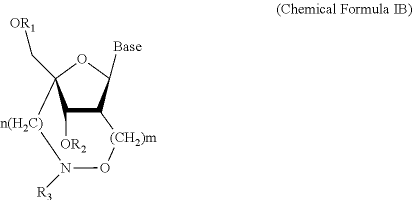

where Base represents an aromatic heterocyclic group or aromatic hydrocarbon ring group optionally having a substituent, R₁ and R₂ are identical or different, and each represent a hydrogen atom, a protective group for a hydroxyl group for nucleic acid synthesis, an alkyl group, an alkenyl group, a cycloalkyl group, an aryl group, an aralkyl group, an acyl group, a sulfonyl group, a silyl group, a phosphate group, a phosphate group protected with a protective group for nucleic acid synthesis, or —P(R₄)R₅ where R₄ and R₅ are identical or different, and each represent a hydroxyl group, a hydroxyl group protected with a protective group for nucleic acid synthesis, a mercapto group, a mercapto group protected with a protective group for nucleic acid synthesis, an amino group, an alkoxy group having 1 to 5 carbon atoms, an alkylthio group having 1 to 5 carbon atoms, a cyanoalkoxy group having 1 to 6 carbon atoms, or an amino group substituted by an alky group having 1 to 5 carbon atoms, R₃ represents a hydrogen atom, an alkyl group, an alkenyl group, a cycloalkyl group, an aryl group, an aralkyl group, an acyl group, a sulfonyl group, or a functional molecule unit substituent, and m denotes an integer of 0 to 2, and n denotes an integer of 0 to 3. In some embodiments, m and n are 0.

In some embodiments, the modified base or nucleoside analogue has the structure as shown in Chemical Formula IB and salts thereof, wherein R₁ is a hydrogen atom, an aliphatic acyl group, an aromatic acyl group, an aliphatic or aromatic sulfonyl group, a methyl group substituted by one to three aryl groups, a methyl group substituted by one to three aryl groups having an aryl ring substituted by a lower alkyl, lower alkoxy, halogen, or cyano group, or a silyl group.

In some embodiments, the modified base or nucleoside analogue has the structure as shown in Chemical Formula IB and salts thereof, wherein R₁ is a hydrogen atom, an acetyl group, a benzoyl group, a methanesulfonyl group, a p-toluenesulfonyl group, a benzyl group, a p-methoxybenzyl group, a trityl group, a dimethoxytrityl group, a monomethoxytrityl group, or a tert-butyldiphenylsilyl group.

In some embodiments, the modified base or nucleoside analogue has the structure as shown in Chemical Formula IB and salts thereof, wherein R₂ is a hydrogen atom, an aliphatic acyl group, an aromatic acyl group, an aliphatic or aromatic sulfonyl group, a methyl group substituted by one to three aryl groups, a methyl group substituted by one to three aryl groups having an aryl ring substituted by a lower alkyl, lower alkoxy, halogen, or cyano group, a silyl group, a phosphoroamidite group, a phosphonyl group, a phosphate group, or a phosphate group protected with a protective group for nucleic acid synthesis.

In some embodiments, the modified base or nucleoside analogue has the structure as shown in Chemical Formula IB and salts thereof, wherein R₂ is a hydrogen atom, an acetyl group, a benzoyl group, a methanesulfonyl group, a p-toluenesulfonyl group, a benzyl group, a p-methoxybenzyl group, a tert-butyldiphenylsilyl group, —P(OC₂H₄CN)(N(i-Pr)₂), —P(OCH₃)(N(i-Pr)₂), a phosphonyl group, or a 2-chlorophenyl- or 4-chlorophenylphosphate group.

In some embodiments, the modified base or nucleoside analogue has the structure as shown in Chemical Formula IB and salts thereof, wherein R₃ is a hydrogen atom, a phenoxyacetyl group, an alkyl group having 1 to 5 carbon atoms, an alkenyl group having 1 to 5 carbon atoms, an aryl group having 6 to 14 carbon atoms, a methyl group substituted by one to three aryl groups, a lower aliphatic or aromatic sulfonyl group such as a methanesulfonyl group or a p-toluenesulfonyl group, an aliphatic acyl group having 1 to 5 carbon atoms such as an acetyl group, or an aromatic acyl group such as a benzoyl group.

In some embodiments, the modified base or nucleoside analogue has the structure as shown in Chemical Formula IB and salts thereof, wherein the functional molecule unit substituent as R₃ is a fluorescent or chemiluminescent labeling molecule, a nucleic acid incision activity functional group, or an intracellular or nuclear transfer signal peptide.

In some embodiments, the modified base or nucleoside analogue has the structure as shown in Chemical Formula IB and salts thereof, wherein Base is a purin-9-yl group, a 2-oxopyrimidin-1-yl group, or a purin-9-yl group or a 2-oxopyrimidin-1-yl group having a substituent selected from the following a group: a hydroxyl group, a hydroxyl group protected with a protective group for nucleic acid synthesis, an alkoxy group having 1 to 5 carbon atoms, a mercapto group, a mercapto group protected with a protective group for nucleic acid synthesis, an alkylthio group having 1 to 5 carbon atoms, an amino group, an amino group protected with a protective group for nucleic acid synthesis, an amino group substituted by an alkyl group having 1 to 5 carbon atoms, an alkyl group having 1 to 5 carbon atoms, and a halogen atom.

In some embodiments, the modified base or nucleoside analogue has the structure as shown in Chemical Formula IB and salts thereof, wherein Base is 6-aminopurin-9-yl (i.e., adeninyl), 6-aminopurin-9-yl having the amino group protected with a protective group for nucleic acid synthesis, 2,6-diaminopurin-9-yl, 2-amino-6-chloropurin-9-yl, 2-amino-6-chloropurin-9-yl having the amino group protected with a protective group for nucleic acid synthesis, 2-amino-6-fluoropurin-9-yl, 2-amino-6-fluoropurin-9-yl having the amino group protected with a protective group for nucleic acid synthesis, 2-amino-6-bromopurin-9-yl, 2-amino-6-bromopurin-9-yl having the amino group protected with a protective group for nucleic acid synthesis, 2-amino-6-hydroxypurin-9-yl (i.e., guaninyl), 2-amino-6-hydroxypurin-9-yl having the amino group protected with a protective group for nucleic acid synthesis, 6-amino-2-methoxypurin-9-yl, 6-amino-2-chloropurin-9-yl, 6-amino-2-fluoropurin-9-yl, 2,6-dimethoxypurin-9-yl, 2,6-dichloropurin-9-yl, 6-mercaptopurin-9-yl, 2-oxo-4-amino-1,2-dihydropyrimidin-1-yl (i.e., cytosinyl), 2-oxo-4-amino-1,2-dihydropyrimidin-1-yl having the amino group protected with a protective group for nucleic acid synthesis, 2-oxo-4-amino-5-fluoro-1,2-dihydropyrimidin-1-yl, 2-oxo-4-amino-5-fluoro-1,2-dihydropyrimidin-1-yl having the amino group protected with a protective group for nucleic acid synthesis, 4-amino-2-oxo-5-chloro-1,2-dihydropyrimidin-1-yl, 2-oxo-4-methoxy-1,2-dihydropyrimidin-1-yl, 2-oxo-4-mercapto-1,2-dihydropyrimidin-1-yl, 2-oxo-4-hydroxy-1,2-dihydropyrimidin-1-yl (i.e., uracinyl), 2-oxo-4-hydroxy-5-methyl-1,2-dihydropyrimidin-1-yl (i.e., thyminyl), 4-amino-5-methyl-2-oxo-1,2-dihydropyrimidin-1-yl (i.e., 5-methylcytosinyl), or 4-amino-5-methyl-2-oxo-1,2-dihydropyrimidin-1-yl having the amino group protected with a protective group for nucleic acid synthesis.

In some embodiments, the modified base or nucleoside analogue has the structure as shown in Chemical Formula IB and salts thereof, wherein m is 0, and n is 1.

In some embodiments, the modified base or nucleoside analogue is a DNA oligonucleotide or RNA oligonucleotide analogue, containing one or two or more of one or more types of unit structures of nucleoside analogues having the structure as shown in Chemical Formula II, or a pharmacologically acceptable salt thereof, provided that a form of linking between respective nucleosides in the oligonucleotide analogue may contain one or two or more phosphorothioate bonds [—OP(O)(S—)O—], phosphorodithioate bonds [—O₂PS₂—], phosphonate bonds [—PO(OH)₂—], phosphoramidate bonds [—O═P(OH)₂—], or mesyl phosphoramidate bonds [—OP(O)(N)(SO₂)(CH₃)O—] aside from a phosphodiester bond [—OP(O₂—)O—] identical with that in a natural nucleic acid, and if two or more of one or more types of these structures are contained, Base may be identical or different between these structures:

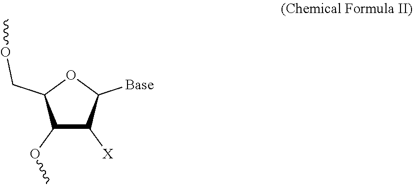

where Base represents an aromatic heterocyclic group or aromatic hydrocarbon ring group optionally having a substituent, and X denotes OMe or F.

In some embodiments, the oligonucleotide analogue or the pharmacologically acceptable salt thereof has the structure as shown in Chemical Formula II, wherein Base is a purin-9-yl group, a 2-oxopyrimidin-1-yl group, or a purin-9-yl group or a 2-oxopyrimidin-1-yl group having a substituent selected from the following a group: a hydroxyl group, a hydroxyl group protected with a protective group for nucleic acid synthesis, an alkoxy group having 1 to 5 carbon atoms, a mercapto group, a mercapto group protected with a protective group for nucleic acid synthesis, an alkylthio group having 1 to 5 carbon atoms, an amino group, an amino group protected with a protective group for nucleic acid synthesis, an amino group substituted by an alkyl group having 1 to 5 carbon atoms, an alkyl group having 1 to 5 carbon atoms, and a halogen atom.

In some embodiments, the oligonucleotide analogue or the pharmacologically acceptable salt thereof has the structure as shown in Chemical Formula II, wherein Base is 6-aminopurin-9-yl (i.e., adeninyl), 6-aminopurin-9-yl having the amino group protected with a protective group for nucleic acid synthesis, 2,6-diaminopurin-9-yl, 2-amino-6-chloropurin-9-yl, 2-amino-6-chloropurin-9-yl having the amino group protected with a protective group for nucleic acid synthesis, 2-amino-6-fluoropurin-9-yl, 2-amino-6-fluoropurin-9-yl having the amino group protected with a protective group for nucleic acid synthesis, 2-amino-6-bromopurin-9-yl, 2-amino-6-bromopurin-9-yl having the amino group protected with a protective group for nucleic acid synthesis, 2-amino-6-hydroxypurin-9-yl (i.e., guaninyl), 2-amino-6-hydroxypurin-9-yl having the amino group protected with a protective group for nucleic acid synthesis, 6-amino-2-methoxypurin-9-yl, 6-amino-2-chloropurin-9-yl, 6-amino-2-fluoropurin-9-yl, 2,6-dimethoxypurin-9-yl, 2,6-dichloropurin-9-yl, 6-mercaptopurin-9-yl, 2-oxo-4-amino-1,2-dihydropyrimidin-1-yl (i.e., cytosinyl), 2-oxo-4-amino-1,2-dihydropyrimidin-1-yl having the amino group protected with a protective group for nucleic acid synthesis, 2-oxo-4-amino-5-fluoro-1,2-dihydropyrimidin-1-yl, 2-oxo-4-amino-5-fluoro-1,2-dihydropyrimidin-1-yl group having the amino group protected with a protective group for nucleic acid synthesis, 4-amino-2-oxo-5-chloro-1,2-dihydropyrimidin-1-yl, 2-oxo-4-methoxy-1,2-dihydropyrimidin-1-yl, 2-oxo-4-mercapto-1,2-dihydropyrimidin-1-yl, 2-oxo-4-hydroxy-1,2-dihydropyrimidin-1-yl (i.e., uracinyl), 2-oxo-4-hydroxy-5-methyl-1,2-dihydropyrimidin-1-yl (i.e., thyminyl), 4-amino-5-methyl-2-oxo-1,2-dihydropyrimidin-1-yl (i.e., 5-methylcytosinyl), or 4-amino-5-methyl-2-oxo-1,2-dihydropyrimidin-1-yl having the amino group protected with a protective group for nucleic acid synthesis.

In some embodiments, the modified base or nucleoside analogue is a DNA oligonucleotide or RNA oligonucleotide analogue, containing one or two or more of one or more types of unit structures of nucleoside analogues having the structure as shown in Chemical Formula IIB, or a pharmacologically acceptable salt thereof, provided that a form of linking between respective nucleosides in the oligonucleotide analogue may contain one or two or more phosphorothioate bonds [—OP(O)(S—)O—], phosphorodithioate bonds [—O₂PS₂—], phosphonate bonds [—PO(OH)₂—], phosphoramidate bonds [—O═P(OH)₂—], or mesyl phosphoramidate bonds [—OP(O)(N)(SO₂)(CH₃)O—] aside from a phosphodiester bond [—OP(O₂—)O—] identical with that in a natural nucleic acid, and if two or more of one or more types of these structures are contained, Base may be identical or different between these structures:

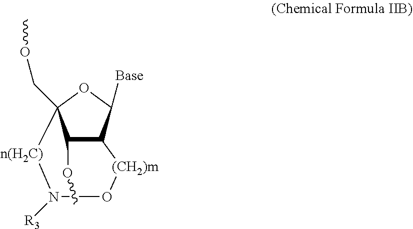

where Base represents an aromatic heterocyclic group or aromatic hydrocarbon ring group optionally having a substituent, R₃ represents a hydrogen atom, an alkyl group, an alkenyl group, a cycloalkyl group, an aryl group, an aralkyl group, an acyl group, a sulfonyl group, a silyl group, or a functional molecule unit substituent, and m denotes an integer of 0 to 2, and n denotes an integer of 0 to 3. In some embodiments, m and n are 0.

In some embodiments, the oligonucleotide analogue or the pharmacologically acceptable salt thereof has the structure as shown in Chemical Formula IIB, wherein R₁ is a hydrogen atom, an aliphatic acyl group, an aromatic acyl group, an aliphatic or aromatic sulfonyl group, a methyl group substituted by one to three aryl groups, a methyl group substituted by one to three aryl groups having an aryl ring substituted by a lower alkyl, lower alkoxy, halogen, or cyano group, or a silyl group.

In some embodiments, the oligonucleotide analogue or the pharmacologically acceptable salt thereof has the structure as shown in Chemical Formula IIB, wherein R₁ is a hydrogen atom, an acetyl group, a benzoyl group, a methanesulfonyl group, a p-toluenesulfonyl group, a benzyl group, a p-methoxybenzyl group, a trityl group, a dimethoxytrityl group, a monomethoxytrityl group, or a tert-butyldiphenylsilyl group.

In some embodiments, the oligonucleotide analogue or the pharmacologically acceptable salt thereof has the structure as shown in Chemical Formula IIB, wherein R₂ is a hydrogen atom, an aliphatic acyl group, an aromatic acyl group, an aliphatic or aromatic sulfonyl group, a methyl group substituted by one to three aryl groups, a methyl group substituted by one to three aryl groups having an aryl ring substituted by a lower alkyl, lower alkoxy, halogen, or cyano group, a silyl group, a phosphoroamidite group, a phosphonyl group, a phosphate group, or a phosphate group protected with a protective group for nucleic acid synthesis.

In some embodiments, the oligonucleotide analogue or the pharmacologically acceptable salt thereof has the structure as shown in Chemical Formula IIB, wherein R₂ is a hydrogen atom, an acetyl group, a benzoyl group, a benzyl group, a p-methoxybenzyl group, a methanesulfonyl group, a p-toluenesulfonyl group, a tert-butyldiphenylsilyl group, —P(OC₂H₄CN)(N(i-Pr)₂), —P(OCH₃)(N(i-Pr)₂), a phosphonyl group, or a 2-chlorophenyl- or 4-chlorophenylphosphate group.

In some embodiments, the oligonucleotide analogue or the pharmacologically acceptable salt thereof has the structure as shown in Chemical Formula IIB, wherein R₃ is a hydrogen atom, a phenoxyacetyl group, an alkyl group having 1 to 5 carbon atoms, an alkenyl group having 1 to 5 carbon atoms, an aryl group having 6 to 14 carbon atoms, a methyl group substituted by one to three aryl groups, a lower aliphatic or aromatic sulfonyl group such as a methanesulfonyl group or a p-toluenesulfonyl group, an aliphatic acyl group having 1 to 5 carbon atoms such as an acetyl group, or an aromatic acyl group such as a benzoyl group.

In some embodiments, the oligonucleotide analogue or the pharmacologically acceptable salt thereof has the structure as shown in Chemical Formula IIB, wherein the functional molecule unit substituent as R₃ is a fluorescent or chemiluminescent labeling molecule, a nucleic acid incision activity functional group, or an intracellular or nuclear transfer signal peptide.

In some embodiments, the oligonucleotide analogue or the pharmacologically acceptable salt thereof has the structure as shown in Chemical Formula IIB, wherein Base is a purin-9-yl group, a 2-oxopyrimidin-1-yl group, or a purin-9-yl group or a 2-oxopyrimidin-1-yl group having a substituent selected from the following a group: a group: A hydroxyl group, a hydroxyl group protected with a protective group for nucleic acid synthesis, an alkoxy group having 1 to 5 carbon atoms, a mercapto group, a mercapto group protected with a protective group for nucleic acid synthesis, an alkylthio group having 1 to 5 carbon atoms, an amino group, an amino group protected with a protective group for nucleic acid synthesis, an amino group substituted by an alkyl group having 1 to 5 carbon atoms, an alkyl group having 1 to 5 carbon atoms, and a halogen atom.

In some embodiments, the oligonucleotide analogue or the pharmacologically acceptable salt thereof has the structure as shown in Chemical Formula IIB, wherein Base is 6-aminopurin-9-yl (i.e., adeninyl), 6-aminopurin-9-yl having the amino group protected with a protective group for nucleic acid synthesis, 2,6-diaminopurin-9-yl, 2-amino-6-chloropurin-9-yl, 2-amino-6-chloropurin-9-yl having the amino group protected with a protective group for nucleic acid synthesis, 2-amino-6-fluoropurin-9-yl, 2-amino-6-fluoropurin-9-yl having the amino group protected with a protective group for nucleic acid synthesis, 2-amino-6-bromopurin-9-yl, 2-amino-6-bromopurin-9-yl having the amino group protected with a protective group for nucleic acid synthesis, 2-amino-6-hydroxypurin-9-yl (i.e., guaninyl), 2-amino-6-hydroxypurin-9-yl having the amino group protected with a protective group for nucleic acid synthesis, 6-amino-2-methoxypurin-9-yl, 6-amino-2-chloropurin-9-yl, 6-amino-2-fluoropurin-9-yl, 2,6-dimethoxypurin-9-yl, 2,6-dichloropurin-9-yl, 6-mercaptopurin-9-yl, 2-oxo-4-amino-1,2-dihydropyrimidin-1-yl (i.e., cytosinyl), 2-oxo-4-amino-1,2-dihydropyrimidin-1-yl having the amino group protected with a protective group for nucleic acid synthesis, 2-oxo-4-amino-5-fluoro-1,2-dihydropyrimidin-1-yl, 2-oxo-4-amino-5-fluoro-1,2-dihydropyrimidin-1-yl group having the amino group protected with a protective group for nucleic acid synthesis, 4-amino-2-oxo-5-chloro-1,2-dihydropyrimidin-1-yl, 2-oxo-4-methoxy-1,2-dihydropyrimidin-1-yl, 2-oxo-4-mercapto-1,2-dihydropyrimidin-1-yl, 2-oxo-4-hydroxy-1,2-dihydropyrimidin-1-yl (i.e., uracinyl), 2-oxo-4-hydroxy-5-methyl-1,2-dihydropyrimidin-1-yl (i.e., thyminyl), 4-amino-5-methyl-2-oxo-1,2-dihydropyrimidin-1-yl (i.e., 5-methylcytosinyl), or 4-amino-5-methyl-2-oxo-1,2-dihydropyrimidin-1-yl having the amino group protected with a protective group for nucleic acid synthesis.

In some embodiments, the oligonucleotide analogue or the pharmacologically acceptable salt thereof has the structure as shown in Chemical Formula IIB, wherein m is 0, and nis 1.

In some embodiments, the dsRNA agent comprises mismatch(es) with the target, within the duplex, or combinations thereof. The mismatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In some embodiments, the dsRNA agent can comprise a phosphorus-containing group at the 5′-end of the sense strand or antisense strand. The 5′-end phosphorus-containing group can be 5′-end phosphate (5′-P), 5′-end phosphorothioate (5′-PS), 5′-end phosphorodithioate (5′-PS₂), 5′-end vinyl phosphonate (5′-VP), 5′-end methylphosphonate (MePhos), 5′-end mesyl phosphoramidate (5′MsPA), or 5′-deoxy-5′-C-malonyl. When the 5′-end phosphorus-containing group is 5′-end vinyl phosphonate (5′-VP), the 5′-VP can be either 5′-E-VP isomer, such as trans-vinyl phosphate or cis-vinyl phosphate, or mixtures thereof. Representative structures of these modifications can be found in, for example, U.S. Pat. No. 10,233,448, which is hereby incorporated by reference in its entirety.

In some embodiments, nucleotide analogues or synthetic nucleotide base comprise a nucleic acid with a modification at a 2′ hydroxyl group of the ribose moiety. In some instances, the modification includes an H, OR, R, halo, SH, SR, NH2, NHR, NR2, or CN, wherein R is an alkyl moiety. Exemplary alkyl moiety includes, but is not limited to, methyl, ethyl, n-propyl, iso-propyl, n-butyl, iso-butyl, tert-butyl, C₁-C₁₀ chain lengths both linear and branched. In some instances, the alkyl moiety further comprises a modification. In some instances, the modification comprises an azo group, a keto group, an aldehyde group, a carboxyl group, a nitro group, a nitroso, group, a nitrile group, a heterocycle (e.g., imidazole, hydrazine or hydroxylamino) group, an isocyanate or cyanate group, or a sulfur containing group (e.g., sulfoxide, sulfone, sulfide, and disulfide). In some instances, the alkyl moiety further comprises additional hetero atom such as O, S, N, Se and each of these hetero atoms can be further substituted with alky groups as described above. In some instances, the carbon of the heterocyclic group is substituted by a nitrogen, oxygen or sulfur. In some instances, the heterocyclic substitution includes but is not limited to, morpholino, imidazole, and pyrrolidino.

In some instances, the modification at the 2′ hydroxyl group is a 2′-O-methyl modification or a 2′-O-methoxyethyl (2′-O-MOE) modification. Exemplary chemical structures of a 2′-O-methyl modification of an adenosine molecule and 2′O-methoxyethyl modification of an uridine are illustrated below.

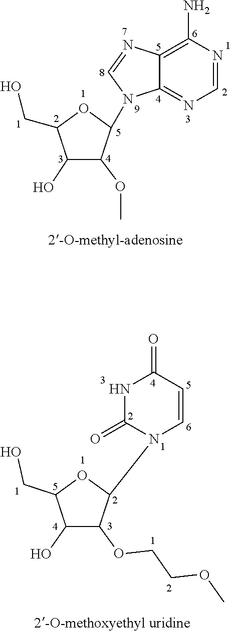 [SMILES: COC1C(O)C(CO)OC1n1cnc2c(N)ncnc21 ; COCCOC1C(O)C(CO)OC1n1ccc(=O)[nH]c1=O]

In some instances, the modification at the 2′ hydroxyl group is a 2′-O-aminopropyl modification in which an extended amine group comprising a propyl linker binds the amine group to the 2′ oxygen. In some instances, this modification neutralizes the phosphate derived overall negative charge of the oligonucleotide molecule by introducing one positive charge from the amine group per sugar and thereby improves cellular uptake properties due to its zwitterionic properties. An exemplary chemical structure of a 2′-O-aminopropyl nucleoside phosphoramidite is illustrated below.

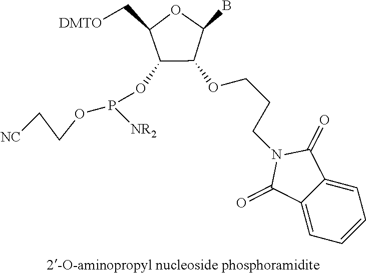

In some instances, the modification at the 2′ hydroxyl group is a locked or bridged ribose modification (e.g., locked nucleic acid or LNA) in which the oxygen molecule bound at the 2′ carbon is linked to the 4′ carbon by a methylene group, thus forming a 2′-C,4′-C-oxy-methylene-linked bicyclic ribonucleotide monomer. Exemplary representations of the chemical structure of LNA are illustrated below. The representation shown to the left highlights the chemical connectivities of an LNA monomer. The representation shown to the right highlights the locked 3′-endo (3E) conformation of the furanose ring of an LNA monomer.

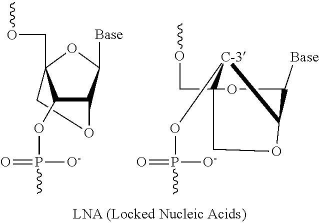

In some instances, the modification at the 2′ hydroxyl group comprises ethylene nucleic acids (ENA) such as for example 2′-4′-ethylene-bridged nucleic acid, which locks the sugar conformation into a C₃′-endo sugar puckering conformation. ENA are part of the bridged nucleic acids class of modified nucleic acids that also comprises LNA. Exemplary chemical structures of the ENA and bridged nucleic acids are illustrated below.

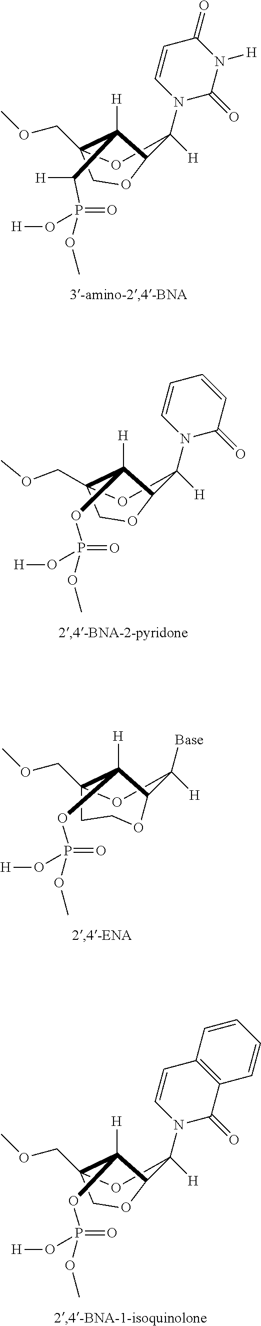

In some embodiments, additional modifications at the 2′ hydroxyl group include 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA).

In some embodiments, nucleotide analogues comprise modified bases such as, but not limited to, 5-propynyluridine, 5-propynylcytidine, 6-methyladenine, 6-methylguanine, N, N, dimethyladenine, 2-propyladenine, 2propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2, 4, 6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, in some cases are or be based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine.

In some embodiments, nucleotide analogues further comprise morpholinos, peptide nucleic acids (PNAs), methylphosphonate nucleotides, thiolphosphonate nucleotides, 2′-fluoro N3-P5′-phosphoramidites, 1′, 5′-anhydrohexitol nucleic acids (HNAs), or a combination thereof. Morpholino or phosphorodiamidate morpholino oligo (PMO) comprises synthetic molecules whose structure mimics natural nucleic acid structure by deviates from the normal sugar and phosphate structures. In some instances, the five-member ribose ring is substituted with a six member morpholino ring containing four carbons, one nitrogen and one oxygen. In some cases, the ribose monomers are linked by a phosphorodiamidate group instead of a phosphate group. In such cases, the backbone alterations remove all positive and negative charges making morpholinos neutral molecules capable of crossing cellular membranes without the aid of cellular delivery agents such as those used by charged oligonucleotides.

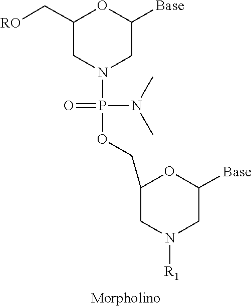

In some embodiments, peptide nucleic acid (PNA) does not contain sugar ring or phosphate linkage and the bases are attached and appropriately spaced by oligoglycine-like molecules, therefore, eliminating a backbone charge.

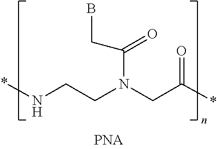

In some embodiments, one or more modifications optionally occur at the internucleotide linkage. In some instances, modified internucleotide linkage include, but is not limited to, phosphorothioates, mesyl phosphoramidate, phosphorodithioates, methylphosphonates, 5′-alkylenephosphonates, 5′-methylphosphonate, 3′-alkylene phosphonates, borontrifluoridates, borano phosphate esters and selenophosphates of 3′-5′ linkage or 2′-5′ linkage, phosphotriesters, thionoalkylphosphotriesters, hydrogen phosphonate linkages, alkyl phosphonates, alkylphosphonothioates, arylphosphonothioates, phosphoroselenoates, phosphorodiselenoates, phosphinates, phosphoramidates, 3′-alkylphosphoramidates, aminoalkylphosphoramidates, thionophosphoramidates, phosphoropiperazidates, phosphoroanilothioates, phosphoroanilidates, ketones, sulfones, sulfonamides, carbonates, carbamates, methylenehydrazos, methylenedimethylhydrazos, formacetals, thioformacetals, oximes, methyleneiminos, methylenemethyliminos, thioamidates, linkages with riboacetyl groups, aminoethyl glycine, silyl or siloxane linkages, alkyl or cycloalkyl linkages with or without heteroatoms of, for example, 1 to 10 carbons that are saturated or unsaturated and/or substituted and/or contain heteroatoms, linkages with morpholino structures, amides, polyamides wherein the bases are attached to the aza nitrogens of the backbone directly or indirectly, and combinations thereof. Phosphorothioate antisense oligonucleotides (PS ASO) are antisense oligonucleotides comprising a phosphorothioate linkage. Mesyl phosphoramidate antisense oligonucleotides (MsPA ASO) are antisense oligonucleotides comprising a mesyl phosphoramidate linkage.

In some instances, the modification is a methyl or thiol modification such as methylphosphonate, mesyl phosphoramidate, or thiolphosphonate modification. In some instances, a modified nucleotide includes, but is not limited to, 2′-fluoro N3-P5′-phosphoramidites.

In some instances, a modified nucleotide includes, but is not limited to, hexitol nucleic acid (or 1′, 5′-anhydrohexitol nucleic acids (HNA)).

In some embodiments, one or more modifications further optionally include modifications of the ribose moiety, phosphate backbone and the nucleoside, or modifications of the nucleotide analogues at the 3′ or the 5′ terminus. For example, the 3′ terminus optionally includes a 3′ cationic group, or by inverting the nucleoside at the 3′-terminus with a 3′-3′ linkage. In another alternative, the 3′-terminus is optionally conjugated with an aminoalkyl group, e.g., a 3′ C5-aminoalkyl dT. In an additional alternative, the 3′-terminus is optionally conjugated with an abasic site, e.g., with an apurinic or apyrimidinic site. In some instances, the 5′-terminus is conjugated with an aminoalkyl group, e.g., a 5′-O-alkylamino substituent. In some cases, the 5′-terminus is conjugated with an abasic site, e.g., with an apurinic or apyrimidinic site.

In some embodiments, the oligonucleotide molecule comprises one or more of the synthetic nucleotide analogues described herein. In some instances, the oligonucleotide molecule comprises 1, 2, 3, 4, 5, 6, 7, 8, 9, 10, 11, 12, 13, 14, 15, 16, 17, 18, 20, 25, or more of the synthetic nucleotide analogues described herein. In some embodiments, the synthetic nucleotide analogues include 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the oligonucleotide molecule comprises 1, 2, 3, 4, 5, 6, 7, 8, 9, 10, 11, 12, 13, 14, 15, 16, 17, 18, 20, 25, or more of the synthetic nucleotide analogues selected from 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the oligonucleotide molecule comprises 1, 2, 3, 4, 5, 6, 7, 8, 9, 10, 11, 12, 13, 14, 15, 16, 17, 18, 20, 25, or more of 2′-O-methyl modified nucleotides. In some instances, the oligonucleotide molecule comprises 1, 2, 3, 4, 5, 6, 7, 8, 9, 10, 11, 12, 13, 14, 15, 16, 17, 18, 20, 25, or more of 2′-O-methoxyethyl (2′-O-MOE) modified nucleotides. In some instances, the oligonucleotide molecule comprises 1, 2, 3, 4, 5, 6, 7, 8, 9, 10, 11, 12, 13, 14, 15, 16, 17, 18, 20, 25, or more of thiolphosphonate nucleotides.

In some instances, the oligonucleotide molecule comprises at least one of: from about 5% to about 100% modification, from about 10% to about 100% modification, from about 20% to about 100% modification, from about 30% to about 100% modification, from about 40% to about 100% modification, from about 50% to about 100% modification, from about 60% to about 100% modification, from about 70% to about 100% modification, from about 80% to about 100% modification, and from about 90% to about 100% modification. In some instances, the oligonucleotide molecule comprises 100% modification.

In some cases, the oligonucleotide molecule comprises at least one of: from about 10% to about 90% modification, from about 20% to about 90% modification, from about 30% to about 90% modification, from about 40% to about 90% modification, from about 50% to about 90% modification, from about 60% to about 90% modification, from about 70% to about 90% modification, and from about 80% to about 100% modification.

In some cases, the oligonucleotide molecule comprises at least one of: from about 10% to about 80% modification, from about 20% to about 80% modification, from about 30% to about 80% modification, from about 40% to about 80% modification, from about 50% to about 80% modification, from about 60% to about 80% modification, and from about 70% to about 80% modification.

In some instances, the oligonucleotide molecule comprises at least one of: from about 10% to about 70% modification, from about 20% to about 70% modification, from about 30% to about 70% modification, from about 40% to about 70% modification, from about 50% to about 70% modification, and from about 60% to about 70% modification.

In some instances, the oligonucleotide molecule comprises at least one of: from about 10% to about 60% modification, from about 20% to about 60% modification, from about 30% to about 60% modification, from about 40% to about 60% modification, and from about 50% to about 60% modification.

In some cases, the oligonucleotide molecule comprises at least one of: from about 10% to about 50% modification, from about 20% to about 50% modification, from about 30% to about 50% modification, and from about 40% to about 50% modification.

In some cases, the oligonucleotide molecule comprises at least one of: from about 10% to about 40% modification, from about 20% to about 40% modification, and from about 30% to about 40% modification.

In some cases, the oligonucleotide molecule comprises at least one of: from about 10% to about 30% modification, and from about 20% to about 30% modification.

In some cases, the oligonucleotide molecule comprises from about 10% to about 20% modification.

In some cases, the oligonucleotide molecule comprises from about 15% to about 90%, from about 20% to about 80%, from about 30% to about 70%, or from about 40% to about 60% modifications.

In additional cases, the oligonucleotide molecule comprises at least about 15%, 20%, 30%, 40%, 50%, 60%, 70%, 80%, 90%, 95%, or 99% modifications.

In some embodiments, the oligonucleotide molecule comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 modifications.

In some instances, the oligonucleotide molecule comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 modified nucleotides.

In some instances, from about 5 to about 100% of the oligonucleotide molecule comprise the synthetic nucleotide analogues described herein. In some instances, about 5%, 10%, 15%, 20%, 25%, 30%, 35%, 40%, 45%, 50%, 55%, 60%, 65%, 70%, 75%, 80%, 85%, 90%, 95% or 100% of the oligonucleotide molecule comprise the synthetic nucleotide analogues described herein. In some instances, about 5% of the oligonucleotide molecule comprises the synthetic nucleotide analogues described herein. In some instances, about 10% of the oligonucleotide molecule comprises the synthetic nucleotide analogues described herein. In some instances, about 15% of the oligonucleotide molecule comprises the synthetic nucleotide analogues described herein. In some instances, about 20% of the oligonucleotide molecule comprises the synthetic nucleotide analogues described herein. In some instances, about 25% of the oligonucleotide molecule comprises the synthetic nucleotide analogues described herein. In some instances, about 30% of the oligonucleotide molecule comprises the synthetic nucleotide analogues described herein. In some instances, about 35% of the oligonucleotide molecule comprises the synthetic nucleotide analogues described herein. In some instances, about 40% of the oligonucleotide molecule comprises the synthetic nucleotide analogues described herein. In some instances, about 45% of the oligonucleotide molecule comprises the synthetic nucleotide analogues described herein. In some instances, about 50% of the oligonucleotide molecule comprises the synthetic nucleotide analogues described herein. In some instances, about 55% of the oligonucleotide molecule comprises the synthetic nucleotide analogues described herein. In some instances, about 60% of the oligonucleotide molecule comprises the synthetic nucleotide analogues described herein. In some instances, about 65% of the oligonucleotide molecule comprises the synthetic nucleotide analogues described herein. In some instances, about 70% of the oligonucleotide molecule comprises the synthetic nucleotide analogues described herein. In some instances, about 75% of the oligonucleotide molecule comprises the synthetic nucleotide analogues described herein. In some instances, about 80% of the oligonucleotide molecule comprises the synthetic nucleotide analogues described herein. In some instances, about 85% of the oligonucleotide molecule comprises the synthetic nucleotide analogues described herein. In some instances, about 90% of the oligonucleotide molecule comprises the synthetic nucleotide analogues described herein. In some instances, about 95% of the oligonucleotide molecule comprises the synthetic nucleotide analogues described herein. In some instances, about 96% of the oligonucleotide molecule comprises the synthetic nucleotide analogues described herein. In some instances, about 97% of the oligonucleotide molecule comprises the synthetic nucleotide analogues described herein. In some instances, about 98% of the oligonucleotide molecule comprises the synthetic nucleotide analogues described herein. In some instances, about 99% of the oligonucleotide molecule comprises the synthetic nucleotide analogues described herein. In some instances, about 100% of the oligonucleotide molecule comprises the synthetic nucleotide analogues described herein. In some embodiments, the synthetic nucleotide analogues include 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof.

In some embodiments, the oligonucleotide molecule comprises from about 1 to about 25 modifications in which the modification comprises an synthetic nucleotide analogues described herein. In some embodiments, the oligonucleotide molecule comprises about 1 modification in which the modification comprises a synthetic nucleotide analogue described herein. In some embodiments, the oligonucleotide molecule comprises about 2 modifications in which the modifications comprise a synthetic nucleotide analogue described herein. In some embodiments, the oligonucleotide molecule comprises about 3 modifications in which the modifications comprise a synthetic nucleotide analogue described herein. In some embodiments, the oligonucleotide molecule comprises about 4 modifications in which the modifications comprise a synthetic nucleotide analogue described herein. In some embodiments, the oligonucleotide molecule comprises about 5 modifications in which the modifications comprise a synthetic nucleotide analogue described herein. In some embodiments, the oligonucleotide molecule comprises about 6 modifications in which the modifications comprise a synthetic nucleotide analogue described herein. In some embodiments, the oligonucleotide molecule comprises about 7 modifications in which the modifications comprise a synthetic nucleotide analogue described herein. In some embodiments, the oligonucleotide molecule comprises about 8 modifications in which the modifications comprise a synthetic nucleotide analogue described herein. In some embodiments, the oligonucleotide molecule comprises about 9 modifications in which the modifications comprise a synthetic nucleotide analogue described herein. In some embodiments, the oligonucleotide molecule comprises about 10 modifications in which the modifications comprise a synthetic nucleotide analogue described herein. In some embodiments, the oligonucleotide molecule comprises about 11 modifications in which the modifications comprise a synthetic nucleotide analogue described herein. In some embodiments, the oligonucleotide molecule comprises about 12 modifications in which the modifications comprise a synthetic nucleotide analogue described herein. In some embodiments, the oligonucleotide molecule comprises about 13 modifications in which the modifications comprise a synthetic nucleotide analogue described herein. In some embodiments, the oligonucleotide molecule comprises about 14 modifications in which the modifications comprise a synthetic nucleotide analogue described herein. In some embodiments, the oligonucleotide molecule comprises about 15 modifications in which the modifications comprise a synthetic nucleotide analogue described herein. In some embodiments, the oligonucleotide molecule comprises about 16 modifications in which the modifications comprise a synthetic nucleotide analogue described herein. In some embodiments, the oligonucleotide molecule comprises about 17 modifications in which the modifications comprise a synthetic nucleotide analogue described herein. In some embodiments, the oligonucleotide molecule comprises about 18 modifications in which the modifications comprise a synthetic nucleotide analogue described herein. In some embodiments, the oligonucleotide molecule comprises about 19 modifications in which the modifications comprise a synthetic nucleotide analogue described herein. In some embodiments, the oligonucleotide molecule comprises about 20 modifications in which the modifications comprise a synthetic nucleotide analogue described herein. In some embodiments, the oligonucleotide molecule comprises about 21 modifications in which the modifications comprise a synthetic nucleotide analogue described herein. In some embodiments, the oligonucleotide molecule comprises about 22 modifications in which the modifications comprise a synthetic nucleotide analogue described herein. In some embodiments, the oligonucleotide molecule comprises about 23 modifications in which the modifications comprise a synthetic nucleotide analogue described herein. In some embodiments, the oligonucleotide molecule comprises about 24 modifications in which the modifications comprise a synthetic nucleotide analogue described herein. In some embodiments, the oligonucleotide molecule comprises about 25 modifications in which the modifications comprise a synthetic nucleotide analogue described herein. In some embodiments, the oligonucleotide molecule comprises about 26 modifications in which the modifications comprise a synthetic nucleotide analogue described herein. In some embodiments, the oligonucleotide molecule comprises about 27 modifications in which the modifications comprise a synthetic nucleotide analogue described herein. In some embodiments, the oligonucleotide molecule comprises about 28 modifications in which the modifications comprise a synthetic nucleotide analogue described herein. In some embodiments, the oligonucleotide molecule comprises about 29 modifications in which the modifications comprise a synthetic nucleotide analogue described herein. In some embodiments, the oligonucleotide molecule comprises about 30 modifications in which the modifications comprise a synthetic nucleotide analogue described herein. In some embodiments, the oligonucleotide molecule comprises about 31 modifications in which the modifications comprise a synthetic nucleotide analogue described herein. In some embodiments, the oligonucleotide molecule comprises about 32 modifications in which the modifications comprise a synthetic nucleotide analogue described herein. In some embodiments, the oligonucleotide molecule comprises about 33 modifications in which the modifications comprise a synthetic nucleotide analogue described herein. In some embodiments, the oligonucleotide molecule comprises about 34 modifications in which the modifications comprise a synthetic nucleotide analogue described herein. In some embodiments, the oligonucleotide molecule comprises about 35 modifications in which the modifications comprise a synthetic nucleotide analogue described herein. In some embodiments, the oligonucleotide molecule comprises about 36 modifications in which the modifications comprise a synthetic nucleotide analogue described herein. In some embodiments, the oligonucleotide molecule comprises about 37 modifications in which the modifications comprise a synthetic nucleotide analogue described herein. In some embodiments, the oligonucleotide molecule comprises about 38 modifications in which the modifications comprise a synthetic nucleotide analogue described herein. In some embodiments, the oligonucleotide molecule comprises about 39 modifications in which the modifications comprise a synthetic nucleotide analogue described herein. In some embodiments, the oligonucleotide molecule comprises about 40 modifications in which the modifications comprise a synthetic nucleotide analogue described herein.

In some embodiments, an oligonucleotide molecule is assembled from two separate polynucleotides wherein one polynucleotide comprises the sense strand and the second polynucleotide comprises the antisense strand of the oligonucleotide molecule. In other embodiments, the sense strand is connected to the antisense strand via a linker molecule, which in some instances is a polynucleotide linker or a non-nucleotide linker.

In some embodiments, an oligonucleotide molecule comprises a sense strand and antisense strand, wherein pyrimidine nucleotides in the sense strand comprises 2′-O-methylpyrimidine nucleotides and purine nucleotides in the sense strand comprise 2′-deoxy purine nucleotides. In some embodiments, an oligonucleotide molecule comprises a sense strand and antisense strand, wherein pyrimidine nucleotides present in the sense strand comprise 2′-deoxy-2′-fluoro pyrimidine nucleotides and wherein purine nucleotides present in the sense strand comprise 2′-deoxy purine nucleotides.

In some embodiments, an oligonucleotide molecule comprises a sense strand and antisense strand, wherein the pyrimidine nucleotides when present in said antisense strand are 2′-deoxy-2′-fluoro pyrimidine nucleotides and the purine nucleotides when present in said antisense strand are 2′-O-methyl purine nucleotides.

In some embodiments, an oligonucleotide molecule comprises a sense strand and antisense strand, wherein the pyrimidine nucleotides when present in said antisense strand are 2′-deoxy-2′-fluoro pyrimidine nucleotides and wherein the purine nucleotides when present in said antisense strand comprise 2′-deoxy-purine nucleotides.

In some embodiments, an oligonucleotide molecule comprises a sense strand and antisense strand, and at least one of sense strand and antisense strands has a plurality of (e.g., two or more, three or more, four or more, five or more, six or more, seven or more, eight or more, etc.) 2′-O-methyl or 2′-deoxy-2′-fluoro modified nucleotides. In some embodiments, at least two, three, four, five, six, or seven out of the a plurality of 2′-O-methyl or 2′-deoxy-2′-fluoro modified nucleotides are consecutive nucleotides. In some embodiments, consecutive 2′-O-methyl or 2′-deoxy-2′-fluoro modified nucleotides are located at the 5′-end of the sense strand and/or the antisense strand. In some embodiments, consecutive 2′-O-methyl or 2′-deoxy-2′-fluoro modified nucleotides are located at the 3′-end of the sense strand and/or the antisense strand. In some embodiments, the sense strand of oligonucleotide molecule includes at least four, at least five, at least six consecutive 2′-O-methyl modified nucleotides at its 5′ end and/or 3′ end, or both. Optionally, in such embodiments, the sense strand of oligonucleotide molecule includes at least one, at least two, at least three, at least four 2′-deoxy-2′-fluoro modified nucleotides at the 3′ end of the at least four, at least five, at least six consecutive 2′-O-methyl modified nucleotides at the polynucleotides' 5′ end, or at the 5′ end of the at least four, at least five, at least six consecutive 2′-O-methyl modified nucleotides at polynucleotides' 3′ end. Also optionally, such at least two, at least three, at least four 2′-deoxy-2′-fluoro modified nucleotides are consecutive nucleotides.

In some embodiments, an oligonucleotide molecule comprises a sense strand and antisense strand, and at least one of sense strand and antisense strand has 2′-O-methyl modified nucleotide located at the 5′ end of the sense strand and/or the antisense strand. In some embodiments, at least one of sense strand and antisense strands has 2′-O-methyl modified nucleotide located at the 3′ end of the sense strand and/or the antisense strand. In some embodiments, the 2′-O-methyl modified nucleotide located at the 5′ end of the sense strand and/or the antisense strand is a purine nucleotide. In some embodiments, the 2′-O-methyl modified nucleotide located at the 5′ end of the sense strand and/or the antisense strand is a pyrimidine nucleotide.

In some embodiments, an oligonucleotide molecule comprises a sense strand and antisense strand, and one of sense strand and antisense strand has at least two consecutive 2′-deoxy-2′-fluoro modified nucleotides located at the 5′ end, while another strand has at least two consecutive 2′-O-methyl modified nucleotides located at the 5′ end. In some embodiments, where the strand has at least two consecutive 2′-deoxy-2′-fluoro modified nucleotides located at the 5′ end, the strand also includes at least two, at least three consecutive 2′-O-methyl modified nucleotides at the 3′ end of the at least two consecutive 2′-deoxy-2′-fluoro modified nucleotides. In some embodiments, one of sense strand and antisense strand has at least two, at least three, at least four, at least five, at least six, or at least seven consecutive 2′-O-methyl modified nucleotides that are linked to a 2′-deoxy-2′-fluoro modified nucleotide on its 5′ end and/or 3′ end. In some embodiments, one of sense strand and antisense strand has at least four, at least five nucleotides that have alternating 2′-O-methyl modified nucleotide and 2′-deoxy-2′-fluoro modified nucleotide.

In some embodiments, the oligonucleotide molecule, such as an siRNA, has the formula as illustrated in Formula III:

Sense-strand (SS)
N₁N₂N₃N₄N₅N₆N₇N₈N₉N₁₀N₁₁N₁₂N₁₃N₁₄N₁₅N₁₆N₁₇N₁₈N₁₉
Antisense-strand (AS),
N₂₁N₂₀N₁₉N₁₈N₁₇N₁₆N₁₅N₁₄N₁₃N₁₂N₁₁N₁₀N₉N₈N₇N₆N₅N₄N₃N₂N₁

wherein each nucleotide represented by N, is independently, A, U, C, or G or a modified nucleotide base, such as those provided for herein. The N₁ nucleotides of the sense strand and the antisense strand represent the 5′ end of the respective strands. For clarity, although Formula III utilizes N₁, N₂, N₃, etc. in both the sense and the antisense strand, the nucleotide bases do not need to be the same and are not intended to be the same. The siRNA that is illustrated in Formula III would be complementary to a target sequence.

For example, in some embodiments, the sense strand comprises a 2′O-methyl modified nucleotide with a phosphorothioate (PS) modified backbone at N₁ and N₂, a 2′-fluoro modified nucleotide at N₃, N₇, N₈, N₉, N₁₂, and N₁₇, and a 2′O-methyl modified nucleotide at N₄, N₅, N₆, N₁₀, N₁₁, N₁₃, N₁₄, N₁₅, N₁₆, N₁₈, and N₁₉.

In some embodiments, the antisense strand comprises a vinyl phosphonate moiety attached to N₁, a 2′fluoro-modified nucleotide with a phosphorothioate (PS) modified backbone at N₂, a 2′O-methyl modified nucleotide at N₃, N₄, N₅, N₆, N₇, N₈, N₉, N₁₀, N₁₁, N₁₂, N₁₃, N₁₅, N₁₆, N₁₇, N₁₈, and N₁₉, a 2′fluoro-modified nucleotide at N₁₄, and a 2′O-methyl modified nucleotide with a phosphorothioate (PS) modified backbone at N₂₀ and N₂₁.

In some embodiments, an oligonucleotide molecule comprises a sense strand and antisense strand, wherein the sense strand includes a terminal cap moiety at the 5′ end, the 3′ end, or both of the 5′ and 3′ ends of the sense strand. In other embodiments, the terminal cap moiety is an inverted deoxy abasic moiety.

In some embodiments, an oligonucleotide molecule comprises a sense strand and an antisense strand, wherein the antisense strand comprises a glyceryl modification at the 3′ end of the antisense strand.

In some embodiments, an oligonucleotide molecule comprises a sense strand and an antisense strand, in which the sense strand comprises one or more, for example, about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or about one or more (e.g., about 1, 2, 3, 4, 5, 6, 7, 8, 9, 10 or more) universal base modified nucleotides, and optionally a terminal cap molecule at the 3′ end, the 5′ end, or both of the 3′- and 5′-ends of the sense strand; and in which the antisense strand comprises about 1 to about 10 or more, specifically about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′ end, the 5′ end, or both of the 3′ and 5′ ends of the antisense strand. In other embodiments, one or more, for example about 1, 2, 3, 4, 5, 6, 7, 8, 9, 10, or more, pyrimidine nucleotides of the sense and/or antisense strand are chemically-modified with 2′-deoxy, 2′-O-methyl and/or 2′-deoxy-2′-fluoro nucleotides, with or without one or more, for example about 1, 2, 3, 4, 5, 6, 7, 8, 9, 10, or more, phosphorothioate, phosphorodithioate, phosphonate, phosphoramidate, or mesyl phosphoramidate internucleotide linkages and/or a terminal cap molecule at the 3′ end, the 5′ end, or both of the 3′ and 5′ ends, being present in the same or different strand.

In some embodiments, an oligonucleotide molecule comprises a sense strand and an antisense strand, in which the sense strand comprises about 1 to about 25, for example, about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages, and/or one or more (e.g., about 1, 2, 3, 4, 5, 6, 7, 8, 9, 10, or more) 2′-deoxy, 2′-O-methyl, 2′-deoxy-2′-fluoro, and/or one or more (e.g., about 1, 2, 3, 4, 5, 6, 7, 8, 9, 10, or more) universal base modified nucleotides, and optionally a terminal cap molecule at the 3′ end, the 5′ end, or both of the 3′ and 5′ ends of the sense strand; and in which the antisense strand comprises about 1 to about 25 or more, for example about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′ end, the 5′ end, or both of the 3′ and 5′ ends of the antisense strand. In other embodiments, one or more, for example about 1, 2, 3, 4, 5, 6, 7, 8, 9, 10, or more, pyrimidine nucleotides of the sense and/or antisense strand are chemically-modified with 2′-deoxy, 2′-O-methyl and/or 2′-deoxy-2′-fluoro nucleotides, with or without about 1 to about 25 or more, for example about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages and/or a terminal cap molecule at the 3′ end, the 5′ end, or both of the 3′ and 5′ ends, being present in the same or different strand.

In some embodiments, an oligonucleotide molecule comprises a sense strand and an antisense strand, in which the antisense strand comprises one or more, for example, about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages, and/or about one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides at the 3′ end, the 5′ end, or both of the 3′ and 5′ ends of the sense strand and/or antisense strand, and optionally a terminal cap molecule at the 3′ end, the 5′ end, or both of the 3′ and 5′ ends of the sense strand. In some embodiments, the antisense strand comprises about 1 to about 10 or more, specifically about 1, 2, 3, 4, 5, 6, 7, 8, 9, 10 or more phosphorothioate, phosphorodithioate, phosphonate, phosphoramidate, or mesyl phosphoramidate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′ end, the 5′ end, or both of the 3′ and 5′ ends of the antisense strand. In other embodiments, one or more, for example about 1, 2, 3, 4, 5, 6, 7, 8, 9, 10, 11, 12, 13, 14, 15, 16, 17, 18, 19, 20, or more pyrimidine nucleotides of the sense and/or antisense strand are chemically-modified with 2′-deoxy, 2′-O-methyl and/or 2′-deoxy-2′-fluoro nucleotides, with or without one or more, for example, about 1, 2, 3, 4, 5, 6, 7, 8, 9, 10 or more phosphorothioate, phosphorodithioate, phosphonate, phosphoramidate, or mesyl phosphoramidate internucleotide linkages and/or a terminal cap molecule at the 3′ end, the 5′ end, or both of the 3′ and 5′ ends, being present in the same or different strand.

In some embodiments, an oligonucleotide molecule comprises a sense strand and an antisense strand, in which the antisense strand comprises about 1 to about 25 or more, for example, about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′ end, the 5′ end, or both of the 3′ and 5′ ends of the sense strand; and the antisense strand comprises about 1 to about 25 or more, for example about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′ end, the 5′ end, or both of the 3′ and 5′ ends of the antisense strand. In other embodiments, one or more, for example about 1, 2, 3, 4, 5, 6, 7, 8, 9, 10 or more pyrimidine nucleotides of the sense and/or antisense strand are chemically-modified with 2′-deoxy, 2′-O-methyl and/or 2′-deoxy-2′-fluoro nucleotides, with or without about 1 to about 5, for example about 1, 2, 3, 4, 5 or more phosphorothioate, phosphorodithioate, phosphonate, phosphoramidate, or mesyl phosphoramidate internucleotide linkages and/or a terminal cap molecule at the 3′ end, the 5′ end, or both of the 3′ and 5′ ends, being present in the same or different strand.

In some embodiments, an oligonucleotide molecule described herein is a chemically-modified short interfering nucleic acid molecule having about 1 to about 25, for example, about 1, 2, 3, 4, 5, 6, 7, 8, 9, 10, 11, 12, 13, 14, 15, 16, 17, 18, 19, 20 or more phosphorothioate, phosphorodithioate, phosphonate, phosphoramidate, or mesyl phosphoramidate internucleotide linkages in each strand of the oligonucleotide molecule. In some embodiments, an oligonucleotide molecule comprises a sense strand and an antisense strand, and the antisense strand comprises a phosphate backbone modification at the 3′ end of the antisense strand. Alternatively and/or additionally, an oligonucleotide molecule comprises a sense strand and an antisense strand, and the sense strand comprises a phosphate backbone modification at the 5′ end of the antisense strand. In some instances, the phosphate backbone modification is a phosphorothioate. In some instances, the phosphate backbone modification is a phosphorodithioate. In some instances, the phosphate backbone modification is a phosphonate. In some instances, the phosphate backbone modification is a phosphoramidate. In some instances, the phosphate backbone modification is a mesyl phosphoramidate. In some embodiments, the sense or antisense strand has three consecutive nucleosides that are coupled via two phosphorothioate backbone. In some embodiments, the sense or antisense strand has three consecutive nucleosides that are coupled via two phosphorodithioate backbone. In some embodiments, the sense or antisense strand has three consecutive nucleosides that are coupled via two phosphonate backbone. In some embodiments, the sense or antisense strand has three consecutive nucleosides that are coupled via two phosphoramidate backbone. In some embodiments, the sense or antisense strand has three consecutive nucleosides that are coupled via two mesyl phosphoramidate backbone.

In another embodiment, an oligonucleotide molecule described herein comprises 2′-5′ internucleotide linkages. In some instances, the 2′-5′ internucleotide linkage(s) is at the 3′ end, the 5′ end, or both of the 3′ and 5′ ends of one or both sequence strands. In addition instances, the 2′-5′ internucleotide linkage(s) is present at various other positions within one or both sequence strands, for example, about 1, 2, 3, 4, 5, 6, 7, 8, 9, 10, or more including every internucleotide linkage of a pyrimidine nucleotide in one or both strands of the oligonucleotide molecule comprise a 2′-5′ internucleotide linkage, or about 1, 2, 3, 4, 5, 6, 7, 8, 9, 10, or more including every internucleotide linkage of a purine nucleotide in one or both strands of the oligonucleotide molecule comprise a 2′-5′ internucleotide linkage.

In some embodiments, an oligonucleotide molecule is a single stranded molecule that mediates RNAi activity in a cell or reconstituted in vitro system, wherein the oligonucleotide molecule comprises a single stranded polynucleotide having complementarity to a target nucleic acid sequence, and wherein one or more pyrimidine nucleotides present in the oligonucleotide molecule are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any purine nucleotides present in the oligonucleotide molecule are 2′-deoxy purine nucleotides (e.g., wherein all purine nucleotides are 2′-deoxy purine nucleotides or alternately a plurality of purine nucleotides are 2′-deoxy purine nucleotides), and a terminal cap modification, that is optionally present at the 3′-end, the 5′-end, or both of the 3′ and 5′-ends of the antisense sequence, the oligonucleotide molecule optionally further comprising about 1 to about 4 (e.g., about 1, 2, 3, or 4) terminal 2′-deoxynucleotides at the 3′-end of the oligonucleotide molecule, wherein the terminal nucleotides further comprise one or more (e.g., 1, 2, 3, or 4) phosphorothioate or mesyl phosphoramidate internucleotide linkages, and wherein the oligonucleotide molecule optionally further comprises a terminal phosphate group, such as a 5′-terminal phosphate group.

In some cases, one or more of the synthetic nucleotide analogues described herein are resistant toward nucleases, such as ribonuclease (e.g., RNase H), deoxyribonuclease (e.g., DNase), or exonuclease (e.g., 5′-3′ exonuclease and 3′-5′ exonuclease), when compared to natural polynucleic acid molecules and endonucleases. In some instances, synthetic nucleotide analogues comprising 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or combinations thereof are resistant toward nucleases such as ribonuclease (e.g., RNase H), deoxyribonuclease (e.g., DNase), or exonuclease (e.g., 5′-3′ exonuclease and 3′-5′ exonuclease). In some instances, a 2′-O-methyl modified oligonucleotide molecule is nuclease resistant (e.g., RNase H, DNase, 5′-3′ exonuclease or 3′-5′ exonuclease resistant). In some instances, a 2′O-methoxyethyl (2′-O-MOE) modified oligonucleotide molecule is nuclease resistant (e.g., RNase H, DNase, 5′-3′ exonuclease or 3′-5′ exonuclease resistant). In some instances, a 2′-O-aminopropyl modified oligonucleotide molecule is nuclease resistant (e.g., RNase H, DNase, 5′-3′ exonuclease or 3′-5′ exonuclease resistant). In some instances, a 2′-deoxy modified oligonucleotide molecule is nuclease resistant (e.g., RNase H, DNase, 5′-3′ exonuclease or 3′-5′ exonuclease resistance). In some instances, a 2′-deoxy-2′-fluoro modified oligonucleotide molecule is nuclease resistant (e.g., RNase H, DNase, 5′-3′ exonuclease or 3′-5′ exonuclease resistant). In some instances, a 2′-O-aminopropyl (2′-O-AP) modified oligonucleotide molecule is nuclease resistant (e.g., RNase H, DNase, 5′-3′ exonuclease or 3′-5′ exonuclease resistant). In some instances, a 2′-O-dimethylaminoethyl (2′-O-DMAOE) modified oligonucleotide molecule is nuclease resistant (e.g., RNase H, DNase, 5′-3′ exonuclease or 3′-5′ exonuclease resistant). In some instances, a 2′-O-dimethylaminopropyl (2′-O-DMAP) modified oligonucleotide molecule is nuclease resistant (e.g., RNase H, DNase, 5′-3′ exonuclease or 3′-5′ exonuclease resistant). In some instances, a 2′-O-dimethylaminoethyloxyethyl (2′-O-DMAEOE) modified oligonucleotide molecule is nuclease resistant (e.g., RNase H, DNase, 5′-3′ exonuclease or 3′-5′ exonuclease resistant). In some instances, a 2′-O—N-methylacetamido (2′-O-NMA) modified oligonucleotide molecule is nuclease resistant (e.g., RNase H, DNase, 5′-3′ exonuclease or 3′-5′ exonuclease resistant). In some instances, an LNA modified oligonucleotide molecule is nuclease resistant (e.g., RNase H, DNase, 5′-3′ exonuclease or 3′-5′ exonuclease resistant). In some instances, an ENA modified oligonucleotide molecule is nuclease resistant (e.g., RNase H, DNase, 5′-3′ exonuclease or 3′-5′ exonuclease resistant). In some instances, an HNA modified oligonucleotide molecule is nuclease resistant (e.g., RNase H, DNase, 5′-3′ exonuclease or 3′-5′ exonuclease resistant). In some instances, morpholinos are nuclease resistant (e.g., RNase H, DNase, 5′-3′ exonuclease or 3′-5′ exonuclease resistant). In some instances, a PNA modified oligonucleotide molecule is resistant to nucleases (e.g., RNase H, DNase, 5′-3′ exonuclease or 3′-5′ exonuclease resistant). In some instances, a methylphosphonate modified oligonucleotide molecule is nuclease resistant (e.g., RNase H, DNase, 5′-3′ exonuclease or 3′-5′ exonuclease resistant). In some instances, a thiolphosphonate modified oligonucleotide molecule is nuclease resistant (e.g., RNase H, DNase, 5′-3′ exonuclease or 3′-5′ exonuclease resistant). In some instances, an oligonucleotide molecule comprising 2′-fluoro N3-P5′-phosphoramidites is nuclease resistant (e.g., RNase H, DNase, 5′-3′ exonuclease or 3′-5′ exonuclease resistant). In some instances, the 5′ conjugates described herein inhibit 5′-3′ exonucleolytic cleavage. In some instances, the 3′ conjugates described herein inhibit 3′-5′ exonucleolytic cleavage.

In some embodiments, one or more of the synthetic nucleotide analogues described herein have increased binding affinity toward their mRNA target relative to an equivalent natural polynucleic acid molecule. The one or more of the synthetic nucleotide analogues comprising 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-0-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, or 2′-fluoro N3-P5′-phosphoramidites have increased binding affinity toward their mRNA target relative to an equivalent natural polynucleic acid molecule. In some instances, a 2′-O-methyl modified oligonucleotide molecule has increased binding affinity toward their mRNA target relative to an equivalent natural polynucleic acid molecule. In some instances, a 2′-O-methoxyethyl (2′-O-MOE) modified oligonucleotide molecule has increased binding affinity toward their mRNA target relative to an equivalent natural polynucleic acid molecule. In some instances, a 2′-O-aminopropyl modified oligonucleotide molecule has increased binding affinity toward an mRNA target relative to an equivalent natural polynucleic acid molecule. In some instances, 2′-deoxy modified oligonucleotide molecule has increased binding affinity toward their mRNA target relative to an equivalent natural polynucleic acid molecule. In some instances, a 2′-deoxy-2′-fluoro modified oligonucleotide molecule has increased binding affinity toward their mRNA target relative to an equivalent natural polynucleic acid molecule. In some instances, a 2′-O-aminopropyl (2′-O-AP) modified oligonucleotide molecule has increased binding affinity toward their mRNA target relative to an equivalent natural polynucleic acid molecule. In some instances, a 2′-O-dimethylaminoethyl (2′-O-DMAOE) modified oligonucleotide molecule has increased binding affinity toward their mRNA target relative to an equivalent natural polynucleic acid molecule. In some instances, a 2′-O-dimethylaminopropyl (2′-O-DMAP) modified oligonucleotide molecule has increased binding affinity toward their mRNA target relative to an equivalent natural polynucleic acid molecule. In some instances, a 2′-O-dimethylaminoethyloxyethyl (2′-O-DMAEOE) modified oligonucleotide molecule has increased binding affinity toward their mRNA target relative to an equivalent natural polynucleic acid molecule. In some instances, a 2′-O—N-methylacetamido (2′-O-NMA) modified oligonucleotide molecule has increased binding affinity toward their mRNA target relative to an equivalent natural polynucleic acid molecule. In some instances, an LNA modified oligonucleotide molecule has increased binding affinity toward their mRNA target relative to an equivalent natural polynucleic acid molecule. In some instances, an ENA modified oligonucleotide molecule has increased binding affinity toward their mRNA target relative to an equivalent natural polynucleic acid molecule. In some instances, a PNA modified oligonucleotide molecule has increased binding affinity toward their mRNA target relative to an equivalent natural polynucleic acid molecule. In some instances, an HNA modified oligonucleotide molecule has increased binding affinity toward their mRNA target relative to an equivalent natural polynucleic acid molecule. In some instances, a morpholino modified oligonucleotide molecule has increased binding affinity toward their mRNA target relative to an equivalent natural polynucleic acid molecule. In some instances, a methylphosphonate nucleotides modified oligonucleotide molecule has increased binding affinity toward their mRNA target relative to an equivalent natural polynucleic acid molecule. In some instances, a thiolphosphonate nucleotides modified oligonucleotide molecule has increased binding affinity toward their mRNA target relative to an equivalent natural polynucleic acid molecule. In some instances, an oligonucleotide molecule comprising 2′-fluoro N3-P5′-phosphoramidites has increased binding affinity toward an mRNA target relative to an equivalent natural polynucleic acid molecule. In some cases, the increased affinity is illustrated with a lower Kd, a higher melt temperature (Tm), or a combination thereof.

In some embodiments, an oligonucleotide molecule described herein is a chirally pure (or stereo pure) polynucleic acid molecule, or a polynucleic acid molecule comprising a single enantiomer. In some instances, the oligonucleotide molecule comprises L-nucleotide. In some instances, the oligonucleotide molecule comprises D-nucleotides. In some instance, an oligonucleotide molecule composition comprises less than 30%, 25%, 20%, 15%, 10%, 5%, 4%, 3%, 2%, 1%, or less of its mirror enantiomer. In some cases, an oligonucleotide molecule composition comprises less than 30%, 25%, 20%, 15%, 10%, 5%, 4%, 3%, 2%, 1%, or less of a racemic mixture.

In some embodiments, an oligonucleotide molecule described herein is further modified to include an aptamer conjugating moiety. In some instances, the aptamer conjugating moiety is a DNA aptamer conjugating moiety. In some instances, the aptamer conjugating moiety is Alphamer, which comprises an aptamer portion that recognizes a specific cell-surface target and a portion that presents a specific epitope for attaching to circulating antibodies.

In additional embodiments, an oligonucleotide molecule described herein is modified to increase its stability. In some embodiment, the oligonucleotide molecule is RNA (e.g., siRNA). In some instances, the oligonucleotide molecule is modified by one or more of the modifications described above to increase its stability. In some cases, the oligonucleotide molecule is modified at the 2′ hydroxyl position, such as by 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modification or by a locked or bridged ribose conformation (e.g., LNA or ENA). In some cases, the oligonucleotide molecule is modified by 2′-O-methyl and/or 2′-O-methoxyethyl ribose. In some cases, the oligonucleotide molecule also includes morpholinos, PNAs, HNA, methylphosphonate nucleotides, thiolphosphonate nucleotides, and/or 2′-fluoro N3-P5′-phosphoramidites to increase its stability. In some instances, the oligonucleotide molecule is a chirally pure (or stereo pure) oligonucleotide molecule. In some instances, the chirally pure (or stereo pure) oligonucleotide molecule is modified to increase its stability. Suitable modifications to the RNA to increase stability for delivery will be apparent to the skilled person.

In some embodiments, the oligonucleotide molecule comprises 2′ modifications. In some embodiments, the nucleotides of the oligonucleotide molecule at positions 3, 7, 8, 9, 12, and 17 from the 5′ end of the sense strand are not modified with a 2′O-methyl modification. In some embodiments, the nucleotides of the oligonucleotide molecule at positions 3, 7, 8, 9, 12, and 17 from the 5′ end of the sense strand are modified with a 2′fluoro modification. In some embodiments, the nucleotides of the oligonucleotide molecule at positions 2 and 14 from the 5′ end of the antisense strand are not modified with a 2′O-methyl modification. In some embodiments, the nucleotides of the oligonucleotide molecule at positions 2 and 14 from the 5′ end of the antisense strand are modified with a 2′fluoro modification. In some embodiments, any of the nucleotides may further comprise a 5′-phosphorothioate group modification. In some embodiments, the nucleotides of the oligonucleotide molecule at positions 1 and 2 from the 5′ end of the sense strand are modified with a 5′-phosphorothioate group modification. In some embodiments, the nucleotides of the oligonucleotide molecule at positions 1, 2, 20, and 21 from the 5′ end of the antisense strand are modified with a 5′-phosphorothioate group modification. In some embodiments, the 5′ end of the sense or antisense strand of the oligonucleotide molecule may further comprise a vinylphosphonate modification. In some embodiments, the nucleotide of the oligonucleotide molecule at position 1 from the 5′ end of the antisense strand is modified with a vinylphosphonate modification.

In some instances, the oligonucleotide molecule is a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. In some instances, the oligonucleotide molecule is assembled from two separate polynucleotides, where one strand is the sense strand and the other is the antisense strand, wherein the antisense and sense strands are self-complementary (e.g., each strand comprises nucleotide sequence that is complementary to nucleotide sequence in the other strand; such as where the antisense strand and sense strand form a duplex or double stranded structure, for example wherein the double stranded region is about 19, 20, 21, 22, 23, or more base pairs); the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof. Alternatively, the oligonucleotide molecule is assembled from a single oligonucleotide, where the self-complementary sense and antisense regions of the oligonucleotide molecule are linked by means of a nucleic acid-based or non-nucleic acid-based linker(s).

In some cases, the oligonucleotide molecule is a polynucleotide with a duplex, asymmetric duplex, hairpin or asymmetric hairpin secondary structure, having self-complementary sense and antisense regions, wherein the antisense region comprises a nucleic acid sequence that is complementary to a nucleic acid sequence in a separate target nucleic acid molecule or a portion thereof and the sense region comprises a nucleic acid sequence corresponding to the target nucleic acid sequence or a portion thereof. In other cases, the oligonucleotide molecule is a circular single-stranded polynucleotide having two or more loop structures and a stem comprising self-complementary sense and antisense regions, wherein the antisense region comprises a nucleic acid sequence that is complementary to a nucleic acid sequence in a target nucleic acid molecule or a portion thereof and the sense region comprises a nucleic acid sequence corresponding to the target nucleic acid sequence or a portion thereof, and wherein the circular polynucleotide is processed either in vivo or in vitro to generate an active oligonucleotide molecule capable of mediating RNAi. In additional cases, the oligonucleotide molecule also comprises a single-stranded polynucleotide comprising a nucleic acid sequence complementary to a nucleic acid sequence in a target nucleic acid molecule or a portion thereof (for example, where such oligonucleotide molecule does not require the presence within the oligonucleotide molecule of a nucleic acid sequence corresponding to the target nucleic acid sequence or a portion thereof), wherein the single stranded polynucleotide further comprises a terminal phosphate group, such as a 5′-phosphate, or 5′, 3′-diphosphate.

In some instances, an asymmetric hairpin is a linear oligonucleotide molecule comprising an antisense region, a loop portion that comprises nucleotides or non-nucleotides, and a sense region that comprises fewer nucleotides than the antisense region to the extent that the sense region has enough complimentary nucleotides to base pair with the antisense region and form a duplex with loop. For example, an asymmetric hairpin oligonucleotide molecule comprises an antisense region having length sufficient to mediate RNAi in a cell or in vitro system (e.g., about 19 to about 22 nucleotides) and a loop region comprising about 4 to about 8 nucleotides, and a sense region having about 3 to about 18 nucleotides that are complementary to the antisense region. In some cases, the asymmetric hairpin oligonucleotide molecule also comprises a 5′-terminal phosphate group that is chemically modified. In additional cases, the loop portion of the asymmetric hairpin oligonucleotide molecule comprises nucleotides, non-nucleotides, linker molecules, or conjugate molecules.

In some embodiments, an asymmetric duplex is an oligonucleotide molecule having two separate strands comprising a sense region and an antisense region, wherein the sense region comprises fewer nucleotides than the antisense region to the extent that the sense region has enough complimentary nucleotides to base pair with the antisense region and form a duplex. For example, an asymmetric duplex oligonucleotide molecule comprises an antisense region having length sufficient to mediate RNAi in a cell or in vitro system (e.g., about 19 to about 22 nucleotides) and a sense region having about 3 to about 19 nucleotides that are complementary to the antisense region.

In some cases, a universal base refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole, as known in the art.

In some embodiments, the dsRNA agents are 5′ phosphorylated or include a phosphoryl analog at the 5′ terminus. 5′-phosphate modifications include those which are compatible with RISC mediated gene silencing. Suitable modifications include: 5′-monophosphate (HO₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); phosphorodithioate [—O₂PS₂—]; phosphonate [—PO(OH)₂—]; phosphoramidate [—O═P(OH)₂—]; mesyl phosphoramidate (CH₃)(SO₂)(N)P(O)₂—O-5′); any additional combination of oxygen/sulfur replaced monophosphate, diphosphate and triphosphates (e.g., 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (R=alkyl=methyl, ethyl, isopropyl, propyl, etc., e.g., RP(OH)(O)—O-5′-, 5′-alkenylphosphonates (i.e. vinyl, substituted vinyl), (OH)₂(O)P-5′-CH2-), 5′-alkyletherphosphonates (R=alkylether=methoxymethyl (MeOCH2-), ethoxymethyl, etc., e.g., RP(OH)(O)—O-5′-). In some embodiments, the modification can be placed in the antisense strand of a dsRNA agent.

Other modifications and patterns of modifications can be found in, for example, U.S. Pat. No. 10,233,448, which is hereby incorporated by reference. Other modifications and patterns of modifications can be found in, for example, Anderson et al., Nucleic Acids Research 2021, 49 (16), 9026-9041, which is hereby incorporated by reference. Other modifications and patterns of modifications can be found in, for example, PCT Publication No. WO2021/030778, which is hereby incorporated by reference. Other modifications and patterns of modifications can be found in, for example, PCT Publication No. WO2021/030763, which is hereby incorporated by reference.

In some embodiments, the sequence of the oligonucleotide molecule is at least 40%, 50%, 55%, 60%, 65%, 70%, 75%, 80%, 85%, 90%, 95%, 98%, 99%, or 99.5% complementary to a target sequence of CD40. In some embodiments, the target sequence of CD40 is a nucleic acid sequence of about 10-50 base pair length, about 15-50 base pair length, 15-40 base pair length, 15-30 base pair length, or 15-25 base pair length sequences in CD40, in which the first nucleotide of the target sequence starts at any nucleotide in CD40 mRNA transcript in the coding region, or in the 5′ or 3′-untranslated region (UTR). For example, the first nucleotide of the target sequence can be selected so that it starts at the nucleic acid location (nal, number starting from the 5′ end of the full length of CD40 mRNA, e.g., the 5′-end first nucleotide is nal 1) 1, nal 2, nal 3, nal 4, nal 5, nal 6, nal 7, nal 8, nal 9, nal 10, nal 11, nal 12, nal 13, nal 14, nal 15, nal 15, nal 16, nal 17, or any other nucleic acid location in the coding or noncoding regions (5′ or 3′-untranslated region) of CD40 mRNA. In some embodiments, the first nucleotide of the target sequence can be selected so that it starts at a location within, or between, nal 10-nal 15, nal 10-nal 20, nal 50-nal 60, nal 55-nal 65, nal 75-nal 85, nal 95-nal 105, nal 135-nal 145, nal 155-nal 165, nal 225-nal 235, nal 265-nal 275, nal 275-nal 245, nal 245-nal 255, nal 285-nal 335, nal 335-nal 345, nal 385-nal 395, nal 515-nal 525, nal 665-nal 675, nal 675-nal 685, nal 695-nal 705, nal 705-nal 715, nal 875-nal 885, nal 885-nal 895, nal 895-nal 905, nal 1035-nal 1045, nal 1045-nal 1055, nal 1125-nal 1135, nal 1135-nal 1145, nal 1145-nal 1155, nal 1155-nal 1165, nal 1125-nal 1135, nal 1155-nal 1165, nal 1225-nal 1235, nal 1235-nal 1245, nal 1275-nal 1245, nal 1245-nal 1255, nal 1265-nal 1275, nal 1125-nal 1135, nal 1155-nal 1165, nal 1225-nal 1235, nal 1235-nal 1245, nal 1275-nal 1245, nal 1245-nal 1255, nal 1265-nal 1275, nal 1275-nal 1285, nal 1335-nal 1345, nal 1345-nal 1355, nal 1525-nal 1535, nal 1535-nal 1545, nal 1605-nal 1615, nal 1615-c.1625, nal 1625-nal 1635, nal 1635-1735, nal 1735-1835, nal 1835-1935, nal. 1836-1856, nal 1935-2000, nal 2000-2100, nal 2100-2200, nal 2200-2260, nal 2260-2400, nal 2400-2500, nal 2500-2600, nal 2600-2700, nal 2700-2800, nal 2800-2500, nal 2500-2600, nal 2600-2700, nal 2700-2800, nal 2800-2860, etc. In some embodiments, the sequence of CD40 mRNA is provided as NCBI Reference Sequence: NM_001250.6 Homo sapiens CD40 molecule (CD40), transcript variant 1, mRNA:

(SEQ ID NO: 979)
AGTGGTCCTGCCGCCTGGTCTCACCTCGCTATGGTTCGTCTGCCTCTGCAGTGCGTC
CTCTGGGGCTGCTTGCTGACCGCTGTCCATCCAGAACCACCCACTGCATGCAGAGAAAAACA
GTACCTAATAAACAGTCAGTGCTGTTCTTTGTGCCAGCCAGGACAGAAACTGGTGAGTGACT
GCACAGAGTTCACTGAAACGGAATGCCTTCCTTGCGGTGAAAGCGAATTCCTAGACACCTGG
AACAGAGAGACACACTGCCACCAGCACAAATACTGCGACCCCAACCTAGGGCTTCGGGTCCA
GCAGAAGGGCACCTCAGAAACAGACACCATCTGCACCTGTGAAGAAGGCTGGCACTGTACGA
GTGAGGCCTGTGAGAGCTGTGTCCTGCACCGCTCATGCTCGCCCGGCTTTGGGGTCAAGCAG
ATTGCTACAGGGGTTTCTGATACCATCTGCGAGCCCTGCCCAGTCGGCTTCTTCTCCAATGT
GTCATCTGCTTTCGAAAAATGTCACCCTTGGACAAGCTGTGAGACCAAAGACCTGGTTGTGC
AACAGGCAGGCACAAACAAGACTGATGTTGTCTGTGGTCCCCAGGATCGGCTGAGAGCCCTG
GTGGTGATCCCCATCATCTTCGGGATCCTGTTTGCCATCCTCTTGGTGCTGGTCTTTATCAA
AAAGGTGGCCAAGAAGCCAACCAATAAGGCCCCCCACCCCAAGCAGGAACCCCAGGAGATCA
ATTTTCCCGACGATCTTCCTGGCTCCAACACTGCTGCTCCAGTGCAGGAGACTTTACATGGA
TGCCAACCGGTCACCCAGGAGGATGGCAAAGAGAGTCGCATCTCAGTGCAGGAGAGACAGTG
AGGCTGCACCCACCCAGGAGTGTGGCCACGTGGGCAAACAGGCAGTTGGCCAGAGAGCCTGG
TGCTGCTGCTGCTGTGGCGTGAGGGTGAGGGGCTGGCACTGACTGGGCATAGCTCCCCGCTT
CTGCCTGCACCCCTGCAGTTTGAGACAGGAGACCTGGCACTGGATGCAGAAACAGTTCACCT
TGAAGAACCTCTCACTTCACCCTGGAGCCCATCCAGTCTCCCAACTTGTATTAAAGACAGAG
GCAGAAGTTTGGTGGTGGTGGTGTTGGGGTATGGTTTAGTAATATCCACCAGACCTTCCGAT
CCAGCAGTTTGGTGCCCAGAGAGGCATCATGGTGGCTTCCCTGCGCCCAGGAAGCCATATAC
ACAGATGCCCATTGCAGCATTGTTTGTGATAGTGAACAACTGGAAGCTGCTTAACTGTCCAT
CAGCAGGAGACTGGCTAAATAAAATTAGAATATATTTATACAACAGAATCTCAAAAACACTG
TTGAGTAAGGAAAAAAAGGCATGCTGCTGAATGATGGGTATGGAACTTTTTAAAAAAGTACA
TGCTTTTATGTATGTATATTGCCTATGGATATATGTATAAATACAATATGCATCATATATTG
ATATAACAAGGGTTCTGGAAGGGTACACAGAAAACCCACAGCTCGAAGAGTGGTGACGTCTG
GGGTGGGGAAGAAGGGTCTGGGGGAGGGTTGGTTAAAGGGAGATTTGGCTTTCCCATAATGC
TTCATCATTTTTCCCAAAAGGAGAGTGAATTCACATAATGCTTATGTAATTAAAAAATCATC
AAACATGTAAAAA.

In some embodiments, the antisense strand of the dsRNA agent is 100% complementary to a target RNA to hybridize thereto and inhibits its expression through RNA interference. The target RNA can be any RNA expressed in a cell. In another embodiment, the cell is a tumor cell, a liver cell, a muscle cell, an immune cell, a heart cell, or a cell of the central nervous system. In another embodiment, the antisense strand of the dsRNA agent is at least 99%, at least 98%, at least 97%, at least 96%, 95%, at least 90%, at least 85%, at least 80%, at least 75%, at least 70%, at least 65%, at least 60%, at least 55%, or at least 50% complementary to a target RNA. In some embodiments, the target RNA is CD40 RNA. In some embodiments, the siRNA molecule is an siRNA that reduces mRNA expression of CD40. In some embodiments, the siRNA molecule is an siRNA that reduces mRNA expression of CD40 and does not reduce expression of other RNAs by more than 50% in an assay described herein at a concentration of no more than 200 nm as described herein.

In some embodiments, the siRNA is linked to a protein, such as a FN3 domain. The siRNA can be linked to multiple FN3 domains that bind to the same target protein or different target proteins. In some embodiments, the linker is attached to the sense strand, which is used to facilitate the linkage of the sense strand to the FN3 domain.

In some embodiments, compositions are provided herein having a formula of (X₁)˜-(X₂)_{q}—(X₃)_{y}-L-X₄, wherein X₁ is a first FN3 domain, X₂ is A second FN3 domain, X₃ is a third FN3 domain or half-life extender molecule, L is a linker, and X₄ is a nucleic acid molecule, such as, but not limited to an siRNA molecule, wherein n, q, and y are each independently 0 or 1. In some embodiments, X₁, X₂, and X₃ bind to different target proteins. In some embodiments, y is 0. In some embodiments, n is 1, q is 0, and y is 0. In some embodiments, n is 1, q is 1, and y is 0. In some embodiments, n is 1, q is 1, and y is 1. In some embodiments, X₃ increases the half-life of the molecule as a whole as compared to a molecule without X₃. In some embodiments, the half-life extending moiety is an FN3 domain that binds to albumin. Examples of such FN3 domains include, but are not limited to, those described in U.S. Patent Application Publication No. 2017/0348397 and U.S. Pat. No. 9,156,887, which are hereby incorporated by reference in their entireties. The FN3 domains may incorporate other subunits, for example, via covalent interaction. In some embodiments, the FN3 domains further comprise a half-life extending moiety. Exemplary half-life extending moieties are albumin, albumin variants, albumin-binding proteins and/or domains, an aliphatic chain that binds to serum proteins, transferrin and fragments and analogues thereof, and Fe regions. Amino acid sequences of the human Fc regions are well known, and include IgG1, IgG2, IgG3, IgG4, IgM, IgA and IgE Fc regions. In some embodiments, the FN3 domains may incorporate a second FN3 domain that binds to a molecule that extends the half-life of the entire molecule, such as, but not limited to, any of the half-life extending moieties described herein. In some embodiments, the second FN3 domain binds to albumin, albumin variants, albumin-binding proteins and/or domains, and fragments and analogues thereof.

In some embodiments, compositions are provided herein having a formula of (X₁)—(X₂)-L-(X₄), wherein X₁ is a first FN3 domain, X₂ is a second FN3 domain, L is a linker, and X₄ is a nucleic acid molecule. In some embodiments, X₄ is an siRNA molecule. In some embodiments, X₁ is a FN3 domain that binds to CD71. In some embodiments, X₂ is a FN3 domain that binds to CD71. In some embodiments X₁ and X₂ do not bind to the same target protein. In some embodiments, X₁ and X₂ bind to the same target protein, but at different binding sites on the protein. In some embodiments, X₁ and X₂ bind to the same target protein. In some embodiments, X₁ and X₂ are FN3 domains that bind to CD71. In some embodiments, the composition does not comprise (e.g. is free of) a compound or protein that binds to ASGPR.

In some embodiments, compositions are provided herein having a formula of C—(X₁)ₙ—(X₂)_{q}[L-X₄]—(X₃)_{y}, wherein X₁ is a first FN3 domain; X₂ is a second FN3 domain; X₃ is a third FN3 domain or half-life extender molecule; L is a linker; X₄ is an oligonucleotide molecule; and C is a polymer, wherein n, q, and y are each independently 0 or 1, are provided.

In some embodiments, compositions are provided herein having a formula of (X₁)ₙ—(X₂)_{q}[L-X₄]—(X₃)_{y}—C, wherein X₁ is a first FN3 domain; X₂ is a second FN3 domain; X₃ is a third FN3 domain or half-life extender molecule; L is a linker; X₄ is an oligonucleotide molecule; and C is a polymer, wherein n, q, and y are each independently 0 or 1, are provided.

In some embodiments, compositions are provided herein having a formula of C—(X₁)ₙ—(X₂)_{q}[L-X₄]L-(X₃)_{y}, wherein X₁ is a first FN3 domain; X₂ is a second FN3 domain; X₃ is a third FN3 domain or half-life extender molecule; L is a linker; X₄ is an oligonucleotide molecule; and C is a polymer, wherein n, q, and y are each independently 0 or 1, are provided.

In some embodiments, compositions are provided herein having a formula of (X₁)˜-(X₂)_{q}[L-X₄]L-(X₃)_{y}—C, wherein X₁ is a first FN3 domain; X₂ is a second FN3 domain; X₃ is a third FN3 domain or half-life extender molecule; L is a linker; X₄ is an oligonucleotide molecule; and C is a polymer, wherein n, q, and y are each independently 0 or 1, are provided.

In some embodiments, compositions or complexes are provided having a formula of A₁-B₁, wherein A₁ has a formula of C-L₁-X_{S} and B₁ has a formula of X_{AS}-L₂-F₁, wherein:
C is a polymer, such as PEG;
L₁ and L₂ are each, independently, a linker;
X_{S} is a 5′ to 3′ oligonucleotide sense strand of a double stranded siRNA molecule;
X_{AS} is a 3′ to 5′ oligonucleotide antisense strand of a double stranded siRNA molecule;
F₁ is a polypeptide comprising at least one FN3 domain;
wherein X_{S} and X_{AS} form a double stranded oligonucleotide molecule to form the composition/complex.

In some embodiments, compositions or complexes are provided having a formula of A₁-B₁, wherein A₁ has a formula of X_{S} and B₁ has a formula of X_{AS}-L₂-F₁.

In some embodiments, compositions or complexes are provided having a formula of A₁-B₁, wherein A₁ has a formula of C-L₁-X_{S} and B₁ has a formula of X_{AS}.

In some embodiments, the sense strand is a sense strand as provided for herein. In some embodiments, the antisense strand is an antisense strand as provided for herein. In some embodiments, the sense and antisense strand form a double stranded siRNA molecule that targets CD40. In some embodiments, the double stranded oligonucleotide is about 21-23 nucleotides base pairs in length. In certain embodiments, C is optional.

In some embodiments, compositions or complexes are provided having a formula of A₁-B₁, wherein A₁ has a formula of F₁-L₁-X_{S} and B₁ has a formula of X_{AS}-L₂-C, wherein:
F₁ is a polypeptide comprising at least one FN3 domain;
L₁ and L₂ are each, independently, a linker;
C is a polymer, such as PEG;
X_{S} is a 5′ to 3′ oligonucleotide sense strand of a double stranded siRNA molecule;
X_{AS} is a 3′ to 5′ oligonucleotide antisense strand of a double stranded siRNA molecule;
wherein X_{S} and X_{AS} form a double stranded oligonucleotide molecule to form the composition/complex. In certain embodiments, C is optional.

In some embodiments, compositions or complexes are provided having a formula of A₁-B₁, wherein A₁ has a formula of X_{S} and B₁ has a formula of X_{AS}-L₂-C.

In some embodiments, compositions or complexes are provided having a formula of A₁-B₁, wherein A₁ has a formula of F₁-L₁-X_{S} and B₁ has a formula of X_{AS}.

In some embodiments, A₁ and B₁ interact with each other through hydrogen bonding. In some embodiments, A₁ and B₁ interact with each other through Watson-Crick base pairing.

In some embodiments, compositions described a polymer (polymer moiety C, or just C). In some embodiments, C can be a molecule that extends the half-life of the molecule. In some embodiments, the polymer is a natural or synthetic polymer, consisting of long chains of branched or unbranched monomers, and/or cross-linked network of monomers in two or three dimensions In some instances, the polymer includes a polysaccharide, lignin, rubber, or polyalkylene oxide (e.g., polyethylene glycol). In some instances, the at least one polymer includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylenterephthalat (PET, PETG), polyethylene terephthalate (PETE), polytetramethylene glycol (PTG), or polyurethane as well as mixtures thereof. As used herein, a mixture refers to the use of different polymers within the same compound as well as in reference to block copolymers. In some cases, block copolymers are polymers wherein at least one section of a polymer is build up from monomers of another polymer. In some instances, the polymer comprises polyalkylene oxide. In some instances, the polymer comprises PEG. In some instances, the polymer comprises polyethylene imide (PEI) or hydroxy ethyl starch (HES).

In some embodiments, C is a PEG moiety. In some embodiments, the PEG moiety is conjugated at the 5′ terminus of the oligonucleotide molecule while the binding moiety is conjugated at the 3′ terminus of the oligonucleotide molecule. In some embodiments, the PEG moiety is conjugated at the 3′ terminus of the oligonucleotide molecule while the binding moiety is conjugated at the 5′ terminus of the oligonucleotide molecule. In some embodiments, the PEG moiety is conjugated to an internal site of the oligonucleotide molecule. In some embodiments, the PEG moiety, the binding moiety, or a combination thereof, are conjugated to an internal site of the oligonucleotide molecule. In some embodiments, the conjugation is a direct conjugation. In some embodiments, the conjugation is via native ligation.

In some embodiments, the polyalkylene oxide (e.g., PEG) is a polydisperse or monodisperse compound. In some embodiments, polydisperse material comprises disperse distribution of different molecular weight of the material, characterized by mean weight (weight average) size and dispersity. In some embodiments, the monodisperse PEG comprises one size of molecules. In some embodiments, C is poly- or monodispersed polyalkylene oxide (e.g., PEG) and the indicated molecular weight represents an average of the molecular weight of the polyalkylene oxide, e.g., PEG, molecules.

In some embodiments, the molecular weight of the polyalkylene oxide (e.g., PEG) is about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da.

In some embodiments, C is polyalkylene oxide (e.g., PEG) and has a molecular weight of about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da. In some embodiments, C is PEG and has a molecular weight of about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da. In some embodiments, the molecular weight of C is about 200 Da. In some embodiments, the molecular weight of C is about 300 Da. In some embodiments, the molecular weight of C is about 400 Da. In some embodiments, the molecular weight of C is about 500 Da. In some embodiments, the molecular weight of C is about 600 Da. In some embodiments, the molecular weight of C is about 700 Da. In some embodiments, the molecular weight of C is about 800 Da. In some embodiments, the molecular weight of C is about 900 Da. In some embodiments, the molecular weight of C is about 1000 Da. In some embodiments, the molecular weight of C is about 1100 Da. In some embodiments, the molecular weight of C is about 1200 Da. In some embodiments, the molecular weight of C is about 1300 Da. In some embodiments, the molecular weight of C is about 1400 Da. In some embodiments, the molecular weight of C is about 1450 Da. In some embodiments, the molecular weight of C is about 1500 Da. In some embodiments, the molecular weight of C is about 1600 Da. In some embodiments, the molecular weight of C is about 1700 Da. In some embodiments, the molecular weight of C is about 1800 Da. In some embodiments, the molecular weight of C is about 1900 Da. In some embodiments, the molecular weight of C is about 2000 Da. In some embodiments, the molecular weight of C is about 2100 Da. In some embodiments, the molecular weight of C is about 2200 Da. In some embodiments, the molecular weight of C is about 2300 Da. In some embodiments, the molecular weight of C is about 2400 Da. In some embodiments, the molecular weight of C is about 2500 Da. In some embodiments, the molecular weight of C is about 2600 Da. In some embodiments, the molecular weight of C is about 2700 Da. In some embodiments, the molecular weight of C is about 2800 Da. In some embodiments, the molecular weight of C is about 2900 Da. In some embodiments, the molecular weight of C is about 3000 Da. In some embodiments, the molecular weight of C is about 3250 Da. In some embodiments, the molecular weight of C is about 3350 Da. In some embodiments, the molecular weight of C is about 3500 Da. In some embodiments, the molecular weight of C is about 3750 Da. In some embodiments, the molecular weight of C is about 4000 Da. In some embodiments, the molecular weight of C is about 4250 Da. In some embodiments, the molecular weight of C is about 4500 Da. In some embodiments, the molecular weight of C is about 4600 Da. In some embodiments, the molecular weight of C is about 4750 Da. In some embodiments, the molecular weight of C is about 5000 Da. In some embodiments, the molecular weight of C is about 5500 Da. In some embodiments, the molecular weight of C is about 6000 Da. In some embodiments, the molecular weight of C is about 6500 Da. In some embodiments, the molecular weight of C is about 7000 Da. In some embodiments, the molecular weight of C is about 7500 Da. In some embodiments, the molecular weight of C is about 8000 Da. In some embodiments, the molecular weight of C is about 10,000 Da. In some embodiments, the molecular weight of C is about 12,000 Da. In some embodiments, the molecular weight of C is about 20,000 Da. In some embodiments, the molecular weight of C is about 35,000 Da. In some embodiments, the molecular weight of C is about 40,000 Da. In some embodiments, the molecular weight of C is about 50,000 Da. In some embodiments, the molecular weight of C is about 60,000 Da. In some embodiments, the molecular weight of C is about 100,000 Da.

In some embodiments, the polyalkylene oxide (e.g., PEG) is a discrete PEG, in which the discrete PEG is a polymeric PEG comprising more than one repeating ethylene oxide units. In some embodiments, a discrete PEG (dPEG) comprises from 2 to 60, from 2 to 50, or from 2 to 48 repeating ethylene oxide units. In some embodiments, a dPEG comprises about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units. In some embodiments, a dPEG comprises about 2 or more repeating ethylene oxide units. In some embodiments, a dPEG comprises about 3 or more repeating ethylene oxide units. In some embodiments, a dPEG comprises about 4 or more repeating ethylene oxide units. In some embodiments, a dPEG comprises about 5 or more repeating ethylene oxide units. In some embodiments, a dPEG comprises about 6 or more repeating ethylene oxide units. In some embodiments, a dPEG comprises about 7 or more repeating ethylene oxide units. In some embodiments, a dPEG comprises about 8 or more repeating ethylene oxide units. In some embodiments, a dPEG comprises about 9 or more repeating ethylene oxide units. In some embodiments, a dPEG comprises about 10 or more repeating ethylene oxide units. In some embodiments, a dPEG comprises about 11 or more repeating ethylene oxide units. In some embodiments, a dPEG comprises about 12 or more repeating ethylene oxide units. In some embodiments, a dPEG comprises about 13 or more repeating ethylene oxide units. In some embodiments, a dPEG comprises about 14 or more repeating ethylene oxide units. In some embodiments, a dPEG comprises about 15 or more repeating ethylene oxide units. In some embodiments, a dPEG comprises about 16 or more repeating ethylene oxide units. In some embodiments, a dPEG comprises about 17 or more repeating ethylene oxide units. In some embodiments, a dPEG comprises about 18 or more repeating ethylene oxide units. In some embodiments, a dPEG comprises about 19 or more repeating ethylene oxide units. In some embodiments, a dPEG comprises about 20 or more repeating ethylene oxide units. In some embodiments, a dPEG comprises about 22 or more repeating ethylene oxide units. In some embodiments, a dPEG comprises about 24 or more repeating ethylene oxide units. In some embodiments, a dPEG comprises about 26 or more repeating ethylene oxide units. In some embodiments, a dPEG comprises about 28 or more repeating ethylene oxide units. In some embodiments, a dPEG comprises about 30 or more repeating ethylene oxide units. In some embodiments, a dPEG comprises about 35 or more repeating ethylene oxide units. In some embodiments, a dPEG comprises about 40 or more repeating ethylene oxide units. In some embodiments, a dPEG comprises about 42 or more repeating ethylene oxide units. In some embodiments, a dPEG comprises about 48 or more repeating ethylene oxide units. In some embodiments, a dPEG comprises about 50 or more repeating ethylene oxide units. In some cases, a dPEG is synthesized as a single molecular weight compound from pure (e.g., about 95%, 98%, 99%, or 99.5%) staring material in a step-wise fashion. In some cases, a dPEG has a specific molecular weight, rather than an average molecular weight. In some cases, a dPEG described herein is a dPEG from Quanta Biodesign, LMD.

In some embodiments, C is an albumin binding domain. In some embodiments, the albumin binding domain specifically binds to serum albumin, e.g., human serum albumin (HSA), to prolong the half-life of the domain or of another therapeutic to which the albumin-binding domain is associated with or linked to. In some embodiments, the human serum albumin-binding domain comprises an initiator methionine (Met) linked to the N-terminus of the molecule. In some embodiments, the human serum albumin-binding domain comprises a cysteine (Cys) linked to a C-terminus or the N-terminus of the domain. The addition of the N-terminal Met and/or the C-terminal Cys may facilitate expression and/or conjugation to another molecule, which can be another half-life extending molecule, such as PEG, a Fc region, and the like.

In some embodiments, the albumin binding domain comprises the amino acid sequence of SEQ ID NOs: 5, 6, 7, 8, 9, 10, 11, 12, 13, 14, 15, 16, 17, 18, 19, 20, 21, 22, or 23, provided in Table 1. In some embodiments, the albumin binding domain (protein) is isolated. In some embodiments, the albumin binding domain comprises an amino acid sequence that is at least, or is, 85%, 86%, 87%, 88%, 89%, 90%, 91%, 92%, 93%, 94%, 95%, 96%, 97%, 98%, or 99% identical to the amino acid sequence of SEQ ID NOs: 5, 6, 7, 8, 9, 10, 11, 12, 13, 14, 15, 16, 17, 18, 19, 20, 21, 22, or 23. In some embodiments, the albumin binding domain comprises an amino acid sequence that is at least, or is, 85%, 86%, 87%, 88%, 89%, 90%, 901%, 92%, 93%, 94%, 95%, 96%, 97%, 98%, or 99% identical to the amino acid sequence of SEQ ID NOs: 5, 6, 7, 8, 9, 10, 11, 12, 13, 14, 15, 16, 17, 18, 19, 20, 21, 22, or 23 provided that the protein has a substitution that corresponds to position 10 of SEQ ID NOs: 5, 6, 7, 8, 9, 10, 11, 12, 13, 14, 15, 16, 17, 18, 19, 20, 21, 22, or 23. In some embodiments, the substitution is A10V. In some embodiments, the substitution is A10G, A10L, A10I, A10T, or A10S. In some embodiments, the substitution at position 10 is any naturally occurring amino acid. In some embodiments, the isolated albumin binding domain comprises an amino acid sequence that has 1, 2, 3, 4, 5, 6, 7, 8, 9, 10, 11, 12, 13, or 14 substitutions when compared to the amino acid sequence of SEQ ID NOs: 5, 6, 7, 8, 9, 10, 11, 12, 13, 14, 15, 16, 17, 18, 19, 20, 21, 22, or 23. In some embodiments, the substitution is at a position that corresponds to position 10 of SEQ ID NOs: 5, 6, 7, 8, 9, 10, 11, 12, 13, 14, 15, 16, 17, 18, 19, 20, 21, 22, or 23. In some embodiments, FN3 domains provided comprise a cysteine residue in at least one residue position corresponding to residue positions 6, 11, 22, 25, 26, 52, 53, 61, 88 or positions 6, 8, 10, 11, 14, 15, 16, 20, 30, 34, 38, 40, 41, 45, 47, 48, 53, 54, 59, 60, 62, 64, 70, 88, 89, 90, 91, or 93 of SEQ ID NOs: 5, 6, 7, 8, 9, 10, 11, 12, 13, 14, 15, 16, 17, 18, 19, 20, 21, 22, or 23, or at a C-terminus. Although the positions are listed in a series, each position can also be chosen individually. In some embodiments, the cysteine is at a position that corresponds to position 6, 53, or 88. In some embodiments, additional examples of albumin binding domains can be found in U.S. Pat. No. 10,925,932, which is hereby incorporated by reference in its entirety. In some embodiments, additional examples of albumin binding domains can be found in U.S. Pat. Nos. 8,969,289, 9,540,424, 10,221,438, 10,934,572, 10,442,851, 11,203,630, 10,766,946, 11,434,275; and in U.S.

Publication Nos. 2022/0204589 and 2023/0145413; each of which is hereby incorporated by reference in its entirety.

TABLE 1
Albumin-binding Domain Sequences

| SEQ ID | |
|---|---|
| NO: | SEQUENCE |
| 5 | MLPAPKNLVASRVTEDSARLSWTAPDAAFDSFNIAYWEPGIGGEAIWLRVPGS |
| | ERSYDLTGLKPGTEYKVWIHGVKGGASSPPLIARFTT |
| 6 | MLPAPKNLVVSRVTEDSARLSWTAPDAAFDSFNIAYWEPGIGGEAIWLRVPGS |
| | ERSYDLIGLKPGTEYKVWIHGVKGGASSPPLIARFTT |
| 7 | MLPAPKNLVVSRVTEDSARLSWTAPDAAFDSFAIAYWEPGIGGEAIWLRVPGS |
| | ERSYDLTGLKPGTEYKVWIHGVKGGASSPPLIARFTT |
| 8 | MLPAPKNLVVSRVTEDSARLSWTAPDAAFDSFNISYWEPGIGGEAIWLRVPGS |
| | ERSYDLTGLKPGTEYKVWIHGVKGGASSPPLIARFTT |
| 9 | MLPAPKNLVVSRVTEDSARLSWTAPDAAFDSENIAYAEPGIGGEAIWLRVPGS |
| | RSYDLTGLKPGTEYKVWIHGVKGGASSPPLIARFTT |
| 10 | MLPAPKNLVVSRVTEDSARLSWTAPDAAFDSENIAYWEAGIGGEAIWLRVPGS |
| | ERSYDLTGLKPGTEYKVWIHGVKGGASSPPLIARFTT |
| 11 | MLPAPKNLVVSRVTEDSARLSWTAPDAAFDSENIAYWEPAIGGEAIWLRVPGS |
| | ERSYDLTGLKPGTEYKVWIHGVKGGASSPPLIARFTT |
| 12 | MLPAPKNLVVSRVTEDSARLSWTAPDAAFDSFNIAYWEPGAGGEAIWLRVPGS |
| | ERSYDLTGLKPGTEYKVWIHGVKGGASSPPLIARFTT |
| 13 | MLPAPKNLVVSRVTEDSARLSWTAPDAAFDSFNIAYWEPGIAGEAIWLRVPGS |
| | ERSYDLTGLKPGTEYKVWIHGVKGGASSPPLIARFTT |
| 14 | MLPAPKNLVVSRVTEDSARLSWTAPDAAFDSENIAYWEPGIGGEAIALRVPGS |
| | ERSYDLTGLKPGTEYKVWIHGVKGGASSPPLIARFTT |
| 15 | MLPAPKNLVVSRVTEDSARLSWTAPDAAFDSENIAYWEPGIGGEAIWLAVPGS |
| | ERSYDLTGLKPGTEYKVWIHGVKGGASSPPLIARFTT |
| 16 | MLPAPKNLVVSRVTEDSARLSWTAPDAAFDSFNIAYWEPGIGGEAIWLRVPGS |
| | ERSYDLTGLKPGTEYAVWIHGVKGGASSPPLIARFTT |
| 17 | MLPAPKNLVVSRVTEDSARLSWTAPDAAFDSFNIAYWEPGIGGEAIWLRVPGS |
| | ERSYDLTGLKPGTEYKVAIHGVKGGASSPPLIARFTT |
| 18 | MLPAPKNLVVSRVTEDSARLSWTAPDAAFDSENIAYWEPGIGGEAIWLRVPGS |
| | ERSYDLTGLKPGTEYKVWIAGVKGGASSPPLIARFTT |
| 19 | MLPAPKNLVVSRVTEDSARLSWTAPDAAFDSENIAYWEPGIGGEAIWLRVPGS |
| | ERSYDLTGLKPGTEYKVWIHGVKGGSSSPPLIARFTT |
| 20 | MLPAPKNLVVSRVTEDSARLSWTAPDAAFDSFNIAYWEPGIGGEAIWLRVPGS |
| | ERSYDLTGLKPGTEYKVWIHGVKGGAASPPLIARFTT |
| 21 | MLPAPKNLVVSRVTEDSARLSWTAPDAAFDSFNIAYWEPGIGGEAIWLRVPGS |
| | ERSYDLTGLKPGTEYKVWIHGVKGGASSAPLIARFTT |
| 22 | MLPAPKNLVVSRVTEDSARLSWTAPDAAFDSFNIAYWEPGIGGEAIWLRVPGS |
| | ERSYDLTGLKPGTEYKVWIHGVKGGASSPPLAARFTT |
| 23 | MLPAPKNLVVSRVTEDSARLSWTAPDAAFDSENIAYWEPGIGGEAIWLRVPGS |
| | ERSYDLTGLKPGTEYKVWIHGVKGGASSPPLIAAFTT |

In some embodiments, C can also be Endoporter, INF-7, TAT, polyarginine, polylysine, or an amphipathic peptide. These moieties can be used in place of or in addition to other half-life extending moieties provided for herein. In some embodiments, C can be a molecule that delivers the complex into the cell, the endosome, or the ER; said molecules are selected from those peptides listed in Table 2.

TABLE 2

| SEQ ID | | |
|---|---|---|
| NO: | NAME | SEQUENCE |
| 24 | TAT | RKKRRQRRR |
| 25 | Penetratin | RQIKIWFQNRRMKWKK |
| 26 | Transportan | GWTLNSAGYLLGKINKALAALAKKIL |
| 27 | MAP | KLALKLALKALKAALKLA |
| 28 | Pep-1 | KETWWETWWTEWSQPKKKRKV |
| 29 | KDEL | KDEL |
| 30 | GALA | WEAALAEALAELAEHLAEALAEALEALAA |
| 31 | HA2 | GDIMGEWGNEIFGAIAGELGC |
| 32 | Aurine 1.2 | GLFDIIKKIAESF |
| 33 | MPG | GALFLGWLGAAGSTMGAPKSKRKV |
| 34 | TP-10 | AGYLLGKINLKALAALAKKIL |
| 35 | EB-1 | LIRLWSHLIHIWFQNRRLKWKKK |
| 36 | HA2- | GLFGAIAGFIENGWEGMIDGRQIKIWFQNRRMKWKK |
| | Penetratin | |
| 37 | Endosomolytic | FFKKLAHALHLLALLALHLAHALKKA |
| 38 | Endosomolytic | LFEAIEGFIENGWGMIDGWYG |
| 39 | Endosomolytic | LFEAIEGFIENGWEGMIDGWYGRKKRRQRRR |
| 40 | Endosomolytic | IGAVLKVLTTGLPALISWIKRKRQQ |
| 41 | ER Targeting | MKLAVTLTLVTLALSSSSASA |
| 42 | ER Targeting | RLIEDICLPRWGCLWEDDKDEL |
| 43 | ER Targeting | MIRTLLLSTLVAGALSK |
| 44 | ER Targeting | ILSSLTVTOLLRRLHQWIK |
| 45 | ER Targeting | MIRTLLLSTLVAGALSKDEL |

In some embodiments, L₁ is any linker that can be used to link the polymer C to the sense strand X_{S} or to link the polypeptide of F₁ to the sense strand X_{S}. In some embodiments, L₁ has a formula of:

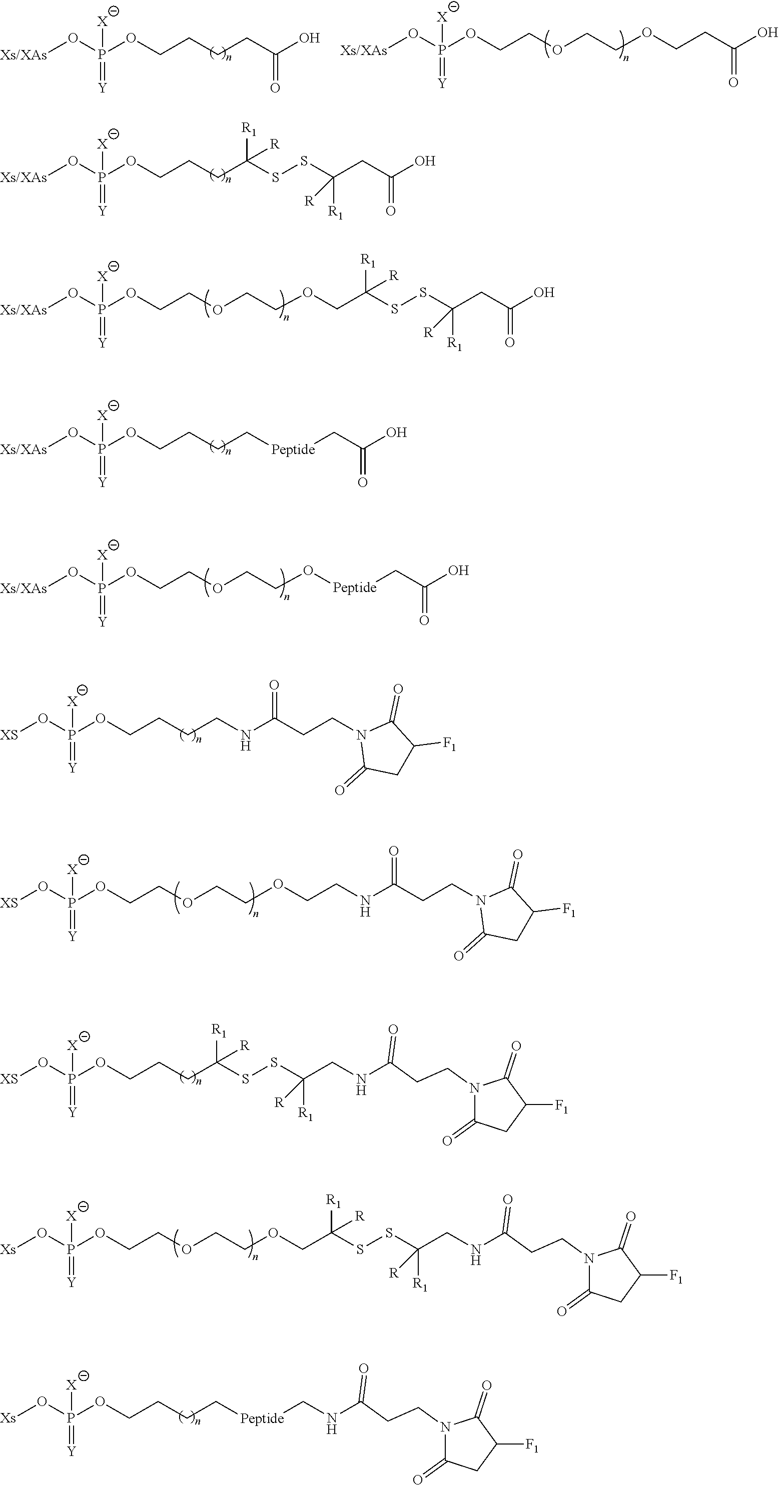

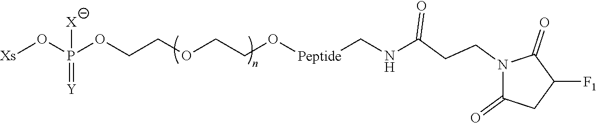

wherein X_{S}, X_{AS}, and F₁ are as defined above.

In some embodiments, n=0-20. In some embodiments, R and R1 are independently methyl. In some embodiments, R and R1 are independently present or both are absent. In some embodiments, X and Y are independently S. In some embodiments, X and Y are independently present or absent. In some embodiments, Peptide is an enzymatically cleavable peptide, such as, but not limited to, Val-Cit, Val-Ala, etc.

In some embodiments, L₂ is any linker that can be used to link the polypeptide of F1 to the antisense strand X_{AS} or to link the polymer C to the antisense strand X_{AS}. In some embodiments, L₂ has a formula of in the complex of:

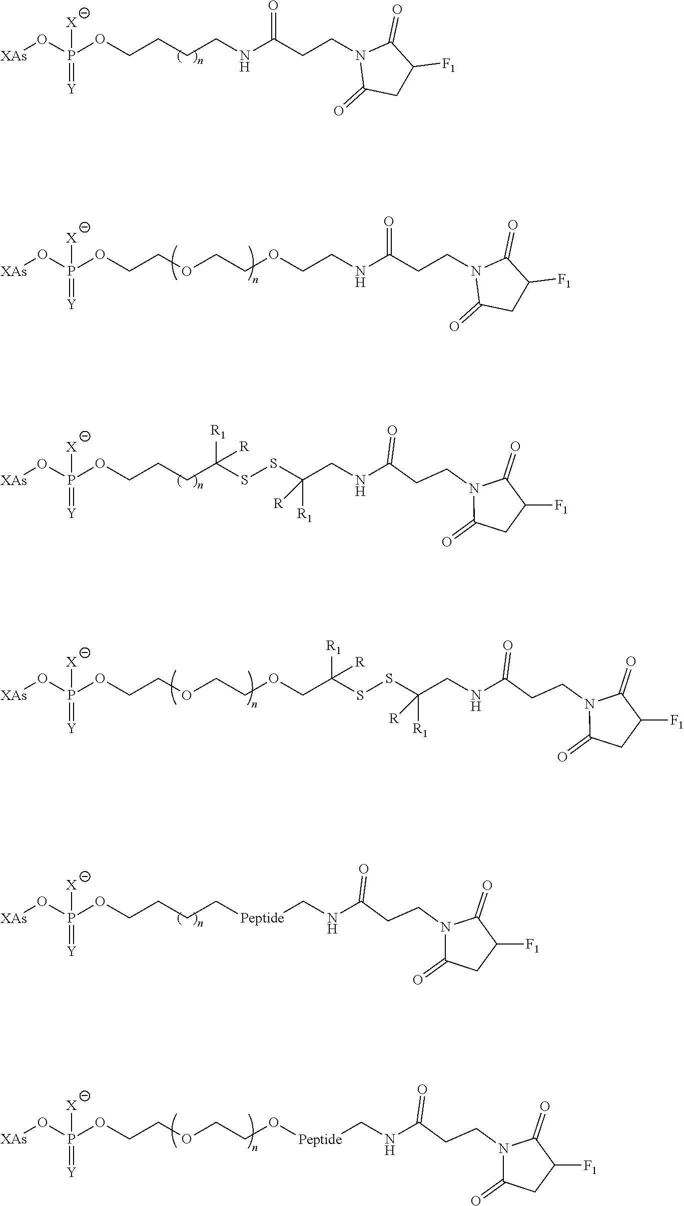

wherein X_{AS} and F₁ are as defined above.

In some embodiments, n=0-20. In some embodiments, R and R1 are independently methyl. In some embodiments, R and R1 are independently present or both are absent. In some embodiments, X and Y are independently S. In some embodiments, X and Y are independently present or absent. In some embodiments, Peptide is an enzymatically cleavable peptide, such as, but not limited to, Val-Cit, Val-Ala, etc.

In some embodiments, the linker is covalently attached to F1 through a cysteine residue present on F1, which can be illustrated as follows:

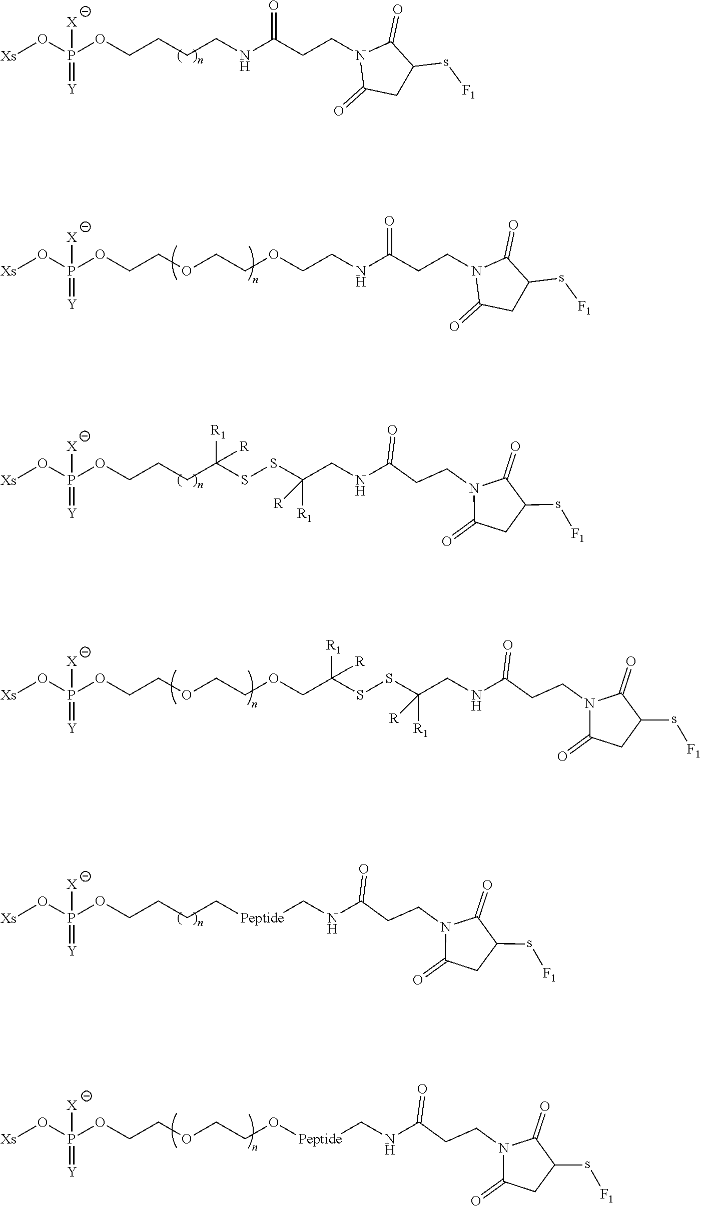

wherein X_{S} is a 5′ to 3′ oligonucleotide sense strand of a double stranded siRNA molecule; X_{AS} is a 3′ to 5′ oligonucleotide antisense strand of a double stranded siRNA molecule; and F₁ is a polypeptide comprising at least one FN3 domain, wherein X_{S} and X_{AS} form a double stranded siRNA molecule.

In some embodiments, A₁-B₁ has a formula of:

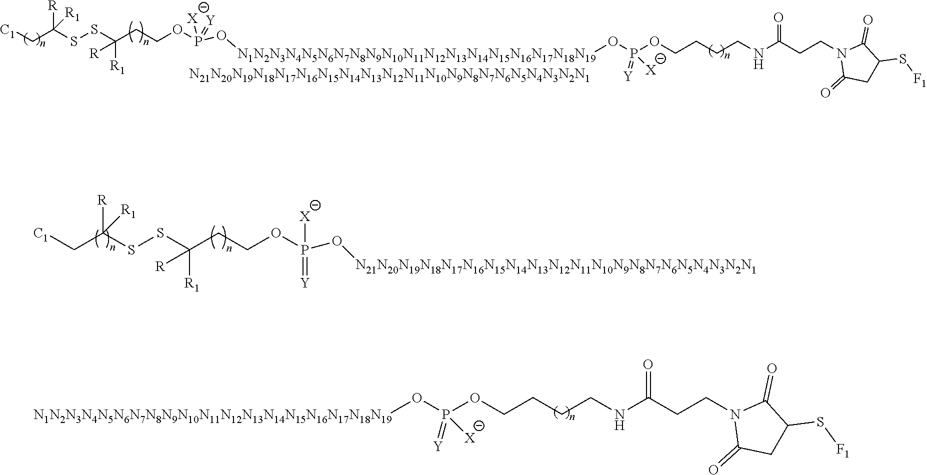

wherein C is the polymer, such as PEG, Endoporter, INF-7, TAT, polyarginine, polylysine, an amphipathic peptide, or as provided for herein; and F₁ is a polypeptide comprising at least one FN3 domain. The sense and antisense strands are represented by the “N” notations, wherein each nucleotide represented by N, is independently, A, U, C, or G or a modified nucleobase, such as those provided for herein. The N₁ nucleotides of the sense strand and the antisense strand represent the 5′ end of the respective strands. For clarity, although Formula III utilizes N₁, N₂, N₃, etc., in both the sense and the antisense strand, the nucleotide bases do not need to be the same and are not intended to be the same. The siRNA that is illustrated in Formula III would be complementary to a target sequence. For example, in some embodiments, the sense strand comprises a 2′O-methyl modified nucleotide with a phosphorothioate (PS) modified backbone at N₁ and N₂, a 2′-fluoro modified nucleotide at N₃, N₇, N₈, N₉, N₁₂, and N₁₇, and a 2′O-methyl modified nucleotide at N₄, N₅, N₆, N₁₀, N₁₁, N₁₃, N₁₄, N₁₅, N₁₆, Nis, and N₁₉.

In some embodiments, the antisense strand comprises a vinyl phosphonate moiety attached to N₁, a 2′fluoro-modified nucleotide with a phosphorothioate (PS) modified backbone at N₂, a 2′O-methyl modified nucleotide at N₃, N₄, N₅, N₆, N₇, N₈, N₉, N₁₀, N₁₁, N₁₂, N₁₃, N₁₅, N₁₆, N₁₇, N₁₈, and N₁₉, a 2′fluoro-modified nucleotide at N₁₄, and a 2′O-methyl modified nucleotide with a phosphorothioate (PS) modified backbone at N₂₀ and N₂₁.

In some embodiments, a compound having a formula of:

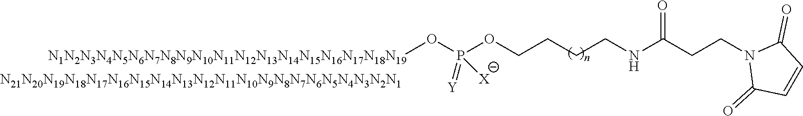

is provided.

In some embodiments, a compound having a formula of:

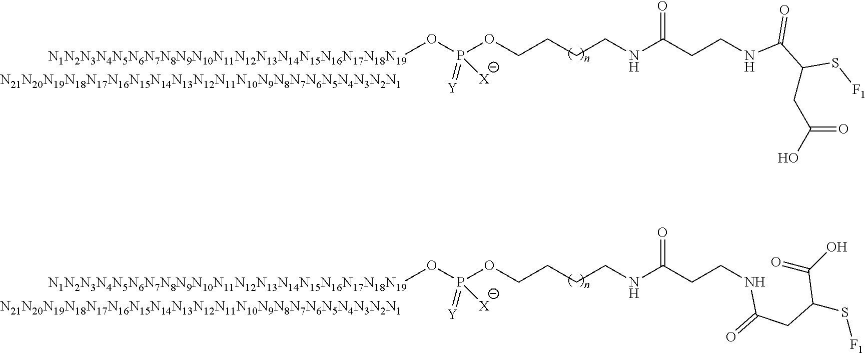

is provided, wherein F₁ is a polypeptide comprising at least one FN3 domain and is conjugated to a linker. The linker illustrated above, is a non-limiting example, and other types of linkers can be used.

In some embodiments, F₁ comprises polypeptide having a formula of (X₁)ₙ—(X₂)_{q}—(X₃)_{y}, wherein X₁ is a first FN3 domain; X₂ is second FN3 domain; X₃ is a third FN3 domain or half-life extender molecule; wherein n, q, and y are each independently 0 or 1, provided that at least one of n, q, and y is 1. In some embodiments, n, q, and y are each 1. In some embodiments, n and q are 1 and y is 0. In some embodiments n and y are 1 and q is 0.

In some embodiments, X₁ is a CD71 binding FN3 domain, such as one provided herein. In some embodiments, X₂ is a CD71 binding FN3 domain. In some embodiments, X₁ and X₂ are different CD71 binding FN3 domains. In some embodiments, the binding domains are the same. In some embodiments, X₃ is a FN3 domain that binds to human serum albumin. In some embodiments, X₃ is an Fc domain without effector function that extends the half-life of a protein. In some embodiments, X₁ is a first CD71 binding FN3 domain, X₂ is a second CD71 binding FN3 domain, and X₃ is an albumin binding FN3 domain. Examples of such polypeptides are provided herein and below. In some embodiments, compositions are provided herein having a formula of C—(X₁)ₙ—(X₂)_{q}—(X₃)_{y}-L-X₄, wherein C is a polymer, such as PEG, Endoporter, INF-7, TAT, polyarginine, polylysine, an amphipathic peptide, or peptides provided in Table 2; X₁ is a first FN3 domain; X₂ is a second FN3 domain; X₃ is a third FN3 domain or half-life extender molecule; L is a linker; and X₄ is a nucleic acid molecule, wherein n, q, and y are each independently 0 or 1.

In some embodiments, compositions are provided herein having a formula of (X₁)˜-(X₂)_{q}—(X₃)_{y}-L-X₄—C, wherein X₁ is a first FN3 domain; X₂ is a second FN3 domain; X₃ is a third FN3 domain or half-life extender molecule; L is a linker; X₄ is a nucleic acid molecule; and C is a polymer, wherein n, q, and y are each independently 0 or 1.

In some embodiments, compositions are provided herein having a formula of X₄-L-(X₁)ₙ—(X₂)_{q}—(X₃)_{y}, wherein X₁ is a first FN3 domain; X₂ is a second FN3 domain; X₃ is a third FN3 domain or half-life extender molecule; L is a linker; and X₄ is a nucleic acid molecule, wherein n, q, and y are each independently 0 or 1.

In some embodiments, compositions are provided herein having a formula of C-X₄-L-(X₁)ₙ—(X₂)_{q}—(X₃)_{y}, wherein C is a polymer; X₁ is a first FN3 domain; X₂ is a second FN3 domain; X₃ is a third FN3 domain or half-life extender molecule; L is a linker; and X₄ is a nucleic acid molecule, wherein n, q, and y are each independently 0 or 1.

In some embodiments, compositions are provided herein having a formula of X₄-L-(X₁)ₙ—(X₂)_{q}—(X₃)_{y}—C, wherein X₁ is a first FN3 domain; X₂ is a second FN3 domain; X₃ is a third FN3 domain or half-life extender molecule; L is a linker; X₄ is a nucleic acid molecule; and C is a polymer, wherein n, q, and y are each independently 0 or 1.

In some embodiments, the CD40 binding siRNA molecule comprises a sequence pair that may follow the sequence: sense strand (5′-3′) nsnsnnnnNfNfNfnnnnnnnnsnsa or (5′-3′) nsnsnnnnNfNfNfnnnnnnnnna; and antisense strand (5′-3′) UfsNfsnnnNfnnnnnnnNfnNfnnnsusu, wherein (n) is 2′-O-Me (methyl), (Nf) is 2′-F (fluoro), (s) is phosphorothioate backbone modification. Each nucleotide in both the sense and antisense strands may be modified independently or in combination at ribosugar and nucleobase positions.

In some embodiments, the siRNA molecule comprises a sequence pair from Table 3A, Table 3B, Table 4A, or Table 4B. In some embodiments, Tables 5A and 5B depict non-limiting examples of sequence pairs wherein the sense strand comprises a linker molecule. In some embodiments, any siRNA molecule provided for herein may comprise a linker molecule as disclosed herein.

In some embodiments, the siRNA molecule comprises a sense strand comprising the nucleic acid sequence of SEQ ID NO: 1890 and an antisense strand comprising the nucleic acid sequence of SEQ ID NO: 2290. In some embodiments, the siRNA molecule comprises a linker at the 3′ end of the sense strand. In some embodiments, the linker is C₆-NH2-Propyl-Mal. In some embodiments, the siRNA molecule comprises the sequence pair H11 as set forth in Table 5B.

In some embodiments, the siRNA molecule comprises a sense strand comprising the nucleic acid sequence of SEQ ID NO: 1893 and an antisense strand comprising the nucleic acid sequence of SEQ ID NO: 2293. In some embodiments, the siRNA molecule comprises a linker at the 3′ end of the sense strand. In some embodiments, the linker is C₆—NH₂—Propyl-Mal. In some embodiments, the siRNA molecule comprises the sequence pair K11 as set forth in Table 5B.

In some embodiments, the siRNA molecule comprises a sense strand comprising the nucleic acid sequence of SEQ ID NO: 1941 and an antisense strand comprising the nucleic acid sequence of SEQ ID NO: 2051. In some embodiments, the siRNA molecule comprises a linker at the 5′ end of the sense strand. In some embodiments, the linker is C₆—NH₂—Propyl-Mal. In some embodiments, the siRNA molecule comprises the sequence pair 010 as set forth in Table 5B.

In some embodiments, the siRNA molecule comprises a sense strand comprising the nucleic acid sequence of SEQ ID NO: 1942 and an antisense strand comprising the nucleic acid sequence of SEQ ID NO: 2052. In some embodiments, the siRNA molecule comprises a linker at the 5′ end of the sense strand. In some embodiments, the linker is C₆—NH₂—Propyl-Mal. In some embodiments, the siRNA molecule comprises the sequence pair P10 as set forth in Table 5B.

In some embodiments, the siRNA molecule comprises a sense strand comprising the nucleic acid sequence of SEQ ID NO: 1944 and an antisense strand comprising the nucleic acid sequence of SEQ ID NO: 2054. In some embodiments, the siRNA molecule comprises a linker at the 5′ end of the sense strand. In some embodiments, the linker is C₆—NH₂—Propyl-Mal. In some embodiments, the siRNA molecule comprises the sequence pair R10 as set forth in Table 5B.

TABLE 3A
siRNA Sense and Antisense Sequences (Modified)

| siRNA | SEQ | | SEQ | |
|---|---|---|---|---|
| Pair | ID NO: | Sense Strand 5′-3′ | ID NO: | Antisense Strand 5′-3′ |
| A1 | 46 | [mA*][mG*][[A][mA][ | 179 | [fU*][fC*][mA][mu][ |
| | | mC][mC][fA][fC][fC] | | mG][mC][mA][mG][mU] |
| | | [mC][mA][fC][mU][mG | | [mG][mG][mG][mU][fG |
| | | ][mC][mA][fU][mG][m | | ][mG][mU][mU][mC][m |
| | | A][idT] | | U][*mU][*mU] |
| B1 | 47 | [mA*][mG*][fA][mA][ | 180 | [fU*][fA*][mU][mu][ |
| | | mA][mA][fA][fC][fA] | | mA][mG][mG][mU][mA] |
| | | [mG][mU][fA][mC][mC | | [mC][mU][mG][mU][fU |
| | | ][mU][mA][fA][mu][m | | ][mU][mU][mU][mC][m |
| | | A][idT] | | U][*mU][*mU] |
| C1 | 48 | [mG*][mA*][[A][mA][ | 181 | [fU*][fC*][mA][mG][ |
| | | mC][mU][fG][fG][fU] | | mU][mC][mA][mC][mU] |
| | | [mG][mA][fG][mu][mG | | [mC][mA][mC][mC][fA |
| | | ][mA][mC][fU][mG][m | | ][mG][mU][mU][mu][m |
| | | A][idT] | | C][*mU][*mU] |
| D1 | 49 | [mA*][mA*][fA][mC][ | 182 | [fU*][fG*][mC][mA][ |
| | | mU][mG][fG][fU][fG] | | mG][mU][mC][mA][mC] |
| | | [mA][mG][fU][mG][mA | | [mU][mC][mA][mC][fC |
| | | ][mC][mU][fG][mC][m | | ][mA][mG][mU][mU][m |
| | | A][idT] | | U][*mU][*mU] |
| E1 | 50 | [mA*][mG*][fU][mU][ | 183 | [fU*][fA*][mU][mu][ |
| | | mC][mA][fC][fU][fG] | | mC][mC][mG][mU][mU] |
| | | [mA][mA][fA][mC][mG | | [mU][mC][mA][mG][fU |
| | | ][mG][mA][fA][mU][m | | ][mG][mA][mA][mC][m |
| | | A][idT] | | U][*mU][*mU] |
| F1 | 51 | [mG*][mG*][fA][mA][ | 184 | [fU*][fC*][mC][mG][ |
| | | mU][mG][fC][fC][fU] | | mC][mA][mA][mG][mG] |
| | | [mU][mC][fC][mU][mU | | [mA][mA][mG][mG][fC |
| | | ][mG][mC][fG][mG][m | | ][mA][mU][mU][mC][m |
| | | A][idT] | | C][*mU][*mU] |
| G1 | 52 | [mU*][mG*][fC][mC][ | 185 | [fU*][fU*][mU][mC][ |
| | | mU][mU][fC][fC][fU] | | mA][mC][mC][mG][mC] |
| | | [mU][mG][fC][mG][mG | | [mA][mA][mG][mG][fA |
| | | ][mU][mG][fA][mA][m | | ][mA][mG][mG][mC][m |
| | | A][idT] | | A][*mU][*mU] |
| H1 | 53 | [mG*][mC*][fC][mU][ | 186 | [fU*][fU*][mU][mu][ |
| | | mU][mC][fC][fU][fU] | | mC][mA][mC][mC][mG] |
| | | [mG][mC][fG][mG][mU | | [mC][mA][mA][mG][fG |
| | | ][mG][mA][fA][mA][m | | ][mA][mA][mG][mG][m |
| | | A][idT] | | C][*mU][*mU] |
| I1 | 54 | [mC*][mC*][fU][mU][ | 187 | [fU*][fC*][mU][mU][ |
| | | mC][mC][fU][fU][fG] | | mU][mC][mA][mC][mC] |
| | | [mC][mG][fG][mU][mG | | [mG][mC][mA][mA][fG |
| | | ][mA][mA][fA][mG][m | | ][mG][mA][mA][mG][m |
| | | A][idT] | | G][*mU][*mU] |
| J1 | 55 | [mC*][mU*][fU][mC][ | 188 | [fU*][fG*][mC][mU][ |
| | | mC][mU][fU][[G][fC] | | mU][mU][mC][mA][mC] |
| | | [mG][mG][fU][mG][mA | | [mC][mG][mC][mA][fA |
| | | ][mA][mA][fG][mC][m | | ][mG][mG][mA][mA][m |
| | | A][idT] | | G][*mU][*mU] |
| K1 | 56 | [mU*][mU*][fC][mC][ | 189 | [fU*][fC*][mG][mC][ |
| | | mU][mU][fG][fC][fG] | | mU][mU][mU][mC][mA] |
| | | [mG][mU][fG][mA][mA | | [mC][mC][mG][mC][fA |
| | | ][mA][mG][fC][mG][m | | ][mA][mG][mG][mA][m |
| | | A][idT] | | A][*mU][*mU] |
| L1 | 57 | [mU*][mC*][fC][mU][ | 190 | [fU*][fU*][mC][mG][ |
| | | mU][mG][fC][fG][[G] | | mC][mU][mU][mU][mC] |
| | | [mU][mG][fA][mA][mA | | [mA][mC][mC][mG][fC |
| | | ][mG][mC][fG][mA][m | | ][mA][mA][mG][mG][m |
| | | A][idT] | | A][*mU][*mU] |
| M1 | 58 | [mC*][mC*][fU][mU][ | 191 | [fU*][fU*][mU][mC][ |
| | | mG][mC][fG][fG][fU] | | mG][mC][mU][mU][mU] |
| | | [mG][mA][fA][mA][mG | | [mC][mA][mC][mC][fG |
| | | ][mC][mG][fA][mA][m | | ][mC][mA][mA][mG][m |
| | | A][idT] | | G][*mU][*mU] |
| N1 | 59 | [mC*][mU*][fU][mG][ | 192 | [fU*][fA*][mU][mU][ |
| | | mC][mG][fG][fU][[G] | | mC][mG][mC][mU][mU] |
| | | [mA][mA][fA][mG][mC | | [mU][mC][mA][mC][fC |
| | | ][mG][mA][fA][mU][m | | ][mG][mC][mA][mA][m |
| | | A][idT] | | G][*mU][*mU] |
| O1 | 60 | [mU*][mU*][[G][mC][ | 193 | [fU*][fA*][mA][mU][ |
| | | mG][mG][fU][fG][[A] | | mU][mC][mG][mC][mU] |
| | | [mA][mA][fG][mC][mG | | [mU][mU][mC][mA][fC |
| | | ][mA][mA][fU][mU][m | | ][mC][mG][mC][mA][m |
| | | A][idT] | | A][*mU][*mU] |
| P1 | 61 | [mU*][mG*][fC][mG][ | 194 | [fU*][fG*][mA][mA][ |
| | | mG][mU][fG][fA][fA] | | mU][mU][mC][mG][mC] |
| | | [mA][mG][fC][mG][mA | | [mU][mU][mU][mC][fA |
| | | ][mA][mU][fU][mC][m | | ][mC][mC][mG][mC][m |
| | | A][idT] | | A][*mU][*mU] |
| Q1 | 62 | [mC*][mG*][fG][mU][ | 195 | [fU*][fA*][mG][mG][ |
| | | mG][mA][fA][[A][[G] | | mA][mA][mU][mU][mC] |
| | | [mC][mG][fA][mA][mU | | [mG][mC][mU][mU][fU |
| | | ][mU][mC][fC][mU][m | | ][mC][mA][mC][mC][m |
| | | A][idT] | | G][*mU][*mU] |
| R1 | 63 | [mG*][mG*][fU][mG][ | 196 | [fU*][fU*][mA][mG][ |
| | | mA][mA][fA][fG][[C] | | mG][mA][mA][mU][mU] |
| | | [mG][mA][fA][mU][mU | | [mC][mG][mC][mU][fU |
| | | ][mC][mC][fU][mA][m | | ][mU][mC][mA][mC][m |
| | | A][idT] | | C][*mU][*mU] |
| S1 | 64 | [mU*][mG*][fA][mA][ | 197 | [fU*][fU*][mC][mU][ |
| | | mA][mG][fC][fG][fA] | | mA][mG][mG][mA][mA] |
| | | [mA][mU][fU][mC][mC | | [mU][mU][mC][mG][fC |
| | | ][mU][mA][fG][mA][m | | ][mU][mU][mU][mC][m |
| | | A][idT] | | A][*mU][*mU] |
| T1 | 65 | [mG*][mA*][fA][mA][ | 198 | [fU*][fG*][mU][mC][ |
| | | mG][mC][fG][fA][fA] | | mU][mA][mG][mG][mA] |
| | | [mU][mU][fC][mC][mU | | [mA][mU][mU][mC][fG |
| | | ][mA][mG][fA][mC][m | | ][mC][mU][mU][mU][m |
| | | A][idT] | | C][*mU][*mU] |
| U1 | 66 | [mA*][mA*][fA][mG][ | 199 | [fU*][fU*][mG][mU][ |
| | | mC][mG][fA][fA][fU] | | mC][mU][mA][mG][mG] |
| | | [mU][mC][fC][mU][mA | | [mA][mA][mU][mU][fC |
| | | ][mG][mA][fC][mA][m | | ][mG][mC][mU][mU][m |
| | | A][idT] | | U][*mU][*mU] |
| V1 | 67 | [mA*][mA*][fG][mC][ | 200 | [fU*][fG*][mU][mG][ |
| | | mG][mA][fA][fU][fU] | | mU][mC][mU][mA][mG] |
| | | [mC][mC][fU][mA][mG | | [mG][mA][mA][mU][fU |
| | | ][mA][mC][fA][mC][m | | ][mC][mG][mC][mU][m |
| | | A][idT] | | U][*mU][*mU] |
| W1 | 68 | [mA*][mG*][fC][mG][ | 201 | [fU*][fG*][mG][mu][ |
| | | mA][mA][fU][fU][fC] | | mG][mU][mC][mU][mA] |
| | | [mC][mU][fA][mG][mA | | [mG][mG][mA][mA][fU |
| | | ][mC][mA][fC][mC][m | | ][mU][mC][mG][mC][m |
| | | A][idT] | | U][*mU][*mU] |
| X1 | 69 | [mC*][mU*][[A][mG][ | 202 | [fU*][fU*][mC][mU][ |
| | | mA][mC][fA][fC][fC] | | mG][mU][mU][mC][mC] |
| | | [mU][mG][fG][mA][mA | | [mA][mG][mG][mU][fG |
| | | ][mC][mA][fG][mA][m | | ][mU][mC][mU][mA][m |
| | | A][idT] | | G][*mU][*mU] |
| Y1 | 70 | [mC*][mA*][fG][mC][ | 203 | [fU*][fG*][mU][mC][ |
| | | mA][mC][fA][fA][fA] | | mG][mC][mA][mG][mU] |
| | | [mU][mA][fC][mU][mG | | [mA][mU][mU][mU][fG |
| | | ][mC][mG][fA][mC][m | | ][mU][mG][mC][mU][m |
| | | A][idT] | | G][*mU][*mU] |
| Z1 | 71 | [mA*][mG*][fC][mA][ | 204 | [fU*][fG*][mG][mU][ |
| | | mC][mA][fA][fA][fu] | | mC][mG][mC][mA][mG] |
| | | [mA][mC][fU][mG][mC | | [mU][mA][mU][mU][fU |
| | | ][mG][mA][fC][mC][m | | ][mG][mU][mG][mC][m |
| | | A][idT] | | U][*mU][*mU] |
| A2 | 72 | [mG*][mC*][fA][mC][ | 205 | [fU*][fG*][mG][mG][ |
| | | mA][mA][fA][fU][fA] | | mU][mC][mG][mC][mA] |
| | | [mC][mU][fG][mC][mG | | [mG][mU][mA][mU][fU |
| | | ][mA][mC][fC][mC][m | | ][mU][mG][mU][mG][m |
| | | A][idT] | | C][*mU][*mU] |
| B2 | 73 | [mU*][mA*][fC][mU][ | 206 | [fU*][fU*][mA][mG][ |
| | | mG][mC][fG][fA][fC] | | mG][mU][mU][mG][mG] |
| | | [mC][mC][fC][mA][mA | | [mG][mG][mU][mC][fG |
| | | ][mC][mC][fU][mA][m | | ][mC][mA][mG][mU][m |
| | | A][idT] | | A][*mU][*mU] |
| C2 | 74 | [mA*][mC*][fU][mG][ | 207 | [fU*][fC*][mU][mA][ |
| | | mC][mG][fA][fC][fC] | | mG][mG][mU][mU][mG] |
| | | [mC][mC][fA][mA][mC | | [mG][mG][mG][mU][fC |
| | | ][mC][mU][fA][mG][m | | ][mG][mC][mA][mG][m |
| | | A][idT] | | U][*mU][*mU] |
| D2 | 75 | [mC*][mU*][fG][mC][ | 208 | [fU*][fC*][mC][mU][ |
| | | mG][mA][fC][fC][fC] | | mA][mG][mG][mU][mU] |
| | | [mC][mA][fA][mC][mC | | [mG][mG][mG][mG][fU |
| | | ][mU][mA][fG][mG][m | | ][mC][mG][mC][mA][m |
| | | A][idT] | | G][*mU][*mU] |
| E2 | 76 | [mG*][mA*][fC][mC][ | 209 | [fU*][fA*][mA][mG][ |
| | | mC][mC][fA][fA][fC] | | mC][mC][mC][mU][mA] |
| | | [mC][mU][fA][mG][mG | | [mG][mG][mU][mU][fG |
| | | ][mG][mC][fU][mU][m | | ][mG][mG][mG][mU][m |
| | | A][idT] | | C][*mU][*mU] |
| F2 | 77 | [mA*][mC*][fC][mC][ | 210 | [fU*][fG*][mA][mA][ |
| | | mC][mA][fA][fC][fC] | | mG][mC][mC][mC][mU] |
| | | [mU][mA][fG][mG][mG | | [mA][mG][mG][mU][fU |
| | | ][mC][mU][fU][mC][m | | ][mG][mG][mG][mG][m |
| | | A][idT] | | U][*mU][*mU] |
| G2 | 78 | [mC*][mC*][fC][mC][ | 211 | [fU*][fC*][mG][mA][ |
| | | mA][mA][fC][fC][fU] | | mA][mG][mC][mC][mC] |
| | | [mA][mG][fG][mG][mC | | [mU][mA][mG][mG][fU |
| | | ][mU][mU][fC][mG][m | | ][mU][mG][mG][mG][m |
| | | A][idT] | | G][*mU][*mU] |
| H2 | 79 | [mC*][mC*][fU][mA][ | 212 | [fU*][fU*][mG][mG][ |
| | | mG][mG][fG][fC][fU] | | mA][mC][mC][mC][mG] |
| | | [mU][mC][fG][mG][mG | | [mA][mA][mG][mC][fC |
| | | ][mU][mC][fC][mA][m | | ][mC][mU][mA][mG][m |
| | | A][idT] | | G][*mU][*mU] |
| I2 | 80 | [mU*][mC*][fG][mG][ | 213 | [fU*][fC*][mC][mC][ |
| | | mG][mU][fC][fC][fA] | | mU][mU][mC][mU][mG] |
| | | [mG][mC][fA][mG][mA | | [mC][mU][mG][mG][fA |
| | | ][mA][mG][fG][mG][m | | ][mC][mC][mC][mG][m |
| | | A][idT] | | A][*mU][*mU] |
| J2 | 81 | [mG*][mA*][fA][mG][ | 214 | [fU*][fC*][mG][mU][ |
| | | mG][mC][fU][fG][fG] | | mA][mC][mA][mG][mU] |
| | | [mC][mA][fC][mU][mG | | [mG][mC][mC][mA][fG |
| | | ][mU][mA][fC][mG][m | | ][mC][mC][mU][mU][m |
| | | A][idT] | | C][*mU][*mU] |
| K2 | 82 | [mG*][mG*][fC][mA][ | 215 | [fU*][fC*][mC][mU][ |
| | | mC][mU][fG][fU][fA] | | mC][mA][mC][mU][mC] |
| | | [mC][mG][fA][mG][mU | | [mG][mU][mA][mC][fA |
| | | ][mG][mA][fG][mG][m | | ][mG][mU][mG][mC][m |
| | | A][idT] | | C][*mU][*mU] |
| L2 | 83 | [mG*][mU*][[A][mC][ | 216 | [fU*][fC*][mA][mC][ |
| | | mG][mA][fG][fU][fG] | | mA][mG][mG][mC][mC] |
| | | [mA][mG][fG][mC][mC | | [mU][mC][mA][mC][fU |
| | | ][mU][mG][fU][mG][m | | ][mC][mG][mU][mA][m |
| | | A][idT] | | C][*mU][*mU] |
| M2 | 84 | [mU*][mG*][fU][mC][ | 217 | [fU*][fC*][mA][mU][ |
| | | mC][mU][fG][fC][[A] | | mG][mA][mG][mC][mG] |
| | | [mC][mC][fG][mC][mU | | [mG][mU][mG][mC][fA |
| | | ][mC][mA][fU][mG][m | | ][mG][mG][mA][mC][m |
| | | A][idT] | | A][*mU][*mU] |
| N2 | 85 | [mU*][mG*][fC][mA][ | 218 | [fU*][fG*][mC][mG][ |
| | | mC][mC][fG][fC][fU] | | mA][mG][mC][mA][mU] |
| | | [mC][mA][fU][mG][mC | | [mG][mA][mG][mC][fG |
| | | ][mU][mC][fG][mC][m | | ][mG][mU][mG][mC][m |
| | | A][idT] | | A][*mU][*mU] |
| O2 | 86 | [mG*][mC*][fA][mC][ | 219 | [fU*][fG*][mG][mC][ |
| | | mC][mG][fC][fU][fC] | | mG][mA][mG][mC][mA] |
| | | [mA][mU][fG][mC][mU | | [mU][mG][mA][mG][fC |
| | | ][mC][mG][fC][mC][m | | ][mG][mG][mU][mG][m |
| | | A][idT] | | C][*mU][*mU] |
| P2 | 87 | [mC*][mC*][fG][mC][ | 220 | [fU*][fC*][mC][mG][ |
| | | mU][mC][fA][fU][fG] | | mG][mG][mC][mG][mA] |
| | | [mC][mU][fC][mG][mC | | [mG][mC][mA][mU][fG |
| | | ][mC][mC][fG][mG][m | | ][mA][mG][mC][mG][m |
| | | A][idT] | | G][*mU][*mU] |
| Q2 | 88 | [mC*][mC*][fC][mG][ | 221 | [fU*][fC*][mU][mU][ |
| | | mG][mC][fU][fU][fU] | | mG][mA][mC][mC][mC] |
| | | [mG][mG][fG][mG][mU | | [mC][mA][mA][mA][EG |
| | | ][mC][mA][fA][mG][m | | ][mC][mC][mG][mG][m |
| | | A][idT] | | G][*mU][*mU] |
| R2 | 89 | [mC*][mU*][fG][mA][ | 222 | [fU*][fG*][mC][mU][ |
| | | mU][mA][fC][fC][[A] | | mC][mG][mC][mA][mG] |
| | | [mU][mC][fU][mG][mC | | [mA][mU][mG][mG][fU |
| | | ][mG][mA][fG][mC][m | | ][mA][mU][mC][mA][m |
| | | A][idT] | | G][*mU][*mU] |
| S2 | 90 | [mU*][mG*][fA][mU][ | 223 | [fU*][fG*][mG][mC][ |
| | | mA][mC][fC][fA][fU] | | mU][mC][mG][mC][mA] |
| | | [mC][mU][fG][mC][mG | | [mG][mA][mU][mG][fG |
| | | ][mA][mG][fC][mC][m | | ][mU][mA][mU][mC][m |
| | | A][idT] | | A][*mU][*mU] |
| T2 | 91 | [mC*][mU*][fG][mC][ | 224 | [fU*][fA*][mA][mG][ |
| | | mC][mC][fA][fG][fU] | | mA][mA][mG][mC][mC] |
| | | [mC][mG][fG][mC][mU | | [mG][mA][mC][mU][fG |
| | | ][mU][mC][fU][mU][m | | ][mG][mG][mC][mA][m |
| | | A][idT] | | G][*mU][*mU] |
| U2 | 92 | [mG*][mU*][fG][mU][ | 225 | [fU*][fU*][mU][mC][ |
| | | mC][mA][fU][fC][fU] | | mG][mA][mA][mA][mG] |
| | | [mG][mC][fU][mU][mU | | [mC][mA][mG][mA][fU |
| | | ][mC][mG][fA][mA][m | | ][mG][mA][mC][mA][m |
| | | A][idT] | | C][*mU][*mU] |
| V2 | 93 | [mU*][mG*][fU][mC][ | 226 | [fU*][fU*][mU][mU][ |
| | | mA][mU][fC][fU][fG] | | mC][mG][mA][mA][mA] |
| | | [mC][mU][fU][mU][mC | | [mG][mC][mA][mG][fA |
| | | ][mG][mA][fA][mA][m | | ][mU][mG][mA][mC][m |
| | | A][idT] | | A][*mU][*mU] |
| W2 | 94 | [mU*][mC*][fA][mU][ | 227 | [fU*][fU*][mU][mU][ |
| | | mC][mU][fG][fC][fU] | | mU][mU][mC][mG][mA] |
| | | [mU][mU][fC][mG][mA | | [mA][mA][mG][mC][fA |
| | | ][mA][mA][fA][mA][m | | ][mG][mA][mU][mG][m |
| | | A][idT] | | A][*mU][*mU] |
| X2 | 95 | [mU*][mU*][fC][mG][ | 228 | [fU*][fA*][mG][mG][ |
| | | mA][mA][fA][fA][fA] | | mG][mU][mG][mA][mC] |
| | | [mU][mG][fU][mC][mA | | [mA][mU][mU][mU][fU |
| | | ][mC][mC][fC][mU][m | | ][mU][mC][mG][mA][m |
| | | A][idT] | | A][*mU][*mU] |
| Y2 | 96 | [mU*][mC*][fG][mA][ | 229 | [fU*][fA*][mA][mG][ |
| | | mA][mA][fA][fA][fU] | | mG][mG][mU][mG][mA] |
| | | [mG][mU][fC][mA][mC | | [mC][mA][mU][mU][fU |
| | | ][mC][mC][fU][mu][m | | ][mU][mU][mC][mG][m |
| | | A][idT] | | A][*mU][*mU] |
| Z2 | 97 | [mA*][mA*][fA][mA][ | 230 | [fU*][fG*][mU][mC][ |
| | | mU][mG][fU][fC][fA] | | mC][mA][mA][mG][mG] |
| | | [mC][mC][fC][mU][mU | | [mG][mU][mG][mA][fC |
| | | ][mG][mG][fA][mC][m | | ][mA][mU][mU][mU][m |
| | | A][idT] | | U][*mU][*mU] |
| A3 | 98 | [mU*][mG*][fA][mU][ | 231 | [fU*][fC*][mG][mA][ |
| | | mC][mC][fC][fC][fA] | | mA][mG][mA][mU][mG] |
| | | [mU][mC][fA][mU][mC | | [mA][mU][mG][mG][fG |
| | | ][mU][mU][fC][mG][m | | ][mG][mA][mU][mC][m |
| | | A][idT] | | A][*mU][*mU] |
| B3 | 99 | [mC*][mC*][fC][mC][ | 232 | [fU*][fA*][mU][mC][ |
| | | mA][mU][fC][fA][fU] | | mC][mC][mG][mA][mA] |
| | | [mC][mU][fU][mC][mG | | [mG][mA][mU][mG][fA |
| | | ][mG][mG][fA][mU][m | | ][mU][mG][mG][mG][m |
| | | A][idT] | | G][*mU][*mU] |
| C3 | 100 | [mA*][mU*][fC][mU][ | 233 | [fU*][fA*][mA][mA][ |
| | | mU][mC][fG][fG][fG] | | mC][mA][mG][mG][mA] |
| | | [mA][mU][fC][mC][mU | | [mU][mC][mC][mC][fG |
| | | ][mG][mU][fU][mU][m | | ][mA][mA][mG][mA][m |
| | | A][idT] | | U][*mU][*mU] |
| D3 | 101 | [mA*][mA*][fG][mA][ | 234 | [fU*][fC*][mU][mU][ |
| | | mA][mG][fC][fC][fA] | | mA][mU][mU][mG][mG] |
| | | [mA][mC][fC][mA][mA | | [mU][mU][mG][mG][fC |
| | | ][mU][mA][fA][mG][m | | ][mU][mU][mC][mU][m |
| | | A][idT] | | U][*mU][*mU] |
| E3 | 102 | [mA*][mA*][fG][mC][ | 235 | [fU*][fG*][mG][mC][ |
| | | mC][mA][fA][fC][fC] | | mC][mU][mU][mA][mU] |
| | | [mA][mA][fU][mA][mA | | [mU][mG][mG][mU][fU |
| | | ][mG][mG][fC][mC][m | | ][mG][mG][mC][mU][m |
| | | A][idT] | | U][*mU][*mU] |
| F3 | 103 | [mA*][mG*][fA][mU][ | 236 | [fU*][fC*][mG][mU][ |
| | | mC][mA][fA][fU][fU] | | mC][mG][mG][mG][mA] |
| | | [mU][mU][fC][mC][mC | | [mA][mA][mA][mU][fU |
| | | ][mG][mA][fC][mG][m | | ][mG][mA][mU][mC][m |
| | | A][idT] | | U][*mU][*mU] |
| G3 | 104 | [mG*][mA*][fU][mC][ | 237 | [fU*][fU*][mC][mG][ |
| | | mA][mA][fU][fU][fU] | | mU][mC][mG][mG][mG] |
| | | [mU][mC][fC][mC][mG | | [mA][mA][mA][mA][fU |
| | | ][mA][mC][fG][mA][m | | ][mU][mG][mA][mU][m |
| | | A][idT] | | C][*mU][*mU] |
| H3 | 105 | [mA*][mU*][fC][mA][ | 238 | [fU*][fA*][mU][mC][ |
| | | mA][mU][fU][fU][fU] | | mG][mU][mC][mG][mG] |
| | | [mC][mC][fC][mG][mA | | [mG][mA][mA][mA][fA |
| | | ][mC][mG][fA][mu][m | | ][mU][mU][mG][mA][m |
| | | A][idT] | | U][*mU][*mU] |
| I3 | 106 | [mU*][mC*][fA][mA][ | 239 | [fU*][fG*][mA][mU][ |
| | | mU][mU][fU][fU][fC] | | mC][mG][mU][mC][mG] |
| | | [mC][mC][fG][mA][mC | | [mG][mG][mA][mA][fA |
| | | ][mG][mA][fU][mC][m | | ][mA][mU][mU][mG][m |
| | | A][idT] | | A][*mU][*mU] |
| J3 | 107 | [mC*][mG*][fA][mC][ | 240 | [fU*][fG*][mA][mG][ |
| | | mG][mA][fU][fC][fU] | | mC][mC][mA][mG][mG] |
| | | [mU][mC][fC][mU][mG | | [mA][mA][mG][mA][fU |
| | | ][mG][mC][fU][mC][m | | ][mC][mG][mU][mC][m |
| | | A][idT] | | G][*mU][*mU] |
| K3 | 108 | [mA*][mC*][fG][mA][ | 241 | [fU*][fU*][mG][mG][ |
| | | mU][mC][fU][fU][fC] | | mA][mG][mC][mC][mA] |
| | | [mC][mU][fG][mG][mC | | [mG][mG][mA][mA][fG |
| | | ][mU][mC][fC][mA][m | | ][mA][mU][mC][mG][m |
| | | A][idT] | | U][*mU][*mU] |
| L3 | 109 | [mG*][mA*][fU][mC][ | 242 | [fU*][fG*][mU][mU][ |
| | | mU][mU][fC][fC][fU] | | mG][mG][mA][mG][mC] |
| | | [mG][mG][fC][mU][mC | | [mC][mA][mG][mG][fA |
| | | ][mC][mA][fA][mC][m | | ][mA][mG][mA][mU][m |
| | | A][idT] | | C][*mU][*mU] |
| M3 | 110 | [mU*][mG*][fC][mA][ | 243 | [fU*][fC*][mA][mu][ |
| | | mG][mG][fA][fG][fA] | | mG][mU][mA][mA][mA] |
| | | [mC][mU][fU][mU][mA | | [mG][mU][mC][mU][fC |
| | | ][mC][mA][fU][mG][m | | ][mC][mU][mG][mC][m |
| | | A][idT] | | A][*mU][*mU] |
| N3 | 111 | [mC*][mA*][fG][mG][ | 244 | [fU*][fU*][mC][mC][ |
| | | mA][mG][fA][fC][fU] | | mA][mU][mG][mU][mA] |
| | | [mU][mU][fA][mC][mA | | [mA][mA][mG][mU][fC |
| | | ][mU][mG][fG][mA][m | | ][mU][mC][mC][mU][m |
| | | A][idT] | | G][*mU][*mU] |
| O3 | 112 | [mG*][mG*][fA][mG][ | 245 | [fU*][fC*][mA][mU][ |
| | | mA][mC][fU][fU][fu] | | mC][mC][mA][mU][mG] |
| | | [mA][mC][fA][mU][mG | | [mU][mA][mA][mA][fG |
| | | ][mG][mA][fU][mG][m | | ][mU][mC][mU][mC][m |
| | | A][idT] | | C][*mU][*mU] |
| P3 | 113 | [mA*][mG*][fG][mA][ | 246 | [fU*][fG*][mA][mC][ |
| | | mU][mG][fG][fC][fA] | | mU][mC][mU][mC][mU] |
| | | [mA][mA][fG][mA][mG | | [mU][mU][mG][mC][fC |
| | | ][mA][mG][fU][mC][m | | ][mA][mU][mC][mC][m |
| | | A][idT] | | U][*mU][*mU] |
| Q3 | 114 | [mG*][mG*][fA][mU][ | 247 | [fU*][fC*][mG][mA][ |
| | | mG][mG][fC][fA][fA] | | mC][mU][mC][mU][mC] |
| | | [mA][mG][fA][mG][mA | | [mU][mU][mU][mG][fC |
| | | ][mG][mU][fC][mG][m | | ][mC][mA][mU][mC][m |
| | | A][idT] | | C][*mU][*mU] |
| R3 | 115 | [mU*][mG*][fG][mC][ | 248 | [fU*][fA*][mU][mG][ |
| | | mA][mA][fA][fG][fA] | | mC][mG][mA][mC][mU] |
| | | [mG][mA][fG][mU][mC | | [mC][mU][mC][mU][fU |
| | | ][mG][mC][fA][mU][m | | ][mU][mG][mC][mC][m |
| | | A][idT] | | A][*mU][*mU] |
| S3 | 116 | [mG*][mG*][fC][mA][ | 249 | [fU*][fG*][mA][mu][ |
| | | mA][mA][fG][fA][fG] | | mG][mC][mG][mA][mC] |
| | | [mA][mG][fU][mC][mG | | [mU][mC][mU][mC][fU |
| | | ][mC][mA][fU][mC][m | | ][mU][mU][mG][mC][m |
| | | A][idT] | | C][*mU][*mU] |
| T3 | 117 | [mG*][mC*][fA][mA][ | 250 | [fU*][fA*][mG][mA][ |
| | | mA][mG][fA][fG][fA] | | mU][mG][mC][mG][mA] |
| | | [mG][mU][fC][mG][mC | | [mC][mU][mC][mU][fC |
| | | ][mA][mU][fC][mU][m | | ][mU][mU][mU][mG][m |
| | | A][idT] | | C][*mU][*mU] |
| U3 | 118 | [mA*][mA*][fA][mG][ | 251 | [fU*][fU*][mG][mA][ |
| | | mA][mG][fA][fG][fU] | | mG][mA][mU][mG][mC] |
| | | [mC][mG][fC][mA][mU | | [mG][mA][mC][mU][fC |
| | | ][mC][mU][fC][mA][m | | ][mU][mC][mU][mU][m |
| | | A][idT] | | U][*mU][*mU] |
| V3 | 119 | [mA*][mA*][fG][mA][ | 252 | [fU*][fC*][mU][mG][ |
| | | mG][mA][fG][fU][fC] | | mA][mG][mA][mU][mG] |
| | | [mG][mC][fA][mU][mC | | [mC][mG][mA][mC][fU |
| | | ][mU][mC][fA][mG][m | | ][mC][mU][mC][mU][m |
| | | A][idT] | | U][*mU][*mU] |
| W3 | 120 | [mA*][mG*][fA][mG][ | 253 | [fU*][fA*][mC][mU][ |
| | | mA][mG][fU][fC][fG] | | mG][mA][mG][mA][mU] |
| | | [mC][mA][fU][mC][mU | | [mG][mC][mG][mA][fC |
| | | ][mC][mA][fG][mU][m | | ][mU][mC][mU][mC][m |
| | | A][idT] | | U][*mU][*mU] |
| X3 | 121 | [mG*][mA*][fG][mA][ | 254 | [fU*][fC*][mA][mC][ |
| | | mG][mU][fC][fG][fC] | | mU][mG][mA][mG][mA] |
| | | [mA][mU][fC][mU][mC | | [mU][mG][mC][mG][fA |
| | | ][mA][mG][fU][mG][m | | ][mC][mU][mC][mU][m |
| | | A][idT] | | C][*mU][*mU] |
| Y3 | 122 | [mA*][mG*][fA][mG][ | 255 | [fU*][fG*][mC][mA][ |
| | | mU][mC][fG][fC][fA] | | mC][mU][mG][mA][mG] |
| | | [mU][mC][fU][mC][mA | | [mA][mU][mG][mC][fG |
| | | ][mG][mU][fG][mC][m | | ][mA][mC][mU][mC][m |
| | | A][idT] | | U][*mU][*mU] |
| Z3 | 123 | [mG*][mA*][fG][mU][ | 256 | [fU*][fU*][mG][mC][ |
| | | mC][mG][fC][fA][fU] | | mA][mC][mU][mG][mA] |
| | | [mC][mU][fC][mA][mG | | [mG][mA][mU][mG][fC |
| | | ][mU][mG][fC][mA][m | | ][mG][mA][mC][mU][m |
| | | A][idT] | | C][*mU][*mU] |
| A4 | 124 | [mG*][mU*][fC][mG][ | 257 | [fU*][fC*][mC][mU][ |
| | | mC][mA][fU][fC][fU] | | mG][mC][mA][mC][mU] |
| | | [mC][mA][fG][mU][mG | | [mG][mA][mG][mA][fU |
| | | ][mC][mA][fG][mG][m | | ][mG][mC][mG][mA][m |
| | | A][idT] | | C][*mU][*mU] |
| B4 | 125 | [mC*][mU*][fG][mC][ | 258 | [fU*][fA*][mC][mC][ |
| | | mU][mG][fU][fG][fG] | | mC][mU][mC][mA][mC] |
| | | [mC][mG][fU][mG][mA | | [mG][mC][mC][mA][fC |
| | | ][mG][mG][fG][mU][m | | ][mA][mG][mC][mA][m |
| | | A][idT] | | G][*mU][*mU] |
| C4 | 126 | [mG*][mG*][fC][mA][ | 259 | [fU*][fC*][mU][mA][ |
| | | mC][mU][fG][fA][fC] | | mU][mG][mC][mC][mC] |
| | | [mU][mG][fG][mG][mC | | [mA][mG][mU][mC][fA |
| | | ][mA][mU][fA][mG][m | | ][mG][mU][mG][mC][m |
| | | A][idT] | | C][*mU][*mU] |
| D4 | 127 | [mG*][mG*][fG][mC][ | 260 | [fU*][fA*][mA][mG][ |
| | | mA][mU][fA][fG][fC] | | mC][mG][mG][mG][mG] |
| | | [mU][mC][fC][mC][mC | | [mA][mG][mC][mU][fA |
| | | ][mG][mC][fU][mU][m | | ][mU][mG][mC][mC][m |
| | | A][idT] | | C][*mU][*mU] |
| E4 | 128 | [mA*][mG*][fA][mA][ | 261 | [fU*][fG*][mG][mU][ |
| | | mC][mC][fU][fC][fU] | | mG][mA][mA][mG][mU] |
| | | [mC][mA][fC][mU][mU | | [mG][mA][mG][mA][fG |
| | | ][mC][mA][fC][mC][m | | ][mG][mU][mU][mC][m |
| | | A][idT] | | U][*mU][*mU] |
| F4 | 129 | [mG*][mA*][fA][mC][ | 262 | [fU*][fG*][mG][mG][ |
| | | mC][mU][fC][fU][fC] | | mU][mG][mA][mA][mG] |
| | | [mA][mC][fU][mU][mC | | [mU][mG][mA][mG][fA |
| | | ][mA][mC][fC][mC][m | | ][mG][mG][mU][mU][m |
| | | A][idT] | | C][*mU][*mU] |
| G4 | 130 | [mA*][mG*][fU][mC][ | 263 | [fU*][fA*][mA][mU][ |
| | | mU][mC][fC][fC][fA] | | mA][mC][mA][mA][mG] |
| | | [mA][mC][fU][mU][mG | | [mU][mU][mG][mG][fG |
| | | ][mU][mA][fU][mU][m | | ][mA][mG][mA][mC][m |
| | | A][idT] | | U][*mU][*mU] |
| H4 | 131 | [mG*][mG*][fU][mU][ | 264 | [fU*][fG*][mG][mu][ |
| | | mU][mA][fG][fU][[A] | | mG][mG][mA][mU][mA] |
| | | [mA][mU][fA][mU][mC | | [mU][mU][mA][mC][fU |
| | | ][mC][mA][fC][mC][m | | ][mA][mA][mA][mC][m |
| | | A][idT] | | C][*mU][*mU] |
| I4 | 132 | [mG*][mU*][fU][mU][ | 265 | [fU*][fU*][mG][mG][ |
| | | mA][mG][fU][fA][fA] | | mU][mG][mG][mA][mU] |
| | | [mU][mA][fU][mC][mC | | [mA][mU][mU][mA][fC |
| | | ][mA][mC][fC][mA][m | | ][mU][mA][mA][mA][m |
| | | A][idT] | | C][*mU][*mU] |
| J4 | 133 | [mU*][mU*][fU][mA][ | 266 | [fU*][fC*][mU][mG][ |
| | | mG][mU][fA][fA][fU] | | mG][mU][mG][mG][mA] |
| | | [mA][mU][fC][mC][mA | | [mU][mA][mU][mU][fA |
| | | ][mC][mC][fA][mG][m | | ][mC][mU][mA][mA][m |
| | | A][idT] | | A][*mU][*mU] |
| K4 | 134 | [mG*][mU*][fA][mA][ | 267 | [fU*][fA*][mG][mG][ |
| | | mU][mA][fU][fC][fC] | | mU][mC][mU][mG][mG] |
| | | [mA][mC][fC][mA][mG | | [mU][mG][mG][mA][fU |
| | | ][mA][mC][fC][mU][m | | ][mA][mU][mU][mA][m |
| | | A][idT] | | C][*mU][*mU] |
| L4 | 135 | [mU*][mA*][fA][mU][ | 268 | [fU*][fA*][mA][mG][ |
| | | mA][mU][fC][fC][fA] | | mG][mU][mC][mU][mG] |
| | | [mC][mC][fA][mG][mA | | [mG][mU][mG][mG][fA |
| | | ][mC][mC][fU][mU][m | | ][mU][mA][mU][mU][m |
| | | A][idT] | | A][*mU][*mU] |
| M4 | 136 | [mC*][mA*][fU][mG][ | 269 | [fU*][fC*][mG][mC][ |
| | | mG][mU][fG][fG][fC] | | mA][mG][mG][mG][mA] |
| | | [mU][mU][fC][mC][mC | | [mA][mG][mC][mC][fA |
| | | ][mU][mG][fC][mG][m | | ][mC][mC][mA][mU][m |
| | | A][idT] | | G][*mU][*mU] |
| N4 | 137 | [mU*][mG*][fC][mG][ | 270 | [fU*][fU*][mA][mU][ |
| | | mC][mC][fC][fA][fG] | | mG][mG][mC][mU][mU] |
| | | [mG][mA][fA][mG][mC | | [mC][mC][mU][mG][fG |
| | | ][mC][mA][fU][mA][m | | ][mG][mC][mG][mC][m |
| | | A][idT] | | A][*mU][*mU] |
| O4 | 138 | [mC*][mG*][fC][mC][ | 271 | [fU*][fU*][mA][mU][ |
| | | mC][mA][fG][fG][fA] | | mA][mU][mG][mG][mC] |
| | | [mA][mG][fC][mC][mA | | [mU][mU][mC][mC][fU |
| | | ][mU][mA][fU][mA][m | | ][mG][mG][mG][mC][m |
| | | A][idT] | | G][*mU][*mU] |
| P4 | 139 | [mA*][mG*][fC][mC][ | 272 | [fU*][fG*][mC][mA][ |
| | | mA][mU][fA][fU][fA] | | mU][mC][mU][mG][mU] |
| | | [mC][mA][fC][mA][mG | | [mG][mU][mA][mU][fA |
| | | ][mA][mU][fG][mC][m | | ][mU][mG][mG][mC][m |
| | | A][idT] | | U][*mU][*mU] |
| Q4 | 140 | [mG*][mC*][fC][mA][ | 273 | [fU*][fG*][mG][mC][ |
| | | mU][mA][fU][fA][fC] | | mA][mU][mC][mU][mG] |
| | | [mA][mC][fA][mG][mA | | [mU][mG][mU][mA][fU |
| | | ][mU][mG][fC][mC][m | | ][mA][mU][mG][mG][m |
| | | A][idT] | | C][*mU][*mU] |
| R4 | 141 | [mU*][mU*][fG][mU][ | 274 | [fU*][fG*][mU][mU][ |
| | | mU][mU][fG][fU][fG] | | mC][mA][mC][mU][mA] |
| | | [mA][mU][fA][mG][mU | | [mU][mC][mA][mC][fA |
| | | ][mG][mA][fA][mC][m | | ][mA][mA][mC][mA][m |
| | | A][idT] | | A][*mU][*mU] |
| S4 | 142 | [mG*][mG*][fA][mA][ | 275 | [fU*][fG*][mA][mC][ |
| | | mG][mC][fU][fG][fC] | | mA][mG][mU][mU][mA] |
| | | [mU][mU][fA][mA][mc | | [mA][mG][mC][mA][fG |
| | | ][mU][mG][fU][mC][m | | ][mC][mU][mU][mC][m |
| | | A][idT] | | C][*mU][*mU] |
| T4 | 143 | [mG*][mC*][fU][mG][ | 276 | [fU*][fG*][mA][mu][ |
| | | mC][mU][fU][fA][[A] | | mG][mG][mA][mC][mA] |
| | | [mC][mU][fG][mU][mC | | [mG][mU][mU][mA][fA |
| | | ][mC][mA][fU][mC][m | | ][mG][mC][mA][mG][m |
| | | A][idT] | | C][*mU][*mU] |
| U4 | 144 | [mA*][mG*][fC][mA][ | 277 | [fU*][fU*][mU][mu][ |
| | | mG][mG][fA][fG][fA] | | mA][mG][mC][mC][mA] |
| | | [mC][mU][fG][mG][mC | | [mG][mU][mC][mU][fC |
| | | ][mU][mA][fA][mA][m | | ][mC][mU][mG][mC][m |
| | | A][idT] | | U][*mU][*mU] |
| V4 | 145 | [mG*][mC*][fA][mG][ | 278 | [fU*][fA*][mU][mU][ |
| | | mG][mA][fG][fA][fC] | | mU][mA][mG][mC][mC] |
| | | [mU][mG][fG][mC][mU | | [mA][mG][mU][mC][fU |
| | | ][mA][mA][fA][mu][m | | ][mC][mC][mU][mG][m |
| | | A][idT] | | C][*mU][*mU] |
| W4 | 146 | [mC*][mA*][fG][mG][ | 279 | [fU*][fU*][mA][mu][ |
| | | mA][mG][fA][fC][fU] | | mU][mU][mA][mG][mC] |
| | | [mG][mG][fC][mU][mA | | [mC][mA][mG][mU][fC |
| | | ][mA][mA][fU][mA][m | | ][mU][mC][mC][mU][m |
| | | A][idT] | | G][*mU][*mU] |
| X4 | 147 | [mA*][mU*][fA][mC][ | 280 | [fU*][fU*][mU][mU][ |
| | | mA][mA][fC][fA][fG] | | mG][mA][mG][mA][mU] |
| | | [mA][mA][fU][mC][mU | | [mU][mC][mU][mG][fU |
| | | ][mC][mA][fA][mA][m | | ][mU][mG][mU][mA][m |
| | | A][idT] | | U][*mU][*mU] |
| Y4 | 148 | [mC*][mA*][fG][mA][ | 281 | [fU*][fC*][mG][mA][ |
| | | mA][mA][fA][fC][fC] | | mG][mC][mU][mG][mU] |
| | | [mC][mA][fC][mA][mG | | [mG][mG][mG][mU][fU |
| | | ][mC][mU][fC][mG][m | | ][mU][mU][mC][mU][m |
| | | A][idT] | | G][*mU][*mU] |
| Z4 | 149 | [mA*][mC*][fC][mC][ | 282 | [fU*][fA*][mC][mU][ |
| | | mA][mC][fA][fG][fC] | | mC][mU][mU][mC][mG] |
| | | [mU][mC][fG][mA][mA | | [mA][mG][mC][mU][fG |
| | | ][mG][mA][fG][mU][m | | ][mU][mG][mG][mG][m |
| | | A][idT] | | U][*mU][*mU] |
| A5 | 150 | [mG*][mC*][fU][mC][ | 283 | [fU*][fC*][mG][mU][ |
| | | mG][mA][fA][fG][fA] | | mC][mA][mC][mC][mA] |
| | | [mG][mU][fG][mG][mU | | [mC][mU][mC][mU][fU |
| | | ][mG][mA][fC][mG][m | | ][mC][mG][mA][mG][m |
| | | A][idT] | | C][*mU][*mU] |
| B5 | 151 | [mU*][mC*][fG][mA][ | 284 | [fU*][fG*][mA][mC][ |
| | | mA][mG][fA][fG][fU] | | mG][mU][mC][mA][mC] |
| | | [mG][mG][fU][mG][mA | | [mC][mA][mC][mU][fC |
| | | ][mC][mG][fU][mC][m | | ][mU][mU][mC][mG][m |
| | | A][idT] | | A][*mU][*mU] |
| C5 | 152 | [mG*][mA*][fA][mG][ | 285 | [fU*][fC*][mA][mG][ |
| | | mA][mG][fU][fG][fG] | | mA][mC][mG][mU][mC] |
| | | [mU][mG][fA][mC][mG | | [mA][mC][mC][mA][fC |
| | | ][mU][mC][fU][mG][m | | ][mU][mC][mU][mU][m |
| | | A][idT] | | C][*mU][*mU] |
| D5 | 153 | [mG*][mG*][fU][mU][ | 286 | [fU*][fA*][mU][mC][ |
| | | mG][mG][fU][fU][fA] | | mU][mC][mC][mC][mU] |
| | | [mA][mA][fG][mG][mG | | [mU][mU][mA][mA][fC |
| | | ][mA][mG][fA][mu][m | | ][mC][mA][mA][mC][m |
| | | A][idT] | | C][*mU][*mU] |
| E5 | 154 | [mU*][mU*][fG][mG][ | 287 | [fU*][fG*][mC][mA][ |
| | | mC][mU][fU][fU][fC] | | mU][mU][mA][mU][mG] |
| | | [mC][mC][fA][mU][mA | | [mG][mG][mA][mA][fA |
| | | ][mA][mU][fG][mC][m | | ][mG][mC][mC][mA][m |
| | | A][idT] | | A][*mU][*mU] |
| F5 | 155 | [mC*][mC*][fC][mU][ | 288 | [fU*][fG*][mA][mA][ |
| | | mG][mC][fC][fC][fA] | | mG][mC][mC][mG][mA] |
| | | [mG][mU][fC][mG][mG | | [mC][mU][mG][mG][fG |
| | | ][mC][mU][fU][mC][m | | ][mC][mA][mG][mG][m |
| | | A][idT] | | G][*mU][*mU] |
| G5 | 156 | [mC*][mC*][fU][mG][ | 289 | [fU*][fA*][mG][mA][ |
| | | mC][mC][fC][fA][fG] | | mA][mG][mC][mC][mG] |
| | | [mU][mC][fG][mG][mC | | [mA][mC][mU][mG][fG |
| | | ][mU][mU][fC][mU][m | | ][mG][mC][mA][mG][m |
| | | A][idT] | | G][*mU][*mU] |
| H5 | 157 | [mU*][mG*][fC][mC][ | 290 | [fU*][fG*][mA][mA][ |
| | | mC][mA][fG][fU][fC] | | mG][mA][mA][mG][mC] |
| | | [mG][mG][fC][mU][mU | | [mC][mG][mA][mC][fU |
| | | ][mC][mU][fU][mC][m | | ][mG][mG][mG][mC][m |
| | | A][idT] | | A][*mU][*mU] |
| I5 | 158 | [mG*][mC*][fC][mC][ | 291 | [fU*][fA*][mG][mA][ |
| | | mA][mG][fU][fC][fG] | | mA][mG][mA][mA][mG] |
| | | [mG][mC][fU][mU][mC | | [mC][mC][mG][mA][fC |
| | | ][mU][mU][fC][mU][m | | ][mU][mG][mG][mG][m |
| | | A][idT] | | C][*mU][*mU] |
| J5 | 159 | [mC*][mC*][fA][mG][ | 292 | [fU*][fG*][mG][mA][ |
| | | mU][mC][fG][fG][fC] | | mG][mA][mA][mG][mA] |
| | | [mU][mU][fC][mU][mU | | [mA][mG][mC][mC][fG |
| | | ][mC][mU][fC][mC][m | | ][mA][mC][mU][mG][m |
| | | A][idT] | | G][*mU][*mU] |
| K5 | 160 | [mC*][mA*][fG][mU][ | 293 | [fU*][fU*][mG][mG][ |
| | | mC][mG][fG][fC][fU] | | mA][mG][mA][mA][mG] |
| | | [mU][mC][fU][mU][mC | | [mA][mA][mG][mC][fC |
| | | ][mU][mC][fC][mA][m | | ][mG][mA][mC][mU][m |
| | | A][idT] | | G][*mU][*mU] |
| L5 | 161 | [mA*][mG*][fU][mC][ | 294 | [fU*][fU*][mU][mG][ |
| | | mG][mG][fC][fU][fU] | | mG][mA][mG][mA][mA] |
| | | [mC][mU][fU][mC][mU | | [mG][mA][mA][mG][fC |
| | | ][mC][mC][fA][mA][m | | ][mC][mG][mA][mC][m |
| | | A][idT] | | U][*mU][*mU] |
| M5 | 162 | [mG*][mC*][fU][mG][ | 295 | [fU*][fC*][mU][mC][ |
| | | mC][mU][fC][fC][fA] | | mC][mU][mG][mC][mA] |
| | | [mG][mU][fG][mC][mA | | [mC][mU][mG][mG][fA |
| | | ][mG][mG][fA][mG][m | | ][mG][mC][mA][mG][m |
| | | A][idT] | | C][*mU][*mU] |
| N5 | 163 | [mC*][mU*][fG][mC][ | 296 | [fU*][fU*][mC][mU][ |
| | | mU][mC][fC][fA][fG] | | mC][mC][mU][mG][mC] |
| | | [mU][mG][fC][mA][mG | | [mA][mC][mU][mG][fG |
| | | ][mG][mA][fG][mA][m | | ][mA][mG][mC][mA][m |
| | | A][idT] | | G][*mU][*mU] |
| O5 | 164 | [mA*][mG*][fU][mC][ | 297 | [fU*][fC*][mU][mG][ |
| | | mG][mC][fA][fU][fC] | | mC][mA][mC][mU][mG] |
| | | [mU][mC][fA][mG][mU | | [mA][mG][mA][mU][fG |
| | | ][mG][mC][fA][mG][m | | ][mC][mG][mA][mC][m |
| | | A][idT] | | U][*mU][*mU] |
| P5 | 165 | [mU*][mC*][fG][mC][ | 298 | [fU*][fU*][mC][mC][ |
| | | mA][mU][fC][fU][fC] | | mU][mG][mC][mA][mC] |
| | | [mA][mG][fU][mG][mC | | [mU][mG][mA][mG][fA |
| | | ][mA][mG][fG][mA][m | | ][mU][mG][mC][mG][m |
| | | A][idT] | | A][*mU][*mU] |
| Q5 | 166 | [mC*][mC*][fC][mA][ | 299 | [fU*][fG*][mA][mG][ |
| | | mG][mU][fC][fG][fG] | | mA][mA][mG][mA][mA] |
| | | [mC][mU][fU][mC][mU | | [mG][mC][mC][mG][fA |
| | | ][mU][mC][fU][mC][m | | ][mC][mU][mG][mG][m |
| | | A][idT] | | G][*mU][*mU] |
| R5 | 167 | [mC*][mC*][fC][mU][ | 300 | [fU*][fG*][mA][mA][ |
| | | mG][mC][fC][fC][fA] | | mG][mC][mC][mG][mA] |
| | | [mG][mU][fC][mG][mG | | [mC][mU][mG][mG][fG |
| | | ][mC][mU][fU][mC][m | | ][mC][mA][mG][mG][m |
| | | A][mU][*mU][*idT] | | G][*mU][*mU] |
| S5 | 168 | [mC*][mC*][fU][mG][ | 301 | [fU*][fA*][mG][mA][ |
| | | mC][mC][fC][fA][fG] | | mA][mG][mC][mC][mG] |
| | | [mU][mC][fG][mG][mC | | [mA][mC][mU][mG][fG |
| | | ][mU][mU][fC][mU][m | | ][mG][mC][mA][mG][m |
| | | A][mU][*mU][*idT] | | G][*mU][*mU] |
| T5 | 169 | [mU*][mG*][fC][mC][ | 302 | [fU*][fG*][mA][mA][ |
| | | mC][mA][fG][fU][fC] | | mG][mA][mA][mG][mC] |
| | | [mG][mG][fC][mU][mU | | [mC][mG][mA][mC][fU |
| | | ][mC][mU][fU][mC][m | | ][mG][mG][mG][mC][m |
| | | A][mU][*mU][*idT] | | A][*mU][*mU] |
| U5 | 170 | [mG*][mC*][fC][mC][ | 303 | [fU*][fA*][mG][mA][ |
| | | mA][mG][fU][fC][fG] | | mA][mG][mA][mA][mG] |
| | | [mG][mC][fU][mU][mC | | [mC][mC][mG][mA][fC |
| | | ][mU][mU][fC][mU][m | | ][mU][mG][mG][mG][m |
| | | A][mU][*mU][*idT] | | C][*mU][*mU] |
| V5 | 171 | [mC*][mC*][fC][mA][ | 304 | [fU*][fG*][mA][mG][ |
| | | mG][mU][fC][fG][fG] | | mA][mA][mG][mA][mA] |
| | | [mC][mU][fU][mC][mU | | [mG][mC][mC][mG][fA |
| | | ][mU][mC][fU][mC][m | | ][mC][mU][mG][mG][m |
| | | A][mU][*mU][*idT] | | G][*mU][*mU] |
| W5 | 172 | [mC*][mC*][fA][mG][ | 305 | [fU*][fG*][mG][mA][ |
| | | mU][mC][fG][fG][fC] | | mG][mA][mA][mG][mA] |
| | | [mU][mU][fC][mU][mu | | [mA][mG][mC][mC][fG |
| | | ][mC][mU][fC][mC][m | | ][mA][mC][mU][mG][m |
| | | A][mU][*mU][*idT] | | G][*mU][*mU] |
| X5 | 173 | [mC*][mA*][fG][mU][ | 306 | [fU*][fU*][mG][mG][ |
| | | mC][mG][fG][fC][fU] | | mA][mG][mA][mA][mG] |
| | | [mU][mC][fU][mU][mC | | [mA][mA][mG][mC][fC |
| | | ][mU][mC][fU][mU][m | | ][mG][mA][mC][mU][m |
| | | A][mU][*mU][*idT] | | G][*mU][*mU] |
| Y5 | 174 | [mG*][mC*][fU][mG][ | 307 | [fU*][fC*][mU][mC][ |
| | | mC][mU][fC][fC][fA] | | mC][mU][mG][mC][mA] |
| | | [mG][mU][fG][mC][mA | | [mC][mU][mG][mG][fA |
| | | ][mG][mG][fA][mG][m | | ][mG][mC][mA][mG][m |
| | | A][mU][*mU][*idT] | | C][*mU][*mU] |
| Z5 | 175 | [mC*][mU*][fG][mC][ | 308 | [fU*][fU*][mC][mU][ |
| | | mU][mC][fC][fA][fG] | | mC][mC][mU][mG][mC] |
| | | [mU][mG][fC][mA][mG | | [mA][mC][mU][mG][fG |
| | | ][mG][mA][fG][mA][m | | ][mA][mG][mC][mA][m |
| | | A][mU][*mU][*idT] | | G][*mU][*mU] |
| A6 | 176 | [mA*][mG*][fU][mC][ | 309 | [fU*][fC*][mU][mG][ |
| | | mG][mC][fA][fU][fC] | | mC][mA][mC][mU][mG] |
| | | [mU][mC][fA][mG][mU | | [mA][mG][mA][mU][fG |
| | | ][mG][mC][fA][mG][m | | ][mC][mG][mA][mC][m |
| | | A][mU][*mU][*idT] | | U][*mU][*mU] |
| B6 | 177 | [mU*][mC*][fG][mC][ | 310 | [fU*][fU*][mC][mC][ |
| | | mA][mU][fC][fU][fC] | | mU][mG][mC][mA][mC] |
| | | [mA][mG][fU][mG][mC | | [mU][mG][mA][mG][fA |
| | | ][mA][mG][fG][mA][m | | ][mU][mG][mC][mG][m |
| | | A][mU][*mU][*idT] | | A][*mU][*mU] |
| C6 | 178 | [mA*][mG*][fU][mC][ | 311 | [mU*][fU*][mU][mG][ |
| | | mG][mG][fC][fU][fU] | | mG][mA][mG][mA][mA] |
| | | [mC][mU][fU][mC][mU | | [mG][mA][mA][mG][fC |
| | | ][mC][mC][fA][mA][m | | ][mC][mG][mA][mC][m |
| | | A][mU][*mU][*idT] | | U][*mU][*mU] |

Abbreviations Key:
n/N = any nucleotide
mN = 2'-O-Methyl substitution
fN = 2'-F substitution
idT = inverted Dt
vinmN or vinmU = 2'-O methyl vinyl phosphonate uridine
* = phosphorothioate
The brackets indicate the individual bases.

TABLE 3B
siRNA Sense and Antisense Sequences (Unmodified)

| SEQ | | SEQ | |
|---|---|---|---|
| ID NO: | Sense Strand 5′-3′ | ID NO: | Antisense Strand 5′-3′ |
| 673 | AGAACCACCCACUGCAUGA | 806 | UCAUGCAGUGGGUGGUUCUUU |
| 674 | AGAAAAACAGUACCUAAUA | 807 | UAUUAGGUACUGUUUUUCUUU |
| 675 | GAAACUGGUGAGUGACUGA | 808 | UCAGUCACUCACCAGUUUCUU |
| 676 | AAACUGGUGAGUGACUGCA | 809 | UGCAGUCACUCACCAGUUUUU |
| 677 | AGUUCACUGAAACGGAAUA | 810 | UAUUCCGUUUCAGUGAACUUU |
| 678 | GGAAUGCCUUCCUUGCGGA | 811 | UCCGCAAGGAAGGCAUUCCUU |
| 679 | UGCCUUCCUUGCGGUGAAA | 812 | UUUCACCGCAAGGAAGGCAUU |
| 680 | GCCUUCCUUGCGGUGAAAA | 813 | UUUUCACCGCAAGGAAGGCUU |
| 681 | CCUUCCUUGCGGUGAAAGA | 814 | UCUUUCACCGCAAGGAAGGUU |
| 682 | CUUCCUUGCGGUGAAAGCA | 815 | UGCUUUCACCGCAAGGAAGUU |
| 683 | UUCCUUGCGGUGAAAGCGA | 816 | UCGCUUUCACCGCAAGGAAUU |
| 684 | UCCUUGCGGUGAAAGCGAA | 817 | UUCGCUUUCACCGCAAGGAUU |
| 685 | CCUUGCGGUGAAAGCGAAA | 818 | UUUCGCUUUCACCGCAAGGUU |
| 686 | CUUGCGGUGAAAGCGAAUA | 819 | UAUUCGCUUUCACCGCAAGUU |
| 687 | UUGCGGUGAAAGCGAAUUA | 820 | UAAUUCGCUUUCACCGCAAUU |
| 688 | UGCGGUGAAAGCGAAUUCA | 821 | UGAAUUCGCUUUCACCGCAUU |
| 689 | CGGUGAAAGCGAAUUCCUA | 822 | UAGGAAUUCGCUUUCACCGUU |
| 690 | GGUGAAAGCGAAUUCCUAA | 823 | UUAGGAAUUCGCUUUCACCUU |
| 691 | UGAAAGCGAAUUCCUAGAA | 824 | UUCUAGGAAUUCGCUUUCAUU |
| 692 | GAAAGCGAAUUCCUAGACA | 825 | UGUCUAGGAAUUCGCUUUCUU |
| 693 | AAAGCGAAUUCCUAGACAA | 826 | UUGUCUAGGAAUUCGCUUUUU |
| 694 | AAGCGAAUUCCUAGACACA | 827 | UGUGUCUAGGAAUUCGCUUUU |
| 695 | AGCGAAUUCCUAGACACCA | 828 | UGGUGUCUAGGAAUUCGCUUU |
| 696 | CUAGACACCUGGAACAGAA | 829 | UUCUGUUCCAGGUGUCUAGUU |
| 697 | CAGCACAAAUACUGCGACA | 830 | UGUCGCAGUAUUUGUGCUGUU |
| 698 | AGCACAAAUACUGCGACCA | 831 | UGGUCGCAGUAUUUGUGCUUU |
| 699 | GCACAAAUACUGCGACCCA | 832 | UGGGUCGCAGUAUUUGUGCUU |
| 700 | UACUGCGACCCCAACCUAA | 833 | UUAGGUUGGGGUCGCAGUAUU |
| 701 | ACUGCGACCCCAACCUAGA | 834 | UCUAGGUUGGGGUCGCAGUUU |
| 702 | CUGCGACCCCAACCUAGGA | 835 | UCCUAGGUUGGGGUCGCAGUU |
| 703 | GACCCCAACCUAGGGCUUA | 836 | UAAGCCCUAGGUUGGGGUCUU |
| 704 | ACCCCAACCUAGGGCUUCA | 837 | UGAAGCCCUAGGUUGGGGUUU |
| 705 | CCCCAACCUAGGGCUUCGA | 338 | UCGAAGCCCUAGGUUGGGGUU |
| 706 | CCUAGGGCUUCGGGUCCAA | 839 | UUGGACCCGAAGCCCUAGGUU |
| 707 | UCGGGUCCAGCAGAAGGGA | 840 | UCCCUUCUGCUGGACCCGAUU |
| 708 | GAAGGCUGGCACUGUACGA | 841 | UCGUACAGUGCCAGCCUUCUU |
| 709 | GGCACUGUACGAGUGAGGA | 842 | UCCUCACUCGUACAGUGCCUU |
| 710 | GUACGAGUGAGGCCUGUGA | 843 | UCACAGGCCUCACUCGUACUU |
| 711 | UGUCCUGCACCGCUCAUGA | 844 | UCAUGAGCGGUGCAGGACAUU |
| 712 | UGCACCGCUCAUGCUCGCA | 845 | UGCGAGCAUGAGCGGUGCAUU |
| 713 | GCACCGCUCAUGCUCGCCA | 846 | UGGCGAGCAUGAGCGGUGCUU |
| 714 | CCGCUCAUGCUCGCCCGGA | 847 | UCCGGGCGAGCAUGAGCGGUU |
| 715 | CCCGGCUUUGGGGUCAAGA | 848 | UCUUGACCCCAAAGCCGGGUU |
| 716 | CUGAUACCAUCUGCGAGCA | 849 | UGCUCGCAGAUGGUAUCAGUU |
| 717 | UGAUACCAUCUGCGAGCCA | 850 | UGGCUCGCAGAUGGUAUCAUU |
| 718 | CUGCCCAGUCGGCUUCUUA | 851 | UAAGAAGCCGACUGGGCAGUU |
| 719 | GUGUCAUCUGCUUUCGAAA | 852 | UUUCGAAAGCAGAUGACACUU |
| 720 | UGUCAUCUGCUUUCGAAAA | 853 | UUUUCGAAAGCAGAUGACAUU |
| 721 | UCAUCUGCUUUCGAAAAAA | 854 | UUUUUUCGAAAGCAGAUGAUU |
| 722 | UUCGAAAAAUGUCACCCUA | 855 | UAGGGUGACAUUUUUCGAAUU |
| 723 | UCGAAAAAUGUCACCCUUA | 856 | UAAGGGUGACAUUUUUCGAUU |
| 724 | AAAAUGUCACCCUUGGACA | 857 | UGUCCAAGGGUGACAUUUUUU |
| 725 | UGAUCCCCAUCAUCUUCGA | 858 | UCGAAGAUGAUGGGGAUCAUU |
| 726 | CCCCAUCAUCUUCGGGAUA | 859 | UAUCCCGAAGAUGAUGGGGUU |
| 727 | AUCUUCGGGAUCCUGUUUA | 860 | UAAACAGGAUCCCGAAGAUUU |
| 728 | AAGAAGCCAACCAAUAAGA | 861 | UCUUAUUGGUUGGCUUCUUUU |
| 729 | AAGCCAACCAAUAAGGCCA | 862 | UGGCCUUAUUGGUUGGCUUUU |
| 730 | AGAUCAAUUUUCCCGACGA | 863 | UCGUCGGGAAAAUUGAUCUUU |
| 731 | GAUCAAUUUUCCCGACGAA | 864 | UUCGUCGGGAAAAUUGAUCUU |
| 732 | AUCAAUUUUCCCGACGAUA | 865 | UAUCGUCGGGAAAAUUGAUUU |
| 733 | UCAAUUUUCCCGACGAUCA | 866 | UGAUCGUCGGGAAAAUUGAUU |
| 734 | CGACGAUCUUCCUGGCUCA | 867 | UGAGCCAGGAAGAUCGUCGUU |
| 735 | ACGAUCUUCCUGGCUCCAA | 868 | UUGGAGCCAGGAAGAUCGUUU |
| 736 | GAUCUUCCUGGCUCCAACA | 869 | UGUUGGAGCCAGGAAGAUCUU |
| 737 | UGCAGGAGACUUUACAUGA | 870 | UCAUGUAAAGUCUCCUGCAUU |
| 738 | CAGGAGACUUUACAUGGAA | 871 | UUCCAUGUAAAGUCUCCUGUU |
| 739 | GGAGACUUUACAUGGAUGA | 872 | UCAUCCAUGUAAAGUCUCCUU |
| 740 | AGGAUGGCAAAGAGAGUCA | 873 | UGACUCUCUUUGCCAUCCUUU |
| 741 | GGAUGGCAAAGAGAGUCGA | 874 | UCGACUCUCUUUGCCAUCCUU |
| 742 | UGGCAAAGAGAGUCGCAUA | 875 | UAUGCGACUCUCUUUGCCAUU |
| 743 | GGCAAAGAGAGUCGCAUCA | 876 | UGAUGCGACUCUCUUUGCCUU |
| 744 | GCAAAGAGAGUCGCAUCUA | 877 | UAGAUGCGACUCUCUUUGCUU |
| 745 | AAAGAGAGUCGCAUCUCAA | 878 | UUGAGAUGCGACUCUCUUUUU |
| 746 | AAGAGAGUCGCAUCUCAGA | 879 | UCUGAGAUGCGACUCUCUUUU |
| 747 | AGAGAGUCGCAUCUCAGUA | 880 | UACUGAGAUGCGACUCUCUUU |
| 748 | GAGAGUCGCAUCUCAGUGA | 881 | UCACUGAGAUGCGACUCUCUU |
| 749 | AGAGUCGCAUCUCAGUGCA | 882 | UGCACUGAGAUGCGACUCUUU |
| 750 | GAGUCGCAUCUCAGUGCAA | 883 | UUGCACUGAGAUGCGACUCUU |
| 751 | GUCGCAUCUCAGUGCAGGA | 884 | UCCUGCACUGAGAUGCGACUU |
| 752 | CUGCUGUGGCGUGAGGGUA | 885 | UACCCUCACGCCACAGCAGUU |
| 753 | GGCACUGACUGGGCAUAGA | 886 | UCUAUGCCCAGUCAGUGCCUU |
| 754 | GGGCAUAGCUCCCCGCUUA | 887 | UAAGCGGGGAGCUAUGCCCUU |
| 755 | AGAACCUCUCACUUCACCA | 888 | UGGUGAAGUGAGAGGUUCUUU |
| 756 | GAACCUCUCACUUCACCCA | 889 | UGGGUGAAGUGAGAGGUUCUU |
| 757 | AGUCUCCCAACUUGUAUUA | 890 | UAAUACAAGUUGGGAGACUUU |
| 758 | GGUUUAGUAAUAUCCACCA | 891 | UGGUGGAUAUUACUAAACCUU |
| 759 | GUUUAGUAAUAUCCACCAA | 892 | UUGGUGGAUAUUACUAAACUU |
| 760 | UUUAGUAAUAUCCACCAGA | 893 | UCUGGUGGAUAUUACUAAAUU |
| 761 | GUAAUAUCCACCAGACCUA | 894 | UAGGUCUGGUGGAUAUUACUU |
| 762 | UAAUAUCCACCAGACCUUA | 895 | UAAGGUCUGGUGGAUAUUAUU |
| 763 | CAUGGUGGCUUCCCUGCGA | 896 | UCGCAGGGAAGCCACCAUGUU |
| 764 | UGCGCCCAGGAAGCCAUAA | 897 | UUAUGGCUUCCUGGGCGCAUU |
| 765 | CGCCCAGGAAGCCAUAUAA | 898 | UUAUAUGGCUUCCUGGGCGUU |
| 766 | AGCCAUAUACACAGAUGCA | 899 | UGCAUCUGUGUAUAUGGCUUU |
| 767 | GCCAUAUACACAGAUGCCA | 900 | UGGCAUCUGUGUAUAUGGCUU |
| 768 | UUGUUUGUGAUAGUGAACA | 901 | UGUUCACUAUCACAAACAAUU |
| 769 | GGAAGCUGCUUAACUGUCA | 902 | UGACAGUUAAGCAGCUUCCUU |
| 770 | GCUGCUUAACUGUCCAUCA | 903 | UGAUGGACAGUUAAGCAGCUU |
| 771 | AGCAGGAGACUGGCUAAAA | 904 | UUUUAGCCAGUCUCCUGCUUU |
| 772 | GCAGGAGACUGGCUAAAUA | 905 | UAUUUAGCCAGUCUCCUGCUU |
| 773 | CAGGAGACUGGCUAAAUAA | 906 | UUAUUUAGCCAGUCUCCUGUU |
| 774 | AUACAACAGAAUCUCAAAA | 907 | UUUUGAGAUUCUGUUGUAUUU |
| 775 | CAGAAAACCCACAGCUCGA | 908 | UCGAGCUGUGGGUUUUCUGUU |
| 776 | ACCCACAGCUCGAAGAGUA | 909 | UACUCUUCGAGCUGUGGGUUU |
| 777 | GCUCGAAGAGUGGUGACGA | 910 | UCGUCACCACUCUUCGAGCUU |
| 778 | UCGAAGAGUGGUGACGUCA | 911 | UGACGUCACCACUCUUCGAUU |
| 779 | GAAGAGUGGUGACGUCUGA | 912 | UCAGACGUCACCACUCUUCUU |
| 780 | GGUUGGUUAAAGGGAGAUA | 913 | UAUCUCCCUUUAACCAACCUU |
| 781 | UUGGCUUUCCCAUAAUGCA | 914 | UGCAUUAUGGGAAAGCCAAUU |
| 782 | CCCUGCCCAGUCGGCUUCA | 915 | UGAAGCCGACUGGGCAGGGUU |
| 783 | CCUGCCCAGUCGGCUUCUA | 916 | UAGAAGCCGACUGGGCAGGUU |
| 784 | UGCCCAGUCGGCUUCUUCA | 917 | UGAAGAAGCCGACUGGGCAUU |
| 785 | GCCCAGUCGGCUUCUUCUA | 918 | UAGAAGAAGCCGACUGGGCUU |
| 786 | CCAGUCGGCUUCUUCUCCA | 919 | UGGAGAAGAAGCCGACUGGUU |
| 787 | CAGUCGGCUUCUUCUCCAA | 920 | UUGGAGAAGAAGCCGACUGUU |
| 788 | AGUCGGCUUCUUCUCCAAA | 921 | UUUGGAGAAGAAGCCGACUUU |
| 789 | GCUGCUCCAGUGCAGGAGA | 922 | UCUCCUGCACUGGAGCAGCUU |
| 790 | CUGCUCCAGUGCAGGAGAA | 923 | UUCUCCUGCACUGGAGCAGUU |
| 791 | AGUCGCAUCUCAGUGCAGA | 924 | UCUGCACUGAGAUGCGACUUU |
| 792 | UCGCAUCUCAGUGCAGGAA | 925 | UUCCUGCACUGAGAUGCGAUU |
| 793 | CCCAGUCGGCUUCUUCUCA | 926 | UGAGAAGAAGCCGACUGGGUU |
| 794 | CCCUGCCCAGUCGGCUUCAUU | 927 | UGAAGCCGACUGGGCAGGGUU |
| 795 | CCUGCCCAGUCGGCUUCUAUU | 928 | UAGAAGCCGACUGGGCAGGUU |
| 796 | UGCCCAGUCGGCUUCUUCAUU | 929 | UGAAGAAGCCGACUGGGCAUU |
| 797 | GCCCAGUCGGCUUCUUCUAUU | 930 | UAGAAGAAGCCGACUGGGCUU |
| 798 | CCCAGUCGGCUUCUUCUCAUU | 931 | UGAGAAGAAGCCGACUGGGUU |
| 799 | CCAGUCGGCUUCUUCUCCAUU | 932 | UGGAGAAGAAGCCGACUGGUU |
| 800 | CAGUCGGCUUCUUCUCCAAUU | 933 | UUGGAGAAGAAGCCGACUGUU |
| 801 | GCUGCUCCAGUGCAGGAGAUU | 934 | UCUCCUGCACUGGAGCAGCUU |
| 802 | CUGCUCCAGUGCAGGAGAAUU | 935 | UUCUCCUGCACUGGAGCAGUU |
| 803 | AGUCGCAUCUCAGUGCAGAUU | 936 | UCUGCACUGAGAUGCGACUUU |
| 804 | UCGCAUCUCAGUGCAGGAAUU | 937 | UUCCUGCACUGAGAUGCGAUU |
| 805 | AGUCGGCUUCUUCUCCAAAUU | 938 | UUUGGAGAAGAAGCCGACUUU |

TABLE 4A
siRNA Sense and Antisense Sequences (Modified)

| siRNA | SEQ | | SEQ | |
|---|---|---|---|---|
| Pair | ID NO: | Sense Strand 5′-3′ | ID NO: | Antisense Strand 5′-3′ |
| D6 | 312 | [mG*][mA*][fG][mU][ | 332 | [vinmU*][fU*][mG][m |
| | | mC][mG][fC][fA][fU] | | C][mA][mC][mU][mG][ |
| | | [mC][mU][fC][mA][mG | | mA][mG][mA][mU][mG] |
| | | ][mU][mG][fC][mA][m | | [fC][mG][mA][mC][mU |
| | | A] | | ][mC][*mU][*mU] |
| E6 | 313 | [mG*][mU*][fC][mG][ | 333 | [vinmU*][fC*][mC][m |
| | | mC][mA][fU][fC][fU] | | U][mG][mC][mA][mC][ |
| | | [mC][mA][fG][mU][mG | | mU][mG][mA][mG][mA] |
| | | ][mC][mA][fG][mG][m | | [fU][mG][mC][mG][mA |
| | | A] | | ][mC][*mU][*mU] |
| F6 | 314 | [mA*][mG*][fA][mA][ | 334 | [vinmU*][fG*][mG][m |
| | | mC][mC][fU][fC][fU] | | U][mG][mA][mA][mG][ |
| | | [mC][mA][fC][mU][mU | | mU][mG][mA][mG][mA] |
| | | ][mC][mA][fC][mC][m | | [fG][mG][mU][mU][mc |
| | | A] | | ][mU][*mU][*mU] |
| G6 | 315 | [mG*][mA*][fA][mC][ | 335 | [vinmU*][fG*][mG][m |
| | | mC][mU][fC][fU][fC] | | G][mU][mG][mA][mA][ |
| | | [mA][mC][fU][mU][mC | | mG][mU][mG][mA][mG] |
| | | ][mA][mC][fC][mC][m | | [fA][mG][mG][mU][mU |
| | | A] | | ][mC][*mU][*mU] |
| H6 | 316 | [mA*][mG*][fU][mC][ | 336 | [vinmU*][fA*][mA][m |
| | | mU][mC][fC][fC][fA] | | U][mA][mC][mA][mA][ |
| | | [mA][mC][fU][mU][mG | | mG][mU][mU][mG][mG] |
| | | ][mU][mA][fU][mu][m | | [fG][mA][mG][mA][mC |
| | | A] | | ][mU][*mU][*mU] |
| I6 | 317 | [mG*][mG*][fA][mA][ | 337 | [vinmU*][fG*][mA][m |
| | | mG][mC][fU][fG][fC] | | C][mA][mG][mU][mU][ |
| | | [mU][mU][fA][mA][mc | | mA][mA][mG][mC][mA] |
| | | ][mU][mG][fU][mC][m | | [fG][mC][mU][mU][mc |
| | | A] | | ][mC][*mU][*mU] |
| J6 | 318 | [mG*][mC*][fA][mG][ | 338 | [vinmU*][fA*][mU][m |
| | | mG][mA][fG][fA][fC] | | U][mU][mA][mG][mC][ |
| | | [mU][mG][fG][mC][mU | | mC][mA][mG][mU][mC] |
| | | ][mA][mA][fA][mU][m | | [fU][mC][mC][mU][mG |
| | | A] | | ][mC][*mU][*mU] |
| K6 | 319 | [mU*][mU*][fA][mU][ | 339 | [vinmU*][fC*][mA][m |
| | | mC][mC][fU][fU][fu] | | A][mG][mA][mA][mA][ |
| | | [mG][mG][fU][mU][mU | | mC][mC][mA][mA][mA] |
| | | ][mC][mU][fU][mG][m | | [fG][mG][mA][mU][mA |
| | | A] | | ][mA][*mU][*mU] |
| L6 | 320 | [mG*][mU*][[G][mU][ | 340 | [vinmU*][fU*][mC][m |
| | | mG][mU][fU][fA][fC] | | A][mC][mU][mG][mC][ |
| | | [mG][mU][fG][mC][mA | | mA][mC][mG][mU][mA] |
| | | ][mG][mU][fG][mA][m | | [fA][mC][mA][mC][mA |
| | | A][mU][mU] | | ][mC][*mU][*mG] |
| M6 | 321 | [mU*][mG*][fU][mU][ | 341 | [vinmU*][fU*][mC][m |
| | | mC][mC][fA][fC][fU] | | U][mC][mA][mG][mC][ |
| | | [mG][mG][fG][mC][mU | | mC][mC][mA][mG][mU] |
| | | ][mG][mA][fG][mA][m | | [fG][mG][mA][mA][mC |
| | | A] | | ][mA][*mU][*mU] |
| N6 | 322 | [mG*][mC*][fG][mA][ | 342 | [vinmU*][fA*][mG][m |
| | | mA][mU][fU][fC][fC] | | G][mU][mG][mU][mC][ |
| | | [mU][mA][fG][mA][mC | | mU][mC][mG][mG][mA] |
| | | ][mA][mC][fC][mU][m | | [fA][mU][mU][mC][mG |
| | | A][mU][mU] | | ][mC][*mU][*mU] |
| O6 | 323 | [mG*][mU*][fG][mU][ | 343 | [vinmU*][fA*][mG][m |
| | | mG][mU][fU][fA][fC] | | U][mC][mA][mC][mU][ |
| | | [mG][mU][fG][mC][mA | | mG][mC][mA][mC][mG] |
| | | ][mG][mU][fG][mA][m | | [fU][mA][mA][mC][mA |
| | | C][mU][mA] | | ][mC][mA][mC][*mU][ |
| | | | | *mG] |
| P6 | 324 | [mA*][mC*][fU][mA][ | 344 | [vinmU*][fG*][mG][m |
| | | mC][mA][fA][fG][fA] | | U][mC][mA][mC][mG][ |
| | | [mC][mU][fC][mG][mU | | mA][mG][mU][mC][mU] |
| | | ][mG][mA][fC][mC][m | | [fU][mG][mU][mA][mG |
| | | A][mU][mu] | | ][mU][*mU][*mU] |
| Q6 | 325 | [fG*][mU*][fG][mU][ | 345 | [vinmU*][fU*][mC][f |
| | | fG][mU][fU][mA][fC] | | A][mC][fU][mG][fC][ |
| | | [mG][fU][mG][fC][mA | | mA][fC][mG][fU][Am] |
| | | ][fG][mU][fG][mA][f | | [fA][mC][fA][mC][fA |
| | | A][mU][mU] | | ][mC][*mU][*mG] |
| R6 | 326 | [fG*][mC*][[G][mA][ | 346 | [vinmU*][fA*][mG][f |
| | | fA][mU][fU][mC][fc] | | G][mU][fG][mu][fC][ |
| | | [mU][fA][mG][fA][mc | | mU][fC][mG][fG][mA] |
| | | ][fA][mC][fC][mU][f | | [fA][mU][fU][mC][fG |
| | | A][mU][mU] | | ][mC][*mU][*mU] |
| S6 | 327 | [fG*][mU*][fG][mU][ | 347 | [vinmU*][fA*][mG][f |
| | | fG][mU][fU][mA][fC] | | U][mC][fA][mC][fu][ |
| | | [mG][fU][mG][fC][mA | | mG][fC][mA][fC][mG] |
| | | ][fG][mU][fG][mA][f | | [fU][mA][fA][mC][fA |
| | | C][mU][mA] | | ][mC][fA][mC][*fU][ |
| | | | | *mG] |
| T6 | 328 | [mU*][mC*][fC][mU][ | 348 | [vinmU*][fU*][mC][m |
| | | mU][mG][fC][fG][fG] | | G][mC][mU][mU][mU][ |
| | | [mU][mG][fA][mA][mA | | mC][mA][mC][mC][mG] |
| | | ][mG][mC][fG][mA][m | | [fC][mA][mA][mG][mG |
| | | A] | | ][mA][*mU][*mU] |
| U6 | 329 | [mG*][mU*][fA][mA][ | 349 | [vinmU*][fA*][mG][m |
| | | mU][mA][fU][fC][fC] | | G][mU][mC][mU][mG][ |
| | | [mA][mC][fC][mA][mG | | mG][mU][mG][mG][mA] |
| | | ][mA][mC][fC][mU][m | | [fU][mA][mU][mU][mA |
| | | A] | | ][mC][*mU][*mU] |
| V6 | 330 | [mU*][mU*][fG][mU][ | 350 | [vinmU*][fG*][mU][m |
| | | mU][mU][fG][fU][fG] | | U][mC][mA][mC][mU][ |
| | | [mA][mU][fA][mG][mU | | mA][mU][mC][mA][mC] |
| | | ][mG][mA][fA][mC][m | | [fA][mA][mA][mC][mA |
| | | A] | | ][mA][*mU][*mU] |
| W6 | 331 | [mG*][mC*][fU][mG][ | 351 | [vinmU*][fG*][mA][m |
| | | mC][mU][fU][[A][[A] | | U][mG][mG][mA][mC][ |
| | | [mC][mU][fG][mU][mC | | mA][mG][mU][mU][mA] |
| | | ][mC][mA][fU][mC][m | | [fA][mG][mC][mA][mG |
| | | A] | | ][mC][*mU][*mU] |
| B7 | 1850 | [mA*][mG*][fC][mA][ | 1960 | [vinmU*][fU*][mU][m |
| | | mG][mG][fA][fG][fA] | | U][mA][mG][mC][mC][ |
| | | [mC][mU][fG][mG][mC | | mA][mG][mU][mC][mU] |
| | | ][mU][mA][fA][mA][m | | [fC][mC][mU][mG][mC |
| | | A] | | ][mU][*mU][*mU] |
| C7 | 1851 | [mC*][mA*][[G][mG][ | 1961 | [vinmu*][fU*][mA][m |
| | | mA][mG][fA][fC][fU] | | U][mU][mU][mA][mG][ |
| | | [mG][mG][fC][mU][mA | | mC][mC][mA][mG][mU] |
| | | ][mA][mA][fU][mA][m | | [fC][mU][mC][mC][mU |
| | | A] | | ][mG][*mU][*mU] |
| P8 | 1890 | [mA*][mG*][fA][mA][ | 2000 | [vin][mU][*fU][*mC] |
| | | mA][mC][fA][fG][fU] | | [mA][mA][mG][mG][mU |
| | | [mU][mC][fA][mC][mC | | ][mG][mA][mA][mC][m |
| | | ][mU][mU][fG][mA][m | | U][fG][mU][mU][Um][ |
| | | A] | | mC][m][*mU][*mU] |
| Q8 | 1891 | [mG*][mA*][fA][mA][ | 2001 | [vin][mU][*fU][*mU] |
| | | mC][mA][fG][fU][fU] | | [mC][mA][mA][mG][mG |
| | | [mC][mA][fC][mC][mU | | ][mU][mG][mA][mA][m |
| | | ][mU][mG][fA][mA][m | | C][fU][mG][mU][mu][ |
| | | A] | | mU][mC][*mU][*mU] |
| R8 | 1892 | [mA*][mA*][fC][mC][ | 2002 | [vin][mu][*[A][*mG] |
| | | mU][mC][fU][fC][fA] | | [mG][mG][mU][mG][mA |
| | | [mC][mU][fU][mC][mA | | ][mA][mG][mU][mG][m |
| | | ][mC][mC][fC][mU][m | | A][fG][mA][mG][mG][ |
| | | A] | | mU][mU][*mU][*mU] |
| S8 | 1893 | [mA*][mC*][fC][mU][ | 2003 | [vin][mU][*fC][*mA] |
| | | mC][mU][fC][fA][fC] | | [mG][mG][mG][mU][mG |
| | | [mU][mU][fC][mA][mC | | ][mA][mA][mG][mU][m |
| | | ][mC][mC][fU][mG][m | | G][fA][mG][mA][mG][ |
| | | A] | | mG][mU][*mU][*mU] |
| T8 | 1894 | [mC*][mC*][fU][mC][ | 2004 | [vin][mU][*fC][*mC] |
| | | mU][mC][fA][fC][fU] | | [mA][mG][mG][mG][mU |
| | | [mU][mC][fA][mC][mC | | ][mG][mA][mA][mG][m |
| | | ][mC][mU][fG][mG][m | | U][fG][mA][mG][mA][ |
| | | A] | | mG][mG][*mU][*mU] |
| U8 | 1895 | [mC*][mU*][fC][mU][ | 2005 | [vin][mU][*fU][*mC] |
| | | mC][mA][fC][fU][fU] | | [mC][mA][mG][mG][mG |
| | | [mC][mA][fC][mC][mC | | ][mU][mG][mA][mA][m |
| | | ][mU][mG][fG][mA][m | | G][fU][mG][mA][mG][ |
| | | A] | | mA][mG][*mU][*mU] |
| V8 | 1896 | [mU*][mC*][fU][mC][ | 2006 | [vin][mU][*fC][*mU] |
| | | mA][mC][fU][fU][fC] | | [mC][mC][mA][mG][mG |
| | | [mA][mC][fC][mC][mU | | ][mG][mU][mG][mA][m |
| | | ][mG][mG][fA][mG][m | | A][fG][mU][mG][mA][ |
| | | A] | | mG][mA][*mU][*mU] |
| W8 | 1897 | [mC*][mU*][fC][mA][ | 2007 | [vin][mU][*fG][*mC] |
| | | mC][mU][fU][fC][fA] | | [mU][mC][mC][mA][mG |
| | | [mC][mC][fC][mU][mG | | ][mG][mG][mU][mG][m |
| | | ][mG][mA][fG][mC][m | | A][fA][mG][mU][mG][ |
| | | A] | | mA][mG][*mU][*mU] |
| X8 | 1898 | [mU*][mC*][fA][mC][ | 2008 | [vinmU*][fG*][mG][m |
| | | mU][mU][fC][fA][fC] | | C][mU][mC][mC][mA][ |
| | | [mC][mC][fU][mG][mG | | mG][mG][mG][mU][mG] |
| | | ][mA][mG][fC][mC][m | | [fA][mA][mG][mU][mG |
| | | A] | | ][mA][*mU][*mU] |
| Y8 | 1899 | [mC*][mA*][fC][mU][ | 2009 | [vinmU*][fG*][mG][m |
| | | mU][mC][fA][fC][fC] | | G][mC][mU][mC][mC][ |
| | | [mC][mU][fG][mG][mA | | mA][mG][mG][mG][mU] |
| | | ][mG][mC][fC][mC][m | | [fG][mA][mA][mG][mU |
| | | A] | | ][mG][*mU][*mU] |
| Z8 | 1900 | [mA*][mC*][fU][mU][ | 2010 | [vinmU*][fU*][mG][m |
| | | mC][mA][fC][fC][fC] | | G][mG][mC][mU][mC][ |
| | | [mU][mG][fG][mA][mG | | mC][mA][mG][mG][mG] |
| | | ][mC][mC][fC][mA][m | | [fU][mG][mA][mA][mG |
| | | A] | | ][mU][*mU][*mU] |
| A9 | 1901 | [mC*][mU*][fU][mC][ | 2011 | [vinmU*][fA*][mU][m |
| | | mA][mC][fC][fC][fU] | | G][mG][mG][mC][mu][ |
| | | [mG][mG][fA][mG][mC | | mC][mC][mA][mG][mG] |
| | | ][mC][mC][fA][mU][m | | [fG][mU][mG][mA][mA |
| | | A] | | ][mG][*mU][*mU] |
| B9 | 1902 | [mU*][mU*][fC][mA][ | 2012 | [vinmU*][fG*][mA][m |
| | | mC][mC][fC][fU][fG] | | U][mG][mG][mG][mC][ |
| | | [mG][mA][fG][mC][mC | | mU][mC][mC][mA][mG] |
| | | ][mC][mA][fU][mC][m | | [fG][mG][mU][mG][mA |
| | | A] | | ][mA][*mU][*mU] |
| C9 | 1903 | [mU*][mC*][fA][mC][ | 2013 | [vinmU*][fG*][mG][m |
| | | mC][mC][fU][fG][fG] | | A][mU][mG][mG][mG][ |
| | | [mA][mG][fC][mC][mC | | mC][mU][mC][mC][mA] |
| | | ][mA][mU][fC][mC][m | | [fG][mG][mG][mU][mG |
| | | A] | | ][mA][*mU][*mU] |
| D9 | 1904 | [mC*][mA*][fC][mC][ | 2014 | [vinmU*][fU*][mG][m |
| | | mC][mU][fG][fG][fA] | | G][mA][mU][mG][mG][ |
| | | [mG][mC][fC][mC][mA | | mG][mC][mU][mC][mC] |
| | | ][mU][mC][fC][mA][m | | [fA][mG][mG][mG][mU |
| | | A] | | ][mG][*mU][*mU] |
| E9 | 1905 | [mA*][mC*][fC][mC][ | 2015 | [vinmU*][fC*][mU][m |
| | | mU][mG][fG][fA][fG] | | G][mG][mA][mU][mG][ |
| | | [mC][mC][fC][mA][mU | | mG][mG][mC][mU][mC] |
| | | ][mC][mC][fA][mG][m | | [fC][mA][mG][mG][mG |
| | | A] | | ][mU][*mU][*mU] |
| F9 | 1906 | [mC*][mC*][fC][mU][ | 2016 | [vinmU*][fA*][mC][m |
| | | mG][mG][fA][fG][fC] | | U][mG][mG][mA][mU][ |
| | | [mC][mC][fA][mU][mC | | mG][mG][mG][mC][mU] |
| | | ][mC][mA][fG][mU][m | | [fC][mC][mA][mG][mG |
| | | A] | | ][mG][*mU][*mU] |
| G9 | 1907 | [mC*][mC*][fU][mG][ | 2017 | [vinmU*][fG*][mA][m |
| | | mG][mA][fG][fC][fC] | | C][mU][mG][mG][mA][ |
| | | [mC][mA][fU][mC][mC | | mU][mG][mG][mG][mC] |
| | | ][mA][mG][fU][mC][m | | [fU][mC][mC][mA][mG |
| | | A] | | ][mG][*mU][*mU] |
| H9 | 1908 | [mC*][mU*][fG][mG][ | 2018 | [vinmU*][fA*][mG][m |
| | | mA][mG][fC][fC][fC] | | A][mC][mU][mG][mG][ |
| | | [mA][mU][fC][mC][mA | | mA][mU][mG][mG][mG] |
| | | ][mG][mU][fC][mU][m | | [fC][mU][mC][mC][mA |
| | | A] | | ][mG][*mU][*mU] |
| i9 | 1909 | [mC*][mU*][fG][mC][ | 2019 | [vinmU*][fU*][mG][m |
| | | mU][mU][fA][fA][fC] | | A][mU][mG][mG][mA][ |
| | | [mU][mG][fU][mC][mC | | mC][mA][mG][mU][mU] |
| | | ][mA][mU][fC][mA][m | | [fA][mA][mG][mC][mA |
| | | A] | | ][mG][*mU][*mU] |
| J9 | 1910 | [mU*][mG*][fC][mU][ | 2020 | [vinmU*][fC*][mU][m |
| | | mU][mA][fA][fC][fU] | | G][mA][mU][mG][mG][ |
| | | [mG][mU][fC][mC][mA | | mA][mC][mA][mG][mU] |
| | | ][mU][mC][fA][mG][m | | [fU][mA][mA][mG][mC |
| | | A] | | ][mA][*mU][*mU] |
| K9 | 1911 | [mG*][mC*][fU][mU][ | 2021 | [vinmU*][fG*][mC][m |
| | | mA][mA][fC][fU][fG] | | U][mG][mA][mU][mG][ |
| | | [mU][mC][fC][mA][mU | | mG][mA][mC][mA][mG] |
| | | ][mC][mA][fG][mC][m | | [fU][mU][mA][mA][mG |
| | | A] | | ][mC][*mU][*mU] |
| L9 | 1912 | [mC*][mU*][fU][mA][ | 2022 | [vinmU*][fU][mG][mC |
| | | mA][mC][fU][fG][fU] | | ][mU][mG][mA][mU][m |
| | | [mC][mC][fA][mU][mC | | G][mG][mA][mC][mA][ |
| | | ][mA][mG][fC][mA][m | | fG][mU][mU][mA][mA] |
| | | A] | | [mG][*mU][*mU] |
| M9 | 1913 | [mU*][mU*][fA][mA][ | 2023 | [vinmU*][fC*][mU][m |
| | | mC][mU][fG][fU][fC] | | G][mC][mU][mG][mA][ |
| | | [mC][mA][fU][mC][mA | | mU][mG][mG][mA][mC] |
| | | ][mG][mC][fA][mG][m | | [fA][mG][mU][mU][mA |
| | | A] | | ][mA][*mU][*mU] |
| N9 | 1914 | [mA*][mA*][fC][mU][ | 2024 | [vinmU*][fU*][mC][m |
| | | mG][mU][fC][fC][fA] | | C][mU][mG][mC][mU][ |
| | | [mU][mC][fA][mG][mC | | mG][mA][mU][mG][mG] |
| | | ][mA][mG][fG][mA][m | | [fA][mC][mA][mG][mU |
| | | A] | | ][mU][*mU][*mU] |
| O9 | 1915 | [mA*][mC*][fU][mG][ | 2025 | [vinmU*][fC*][mU][m |
| | | mU][mC][fC][fA][fU] | | C][mC][mU][mG][mC][ |
| | | [mC][mA][fG][mC][mA | | mU][mG][mA][mU][mG] |
| | | ][mG][mG][fA][mG][m | | [fG][mA][mC][mA][mG |
| | | A] | | ][mU][*mU][*mU] |
| P9 | 1916 | [mC*][mU*][fG][mU][ | 2026 | [vinmU*][fU*][mC][m |
| | | mC][mC][fA][fU][fC] | | U][mC][mC][mU][mG][ |
| | | [mA][mG][fC][mA][mG | | mC][mU][mG][mA][mU] |
| | | ][mG][mA][fG][mA][m | | [fG][mG][mA][mC][mA |
| | | A] | | ][mG][*mU][*mU] |
| Q9 | 1917 | [mG*][mU*][fC][mC][ | 2027 | [vinmU*][[A][mG][mU |
| | | mA][mU][[fC][fA][G] | | ][mC][mU][mC][mC][m |
| | | [mC][mA][fG][mG][mA | | U][mG][mC][mU][mG][ |
| | | ][mG][mA][fC][mU][m | | fA][mU][mG][mG][mA] |
| | | A] | | [mC][*mU][*mU] |
| R9 | 1918 | [mU*][mC*][fC][mA][ | 2028 | [vinmU*][fC*][mA][m |
| | | mU][mC][fA][fG][fC] | | G][mU][mC][mU][mC][ |
| | | [mA][mG][fG][mA][mG | | mC][mU][mG][mC][mU] |
| | | ][mA][mC][fU][mG][m | | [fG][mA][mU][mG][mG |
| | | A] | | ][mA][*mU][*mU] |
| S9 | 1919 | [mA*][mU*][fC][mA][ | 2029 | [vinmU*][fA*][mG][m |
| | | mG][mC][fA][fG][fG] | | C][mC][mA][mG][mU][ |
| | | [mA][mG][fA][mC][mU | | mC][mU][mC][mC][mU] |
| | | ][mG][mG][fC][mU][m | | [fG][mC][mU][mG][mA |
| | | A] | | ][mU][*mU][*mU] |
| T9 | 1920 | [mU*][mC*][fA][mG][ | 2030 | [vinmU*][fU*][mA][m |
| | | mC][mA][fG][fG][fA] | | G][mC][mC][mA][mG][ |
| | | [mG][mA][fC][mU][mG | | mU][mC][mU][mC][mC] |
| | | ][mG][mC][fU][mA][m | | [fU][mG][mC][mU][mG |
| | | A] | | ][mA][*mU][*mU] |
| U9 | 1921 | [mG*][mG*][fA][mG][ | 2031 | [vinmU*][fU*][mU][m |
| | | mA][mC][fU][fG][fG] | | U][mA][mU][mU][mu][ |
| | | [mC][mU][fA][mA][mA | | mA][mG][mC][mC][mA] |
| | | ][mU][mA][fA][mA][m | | [fG][mU][mC][mU][mC |
| | | A] | | ][mC][*mU][*mU] |
| V9 | 1922 | [mG*][mA*][fC][mU][ | 2032 | [vinmU*][fA*][mA][m |
| | | mG][mG][fC][fU][fA] | | U][mU][mU][mU][mA][ |
| | | [mA][mA][fU][mA][mA | | mU][mU][mU][mA][mG] |
| | | ][mA][mA][fU][mu][m | | [fC][mC][mA][mG][mU |
| | | A] | | ][mC][*mU][*mU] |
| W9 | 1923 | [mC*][mU*][fG][mG][ | 2033 | [vinmU*][fC*][mU][m |
| | | mC][mU][fA][fA][fA] | | A][mA][mU][mU][mu][ |
| | | [mU][mA][fA][mA][mA | | mU][mA][mU][mU][mU] |
| | | ][mU][mU][fA][mG][m | | [fA][mG][mC][mC][mA |
| | | A] | | ][mG][*mU][*mU] |
| X9 | 1924 | [mU*][mG*][fG][mC][ | 2034 | [vinmU*][fU*][mC][m |
| | | mU][mA][fA][fA][fU] | | U][mA][mA][mU][mu][ |
| | | [mA][mA][fA][mA][mU | | mU][mU][mA][mU][mU] |
| | | ][mU][mA][fG][mA][m | | [fU][mA][mG][mC][mC |
| | | A] | | ][mA][*mU][*mU] |
| Y9 | 1925 | [mG*][mG*][fC][mU][ | 2035 | [vinmU*][fU*][mU][m |
| | | mA][mA][fA][fU][fA] | | C][mU][mA][mA][mU][ |
| | | [mA][mA][fA][mU][mU | | mU][mU][mU][mA][mU] |
| | | ][mA][mG][fA][mA][m | | [fU][mU][mA][mG][mC |
| | | A] | | ][mC][*mU][*mU] |
| Z9 | 1926 | [mG*][mC*][fU][mA][ | 2036 | [vinmu*][fA*][mU][m |
| | | mA][mA][fU][fA][fA] | | U][mC][mU][mA][mA][ |
| | | [mA][mA][fU][mU][mA | | mU][mU][mU][mU][mA] |
| | | ][mG][mA][fA][mU][m | | [fU][mU][mU][mA][mG |
| | | A] | | ][mC][*mU][*mU] |
| A10 | 1927 | [mA*][mA*][fA][mU][ | 2037 | [vinmU*][fU*][mA][m |
| | | mA][mA][fA][fA][fU] | | U][mA][mU][mU][mC][ |
| | | [mU][mA][fG][mA][mA | | mU][mA][mA][mU][mU] |
| | | ][mU][mA][fU][mA][m | | [fU][mU][mA][mU][mu |
| | | A] | | ][mU][*mU][*mU] |
| B10 | 1928 | [mA*][mU*][fA][mA][ | 2038 | [vinmU*][fA*][mA][m |
| | | mA][mA][fU][fU][[A] | | U][mA][mU][mA][mU][ |
| | | [mG][mA][fA][mU][mA | | mU][mC][mU][mA][mA] |
| | | ][mU][mA][fU][mU][m | | [fU][mU][mU][mU][mA |
| | | A] | | ][mU][*mU][*mU] |
| F10 | 1932 | [mG*][mA*][fG][mU][ | 2042 | [vinmU*][fU*][mG][m |
| | | mC][mG][fC][fA][fU] | | C][mA][mC][mU][mG][ |
| | | [mC][mU][fC][mA][mG | | mA][mG][mA][mU][mG] |
| | | ][mU][mG][fC][mA][m | | [fC][mG][mA][mC][mU |
| | | A] | | ][mC][*mU][*mU] |
| G10 | 1933 | [mG*][mU*][fC][mG][ | 2043 | [vinmU*][fC*][mC][m |
| | | mC][mA][fU][fC][fU] | | U][mG][mC][mA][mC][ |
| | | [mC][mA][fG][mU][mG | | mU][mG][mA][mG][mA] |
| | | ][mC][mA][fG][mG][m | | [fU][mG][mC][mG][mA |
| | | A] | | ][mC][*mU][*mU] |
| H10 | 1934 | [mA*][mG*][fA][mA][ | 2044 | [vinmU*][fG*][mG][m |
| | | mC][mC][fU][fC][fu] | | U][mG][mA][mA][mG][ |
| | | [mC][mA][fC][mU][mU | | mU][mG][mA][mG][mA] |
| | | ][mC][mA][fC][mC][m | | [fG][mG][mU][mU][mC |
| | | A] | | ][mU][*mU][*mU] |
| I10 | 1935 | [mG*][mC*][fA][mG][ | 2045 | [vinmU*][fA*][mU][m |
| | | mG][mA][fG][fA][fC] | | U][mU][mA][mG][mC][ |
| | | [mU][mG][fG][mC][mU | | mC][mA][mG][mU][mC] |
| | | ][mA][mA][fA][mU][m | | [fU][mC][mC][mU][mG |
| | | A] | | ][mC][*mU][*mU] |
| J10 | 1936 | [mA*][mA*][fC][mA][ | 2046 | [vinmU*][fU*][mG][m |
| | | mG][mU][fA][fC][fC] | | U][mU][mU][mA][mu][ |
| | | [mU][mA][fA][mU][mA | | mU][mA][mG][mG][mU] |
| | | ][mA][mA][fC][mA][m | | [fA][mC][mU][mG][mU |
| | | A] | | ][mU][*mU][*mU] |
| K10 | 1937 | [fG*][mU*][fC][mG][ | 2047 | [vinmU*][fC*][mC][f |
| | | fC][mA][fU][mC][fU] | | U][mG][fC][mA][fC][ |
| | | [mC][fA][mG][fU][mG | | mU][fG][mA][fG][mA] |
| | | ][fC][mA][fG][mG][f | | [fU][mG][fC][mG][fA |
| | | A] | | ][mC][*mU][*mU] |
| L10 | 1938 | [fG*][mU*][fC][mG][ | 2048 | [vinmU*][fC*][mC][f |
| | | fC][mA][fU][fC][fU] | | U][mG][[C][fA][fC][ |
| | | [mC][fA][mG][mU][mG | | mU][fG][mA][mG][mA] |
| | | ][fC][mA][fG][mG][m | | [fU][mG][fC][mG][fA |
| | | A] | | ][mC][*fU][*mU] |
| M10 | 1939 | [mG][mA][[A][mC][mC | 2049 | [vinmU*][fG*][mG][m |
| | | ][mU][fC][fU][fC][m | | G][mU][mG][mA][mA][ |
| | | A][mC][fU][mU][mC][ | | mG][mU][mG][mA][mG] |
| | | mA][mC][fC][*mC][*m | | [fA][mG][mG][mU][mU |
| | | A] | | ][mC][*mU][*mU] |
| N10 | 1940 | [mG][mC][fA][mG][mG | 2050 | [vinmu*][fA*][mU][m |
| | | ][mA][fG][fA][fC][m | | U][mU][mA][mG][mC][ |
| | | U][mG][fG][mC][mu][ | | mC][mA][mG][mU][mC] |
| | | mA][mA][fA][*mu][*m | | [fU][mC][mC][mU][mG |
| | | A] | | ][mC][*mU][*mU] |
| O10 | 1941 | [mA][mG][fA][mA][mA | 2051 | [vinmU*][fU*][mC][m |
| | | ][mC][fA][fG][fU][m | | A][mA][mG][mG][mU][ |
| | | U][mC][fA][mC][mC][ | | mG][mA][mA][mC][mU] |
| | | mU][mU][fG][*mA][*m | | [fG][mU][mU][mU][mC |
| | | A] | | ][m][*mU][*mU] |
| P10 | 1942 | [mG][mA][fA][mA][mC | 2052 | [vinmU*][fU*][mU][m |
| | | ][mA][fG][fU][fU][m | | C][mA][mA][mG][mG][ |
| | | C][mA][fC][mC][mU][ | | mU][mG][mA][mA][mC] |
| | | mU][mG][fA][*mA][*m | | [fU][mG][mU][mU][mU |
| | | A] | | ][mC][*mU][*mU] |
| Q10 | 1943 | [mG][mA][fA][mA][mC | 2053 | [vinmU*][fU*][mU][m |
| | | ][mA][fG][fU][fU][m | | C][mA][mA][mG][mG][ |
| | | C][mA][fC][mC][mU][ | | mU][mG][mA][mA][mC] |
| | | mU][mG][fA][*mA][*m | | [fU][mG][mU][mU][mU |
| | | A] | | ][mC][*mU][*mU] |
| R10 | 1944 | [mA][mC][fC][mU][mC | 2054 | [vinmU*][fC*][mA][m |
| | | ][mU][fC][fA][fC][m | | G][mG][mG][mU][mG][ |
| | | U][mU][fC][mA][mC][ | | mA][mA][mG][mU][mG] |
| | | mC][mC][fU][*mG][*m | | [fA][mG][mA][mG][mG |
| | | A] | | ][mU][*mU][*mU] |
| S10 | 1945 | [mA][mC][fU][mU][mC | 2055 | [vinmU*][fU*][mG][m |
| | | ][mA][fC][fC][fC][m | | G][mG][mC][mU][mC][ |
| | | U][mG][fG][mA][mG][ | | mC][mA][mG][mG][mG] |
| | | mC][mC][fC][*mA][*m | | [fU][mG][mA][mA][mG |
| | | A] | | ][mU][*mU][*mU] |
| T10 | 1946 | [mG*][mA*][fA][mA][ | 2056 | [vinmU*][fU*][mU][m |
| | | mC][mA][fG][fU][fU] | | C][mA][mA][mG][mG][ |
| | | [mC][mA][fC][mC][mU | | mU][mG][mA][mA][mC] |
| | | ][mU][mG][fA][mA][m | | [fU][mG][mU][mU][mU |
| | | A] | | ][mC][*mU][*mU] |
| U10 | 1947 | [mG][mU][fC][mG][mC | 2057 | [vinmU*][fC*][mC][m |
| | | ][mA][fU][fC][fu][m | | U][mG][mC][mA][mC][ |
| | | C][mA][fG][mU][mG][ | | mU][mG][mA][mG][mA] |
| | | mC][mA][fG][*mG][*m | | [fU][mG][mC][mG][mA |
| | | A] | | ][mC][*mU][*mU] |
| V10 | 1948 | [mG][mU][fC][mG][mC | 2058 | [vinmU*][fC*][mC][m |
| | | ][mA][fU][fC][fU][m | | U][mG][mC][mA][mC][ |
| | | C][mA][fG][mU][mG][ | | mU][mG][mA][mG][mA] |
| | | mC][mA][fG][*mG][*m | | [fU][mG][mC][mG][mA |
| | | A] | | ][mC][*mU][*mU] |
| W10 | 1949 | [mG][mU][fC][mG][mC | 2059 | [vinmU*][fC*][mC][m |
| | | ][mA][fU][fC][fU][m | | U][mG][mC][mA][mC][ |
| | | C][mA][fG][mU][mG][ | | mU][mG][mA][mG][mA] |
| | | mC][mA][fG][*mG][*m | | [fU][mG][mC][mG][mA |
| | | A] | | ][mC][*mU][*mU] |
| X10 | 1950 | [mG][mU][fC][mG][mc | 2060 | [vinmU*][fC*][mC][m |
| | | ][mA][fU][fC][fU][m | | U][mG][mC][mA][mC][ |
| | | C][mA][fG][mU][mG][ | | mU][mG][mA][mG][mA] |
| | | mC][mA][fG][*mG][*m | | [fU][mG][mC][mG][mA |
| | | A] | | ][mC][*mU][*mU] |
| Y10 | 1951 | [mG][mU][fC][mG][mC | 2061 | [vinmU*][fC*][mC][m |
| | | ][mA][fU][fC][fU][m | | U][mG][mC][mA][mC][ |
| | | C][mA][fG][mU][mG][ | | mU][mG][mA][mG][mA] |
| | | mC][mA][fG][*mG][*m | | [fU][mG][mC][mG][mA |
| | | A] | | ][mC][*mU][*mU] |
| Z10 | 1952 | [mG][mU][fC][mG][mC | 2062 | [vinmU*][fC*][mC][m |
| | | ][mA][fU][fC][fU][m | | U][mG][mC][mA][mC][ |
| | | C][mA][fG][mU][mG][ | | mU][mG][mA][mG][mA] |
| | | mC][mA][fG][*mG][*m | | [fU][mG][mC][mG][mA |
| | | A] | | ][mC][*mU][*mU] |
| A11 | 1953 | [*][Mg][Mu][Fc][Mg] | 2063 | [vinmU*][fC*][mC][m |
| | | [Mc][Ma][Fu][Fc][Fu | | U][mG][mC][mA][mC][ |
| | | ][Mc][Ma][Fg][Mu][M | | mU][mG][mA][mG][mA] |
| | | g][Mc][Ma][Fg][*mG] | | [fU][mG][mC][mG][mA |
| | | [*mA] | | ][mC][*mU][*mU] |
| B11 | 1954 | [mG][mA][fA][mA][mC | 2064 | [vinmU*][fU*][mU][m |
| | | ][mA][fG][fU][fU][m | | C][mA][mA][mG][mG][ |
| | | C][mA][fC][mC][mu][ | | mU][mG][mA][mA][mC] |
| | | mU][mG][fA][*mA][*m | | [fU][mG][mU][mU][mU |
| | | A] | | ][mC][*mU][*mU] |
| C11 | 1955 | [mG][mA][fA][mA][mC | 2065 | [vinmU*][fU*][mU][m |
| | | ][mA][fG][fU][fU][m | | C][mA][mA][mG][mG][ |
| | | C][mA][fC][mC][mU][ | | mU][mG][mA][mA][mC] |
| | | mU][mG][fA][*mA][*m | | [fU][mG][mU][mU][mU |
| | | A] | | ][mC][*mU][*mU] |
| D11 | 1956 | [mG][mA][fA][mA][mC | 2066 | [vinmU*][fU*][mU][m |
| | | ][mA][fG][fU][fU][m | | C][mA][mA][mG][mG][ |
| | | C][mA][fC][mC][mU][ | | mU][mG][mA][mA][mC] |
| | | mU][mG][fA][*mA][*m | | [fU][mG][mU][mU][mU |
| | | A] | | ][mC][*mU][*mU] |
| E11 | 1957 | [mG][mA][fA][mA][mC | 2067 | [vinmU*][fU*][mU][m |
| | | ][mA][fG][fU][fU][m | | C][mA][mA][mG][mG][ |
| | | C][mA][fC][mC][mU][ | | mU][mG][mA][mA][mC] |
| | | mU][mG][fA][*mA][*m | | [fU][mG][mU][mU][mu |
| | | A] | | ][mC][*mU][*mU] |
| F11 | 1958 | [mG][mA][fA][mA][mC | 2068 | [vinmU*][fU*][mU][m |
| | | ][mA][[G][fU][fU][m | | C][mA][mA][mG][mG][ |
| | | C][mA][fC][mC][mU][ | | mU][mG][mA][mA][mC] |
| | | mU][mG][fA][*mA][*m | | [fU][mG][mU][mU][mu |
| | | A] | | ][mC][*mU][*mU] |
| G11 | 1959 | [$][mG][mU][[C][mG] | 2069 | [vinmU*][fC*][mC][m |
| | | [mC][mA][fU][fC][fU | | U][mG][mC][mA][mC][ |
| | | ][mC][mA][fG][mU][m | | mU][mG][mA][mG][mA] |
| | | G][mC][mA][fG][*mG] | | [fU][mG][mC][mG][mA |
| | | [*mA] | | ][mC][*mU][*mU] |
| H11 | 1890 | [mA*][mG*][fA][mA][ | 2290 | [vinmU*][fU*][mC][m |
| | | mA][mC][fA][fG][fU] | | A][mA][mG][mG][mU][ |
| | | [mU][mC][fA][mC][mC | | mG][mA][mA][mC][mU] |
| | | ][mU][mU][fG][mA][m | | [fG][mU][mU][mU][mc |
| | | A] | | ][mU][*mU][*mU] |
| I11 | 1891 | [mG*][mA*][fA][mA][ | 2291 | [vinmU*][fU*][mU][m |
| | | mC][mA][fG][fU][fU] | | C][mA][mA][mG][mG][ |
| | | [mC][mA][fC][mC][mU | | mU][mG][mA][mA][mC] |
| | | ][mU][mG][fA][mA][m | | [fU][mG][mU][mU][mu |
| | | A] | | ][mC][*mU][*mU] |
| J11 | 1892 | [mA*][mA*][fC][mC][ | 2292 | [vinmU*][fA*][mG][m |
| | | mU][mC][fU][fC][fA] | | G][mG][mU][mG][mA][ |
| | | [mC][mU][fU][mC][mA | | mA][mG][mU][mG][mA] |
| | | ][mC][mC][fC][mU][m | | [fG][mA][mG][mG][mU |
| | | A] | | ][mU][*mU][*mU] |
| K11 | 1893 | [mA*][mC*][fC][mU][ | 2293 | [vinmU*][fC*][mA][m |
| | | mC][mU][fC][fA][fC] | | G][mG][mG][mU][mG][ |
| | | [mU][mU][fC][mA][mC | | mA][mA][mG][mU][mG] |
| | | ][mC][mC][fU][mG][m | | [fA][mG][mA][mG][mG |
| | | A] | | ][mU][*mU][*mU] |
| L11 | 1894 | [mC*][mC*][fU][mC][ | 2294 | [vinmU*][fC*][mC][m |
| | | mU][mC][fA][fC][fu] | | A][mG][mG][mG][mU][ |
| | | [mU][mC][fA][mC][mC | | mG][mA][mA][mG][mU] |
| | | ][mC][mU][fG][mG][m | | [fG][mA][mG][mA][mG |
| | | A] | | ][mG][*mU][*mU] |
| M11 | 1895 | [mC*][mU*][fC][mU][ | 2295 | [vinmU*][fU*][mC][m |
| | | mC][mA][fC][fU][fU] | | C][mA][mG][mG][mG][ |
| | | [mC][mA][fC][mC][mC | | mU][mG][mA][mA][mG] |
| | | ][mU][mG][fG][mA][m | | [fU][mG][mA][mG][mA |
| | | A] | | ][mG][*mU][*mU] |
| N11 | 1896 | [mU*][mC*][fU][mC][ | 2296 | [vinmU*][fC*][mU][m |
| | | mA][mC][fU][fU][fC] | | C][mC][mA][mG][mG][ |
| | | [mA][mC][fC][mC][mU | | mG][mU][mG][mA][mA] |
| | | ][mG][mG][fA][mG][m | | [fG][mU][mG][mA][mG |
| | | A] | | ][mA][*mU][*mU] |
| O11 | 1897 | [mC*][mU*][fC][mA][ | 2297 | [vinmU*][fG*][mC][m |
| | | mC][mU][fU][fC][[A] | | U][mC][mC][mA][mG][ |
| | | [mC][mC][fC][mU][mG | | mG][mG][mU][mG][mA] |
| | | ][mG][mA][fG][mC][m | | [fA][mG][mU][mG][mA |
| | | A] | | ][mG][*mU][*mU] |
| P11 | 2298 | [mA*][mG*][fG][mA][ | 2299 | [vinmU*][fU*][mU][m |
| | | mG][mA][fC][fU][fG] | | A][mU][mU][mU][mA][ |
| | | [mG][mC][fU][mA][mA | | mG][mC][mC][mA][mG] |
| | | ][mA][mU][fA][mA][m | | [fU][mC][mU][mC][mC |
| | | A] | | ][mU][*mU][*mU] |
| Q11 | 2302 | [fG*][mU*][fC][mG][ | 2303 | [vinmU*][fC*][mC][f |
| | | fC][mA][fU][[C][fU] | | U][mG][fC][fA][fC][ |
| | | [mC][fA][mG][mU][mG | | mU][fG][mA][mG][mA] |
| | | ][fC][mA][fG][mG][m | | [fU][mG][fC][mG][fA |
| | | A] | | ][mC][*fU][*mU] |
| R11 | 2304 | [fG*][mU*][fC][mG][ | 2305 | [vinmU*][fC*][mC][f |
| | | fC][mA][fU][mC][fU] | | U][mG][fC][mA][fc][ |
| | | [mC][fA][mG][fU][mG | | mU][fG][mA][fG][mA] |
| | | ][fC][mA][fG][mG][f | | [fU][mG][fC][mG][fA |
| | | A] | | ][mC][*mU][*mU] |

Abbreviations Key:
n/N = any nucleotide
mN = 2′-O-Methyl substitution
fN = 2′-F substitution
idT = inverted Dt
vinmN or vinmU = 2′-O methyl vinyl phosphonate uridine
* = phosphorothioate
Nr = Nucleotides A, C, G, U with 2′-OH ribose sugar
[PO] = Phosphate group (PO4)
[$] = PO₄-C₆-NH₂-Palmitate
The brackets indicate the individual bases.

TABLE 4B
siRNA Sense and Antisense Sequences (Unmodified)

| SEQ | | SEQ | |
|---|---|---|---|
| ID NO: | Sense Strand 5′-3′ | ID NO: | Antisense Strand 5′-3′ |
| 939 | GAGUCGCAUCUCAGUGCAA | 959 | UUGCACUGAGAUGCGACUC |
| | | | UU |
| 940 | GUCGCAUCUCAGUGCAGGA | 960 | UCCUGCACUGAGAUGCGAC |
| | | | UU |
| 941 | AGAACCUCUCACUUCACCA | 961 | UGGUGAAGUGAGAGGUUCU |
| | | | UU |
| 942 | GAACCUCUCACUUCACCCA | 962 | UGGGUGAAGUGAGAGGUUC |
| | | | UU |
| 943 | AGUCUCCCAACUUGUAUUA | 963 | UAAUACAAGUUGGGAGACU |
| | | | UU |
| 944 | GGAAGCUGCUUAACUGUCA | 964 | UGACAGUUAAGCAGCUUCC |
| | | | UU |
| 945 | GCAGGAGACUGGCUAAAUA | 965 | UAUUUAGCCAGUCUCCUGC |
| | | | UU |
| 946 | UUAUCCUUUGGUUUCUUGA | 966 | UCAAGAAACCAAAGGAUAA |
| | | | UU |
| 947 | GUGUGUUACGUGCAGUGAA | 967 | UUCACUGCACGUAACACAC |
| | UU | | UG |
| 948 | UGUUCCACUGGGCUGAGAA | 968 | UUCUCAGCCCAGUGGAACA |
| | | | UU |
| 949 | GCGAAUUCCUAGACACCUA | 969 | UAGGUGUCUCGGAAUUCGC |
| | UU | | UU |
| 950 | GUGUGUUACGUGCAGUGAC | 970 | UAGUCACUGCACGUAACAC |
| | UA | | ACUG |
| 951 | ACUACAAGACUCGUGACCA | 971 | UGGUCACGAGUCUUGUAGU |
| | UU | | UU |
| 952 | GUGUGUUACGUGCAGUGAA | 972 | UUCACUGCACGUAACACAC |
| | UU | | UG |
| 953 | GCGAAUUCCUAGACACCUA | 973 | UAGGUGUCUCGGAAUUCGC |
| | UU | | UU |
| 954 | GUGUGUUACGUGCAGUGAC | 974 | UAGUCACUGCACGUAACAC |
| | UA | | ACUG |
| 955 | UCCUUGCGGUGAAAGCGAA | 975 | UUCGCUUUCACCGCAAGGA |
| | | | UU |
| 956 | GUAAUAUCCACCAGACCUA | 976 | UAGGUCUGGUGGAUAUUAC |
| | | | UU |
| 957 | UUGUUUGUGAUAGUGAACA | 977 | UGUUCACUAUCACAAACAA |
| | | | UU |
| 958 | GCUGCUUAACUGUCCAUCA | 978 | UGAUGGACAGUUAAGCAGC |
| | | | UU |
| 2070 | AGCAGGAGACUGGCUAAAA | 2180 | UUUUAGCCAGUCUCCUGCU |
| | | | UU |
| 2071 | CAGGAGACUGGCUAAAUAA | 2181 | UUAUUUAGCCAGUCUCCUG |
| | | | UU |
| 2110 | AGAAACAGUUCACCUUGAA | 2220 | UUCAAGGUGAACUGUUUCU |
| | | | UU |
| 2111 | GAAACAGUUCACCUUGAAA | 2221 | UUUCAAGGUGAACUGUUUC |
| | | | UU |
| 2112 | AACCUCUCACUUCACCCUA | 2222 | UAGGGUGAAGUGAGAGGUU |
| | | | UU |
| 2113 | ACCUCUCACUUCACCCUGA | 2223 | UCAGGGUGAAGUGAGAGGU |
| | | | UU |
| 2114 | CCUCUCACUUCACCCUGGA | 2224 | UCCAGGGUGAAGUGAGAGG |
| | | | UU |
| 2115 | CUCUCACUUCACCCUGGAA | 2225 | UUCCAGGGUGAAGUGAGAG |
| | | | UU |
| 2116 | UCUCACUUCACCCUGGAGA | 2226 | UCUCCAGGGUGAAGUGAGA |
| | | | UU |
| 2117 | CUCACUUCACCCUGGAGCA | 2227 | UGCUCCAGGGUGAAGUGAG |
| | | | UU |
| 2118 | UCACUUCACCCUGGAGCCA | 2228 | UGGCUCCAGGGUGAAGUGA |
| | | | UU |
| 2119 | CACUUCACCCUGGAGCCCA | 2229 | UGGGCUCCAGGGUGAAGUG |
| | | | UU |
| 2120 | ACUUCACCCUGGAGCCCAA | 2230 | UUGGGCUCCAGGGUGAAGU |
| | | | UU |
| 2121 | CUUCACCCUGGAGCCCAUA | 2231 | UAUGGGCUCCAGGGUGAAG |
| | | | UU |
| 2122 | UUCACCCUGGAGCCCAUCA | 2232 | UGAUGGGCUCCAGGGUGAA |
| | | | UU |
| 2123 | UCACCCUGGAGCCCAUCCA | 2233 | UGGAUGGGCUCCAGGGUGA |
| | | | UU |
| 2124 | CACCCUGGAGCCCAUCCAA | 2234 | UUGGAUGGGCUCCAGGGUG |
| | | | UU |
| 2125 | ACCCUGGAGCCCAUCCAGA | 2235 | UCUGGAUGGGCUCCAGGGU |
| | | | UU |
| 2126 | CCCUGGAGCCCAUCCAGUA | 2236 | UACUGGAUGGGCUCCAGGG |
| | | | UU |
| 2127 | CCUGGAGCCCAUCCAGUCA | 2237 | UGACUGGAUGGGCUCCAGG |
| | | | UU |
| 2128 | CUGGAGCCCAUCCAGUCUA | 2238 | UAGACUGGAUGGGCUCCAG |
| | | | UU |
| 2129 | CUGCUUAACUGUCCAUCAA | 2239 | UUGAUGGACAGUUAAGCAG |
| | | | UU |
| 2130 | UGCUUAACUGUCCAUCAGA | 2240 | UCUGAUGGACAGUUAAGCA |
| | | | UU |
| 2131 | GCUUAACUGUCCAUCAGCA | 2241 | UGCUGAUGGACAGUUAAGC |
| | | | UU |
| 2132 | CUUAACUGUCCAUCAGCAA | 2242 | UUGCUGAUGGACAGUUAAG |
| | | | UU |
| 2133 | UUAACUGUCCAUCAGCAGA | 2243 | UCUGCUGAUGGACAGUUAA |
| | | | UU |
| 2134 | AACUGUCCAUCAGCAGGAA | 2244 | UUCCUGCUGAUGGACAGUU |
| | | | UU |
| 2135 | ACUGUCCAUCAGCAGGAGA | 2245 | UCUCCUGCUGAUGGACAGU |
| | | | UU |
| 2136 | CUGUCCAUCAGCAGGAGAA | 2246 | UUCUCCUGCUGAUGGACAG |
| | | | UU |
| 2137 | GUCCAUCAGCAGGAGACUA | 2247 | UAGUCUCCUGCUGAUGGAC |
| | | | UU |
| 2138 | UCCAUCAGCAGGAGACUGA | 2248 | UCAGUCUCCUGCUGAUGGA |
| | | | UU |
| 2139 | AUCAGCAGGAGACUGGCUA | 2249 | UAGCCAGUCUCCUGCUGAU |
| | | | UU |
| 2140 | UCAGCAGGAGACUGGCUAA | 2250 | UUAGCCAGUCUCCUGCUGA |
| | | | UU |
| 2141 | GGAGACUGGCUAAAUAAAA | 2251 | UUUUAUUUAGCCAGUCUCC |
| | | | UU |
| 2142 | GACUGGCUAAAUAAAAUUA | 2252 | UAAUUUUAUUUAGCCAGUC |
| | | | UU |
| 2143 | CUGGCUAAAUAAAAUUAGA | 2253 | UCUAAUUUUAUUUAGCCAG |
| | | | UU |
| 2144 | UGGCUAAAUAAAAUUAGAA | 2254 | UUCUAAUUUUAUUUAGCCA |
| | | | UU |
| 2145 | GGCUAAAUAAAAUUAGAAA | 2255 | UUUCUAAUUUUAUUUAGCC |
| | | | UU |
| 2146 | GCUAAAUAAAAUUAGAAUA | 2256 | UAUUCUAAUUUUAUUUAGC |
| | | | UU |
| 2147 | AAAUAAAAUUAGAAUAUAA | 2257 | UUAUAUUCUAAUUUUAUUU |
| | | | UU |
| 2148 | AUAAAAUUAGAAUAUAUUA | 2258 | UAAUAUAUUCUAAUUUUAU |
| | | | UU |
| 2152 | GAGUCGCAUCUCAGUGCAA | 2262 | UUGCACUGAGAUGCGACUC |
| | | | UU |
| 2153 | GUCGCAUCUCAGUGCAGGA | 2263 | UCCUGCACUGAGAUGCGAC |
| | | | UU |
| 2154 | AGAACCUCUCACUUCACCA | 2264 | UGGUGAAGUGAGAGGUUCU |
| | | | UU |
| 2155 | GCAGGAGACUGGCUAAAUA | 2265 | UAUUUAGCCAGUCUCCUGC |
| | | | UU |
| 2156 | AACAGUACCUAAUAAACAA | 2266 | UUGUUUAUUAGGUACUGUU |
| | | | UU |
| 2157 | GUCGCAUCUCAGUGCAGGA | 2267 | UCCUGCACUGAGAUGCGAC |
| | | | UU |
| 2158 | GUCGCAUCUCAGUGCAGGA | 2268 | UCCUGCACUGAGAUGCGAC |
| | | | UU |
| 2159 | GAACCUCUCACUUCACCCA | 2269 | UGGGUGAAGUGAGAGGUUC |
| | | | UU |
| 2160 | GCAGGAGACUGGCUAAAUA | 2270 | UAUUUAGCCAGUCUCCUGC |
| | | | UU |
| 2161 | AGAAACAGUUCACCUUGAA | 2271 | UUCAAGGUGAACUGUUUCU |
| | | | UU |
| 2162 | GAAACAGUUCACCUUGAAA | 2272 | UUUCAAGGUGAACUGUUUC |
| | | | UU |
| 2163 | GAAACAGUUCACCUUGAAA | 2273 | UUUCAAGGUGAACUGUUUC |
| | | | UU |
| 2164 | ACCUCUCACUUCACCCUGA | 2274 | UCAGGGUGAAGUGAGAGGU |
| | | | UU |
| 2165 | ACUUCACCCUGGAGCCCAA | 2275 | UUGGGCUCCAGGGUGAAGU |
| | | | UU |
| 2166 | GAAACAGUUCACCUUGAAA | 2276 | UUUCAAGGUGAACUGUUUC |
| | | | UU |
| 2167 | GUCGCAUCUCAGUGCAGGA | 2277 | UCCUGCACUGAGAUGCGAC |
| | | | UU |
| 2168 | GUCGCAUCUCAGUGCAGGA | 2278 | UCCUGCACUGAGAUGCGAC |
| | | | UU |
| 2169 | GUCGCAUCUCAGUGCAGGA | 2279 | UCCUGCACUGAGAUGCGAC |
| | | | UU |
| 2170 | GUCGCAUCUCAGUGCAGGA | 2280 | UCCUGCACUGAGAUGCGAC |
| | | | UU |
| 2171 | GUCGCAUCUCAGUGCAGGA | 2281 | UCCUGCACUGAGAUGCGAC |
| | | | UU |
| 2172 | GUCGCAUCUCAGUGCAGGA | 2282 | UCCUGCACUGAGAUGCGAC |
| | | | UU |
| 2173 | GUCGCAUCUCAGUGCAGGA | 2283 | UCCUGCACUGAGAUGCGAC |
| | | | UU |
| 2174 | GAAACAGUUCACCUUGAAA | 2284 | UUUCAAGGUGAACUGUUUC |
| | | | UU |
| 2175 | GAAACAGUUCACCUUGAAA | 2285 | UUUCAAGGUGAACUGUUUC |
| | | | UU |
| 2176 | GAAACAGUUCACCUUGAAA | 2286 | UUUCAAGGUGAACUGUUUC |
| | | | UU |
| 2177 | GAAACAGUUCACCUUGAAA | 2287 | UUUCAAGGUGAACUGUUUC |
| | | | UU |
| 2178 | GAAACAGUUCACCUUGAAA | 2288 | UUUCAAGGUGAACUGUUUC |
| | | | UU |
| 2179 | GUCGCAUCUCAGUGCAGGA | 2289 | UCCUGCACUGAGAUGCGAC |
| | | | UU |
| 2110 | AGAAACAGUUCACCUUGAA | 2220 | UUCAAGGUGAACUGUUUCU |
| | | | UU |
| 2111 | GAAACAGUUCACCUUGAAA | 2221 | UUUCAAGGUGAACUGUUUC |
| | | | UU |
| 2112 | AACCUCUCACUUCACCCUA | 2222 | UAGGGUGAAGUGAGAGGUU |
| | | | UU |
| 2113 | ACCUCUCACUUCACCCUGA | 2223 | UCAGGGUGAAGUGAGAGGU |
| | | | UU |
| 2114 | CCUCUCACUUCACCCUGGA | 2224 | UCCAGGGUGAAGUGAGAGG |
| | | | UU |
| 2115 | CUCUCACUUCACCCUGGAA | 2225 | UUCCAGGGUGAAGUGAGAG |
| | | | UU |
| 2116 | UCUCACUUCACCCUGGAGA | 2226 | UCUCCAGGGUGAAGUGAGA |
| | | | UU |
| 2117 | CUCACUUCACCCUGGAGCA | 2227 | UGCUCCAGGGUGAAGUGAG |
| | | | UU |
| 2300 | AGGAGACUGGCUAAAUAAA | 2301 | UUUAUUUAGCCAGUCUCCU |
| | | | UU |
| 2306 | GUCGCAUCUCAGUGCAGGA | 2307 | UCCUGCACUGAGAUGCGAC |
| | | | UU |
| 2308 | GUCGCAUCUCAGUGCAGGA | 2309 | UCCUGCACUGAGAUGCGAC |
| | | | UU |

TABLE 5A
siRNA Pairs with Linkers

| siRNA | SEQ | | SEQ | |
|---|---|---|---|---|
| Pair | ID NO: | Sense Strand 5′-3′ | ID NO: | Antisense Strand 5′-3′ |
| X6 | 312 | [mG*] [mA*] [fG] [mU] [mC] | 332 | [vinmU*] [fU*] [mG] [mC] |
| | | [mG] [fC] [fA] [fU] [mC] | | [mA] [mC] [mU] [mG] [mA] |
| | | [mU] [fC] [mA] [mG] [mU] | | [mG] [mA] [mU] [mG] [fC] |
| | | [mG] [fC] [mA] [mA] | | [mG] [mA] [mC] [mU] [mC] |
| | | | | [*mU] [*mU] |
| Y6 | 313 | [mG*] [mU*] [fC] [mG] [mC] | 333 | [vinmU*] [fC*] [mC] [mU] |
| | | [mA] [fU] [fC] [fU] [mC] | | [mG] [mC] [mA] [mC] [mU] |
| | | [mA] [fG] [mU] [mG] [mC] | | [mG] [mA] [mG] [mA] [fU] |
| | | [mA] [fG] [mG] [mA] | | [mG] [mC] [mG] [mA] [mC] |
| | | | | [*mU] [*mU] |
| Z6 | 314 | [mA*] [mG*] [fA] [mA] [mC] | 334 | [vinmu*] [fG*] [mG] [mU] |
| | | [mC] [fU] [fC] [fU] [mC] | | [mG] [mA] [mA] [mG] [mU] |
| | | [mA] [fC] [mU] [mU] [mC] | | [mG] [mA] [mG] [mA] [fG] |
| | | [mA] [fC] [mC] [mA] | | [mG] [mU] [mU] [mC] [mu] |
| | | | | [*mU] [*mU] |
| A7 | 318 | [mG*] [mC*] [fA] [mG] [mG] | 338 | [vinmu*] [fA*] [mU] [mU] |
| | | [mA] [fG] [fA] [fC] [mU] | | [mU] [mA] [mG] [mC] [mC] |
| | | [mG] [fG] [mC] [mU] [mA] | | [mA] [mG] [mU] [mC] [fU] |
| | | [mA] [fA] [mU] [mA] | | [mC] [mC] [mU] [mG] [mC] |
| | | | | [*mU] [*mU] |

wherein the sense strands in Table 5A comprise the following 3′ linker: 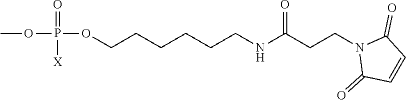
Abbreviations Key:
n/N = any nucleotide
mN = 2′-O-Methyl substitution
fN = 2′-F substitution
idT = inverted Dt
vinmN or vinmU = 2′-O methyl vinyl phosphonate uridine
X = O or S
* = phosphorothioate
The brackets indicate the individual bases.

TABLE 5B
siRNA Pairs with Linkers

| SiRNA | SEQ | | SEQ | | Linker |
|---|---|---|---|---|---|
| Pair | ID NO: | Sense Strand 5′-3′ | ID NO: | Antisense Strand 5′-3′ | Location |
| H11 | 1890 | [mA*] [mG*] [fA] | 2290 | [vinmU*] [fU*] [mC] | 3′ end of |
| | | [mA] [mA] [mC] [fA] | | [mA] [mA] [mG] [mG] | sense |
| | | [fG] [fU] [mU] [mC] | | [mU] [mG] [mA] | strand |
| | | [fA] [mC] [mC] [mU] | | [mA] [mC] [mU] [fG] | |
| | | [mU] [fG] [mA] [mA] | | [mU] [mU] [mU] [mC] | |
| | | | | [mU] [*mU] [*mU] | |
| K11 | 1893 | [mA*] [mC*] [fC] | 2293 | [vinmU*] [fC*] [mA] | 3′ end of |
| | | [mU] [mC] [mu] [fC] | | [mG] [mG] [mG] [mU] | sense |
| | | [fA] [fC] [mU] [mu] | | [mG] [mA] [mA] | strand |
| | | [fC] [mA] [mC] [mC] | | [mG] [mU] [mG] [fA] | |
| | | [mC] [fU] [mG] [mA] | | [mG] [mA] [mG] [mG] | |
| | | | | [mU] [*mU] [*mU] | |
| O10 | 1941 | [mA] [mG] [fA] [mA] | 2051 | [vinmU*] [fU*] [mC] | 5′ end of |
| | | [mA] [mC] [fA] [fG] | | [mA] [mA] [mG] | sense |
| | | [fU] [mU] [mC] [fA] | | [mG] [mU] [mG] [mA] | strand |
| | | [mC] [mC] [mU] [mU] | | [mA] [mC] [mU] [fG] | |
| | | [fG] [*mA] [*mA] | | [mU] [mU] [mU] [mC] | |
| | | | | [mU] [*mU] [*mU] | |
| P10 | 1942 | [mG] [mA] [fA] [mA] | 2052 | [vinmU*] [fU*] [mU] | 5′ end of |
| | | [mC] [mA] [fG] [fU] | | [mC] [mA] [mA] [mG] | sense |
| | | [fU] [mC] [mA] [fC] | | [mG] [mU] [mG] | strand |
| | | [mC] [mU] [mU] [mG] | | [mA] [mA] [mC] [fU] | |
| | | [fA] [*mA] [*mA] | | [mG] [mU] [mU] [mU] | |
| | | | | [mC] [*mU] [*mU] | |
| R10 | 1944 | [mA] [mC] [fC] [mU] | 2054 | [vinmU*] [fC*] [mA] | 5′ end of |
| | | [mC] [mU] [fC] [fA] | | [mG] [mG] [mG] | sense |
| | | [fC] [mU] [mU] [fC] | | [mU] [mG] [mA] [mA] | strand |
| | | [mA] [mC] [mC] [mC] | | [mG] [mU] [mG] [fA] | |
| | | [fU] [*mG] [*mA] | | [mG] [mA] [mG] [mG] | |
| | | | | [mU] [*mU] [*mU] | |

wherein the sense strands in Table 5B comprise the following linker: 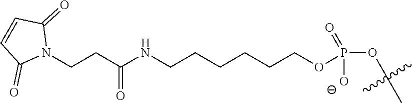 [SMILES: *OP(=O)([O-])OCCCCCCNC(=O)CCN1C(=O)C=CC1=O]
Abbreviations Key:
n/N = any nucleotide
mN = 2′-O-Methyl substitution
fN = 2′-F substitution
idT = inverted Dt
vinmN or vinmU = 2′-O methyl vinyl phosphonate uridine
X = O or S
* = phosphorothioate
The brackets indicate the individual bases.

In some embodiments, the polynucleotides illustrated above include those that do not include a 2′-O methyl vinyl phosphonate uridine as the 5′ nucleotide on the antisense strand of the siRNA.

In some embodiments, a polynucleotide is as provided for herein. In some embodiments, the polynucleotide comprises a first strand and a second strand for a portion that comprises a duplex. In some embodiments, the polynucleotide comprises a sense strand and an antisense strand. In some embodiments, the polynucleotide comprises the sequences as illustrated in Table 3A, Table 3B, Table 4A, Table 4B, Table 5A, or Table 5B. In some embodiments, the polynucleotide comprises the sequences as illustrated in Table 3A, Table 4A, or Table 5A, but without the base modifications. In some embodiments, a polynucleotide comprises an siRNA pair as provided herein. In some embodiments, the siRNA pair is not conjugated to an FN3 domain.

In some embodiments, an oligonucleotide molecule described herein is constructed using chemical synthesis and/or enzymatic ligation reactions using procedures known in the art. For example, an oligonucleotide molecule is chemically synthesized using naturally occurring nucleotides or variously modified nucleotides designed to increase the biological stability of the molecules or to increase the physical stability of the duplex formed between the oligonucleotide molecule and target nucleic acids. Alternatively, the oligonucleotide molecule is produced biologically using an expression vector into which a oligonucleotide molecule has been subcloned in an antisense orientation (i.e., RNA transcribed from the inserted oligonucleotide molecule will be of an antisense orientation to a target polynucleic acid molecule of interest).

In some embodiments, an oligonucleotide molecule is synthesized via a tandem synthesis methodology, wherein both strands are synthesized as a single contiguous oligonucleotide fragment or strand separated by a cleavable linker which is subsequently cleaved to provide separate fragments or strands that hybridize and permit purification of the duplex.

In some instances, an oligonucleotide molecule is also assembled from two distinct nucleic acid strands or fragments wherein one fragment includes the sense region and the second fragment includes the antisense region of the molecule.

In some instances, while chemical modification of the oligonucleotide molecule internucleotide linkages with phosphorothioate, phosphorodithioate, phosphonate, phosphoramidate, or mesyl phosphoramidate, linkages improves stability. Excessive modifications sometimes cause toxicity or decreased activity. Therefore, when designing nucleic acid molecules, the amount of these internucleotide linkages in some cases is minimized. In such cases, the reduction in the concentration of these linkages lowers toxicity, increases efficacy and higher specificity of these molecules.

As described herein, in some embodiments, any nucleic acid molecule disclosed herein can be modified to include a linker at the 5′ end of the of the sense strand of the dsRNA. In some embodiments, any nucleic acid molecules disclosed herein can be modified to include a vinyl phosphonate or modified vinyl phosphonate at the 5′ end of the antisense strand of the dsRNA. In some embodiments, any nucleic acid molecule disclosed herein can be modified to include a linker at the 3′ end of the of the sense strand of the dsRNA. In some embodiments, any nucleic acid molecule disclosed herein can be modified to include a vinyl phosphonate at the 3′ end of the antisense strand of the dsRNA. The linker can be used to link the dsRNA to the FN3 domain. The linker can covalently attach, for example, to a cysteine residue on the FN3 domain that is there naturally or that has been substituted as described herein, and for example, in U.S. Pat. No. 10,196,446, which is hereby incorporated by reference in its entirety.

In some embodiments, the siRNA pairs of A1-W6 and B₇-G11 as shown in Tables 3A and 4A provided for above comprise a linker at the 3′ end of the sense strand. In some embodiments, the siRNA pairs of A1-W6 and B₇-G11 as shown in Tables 3A and 4A provided for above comprise a vinyl phosphonate at the 5′ end of the sense strand.

Non-limiting examples of structures of linkers (L) are illustrated in Table 6A and Table 6B, below.

TABLE 6A
Exemplary Linker (L) Structures

| Linker Structure | Linker Name |
|---|---|
| 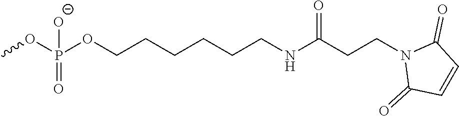 | Mal-C₂H₄C(O)(NH)- (CH₂)₆ or C₆-NH₂- Propyl-Mal |
| 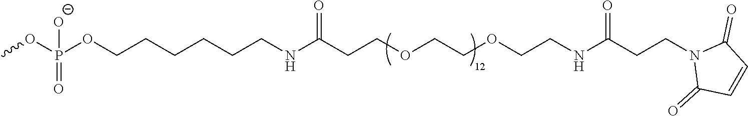 | Mal- (PEG)₁₂(NH)(CH₂)₆ |
| 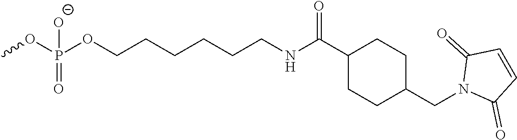 | Mal-NH-(CH₂)₆ or Aminohexyl linker- (CH₂)₆- |
| 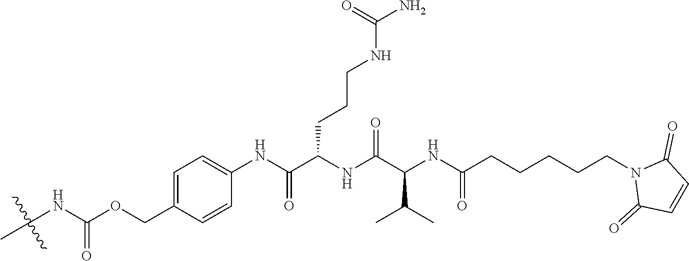 [SMILES: *NC(=O)OCc1ccc(NC(=O)[C@H](CCCNC(N)=O)NC(=O)[C@@H](NC(=O)CCCCCN2C(=O)C=CC2=O)C(C)C)cc1] | Val-Cit-PABA |

TABLE 6B
Exemplary Linker (L) Structures
Linker structures: 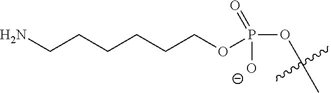 [SMILES: *OP(=O)([O-])OCCCCCCN]
C6-NH2 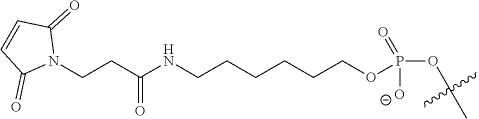 [SMILES: *OP(=O)([O-])OCCCCCCNC(=O)CCN1C(=O)C=CC1=O]
C6-NH2-Propyl-Mal 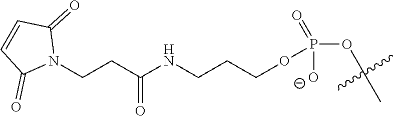 [SMILES: *OP(=O)([O-])OCCCNC(=O)CCN1C(=O)C=CC1=O]
C3-NH2-Propyl-Mal 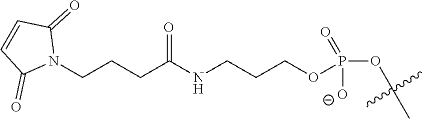 [SMILES: *OP(=O)([O-])OCCCNC(=O)CCCN1C(=O)C=CC1=O]
C3-NH2-Butyryl-Mal 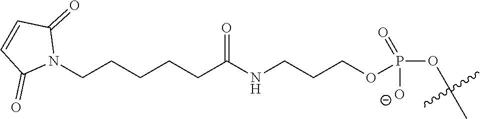 [SMILES: *OP(=O)([O-])OCCCNC(=O)CCCCCN1C(=O)C=CC1=O]
C3-NH2-Hexyl-Mal 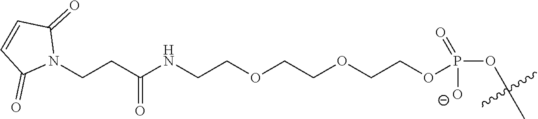 [SMILES: *OP(=O)([O-])OCCOCCOCCNC(=O)CCN1C(=O)C=CC1=O]
PEG2-NH2-Propyl-Mal 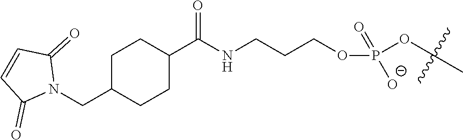 [SMILES: *OP(=O)([O-])OCCCNC(=O)C1CCC(CN2C(=O)C=CC2=O)CC1]
C3-NH2-Cyclohexyl-Mal 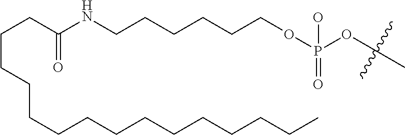 [SMILES: *OP(=O)(O)OCCCCCCNC(=O)CCCCCCCCCCCCCCC]
5′-C6-NH2-Palmitate

Other linkers can also be used, such as linkers formed with click chemistry, amide coupling, reductive amination, oxime, and enzymatic couplings such as transglutaminase and sortage conjugations. The linkers provided here are exemplary in nature and other linkers made with other such methods can also be used. For example, linkers connected through phosphate groups can be phosphorothioates or phosphorodithioates.

When connected to the siRNA, the structures, L-(X₄) can be represented by one of the following formulas:

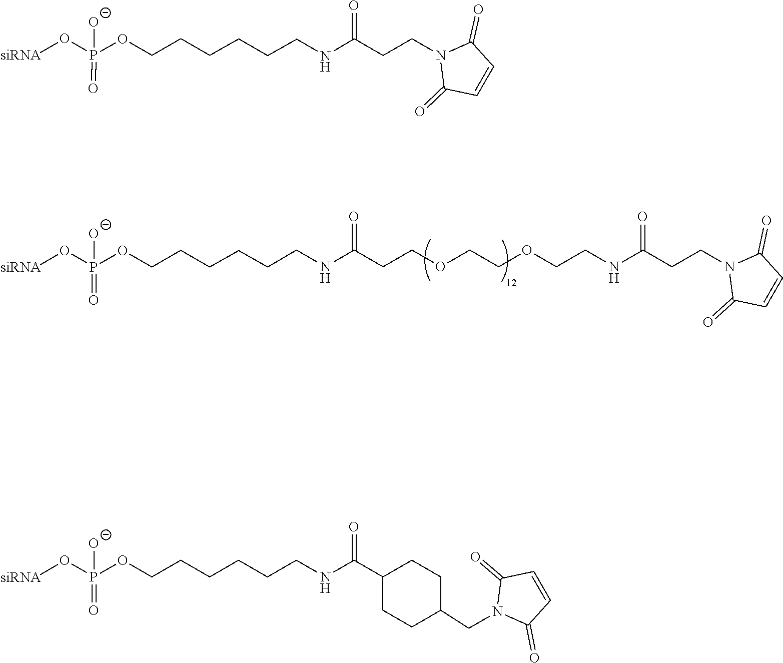

Although certain siRNA sequences are illustrated herein with certain modified nucleobases, the sequences without such modifications are also provided herein. That is, the sequence can comprise the sequences illustrated in the tables provided herein without any modifications. The unmodified siRNA sequences can still comprise, in some embodiments, a linker at the 5′ end of the of the sense strand of the dsRNA. In some embodiments, the nucleic acid molecules can be modified to include a vinyl phosphonate at the 5′ end of the of the antisense strand of the dsRNA. In some embodiments, the nucleic acid molecule can be modified to include a linker at the 3′ end of the of the sense strand of the dsRNA. In some embodiments, the nucleic acid molecule can be modified to include a vinyl phosphonate at the 3′ end of the of the antisense strand of the dsRNA. The linker can be as provided herein.

In some embodiments, the FN3 proteins comprising a polypeptide that binds CD71 are provided. In some embodiments, the polypeptide comprises an FN3 domain that binds to CD71. In some embodiments, the polypeptide comprises an amino acid sequence of SEQ ID NOs: 360-644, 663-672, or 1395-1849 are provided. In some embodiments, the polypeptide that binds CD71 comprises a sequence of SEQ ID NOs: 360-644, 663-672, or 1395-1849. The sequence of CD71 protein that the polypeptides can bind to can be, for example, SEQ ID Nos: 3 or 4. In some embodiments, the FN3 domain that binds to CD71 specifically binds to CD71.

In some embodiments, the FN3 domain that binds CD71 is based on Tencon sequence of SEQ ID NO: 1 or Tencon 27 sequence of SEQ ID NO: 2 (LPAPKNLVVSRVTEDSARLSWTAPDAAFDSFLIQYQESEKVGEAIVLTVPGSERSYD LTGLKPGTEYTVSIYGVKGGHRSNPLSAIFTT), optionally having substitutions at residues positions 11, 14, 17, 37, 46, 73, or 86 (residue numbering corresponding to SEQ ID NO: 2).

In some embodiments, an isolated FN3 domain that binds CD71 comprises the amino acid sequence of SEQ ID NOs: 360-644, 663-672, or 1395-1849.

In some embodiments, proteins comprising a polypeptide comprising an amino acid sequence of SEQ ID NO: 360 are provided. SEQ ID NO: 360 is a consensus sequence based on the sequences of SEQ ID NO: 361, SEQ ID NO: 362, SEQ ID NO: 363, and SEQ ID NO: 364. The sequence of SEQ ID NO: 360 is:

MLPAPKNLVVSRVTEDSARLSWTAPDAAFDSFX₁IX₂YX₃EX₄X₅X₆X₇GE
AIX₈LX₉VPGSERSYDLTGLKPGTEYX₁₀VX₁₁IX₁₂X₁₃VKGGX₁₄X₁₅SX₁₆P
LX₁₇AX₁₈FTT,

wherein X₈, X₉, X₁₇, and X₁₈ are each, independently, any amino acid other than methionine or proline, and
X₁ is selected from D, F, Y, or H,
X₂ is selected from Y, G, A, or V,
X₃ is selected from I, T, L, A, or H,
X₄ is selected from S, Y or P,
X₅ is selected from Y, G, Q, or R,
X₆ is selected from G or P,
X₇ is selected from A, Y, P, D, or S,
X₁₀ is selected from W, N, S, or E,
X₁₁ is selected from L, Y, or G,
X₁₂ is selected from D, Q, H, or V,
X₁₃ is selected from G or S,
X₁₄ is selected from R, G, F, L, or D,
X₁₅ is selected from W, S, P, or L, and
X₁₆ is selected from T, V, M, or S.

In some embodiments:
X₁ is selected from D, F, Y, or H,
X₂ is selected from G, A, or V,
X₃ is selected from T, L, A, or H,
X₄ is selected from Y or P,
X₅ is selected from G, Q, or R,
X₆ is selected from G or P,
X₇ is selected from Y, P, D, or S,
X₁₀ is selected from W, N, S, or E,
X₁₁ is selected from L, Y, or G,
X₁₂ is selected from Q, H, or V,
X₁₃ is selected from G or S,
X₁₄ is selected from G, F, L, or D,
X₁₅ is selected from S, P, or L, and
X₁₆ is selected from V, M, or S.

In some embodiments, X₁, X₂, X₃, X₄, X₅, X₆, X₇, X₁₀, X₁₁, X₁₂, X₁₃, X₁₄, X₁₅, and X₁₆ are as shown in the sequence of SEQ ID NO: 361. In some embodiments, X₁, X₂, X₃, X₄, X₅, X₆, X₇, X₁₀, X₁₁, X₁₂, X₁₃, X₁₄, X₁₅, and X₁₆ are as shown in the sequence of SEQ ID NO: 362. In some embodiments, X₁, X₂, X₃, X₄, X₅, X₆, X₇, X₁₀, X₁₁, X₁₂, X₁₃, X₁₄, X₁₅, and X₁₆ are as shown in the sequence of SEQ ID NO: 363. In some embodiments, X₁, X₂, X₃, X₄, X₅, X₆, X₇, X₁₀, X₁₁, X₁₂, X₁₃, X₁₄, X₁₅, and X₁₆ are as shown in the sequence of SEQ ID NO: 364.

In some embodiments, X₈, X₉, X₁₇, and X₁₈ is, independently, alanine, arginine, asparagine, aspartic acid, cysteine, glutamine, glutamic acid, glycine, histidine, isoleucine, leucine, lysine, phenylalanine, serine, threonine, tryptophan, tyrosine, or valine. In some embodiments, X₈, X₉, X₁₇, and X₁₈ is, independently, not alanine, arginine, asparagine, aspartic acid, cysteine, glutamine, glutamic acid, glycine, histidine, isoleucine, leucine, lysine, phenylalanine, serine, threonine, tryptophan, tyrosine, or valine. In some embodiments, X₈, X₉, X₁₇, and X₁₈ is, independently, alanine. In some embodiments, X₈, X₉, X₁₇, and X₁₈ is, independently, arginine. In some embodiments, X₈, X₉, X₁₇, and X₁₈ is, independently asparagine. In some embodiments, X₈, X₉, X₁₇, and X₁₈ is, independently, aspartic acid. In some embodiments, X₈, X₉, X₁₇, and X₁₈ is, independently, cysteine. In some embodiments, X₈, X₉, X₁₇, and X₁₈ is, independently, glutamine. In some embodiments, X_{S}, X₉, X₁₇, and X₁₈ is, independently, glutamic acid. In some embodiments, X₈, X₉, X₁₇, and X₁₈ is, independently, glycine. In some embodiments, X₈, X₉, X₁₇, and X₁₈ is, independently, histidine. In some embodiments, X₈, X₉, X₁₇, and X₁₈ is, independently, isoleucine. In some embodiments, X₈, X₉, X₁₇, and X₁₈ is, independently, leucine. In some embodiments, X₈, X₉, X₁₇, and X₁₈ is, independently, lysine. In some embodiments, X₈, X₉, X₁₇, and X₁₈ is, independently, phenylalanine. In some embodiments, X₈, X₉, X₁₇, and X₁₈ is, independently serine. In some embodiments, X₈, X₉, X₁₇, and X₁₈ is, independently, threonine. In some embodiments, X₈, X₉, X₁₇, and X₁₈ is, independently, tryptophan. In some embodiments, X_{S}, X₉, X₁₇, and X₁₈ is, independently, tyrosine. In some embodiments, X₈, X₉, X₁₇, and X₁ₛ is, independently valine.

In some embodiments, the sequence is set forth as shown in in the sequence of SEQ ID NO: 361, except that the positions that correspond to the positions of X₈, X₉, X₁₇, and X₁₈ can be any other amino acid residue as set forth above, except that in some embodiments, X₈ is not V, X₉ is not T, X₁₇ is not S, and X₁₈ is not I.

In some embodiments, the sequence is set forth as shown in in the sequence of SEQ ID NO: 362, except that the positions that correspond to the positions of X₈, X₉, X₁₇, and X₁₈ can be any other amino acid residue as set forth above, except that in some embodiments, X₈ is not V, X₉ is not T, X₁₇ is not S, and X₁₈ is not I.

In some embodiments, the sequence is set forth as shown in in the sequence of SEQ ID NO: 363, except that the positions that correspond to the positions of X₈, X₉, X₁₇, and X₁₈ can be any other amino acid residue as set forth above, except that in some embodiments, X_{S} is not V, X₉ is not T, X₁₇ is not S, and X₁₈ is not I.

In some embodiments, the sequence is set forth as shown in in the sequence of SEQ ID NO: 364, except that the positions that correspond to the positions of X₈, X₉, X₁₇, and X₁₈ can be any other amino acid residue as set forth above, except that in some embodiments, X_{S} is not V, X₉ is not T, X₁₇ is not S, and X₁₈ is not I.

In some embodiments, proteins comprising a polypeptide comprising an amino acid sequence that is at least 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to a sequence of SEQ ID NO: 360. In some embodiments, the protein is at least 85%, 86%, 87%, 88%, 89%, 90%, 91%, 92%, 93%, 94%, 95%, 96%, 97%, 98% or 99% identical to a sequence of SEQ ID NO: 360. In some embodiments, the protein is at least 90%, 91%, 92%, 93%, 94%, 95%, 96%, 97%, 98% or 99% identical to a sequence of SEQ ID NO: 360. In some embodiments, the protein is at least 95%, 96%, 97%, 98% or 99% identical to a sequence of SEQ ID NO: 360.

Sequences of SEQ ID NOs: 361-364 are listed in Table 7, below.

TABLE 7
CD71-binding FN3 Domain Sequences

| SEQ ID | |
|---|---|
| NO: | SEQUENCE |
| 361 | MLPAPKNLVVSRVTEDSARLSWTAPDAAFDSFYIAYAEPRPDGEAIVLTVPGS |
| | ERSYDLTGLKPGTEYSVLIHGVKGGLLSSPLSAIFTT |
| 362 | MLPAPKNLVVSRVTEDSARLSWTAPDAAFDSFFIGYLEPQPPGEAIVLTVPGS |
| | ERSYDLTGLKPGTEYNVTIQGVKGGFPSMPLSAIFTT |
| 363 | MLPAPKNLVVSRVTEDSARLSWTAPDAAFDSFHIVYHEPRPSGEAIVLTVPGS |
| | ERSYDLTGLKPGTEYEVGIVSVKGGDLSVPLSAIFTT |
| 364 | MLPAPKNLVVSRVTEDSARLSWTAPDAAFDSFDIGYTEYGGYGEAIVLTVPGS |
| | ERSYDLTGLKPGTEYWVLIQGVKGGGSSVPLSAIFTT |

Percent identity can be determined using the default parameters to align two sequences using BlastP available through the NCBI website.

As provided herein, in some embodiments, the FN3 domain that binds to CD71 binds to human mature CD71 or the human mature CD71 extracellular domain. In some embodiments, the human mature CD71 is SEQ ID NO: 3, and the human mature CD71 extracellular binding domain is SEQ ID NO: 4, each of which is provided below in Table 8.

TABLE 8
CD71 Sequences

| SEQ | |
|---|---|
| ID NO: | SEQUENCE |
| 3 | MMDQARSAFSNLFGGEPLSYTRFSLARQVDGDNSHVEMKLAVDEEENADNNTKA |
| | NVTKPKRCSGSICYGTIAVIVFFLIGFMIGYLGYCKGVEPKTECERLAGTESPV |
| | REEPGEDFPAARRLYWDDLKRKLSEKLDSTDFTGTIKLLNENSYVPREAGSQKD |
| | ENLALYVENQFREFKLSKVWRDQHFVKIQVKDSAQNSVIIVDKNGRLVYLVENP |
| | GGYVAYSKAATVTGKLVHANFGTKKDFEDLYTPVNGSIVIVRAGKITFAEKVAN |
| | AESLNAIGVLIYMDQTKFPIVNAELSFFGHAHLGTGDPYTPGFPSENHTQFPPS |
| | RSSGLPNIPVQTISRAAAEKLFGNMEGDCPSDWKTDSTCRMVTSESKNVKLTVS |
| | NVLKEIKILNIFGVIKGFVEPDHYVVVGAQRDAWGPGAAKSGVGTALLLKLAQM |
| | FSDMVLKDGFQPSRSIIFASWSAGDEGSVGATEWLEGYLSSLHLKAFTYINLDK |
| | AVLGTSNFKVSASPLLYTLIEKTMQNVKHPVTGQFLYQDSNWASKVEKLILDNA |
| | AFPFLAYSGIPAVSFCFCEDTDYPYLGTTMDTYKELIERIPELNKVARAAAEVA |
| | GQFVIKLTHDVELNLDYERYNSQLLSFVRDLNQYRADIKEMGLSLQWLYSARGD |
| | FFRATSRLTTDFGNAEKTDRFVMKKLNDRVMRVEYHFLSPYVSPKESPFRHVEW |
| | GSGSHTLPALLENLKLRKQNNGAFNETLERNQLALATWTIQGAANALSGDVWDI |
| | DNEF |
| 4 | CKGVEPKTECERLAGTESPVREEPGEDFPAARRLYWDDLKRKLSEKLDSTDFTG |
| | TIKLLNENSYVPREAGSQKDENLALYVENQFREFKLSKVWRDQHFVKIQVKDSA |
| | QNSVIIVDKNGRLVYLVENPGGYVAYSKAATVTGKLVHANFGTKKDFEDLYTPV |
| | NGSIVIVRAGKITFAEKVANAESLNAIGVLIYMDQTKFPIVNAELSFFGHAHLG |
| | TGDPYTPGFPSFNHTQFPPSRSSGLPNIPVQTISRAAAEKLEGNMEGDCPSDWK |
| | TDSTCRMVTSESKNVKLTVSNVLKEIKILNIFGVIKGFVEPDHYVVVGAQRDAW |
| | GPGAAKSGVGTALLLKLAQMFSDMVLKDGFQPSRSIIFASWSAGDEGSVGATEW |
| | LEGYLSSLHLKAFTYINLDKAVLGTSNFKVSASPLLYTLIEKTMQNVKHPVTGQ |
| | FLYQDSNWASKVEKLTLDNAAFPFLAYSGIPAVSFCFCEDTDYPYLGTTMDTYK |
| | ELIERIPELNKVARAAAEVAGQFVIKLTHDVELNLDYERYNSQLLSFVRDLNQY |
| | RADIKEMGLSLQWLYSARGDFFRATSRLTTDFGNAEKTDRFVMKKLNDRVMRVE |
| | YHFLSPYVSPKESPFRHVFWGSGSHTLPALLENLKLRKQNNGAFNETLERNQLA |
| | LATWTIQGAANALSGDVWDIDNEF |

As provided herein, the FN3 domains can bind to the CD71 protein. Also provided, even if not explicitly stated, is that the domains can also specifically bind to the CD71 protein. Thus, for example, an FN3 domain that binds to CD71 would also encompass an FN3 domain protein that specifically binds to CD71. These molecules can be used, for example, in therapeutic and diagnostic applications and in imaging. In some embodiments, polynucleotides encoding the FN3 domains disclosed herein or complementary nucleic acids thereof, vectors, host cells, and methods of making and using them are provided. In some embodiments, an isolated FN3 domain that binds or specifically binds CD71 is provided.

In some embodiments, the FN3 domain may bind CD71 with a dissociation constant (K_{D}) of less than about 1×10⁻⁷ M, for example less than about 1×10⁻⁸ M, less than about 1×10-9 M, less than about 1×10⁻¹⁰ M, less than about 1×10⁻¹ M, less than about 1×10⁻² M, or less than about 1×10⁻³ M as determined by surface plasmon resonance or the Kinexa method, as practiced by those of skill in the art. The measured affinity of a particular FN3 domain-antigen interaction can vary if measured under different conditions (e.g., osmolarity, pH). Thus, measurements of affinity and other antigen-binding parameters (e.g., K_{D}, Kₒₙ, K_{off}) are made with standardized solutions of protein scaffold and antigen, and a standardized buffer, such as the buffers described herein.

In some embodiments, the FN3 domain may bind CD71 at least 5-fold above the signal obtained for a negative control in a standard solution ELISA assay.

In some embodiments, the FN3 domain that binds or specifically binds CD71 comprises an initiator methionine (Met) linked to the N-terminus of the molecule. In some embodiments, the FN3 domain that binds or specifically binds CD71 comprises a cysteine (Cys) linked to a C-terminus of the FN3 domain. The addition of the N-terminal Met and/or the C-terminal Cys may facilitate expression and/or conjugation to extend half-life and to provide other functions of molecules.

The FN3 domain can also contain cysteine substitutions, such as those that are described in U.S. Pat. No. 10,196,446, which is hereby incorporated by reference in its entirety. Briefly, in some embodiments, the polypeptides provided herein can comprise at least one cysteine substitution at a position selected from the group consisting of residues 6, 8, 10, 11, 14, 15, 16, 20, 30, 34, 38, 40, 41, 45, 47, 48, 53, 54, 59, 60, 62, 64, 70, 88, 89, 90, 91, and 93 of the FN3 domain based on SEQ ID NO: 1 or SEQ ID NO: 1 of U.S. Pat. No. 10,196,446

(SEQ ID NO: 2311)
LPAPKNLVVSEVTEDSLRLSWTAPDAAFDSFLIQYQESE
KVGEAINLTVPGSERSYDLTGLKPGTEYTVSIYGVKGGH
RSNPLSAEFTT,

which is hereby incorporated by reference in its entirety, and the equivalent positions in related FN3 domains.

In some embodiments, the substitution is at residue 6. In some embodiments, the substitution is at residue 8. In some embodiments, the substitution is at residue 10. In some embodiments, the substitution is at residue 11. In some embodiments, the substitution is at residue 14. In some embodiments, the substitution is at residue 15. In some embodiments, the substitution is at residue 16. In some embodiments, the substitution is at residue 20. In some embodiments, the substitution is at residue 30. In some embodiments, the substitution is at residue 34. In some embodiments, the substitution is at residue 38. In some embodiments, the substitution is at residue 40. In some embodiments, the substitution is at residue 41. In some embodiments, the substitution is at residue 45. In some embodiments, the substitution is at residue 47. In some embodiments, the substitution is at residue 48. In some embodiments, the substitution is at residue 53. In some embodiments, the substitution is at residue 54. In some embodiments, the substitution is at residue 59. In some embodiments, the substitution is at residue 60. In some embodiments, the substitution is at residue 62. In some embodiments, the substitution is at residue 64. In some embodiments, the substitution is at residue 70. In some embodiments, the substitution is at residue 88. In some embodiments, the substitution is at residue 89. In some embodiments, the substitution is at residue 90. In some embodiments, the substitution is at residue 91. In some embodiments, the substitution is at residue 93.

A cysteine substitution at a position in the domain or protein comprises a replacement of the existing amino acid residue with a cysteine residue. In some embodiments, instead of a substitution a cysteine is inserted into the sequence adjacent to the positions listed above. Other examples of cysteine modifications can be found in, for example, U.S. Patent Application Publication No. 2017/0362301, which is hereby incorporated by reference in its entirety. The alignment of the sequences can be performed using BlastP using the default parameters at, for example, the NCBI website.

In some embodiments, a cysteine residue is inserted at any position in the domain or protein.

In some embodiments, the FN3 domain that binds CD71 is internalized into a cell. In some embodiments, internalization of the FN3 domain may facilitate delivery of a detectable label or therapeutic into a cell. In some embodiments, internalization of the FN3 domain may facilitate delivery of a cytotoxic agent into a cell. The cytotoxic agent can act as a therapeutic agent. In some embodiments, internalization of the FN3 domain may facilitate the delivery of any detectable label, therapeutic, and/or cytotoxic agent disclosed herein into a cell. In some embodiments, internalization of the FN3 domain may facilitate delivery of a oligonucleotide into a cell. In some embodiments, the cell is a tumor cell. In some embodiments, the cell is a liver cell. In some embodiments, the cell is a muscle cell. In some embodiments, the cell is an immune cell. In some embodiments, the cell is a cell of the central nervous system. In some embodiments, the cell is a heart cell. In some embodiments, the therapeutic is an siRNA molecule as provided for herein. The FN3 domains that bind CD71 conjugated to a detectable label can be used to evaluate expression of CD71 on samples such as tumor tissue in vivo or in vitro. The FN3 domains that bind CD71 conjugated to a detectable label can be used to evaluate expression of CD71 on samples blood, immune cells, or muscle cells in vivo or in vitro.

In some embodiments, an isolated FN3 domain that binds CD71 comprises the amino acid sequence of SEQ ID NOs: 360-644, 663-672, or 1395-1849.

In some embodiments, an isolated FN3 domain that binds CD71 comprises the amino acid sequence of SEQ ID NO: 365. In some embodiments, an isolated FN3 domain that binds CD71 comprises the amino acid sequence of SEQ ID NO: 366. In some embodiments, an isolated FN3 domain that binds CD71 comprises the amino acid sequence of SEQ ID NO: 367. In some embodiments, an isolated FN3 domain that binds CD71 comprises the amino acid sequence of SEQ ID NO: 368. In some embodiments, an isolated FN3 domain that binds CD71 comprises the amino acid sequence of SEQ ID NO: 369. In some embodiments, an isolated FN3 domain that binds CD71 comprises the amino acid sequence of SEQ ID NO: 370. In some embodiments, an isolated FN3 domain that binds CD71 comprises the amino acid sequence of SEQ ID NO: 371. In some embodiments, an isolated FN3 domain that binds CD71 comprises the amino acid sequence of SEQ ID NO: 372. In some embodiments, an isolated FN3 domain that binds CD71 comprises the amino acid sequence of SEQ ID NO: 373. In some embodiments, an isolated FN3 domain that binds CD71 comprises the amino acid sequence of SEQ ID NO: 374. In some embodiments, an isolated FN3 domain that binds CD71 comprises the amino acid sequence of SEQ ID NO: 375. In some embodiments, an isolated FN3 domain that binds CD71 comprises the amino acid sequence of SEQ ID NO: 376. In some embodiments, an isolated FN3 domain that binds CD71 comprises the amino acid sequence of SEQ ID NO: 377. In some embodiments, an isolated FN3 domain that binds CD71 comprises the amino acid sequence of SEQ ID NO: 378. In some embodiments, an isolated FN3 domain that binds CD71 comprises the amino acid sequence of SEQ ID NO: 379. In some embodiments, an isolated FN3 domain that binds CD71 comprises the amino acid sequence of SEQ ID NO: 380. In some embodiments, an isolated FN3 domain that binds CD71 comprises the amino acid sequence of SEQ ID NO: 381. In some embodiments, an isolated FN3 domain that binds CD71 comprises the amino acid sequence of SEQ ID NO: 382. In some embodiments, an isolated FN3 domain that binds CD71 comprises the amino acid sequence of SEQ ID NO: 383. In some embodiments, an isolated FN3 domain that binds CD71 comprises the amino acid sequence of SEQ ID NO: 384. In some embodiments, an isolated FN3 domain that binds CD71 comprises the amino acid sequence of SEQ ID NO: 385. In some embodiments, an isolated FN3 domain that binds CD71 comprises the amino acid sequence of SEQ ID NO: 386. In some embodiments, an isolated FN3 domain that binds CD71 comprises the amino acid sequence of SEQ ID NO: 387. In some embodiments, an isolated FN3 domain that binds CD71 comprises the amino acid sequence of SEQ ID NO: 388. In some embodiments, an isolated FN3 domain that binds CD71 comprises the amino acid sequence of SEQ ID NO: 389. In some embodiments, an isolated FN3 domain that binds CD71 comprises the amino acid sequence of SEQ ID NO: 390. In some embodiments, an isolated FN3 domain that binds CD71 comprises the amino acid sequence of SEQ ID NO: 391. In some embodiments, an isolated FN3 domain that binds CD71 comprises the amino acid sequence of SEQ ID NO: 392. In some embodiments, an isolated FN3 domain that binds CD71 comprises the amino acid sequence of SEQ ID NO: 393. In some embodiments, an isolated FN3 domain that binds CD71 comprises the amino acid sequence of SEQ ID NO: 394. In some embodiments, an isolated FN3 domain that binds CD71 comprises the amino acid sequence of SEQ ID NO: 395. In some embodiments, an isolated FN3 domain that binds CD71 comprises the amino acid sequence of SEQ ID NO: 396. In some embodiments, an isolated FN3 domain that binds CD71 comprises the amino acid sequence of SEQ ID NO: 397. In some embodiments, an isolated FN3 domain that binds CD71 comprises the amino acid sequence of SEQ ID NO: 398. In some embodiments, an isolated FN3 domain that binds CD71 comprises the amino acid sequence of SEQ ID NO: 399. In some embodiments, an isolated FN3 domain that binds CD71 comprises the amino acid sequence of SEQ ID NO: 400. In some embodiments, an isolated FN3 domain that binds CD71 comprises the amino acid sequence of SEQ ID NO: 401. In some embodiments, an isolated FN3 domain that binds CD71 comprises the amino acid sequence of SEQ ID NO: 402. In some embodiments, an isolated FN3 domain that binds CD71 comprises the amino acid sequence of SEQ ID NO: 403. In some embodiments, an isolated FN3 domain that binds CD71 comprises the amino acid sequence of SEQ ID NO: 404. In some embodiments, an isolated FN3 domain that binds CD71 comprises the amino acid sequence of SEQ ID NO: 405. In some embodiments, an isolated FN3 domain that binds CD71 comprises the amino acid sequence of SEQ ID NO: 406. In some embodiments, an isolated FN3 domain that binds CD71 comprises the amino acid sequence of SEQ ID NO: 407. In some embodiments, an isolated FN3 domain that binds CD71 comprises the amino acid sequence of SEQ ID NO: 408. In some embodiments, an isolated FN3 domain that binds CD71 comprises the amino acid sequence of SEQ ID NO: 409. In some embodiments, an isolated FN3 domain that binds CD71 comprises the amino acid sequence of SEQ ID NO: 410. In some embodiments, an isolated FN3 domain that binds CD71 comprises the amino acid sequence of SEQ ID NO: 411. In some embodiments, an isolated FN3 domain that binds CD71 comprises the amino acid sequence of SEQ ID NO: 412. In some embodiments, an isolated FN3 domain that binds CD71 comprises the amino acid sequence of SEQ ID NO: 413. In some embodiments, an isolated FN3 domain that binds CD71 comprises the amino acid sequence of SEQ ID NO: 414. In some embodiments, an isolated FN3 domain that binds CD71 comprises the amino acid sequence of SEQ ID NO: 415. In some embodiments, an isolated FN3 domain that binds CD71 comprises the amino acid sequence of SEQ ID NO: 416. In some embodiments, an isolated FN3 domain that binds CD71 comprises the amino acid sequence of SEQ ID NO: 417. In some embodiments, an isolated FN3 domain that binds CD71 comprises the amino acid sequence of SEQ ID NO: 418. In some embodiments, an isolated FN3 domain that binds CD71 comprises the amino acid sequence of SEQ ID NO: 419. In some embodiments, an isolated FN3 domain that binds CD71 comprises the amino acid sequence of SEQ ID NO: 420. In some embodiments, an isolated FN3 domain that binds CD71 comprises the amino acid sequence of SEQ ID NO: 421. In some embodiments, an isolated FN3 domain that binds CD71 comprises the amino acid sequence of SEQ ID NO: 422. In some embodiments, an isolated FN3 domain that binds CD71 comprises the amino acid sequence of SEQ ID NO: 423. In some embodiments, an isolated FN3 domain that binds CD71 comprises the amino acid sequence of SEQ ID NO: 424. In some embodiments, an isolated FN3 domain that binds CD71 comprises the amino acid sequence of SEQ ID NO: 425. In some embodiments, an isolated FN3 domain that binds CD71 comprises the amino acid sequence of SEQ ID NO: 426. In some embodiments, an isolated FN3 domain that binds CD71 comprises the amino acid sequence of SEQ ID NO: 427. In some embodiments, an isolated FN3 domain that binds CD71 comprises the amino acid sequence of SEQ ID NO: 428. In some embodiments, an isolated FN3 domain that binds CD71 comprises the amino acid sequence of SEQ ID NO: 429. In some embodiments, an isolated FN3 domain that binds CD71 comprises the amino acid sequence of SEQ ID NO: 430. In some embodiments, an isolated FN3 domain that binds CD71 comprises the amino acid sequence of SEQ ID NO: 431. In some embodiments, an isolated FN3 domain that binds CD71 comprises the amino acid sequence of SEQ ID NO: 432. In some embodiments, an isolated FN3 domain that binds CD71 comprises the amino acid sequence of SEQ ID NO: 433. In some embodiments, an isolated FN3 domain that binds CD71 comprises the amino acid sequence of SEQ ID NO: 434. In some embodiments, an isolated FN3 domain that binds CD71 comprises the amino acid sequence of SEQ ID NO: 435. In some embodiments, an isolated FN3 domain that binds CD71 comprises the amino acid sequence of SEQ ID NO: 436. In some embodiments, an isolated FN3 domain that binds CD71 comprises the amino acid sequence of SEQ ID NO: 437. In some embodiments, an isolated FN3 domain that binds CD71 comprises the amino acid sequence of SEQ ID NO: 438. In some embodiments, an isolated FN3 domain that binds CD71 comprises the amino acid sequence of SEQ ID NO: 439. In some embodiments, an isolated FN3 domain that binds CD71 comprises the amino acid sequence of SEQ ID NO: 440. In some embodiments, an isolated FN3 domain that binds CD71 comprises the amino acid sequence of SEQ ID NO: 441. In some embodiments, an isolated FN3 domain that binds CD71 comprises the amino acid sequence of SEQ ID NO: 442. In some embodiments, an isolated FN3 domain that binds CD71 comprises the amino acid sequence of SEQ ID NO: 443. In some embodiments, an isolated FN3 domain that binds CD71 comprises the amino acid sequence of SEQ ID NO: 444. In some embodiments, an isolated FN3 domain that binds CD71 comprises the amino acid sequence of SEQ ID NO: 445. In some embodiments, an isolated FN3 domain that binds CD71 comprises the amino acid sequence of SEQ ID NO: 446. In some embodiments, an isolated FN3 domain that binds CD71 comprises the amino acid sequence of SEQ ID NO: 447. In some embodiments, an isolated FN3 domain that binds CD71 comprises the amino acid sequence of SEQ ID NO: 448. In some embodiments, an isolated FN3 domain that binds CD71 comprises the amino acid sequence of SEQ ID NO: 449. In some embodiments, an isolated FN3 domain that binds CD71 comprises the amino acid sequence of SEQ ID NO: 450. In some embodiments, an isolated FN3 domain that binds CD71 comprises the amino acid sequence of SEQ ID NO: 451. In some embodiments, an isolated FN3 domain that binds CD71 comprises the amino acid sequence of SEQ ID NO: 452. In some embodiments, an isolated FN3 domain that binds CD71 comprises the amino acid sequence of SEQ ID NO: 453. In some embodiments, an isolated FN3 domain that binds CD71 comprises the amino acid sequence of SEQ ID NO: 454. In some embodiments, an isolated FN3 domain that binds CD71 comprises the amino acid sequence of SEQ ID NO: 455. In some embodiments, an isolated FN3 domain that binds CD71 comprises the amino acid sequence of SEQ ID NO: 456. In some embodiments, an isolated FN3 domain that binds CD71 comprises the amino acid sequence of SEQ ID NO: 457. In some embodiments, an isolated FN3 domain that binds CD71 comprises the amino acid sequence of SEQ ID NO: 458. In some embodiments, an isolated FN3 domain that binds CD71 comprises the amino acid sequence of SEQ ID NO: 459. In some embodiments, an isolated FN3 domain that binds CD71 comprises the amino acid sequence of SEQ ID NO: 460. In some embodiments, an isolated FN3 domain that binds CD71 comprises the amino acid sequence of SEQ ID NO: 461. In some embodiments, an isolated FN3 domain that binds CD71 comprises the amino acid sequence of SEQ ID NO: 462. In some embodiments, an isolated FN3 domain that binds CD71 comprises the amino acid sequence of SEQ ID NO: 463. In some embodiments, an isolated FN3 domain that binds CD71 comprises the amino acid sequence of SEQ ID NO: 464. In some embodiments, an isolated FN3 domain that binds CD71 comprises the amino acid sequence of SEQ ID NO: 465. In some embodiments, an isolated FN3 domain that binds CD71 comprises the amino acid sequence of SEQ ID NO: 466. In some embodiments, an isolated FN3 domain that binds CD71 comprises the amino acid sequence of SEQ ID NO: 467. In some embodiments, an isolated FN3 domain that binds CD71 comprises the amino acid sequence of SEQ ID NO: 468. In some embodiments, an isolated FN3 domain that binds CD71 comprises the amino acid sequence of SEQ ID NO: 469. In some embodiments, an isolated FN3 domain that binds CD71 comprises the amino acid sequence of SEQ ID NO: 470. In some embodiments, an isolated FN3 domain that binds CD71 comprises the amino acid sequence of SEQ ID NO: 471. In some embodiments, an isolated FN3 domain that binds CD71 comprises the amino acid sequence of SEQ ID NO: 472. In some embodiments, an isolated FN3 domain that binds CD71 comprises the amino acid sequence of SEQ ID NO: 473. In some embodiments, an isolated FN3 domain that binds CD71 comprises the amino acid sequence of SEQ ID NO: 474. In some embodiments, an isolated FN3 domain that binds CD71 comprises the amino acid sequence of SEQ ID NO: 475. In some embodiments, an isolated FN3 domain that binds CD71 comprises the amino acid sequence of SEQ ID NO: 476. In some embodiments, an isolated FN3 domain that binds CD71 comprises the amino acid sequence of SEQ ID NO: 477. In some embodiments, an isolated FN3 domain that binds CD71 comprises the amino acid sequence of SEQ ID NO: 478. In some embodiments, an isolated FN3 domain that binds CD71 comprises the amino acid sequence of SEQ ID NO: 479. In some embodiments, an isolated FN3 domain that binds CD71 comprises the amino acid sequence of SEQ ID NO: 480. In some embodiments, an isolated FN3 domain that binds CD71 comprises the amino acid sequence of SEQ ID NO: 481. In some embodiments, an isolated FN3 domain that binds CD71 comprises the amino acid sequence of SEQ ID NO: 482. In some embodiments, an isolated FN3 domain that binds CD71 comprises the amino acid sequence of SEQ ID NO: 483. In some embodiments, an isolated FN3 domain that binds CD71 comprises the amino acid sequence of SEQ ID NO: 484. In some embodiments, an isolated FN3 domain that binds CD71 comprises the amino acid sequence of SEQ ID NO: 485. In some embodiments, an isolated FN3 domain that binds CD71 comprises the amino acid sequence of SEQ ID NO: 486. In some embodiments, an isolated FN3 domain that binds CD71 comprises the amino acid sequence of SEQ ID NO: 487. In some embodiments, an isolated FN3 domain that binds CD71 comprises the amino acid sequence of SEQ ID NO: 488. In some embodiments, an isolated FN3 domain that binds CD71 comprises the amino acid sequence of SEQ ID NO: 489. In some embodiments, an isolated FN3 domain that binds CD71 comprises the amino acid sequence of SEQ ID NO: 490. In some embodiments, an isolated FN3 domain that binds CD71 comprises the amino acid sequence of SEQ ID NO: 491. In some embodiments, an isolated FN3 domain that binds CD71 comprises the amino acid sequence of SEQ ID NO: 492. In some embodiments, an isolated FN3 domain that binds CD71 comprises the amino acid sequence of SEQ ID NO: 493. In some embodiments, an isolated FN3 domain that binds CD71 comprises the amino acid sequence of SEQ ID NO: 494. In some embodiments, an isolated FN3 domain that binds CD71 comprises the amino acid sequence of SEQ ID NO: 495. In some embodiments, an isolated FN3 domain that binds CD71 comprises the amino acid sequence of SEQ ID NO: 496. In some embodiments, an isolated FN3 domain that binds CD71 comprises the amino acid sequence of SEQ ID NO: 497. In some embodiments, an isolated FN3 domain that binds CD71 comprises the amino acid sequence of SEQ ID NO: 498. In some embodiments, an isolated FN3 domain that binds CD71 comprises the amino acid sequence of SEQ ID NO: 499. In some embodiments, an isolated FN3 domain that binds CD71 comprises the amino acid sequence of SEQ ID NO: 500. In some embodiments, an isolated FN3 domain that binds CD71 comprises the amino acid sequence of SEQ ID NO: 501. In some embodiments, an isolated FN3 domain that binds CD71 comprises the amino acid sequence of SEQ ID NO: 502. In some embodiments, an isolated FN3 domain that binds CD71 comprises the amino acid sequence of SEQ ID NO: 503. In some embodiments, an isolated FN3 domain that binds CD71 comprises the amino acid sequence of SEQ ID NO: 504. In some embodiments, an isolated FN3 domain that binds CD71 comprises the amino acid sequence of SEQ ID NO: 505. In some embodiments, an isolated FN3 domain that binds CD71 comprises the amino acid sequence of SEQ ID NO: 506. In some embodiments, an isolated FN3 domain that binds CD71 comprises the amino acid sequence of SEQ ID NO: 507. In some embodiments, an isolated FN3 domain that binds CD71 comprises the amino acid sequence of SEQ ID NO: 508. In some embodiments, an isolated FN3 domain that binds CD71 comprises the amino acid sequence of SEQ ID NO: 509. In some embodiments, an isolated FN3 domain that binds CD71 comprises the amino acid sequence of SEQ ID NO: 510. In some embodiments, an isolated FN3 domain that binds CD71 comprises the amino acid sequence of SEQ ID NO: 511. In some embodiments, an isolated FN3 domain that binds CD71 comprises the amino acid sequence of SEQ ID NO: 512. In some embodiments, an isolated FN3 domain that binds CD71 comprises the amino acid sequence of SEQ ID NO: 513. In some embodiments, an isolated FN3 domain that binds CD71 comprises the amino acid sequence of SEQ ID NO: 514. In some embodiments, an isolated FN3 domain that binds CD71 comprises the amino acid sequence of SEQ ID NO: 515. In some embodiments, an isolated FN3 domain that binds CD71 comprises the amino acid sequence of SEQ ID NO: 516. In some embodiments, an isolated FN3 domain that binds CD71 comprises the amino acid sequence of SEQ ID NO: 517. In some embodiments, an isolated FN3 domain that binds CD71 comprises the amino acid sequence of SEQ ID NO: 518. In some embodiments, an isolated FN3 domain that binds CD71 comprises the amino acid sequence of SEQ ID NO: 519. In some embodiments, an isolated FN3 domain that binds CD71 comprises the amino acid sequence of SEQ ID NO: 520. In some embodiments, an isolated FN3 domain that binds CD71 comprises the amino acid sequence of SEQ ID NO: 521. In some embodiments, an isolated FN3 domain that binds CD71 comprises the amino acid sequence of SEQ ID NO: 522. In some embodiments, an isolated FN3 domain that binds CD71 comprises the amino acid sequence of SEQ ID NO: 523. In some embodiments, an isolated FN3 domain that binds CD71 comprises the amino acid sequence of SEQ ID NO: 524. In some embodiments, an isolated FN3 domain that binds CD71 comprises the amino acid sequence of SEQ ID NO: 525. In some embodiments, an isolated FN3 domain that binds CD71 comprises the amino acid sequence of SEQ ID NO: 526. In some embodiments, an isolated FN3 domain that binds CD71 comprises the amino acid sequence of SEQ ID NO: 527. In some embodiments, an isolated FN3 domain that binds CD71 comprises the amino acid sequence of SEQ ID NO: 528. In some embodiments, an isolated FN3 domain that binds CD71 comprises the amino acid sequence of SEQ ID NO: 529. In some embodiments, an isolated FN3 domain that binds CD71 comprises the amino acid sequence of SEQ ID NO: 530. In some embodiments, an isolated FN3 domain that binds CD71 comprises the amino acid sequence of SEQ ID NO: 531. In some embodiments, an isolated FN3 domain that binds CD71 comprises the amino acid sequence of SEQ ID NO: 532. In some embodiments, an isolated FN3 domain that binds CD71 comprises the amino acid sequence of SEQ ID NO: 533. In some embodiments, an isolated FN3 domain that binds CD71 comprises the amino acid sequence of SEQ ID NO: 534. In some embodiments, an isolated FN3 domain that binds CD71 comprises the amino acid sequence of SEQ ID NO: 535. In some embodiments, an isolated FN3 domain that binds CD71 comprises the amino acid sequence of SEQ ID NO: 536. In some embodiments, an isolated FN3 domain that binds CD71 comprises the amino acid sequence of SEQ ID NO: 537. In some embodiments, an isolated FN3 domain that binds CD71 comprises the amino acid sequence of SEQ ID NO: 538. In some embodiments, an isolated FN3 domain that binds CD71 comprises the amino acid sequence of SEQ ID NO: 539. In some embodiments, an isolated FN3 domain that binds CD71 comprises the amino acid sequence of SEQ ID NO: 540. In some embodiments, an isolated FN3 domain that binds CD71 comprises the amino acid sequence of SEQ ID NO: 541. In some embodiments, an isolated FN3 domain that binds CD71 comprises the amino acid sequence of SEQ ID NO: 542. In some embodiments, an isolated FN3 domain that binds CD71 comprises the amino acid sequence of SEQ ID NO: 543. In some embodiments, an isolated FN3 domain that binds CD71 comprises the amino acid sequence of SEQ ID NO: 544. In some embodiments, an isolated FN3 domain that binds CD71 comprises the amino acid sequence of SEQ ID NO: 545. In some embodiments, an isolated FN3 domain that binds CD71 comprises the amino acid sequence of SEQ ID NO: 546. In some embodiments, an isolated FN3 domain that binds CD71 comprises the amino acid sequence of SEQ ID NO: 547. In some embodiments, an isolated FN3 domain that binds CD71 comprises the amino acid sequence of SEQ ID NO: 548. In some embodiments, an isolated FN3 domain that binds CD71 comprises the amino acid sequence of SEQ ID NO: 549. In some embodiments, an isolated FN3 domain that binds CD71 comprises the amino acid sequence of SEQ ID NO: 550. In some embodiments, an isolated FN3 domain that binds CD71 comprises the amino acid sequence of SEQ ID NO: 551. In some embodiments, an isolated FN3 domain that binds CD71 comprises the amino acid sequence of SEQ ID NO: 552. In some embodiments, an isolated FN3 domain that binds CD71 comprises the amino acid sequence of SEQ ID NO: 553. In some embodiments, an isolated FN3 domain that binds CD71 comprises the amino acid sequence of SEQ ID NO: 554. In some embodiments, an isolated FN3 domain that binds CD71 comprises the amino acid sequence of SEQ ID NO: 555. In some embodiments, an isolated FN3 domain that binds CD71 comprises the amino acid sequence of SEQ ID NO: 556. In some embodiments, an isolated FN3 domain that binds CD71 comprises the amino acid sequence of SEQ ID NO: 557. In some embodiments, an isolated FN3 domain that binds CD71 comprises the amino acid sequence of SEQ ID NO: 558. In some embodiments, an isolated FN3 domain that binds CD71 comprises the amino acid sequence of SEQ ID NO: 559. In some embodiments, an isolated FN3 domain that binds CD71 comprises the amino acid sequence of SEQ ID NO: 560. In some embodiments, an isolated FN3 domain that binds CD71 comprises the amino acid sequence of SEQ ID NO: 561. In some embodiments, an isolated FN3 domain that binds CD71 comprises the amino acid sequence of SEQ ID NO: 562. In some embodiments, an isolated FN3 domain that binds CD71 comprises the amino acid sequence of SEQ ID NO: 563. In some embodiments, an isolated FN3 domain that binds CD71 comprises the amino acid sequence of SEQ ID NO: 564. In some embodiments, an isolated FN3 domain that binds CD71 comprises the amino acid sequence of SEQ ID NO: 565. In some embodiments, an isolated FN3 domain that binds CD71 comprises the amino acid sequence of SEQ ID NO: 566. In some embodiments, an isolated FN3 domain that binds CD71 comprises the amino acid sequence of SEQ ID NO: 567. In some embodiments, an isolated FN3 domain that binds CD71 comprises the amino acid sequence of SEQ ID NO: 568. In some embodiments, an isolated FN3 domain that binds CD71 comprises the amino acid sequence of SEQ ID NO: 569. In some embodiments, an isolated FN3 domain that binds CD71 comprises the amino acid sequence of SEQ ID NO: 570. In some embodiments, an isolated FN3 domain that binds CD71 comprises the amino acid sequence of SEQ ID NO: 571. In some embodiments, an isolated FN3 domain that binds CD71 comprises the amino acid sequence of SEQ ID NO: 572. In some embodiments, an isolated FN3 domain that binds CD71 comprises the amino acid sequence of SEQ ID NO: 573. In some embodiments, an isolated FN3 domain that binds CD71 comprises the amino acid sequence of SEQ ID NO: 574. In some embodiments, an isolated FN3 domain that binds CD71 comprises the amino acid sequence of SEQ ID NO: 575. In some embodiments, an isolated FN3 domain that binds CD71 comprises the amino acid sequence of SEQ ID NO: 576. In some embodiments, an isolated FN3 domain that binds CD71 comprises the amino acid sequence of SEQ ID NO: 577. In some embodiments, an isolated FN3 domain that binds CD71 comprises the amino acid sequence of SEQ ID NO: 578. In some embodiments, an isolated FN3 domain that binds CD71 comprises the amino acid sequence of SEQ ID NO: 579. In some embodiments, an isolated FN3 domain that binds CD71 comprises the amino acid sequence of SEQ ID NO: 580. In some embodiments, an isolated FN3 domain that binds CD71 comprises the amino acid sequence of SEQ ID NO: 581. In some embodiments, an isolated FN3 domain that binds CD71 comprises the amino acid sequence of SEQ ID NO: 582. In some embodiments, an isolated FN3 domain that binds CD71 comprises the amino acid sequence of SEQ ID NO: 583. In some embodiments, an isolated FN3 domain that binds CD71 comprises the amino acid sequence of SEQ ID NO: 584. In some embodiments, an isolated FN3 domain that binds CD71 comprises the amino acid sequence of SEQ ID NO: 585. In some embodiments, an isolated FN3 domain that binds CD71 comprises the amino acid sequence of SEQ ID NO: 586. In some embodiments, an isolated FN3 domain that binds CD71 comprises the amino acid sequence of SEQ ID NO: 587. In some embodiments, an isolated FN3 domain that binds CD71 comprises the amino acid sequence of SEQ ID NO: 588. In some embodiments, an isolated FN3 domain that binds CD71 comprises the amino acid sequence of SEQ ID NO: 589. In some embodiments, an isolated FN3 domain that binds CD71 comprises the amino acid sequence of SEQ ID NO: 590. In some embodiments, an isolated FN3 domain that binds CD71 comprises the amino acid sequence of SEQ ID NO: 591. In some embodiments, an isolated FN3 domain that binds CD71 comprises the amino acid sequence of SEQ ID NO: 592. In some embodiments, an isolated FN3 domain that binds CD71 comprises the amino acid sequence of SEQ ID NO: 593. In some embodiments, an isolated FN3 domain that binds CD71 comprises the amino acid sequence of SEQ ID NO: 594. In some embodiments, an isolated FN3 domain that binds CD71 comprises the amino acid sequence of SEQ ID NO: 595. In some embodiments, an isolated FN3 domain that binds CD71 comprises the amino acid sequence of SEQ ID NO: 596. In some embodiments, an isolated FN3 domain that binds CD71 comprises the amino acid sequence of SEQ ID NO: 597. In some embodiments, an isolated FN3 domain that binds CD71 comprises the amino acid sequence of SEQ ID NO: 598. In some embodiments, an isolated FN3 domain that binds CD71 comprises the amino acid sequence of SEQ ID NO: 599. In some embodiments, an isolated FN3 domain that binds CD71 comprises the amino acid sequence of SEQ ID NO: 600. In some embodiments, an isolated FN3 domain that binds CD71 comprises the amino acid sequence of SEQ ID NO: 601. In some embodiments, an isolated FN3 domain that binds CD71 comprises the amino acid sequence of SEQ ID NO: 602. In some embodiments, an isolated FN3 domain that binds CD71 comprises the amino acid sequence of SEQ ID NO: 603. In some embodiments, an isolated FN3 domain that binds CD71 comprises the amino acid sequence of SEQ ID NO: 604. In some embodiments, an isolated FN3 domain that binds CD71 comprises the amino acid sequence of SEQ ID NO: 605. In some embodiments, an isolated FN3 domain that binds CD71 comprises the amino acid sequence of SEQ ID NO: 606. In some embodiments, an isolated FN3 domain that binds CD71 comprises the amino acid sequence of SEQ ID NO: 607. In some embodiments, an isolated FN3 domain that binds CD71 comprises the amino acid sequence of SEQ ID NO: 608. In some embodiments, an isolated FN3 domain that binds CD71 comprises the amino acid sequence of SEQ ID NO: 609. In some embodiments, an isolated FN3 domain that binds CD71 comprises the amino acid sequence of SEQ ID NO: 610. In some embodiments, an isolated FN3 domain that binds CD71 comprises the amino acid sequence of SEQ ID NO: 611. In some embodiments, an isolated FN3 domain that binds CD71 comprises the amino acid sequence of SEQ ID NO: 612. In some embodiments, an isolated FN3 domain that binds CD71 comprises the amino acid sequence of SEQ ID NO: 613. In some embodiments, an isolated FN3 domain that binds CD71 comprises the amino acid sequence of SEQ ID NO: 614. In some embodiments, an isolated FN3 domain that binds CD71 comprises the amino acid sequence of SEQ ID NO: 615. In some embodiments, an isolated FN3 domain that binds CD71 comprises the amino acid sequence of SEQ ID NO: 616. In some embodiments, an isolated FN3 domain that binds CD71 comprises the amino acid sequence of SEQ ID NO: 617. In some embodiments, an isolated FN3 domain that binds CD71 comprises the amino acid sequence of SEQ ID NO: 618. In some embodiments, an isolated FN3 domain that binds CD71 comprises the amino acid sequence of SEQ ID NO: 619. In some embodiments, an isolated FN3 domain that binds CD71 comprises the amino acid sequence of SEQ ID NO: 620. In some embodiments, an isolated FN3 domain that binds CD71 comprises the amino acid sequence of SEQ ID NO: 621. In some embodiments, an isolated FN3 domain that binds CD71 comprises the amino acid sequence of SEQ ID NO: 622. In some embodiments, an isolated FN3 domain that binds CD71 comprises the amino acid sequence of SEQ ID NO: 623. In some embodiments, an isolated FN3 domain that binds CD71 comprises the amino acid sequence of SEQ ID NO: 624. In some embodiments, an isolated FN3 domain that binds CD71 comprises the amino acid sequence of SEQ ID NO: 625. In some embodiments, an isolated FN3 domain that binds CD71 comprises the amino acid sequence of SEQ ID NO: 626. In some embodiments, an isolated FN3 domain that binds CD71 comprises the amino acid sequence of SEQ ID NO: 627. In some embodiments, an isolated FN3 domain that binds CD71 comprises the amino acid sequence of SEQ ID NO: 628. In some embodiments, an isolated FN3 domain that binds CD71 comprises the amino acid sequence of SEQ ID NO: 629. In some embodiments, an isolated FN3 domain that binds CD71 comprises the amino acid sequence of SEQ ID NO: 630. In some embodiments, an isolated FN3 domain that binds CD71 comprises the amino acid sequence of SEQ ID NO: 631. In some embodiments, an isolated FN3 domain that binds CD71 comprises the amino acid sequence of SEQ ID NO: 632. In some embodiments, an isolated FN3 domain that binds CD71 comprises the amino acid sequence of SEQ ID NO: 633. In some embodiments, an isolated FN3 domain that binds CD71 comprises the amino acid sequence of SEQ ID NO: 634. In some embodiments, an isolated FN3 domain that binds CD71 comprises the amino acid sequence of SEQ ID NO: 635. In some embodiments, an isolated FN3 domain that binds CD71 comprises the amino acid sequence of SEQ ID NO: 636. In some embodiments, an isolated FN3 domain that binds CD71 comprises the amino acid sequence of SEQ ID NO: 637. In some embodiments, an isolated FN3 domain that binds CD71 comprises the amino acid sequence of SEQ ID NO: 638. In some embodiments, an isolated FN3 domain that binds CD71 comprises the amino acid sequence of SEQ ID NO: 639. In some embodiments, an isolated FN3 domain that binds CD71 comprises the amino acid sequence of SEQ ID NO: 640. In some embodiments, an isolated FN3 domain that binds CD71 comprises the amino acid sequence of SEQ ID NO: 641. In some embodiments, an isolated FN3 domain that binds CD71 comprises the amino acid sequence of SEQ ID NO: 642. In some embodiments, an isolated FN3 domain that binds CD71 comprises the amino acid sequence of SEQ ID NO: 643. In some embodiments, an isolated FN3 domain that binds CD71 comprises the amino acid sequence of SEQ ID NO: 644. In some embodiments, an isolated FN3 domain that binds CD71 comprises the amino acid sequence of SEQ ID NO: 663. In some embodiments, an isolated FN3 domain that binds CD71 comprises the amino acid sequence of SEQ ID NO: 664. In some embodiments, an isolated FN3 domain that binds CD71 comprises the amino acid sequence of SEQ ID NO: 665. In some embodiments, an isolated FN3 domain that binds CD71 comprises the amino acid sequence of SEQ ID NO: 666. In some embodiments, an isolated FN3 domain that binds CD71 comprises the amino acid sequence of SEQ ID NO: 667. In some embodiments, an isolated FN3 domain that binds CD71 comprises the amino acid sequence of SEQ ID NO: 668. In some embodiments, an isolated FN3 domain that binds CD71 comprises the amino acid sequence of SEQ ID NO: 669. In some embodiments, an isolated FN3 domain that binds CD71 comprises the amino acid sequence of SEQ ID NO: 670. In some embodiments, an isolated FN3 domain that binds CD71 comprises the amino acid sequence of SEQ ID NO: 671. In some embodiments, an isolated FN3 domain that binds CD71 comprises the amino acid sequence of SEQ ID NO: 672. In some embodiments, an isolated FN3 domain that binds CD71 comprises the amino acid sequence of SEQ ID NO: 1395. In some embodiments, an isolated FN3 domain that binds CD71 comprises the amino acid sequence of SEQ ID NO: 1396. In some embodiments, an isolated FN3 domain that binds CD71 comprises the amino acid sequence of SEQ ID NO: 1397. In some embodiments, an isolated FN3 domain that binds CD71 comprises the amino acid sequence of SEQ ID NO: 1398. In some embodiments, an isolated FN3 domain that binds CD71 comprises the amino acid sequence of SEQ ID NO: 1399. In some embodiments, an isolated FN3 domain that binds CD71 comprises the amino acid sequence of SEQ ID NO: 1400. In some embodiments, an isolated FN3 domain that binds CD71 comprises the amino acid sequence of SEQ ID NO: 1401. In some embodiments, an isolated FN3 domain that binds CD71 comprises the amino acid sequence of SEQ ID NO: 1402. In some embodiments, an isolated FN3 domain that binds CD71 comprises the amino acid sequence of SEQ ID NO: 1403. In some embodiments, an isolated FN3 domain that binds CD71 comprises the amino acid sequence of SEQ ID NO: 1404. In some embodiments, an isolated FN3 domain that binds CD71 comprises the amino acid sequence of SEQ ID NO: 1405. In some embodiments, an isolated FN3 domain that binds CD71 comprises the amino acid sequence of SEQ ID NO: 1406. In some embodiments, an isolated FN3 domain that binds CD71 comprises the amino acid sequence of SEQ ID NO: 1407. In some embodiments, an isolated FN3 domain that binds CD71 comprises the amino acid sequence of SEQ ID NO: 1408. In some embodiments, an isolated FN3 domain that binds CD71 comprises the amino acid sequence of SEQ ID NO: 1409. In some embodiments, an isolated FN3 domain that binds CD71 comprises the amino acid sequence of SEQ ID NO: 1410. In some embodiments, an isolated FN3 domain that binds CD71 comprises the amino acid sequence of SEQ ID NO: 1411. In some embodiments, an isolated FN3 domain that binds CD71 comprises the amino acid sequence of SEQ ID NO: 1412. In some embodiments, an isolated FN3 domain that binds CD71 comprises the amino acid sequence of SEQ ID NO: 1413. In some embodiments, an isolated FN3 domain that binds CD71 comprises the amino acid sequence of SEQ ID NO: 1414. In some embodiments, an isolated FN3 domain that binds CD71 comprises the amino acid sequence of SEQ ID NO: 1415. In some embodiments, an isolated FN3 domain that binds CD71 comprises the amino acid sequence of SEQ ID NO: 1416. In some embodiments, an isolated FN3 domain that binds CD71 comprises the amino acid sequence of SEQ ID NO: 1417. In some embodiments, an isolated FN3 domain that binds CD71 comprises the amino acid sequence of SEQ ID NO: 1418. In some embodiments, an isolated FN3 domain that binds CD71 comprises the amino acid sequence of SEQ ID NO: 1419. In some embodiments, an isolated FN3 domain that binds CD71 comprises the amino acid sequence of SEQ ID NO: 1420. In some embodiments, an isolated FN3 domain that binds CD71 comprises the amino acid sequence of SEQ ID NO: 1421. In some embodiments, an isolated FN3 domain that binds CD71 comprises the amino acid sequence of SEQ ID NO: 1422. In some embodiments, an isolated FN3 domain that binds CD71 comprises the amino acid sequence of SEQ ID NO: 1423. In some embodiments, an isolated FN3 domain that binds CD71 comprises the amino acid sequence of SEQ ID NO: 1424. In some embodiments, an isolated FN3 domain that binds CD71 comprises the amino acid sequence of SEQ ID NO: 1425. In some embodiments, an isolated FN3 domain that binds CD71 comprises the amino acid sequence of SEQ ID NO: 1426. In some embodiments, an isolated FN3 domain that binds CD71 comprises the amino acid sequence of SEQ ID NO: 1427. In some embodiments, an isolated FN3 domain that binds CD71 comprises the amino acid sequence of SEQ ID NO: 1428. In some embodiments, an isolated FN3 domain that binds CD71 comprises the amino acid sequence of SEQ ID NO: 1429. In some embodiments, an isolated FN3 domain that binds CD71 comprises the amino acid sequence of SEQ ID NO: 1430. In some embodiments, an isolated FN3 domain that binds CD71 comprises the amino acid sequence of SEQ ID NO: 1431. In some embodiments, an isolated FN3 domain that binds CD71 comprises the amino acid sequence of SEQ ID NO: 1432. In some embodiments, an isolated FN3 domain that binds CD71 comprises the amino acid sequence of SEQ ID NO: 1433. In some embodiments, an isolated FN3 domain that binds CD71 comprises the amino acid sequence of SEQ ID NO: 1434. In some embodiments, an isolated FN3 domain that binds CD71 comprises the amino acid sequence of SEQ ID NO: 1435. In some embodiments, an isolated FN3 domain that binds CD71 comprises the amino acid sequence of SEQ ID NO: 1436. In some embodiments, an isolated FN3 domain that binds CD71 comprises the amino acid sequence of SEQ ID NO: 1437. In some embodiments, an isolated FN3 domain that binds CD71 comprises the amino acid sequence of SEQ ID NO: 1438. In some embodiments, an isolated FN3 domain that binds CD71 comprises the amino acid sequence of SEQ ID NO: 1439. In some embodiments, an isolated FN3 domain that binds CD71 comprises the amino acid sequence of SEQ ID NO: 1440. In some embodiments, an isolated FN3 domain that binds CD71 comprises the amino acid sequence of SEQ ID NO: 1441. In some embodiments, an isolated FN3 domain that binds CD71 comprises the amino acid sequence of SEQ ID NO: 1442. In some embodiments, an isolated FN3 domain that binds CD71 comprises the amino acid sequence of SEQ ID NO: 1443. In some embodiments, an isolated FN3 domain that binds CD71 comprises the amino acid sequence of SEQ ID NO: 1444. In some embodiments, an isolated FN3 domain that binds CD71 comprises the amino acid sequence of SEQ ID NO: 1445. In some embodiments, an isolated FN3 domain that binds CD71 comprises the amino acid sequence of SEQ ID NO: 1446. In some embodiments, an isolated FN3 domain that binds CD71 comprises the amino acid sequence of SEQ ID NO: 1447. In some embodiments, an isolated FN3 domain that binds CD71 comprises the amino acid sequence of SEQ ID NO: 1448. In some embodiments, an isolated FN3 domain that binds CD71 comprises the amino acid sequence of SEQ ID NO: 1449. In some embodiments, an isolated FN3 domain that binds CD71 comprises the amino acid sequence of SEQ ID NO: 1450. In some embodiments, an isolated FN3 domain that binds CD71 comprises the amino acid sequence of SEQ ID NO: 1451. In some embodiments, an isolated FN3 domain that binds CD71 comprises the amino acid sequence of SEQ ID NO: 1452. In some embodiments, an isolated FN3 domain that binds CD71 comprises the amino acid sequence of SEQ ID NO: 1453. In some embodiments, an isolated FN3 domain that binds CD71 comprises the amino acid sequence of SEQ ID NO: 1454. In some embodiments, an isolated FN3 domain that binds CD71 comprises the amino acid sequence of SEQ ID NO: 1455. In some embodiments, an isolated FN3 domain that binds CD71 comprises the amino acid sequence of SEQ ID NO: 1456. In some embodiments, an isolated FN3 domain that binds CD71 comprises the amino acid sequence of SEQ ID NO: 1457. In some embodiments, an isolated FN3 domain that binds CD71 comprises the amino acid sequence of SEQ ID NO: 1458. In some embodiments, an isolated FN3 domain that binds CD71 comprises the amino acid sequence of SEQ ID NO: 1459. In some embodiments, an isolated FN3 domain that binds CD71 comprises the amino acid sequence of SEQ ID NO: 1460. In some embodiments, an isolated FN3 domain that binds CD71 comprises the amino acid sequence of SEQ ID NO: 1461. In some embodiments, an isolated FN3 domain that binds CD71 comprises the amino acid sequence of SEQ ID NO: 1462. In some embodiments, an isolated FN3 domain that binds CD71 comprises the amino acid sequence of SEQ ID NO: 1463. In some embodiments, an isolated FN3 domain that binds CD71 comprises the amino acid sequence of SEQ ID NO: 1464. In some embodiments, an isolated FN3 domain that binds CD71 comprises the amino acid sequence of SEQ ID NO: 1465. In some embodiments, an isolated FN3 domain that binds CD71 comprises the amino acid sequence of SEQ ID NO: 1466. In some embodiments, an isolated FN3 domain that binds CD71 comprises the amino acid sequence of SEQ ID NO: 1467. In some embodiments, an isolated FN3 domain that binds CD71 comprises the amino acid sequence of SEQ ID NO: 1468. In some embodiments, an isolated FN3 domain that binds CD71 comprises the amino acid sequence of SEQ ID NO: 1469. In some embodiments, an isolated FN3 domain that binds CD71 comprises the amino acid sequence of SEQ ID NO: 1470. In some embodiments, an isolated FN3 domain that binds CD71 comprises the amino acid sequence of SEQ ID NO: 1471. In some embodiments, an isolated FN3 domain that binds CD71 comprises the amino acid sequence of SEQ ID NO: 1472. In some embodiments, an isolated FN3 domain that binds CD71 comprises the amino acid sequence of SEQ ID NO: 1473. In some embodiments, an isolated FN3 domain that binds CD71 comprises the amino acid sequence of SEQ ID NO: 1474. In some embodiments, an isolated FN3 domain that binds CD71 comprises the amino acid sequence of SEQ ID NO: 1475. In some embodiments, an isolated FN3 domain that binds CD71 comprises the amino acid sequence of SEQ ID NO: 1476. In some embodiments, an isolated FN3 domain that binds CD71 comprises the amino acid sequence of SEQ ID NO: 1477. In some embodiments, an isolated FN3 domain that binds CD71 comprises the amino acid sequence of SEQ ID NO: 1478. In some embodiments, an isolated FN3 domain that binds CD71 comprises the amino acid sequence of SEQ ID NO: 1479. In some embodiments, an isolated FN3 domain that binds CD71 comprises the amino acid sequence of SEQ ID NO: 1480. In some embodiments, an isolated FN3 domain that binds CD71 comprises the amino acid sequence of SEQ ID NO: 1481. In some embodiments, an isolated FN3 domain that binds CD71 comprises the amino acid sequence of SEQ ID NO: 1482. In some embodiments, an isolated FN3 domain that binds CD71 comprises the amino acid sequence of SEQ ID NO: 1483. In some embodiments, an isolated FN3 domain that binds CD71 comprises the amino acid sequence of SEQ ID NO: 1484. In some embodiments, an isolated FN3 domain that binds CD71 comprises the amino acid sequence of SEQ ID NO: 1485. In some embodiments, an isolated FN3 domain that binds CD71 comprises the amino acid sequence of SEQ ID NO: 1486. In some embodiments, an isolated FN3 domain that binds CD71 comprises the amino acid sequence of SEQ ID NO: 1487. In some embodiments, an isolated FN3 domain that binds CD71 comprises the amino acid sequence of SEQ ID NO: 1488. In some embodiments, an isolated FN3 domain that binds CD71 comprises the amino acid sequence of SEQ ID NO: 1489. In some embodiments, an isolated FN3 domain that binds CD71 comprises the amino acid sequence of SEQ ID NO: 1490. In some embodiments, an isolated FN3 domain that binds CD71 comprises the amino acid sequence of SEQ ID NO: 1491. In some embodiments, an isolated FN3 domain that binds CD71 comprises the amino acid sequence of SEQ ID NO: 1492. In some embodiments, an isolated FN3 domain that binds CD71 comprises the amino acid sequence of SEQ ID NO: 1493. In some embodiments, an isolated FN3 domain that binds CD71 comprises the amino acid sequence of SEQ ID NO: 1494. In some embodiments, an isolated FN3 domain that binds CD71 comprises the amino acid sequence of SEQ ID NO: 1495. In some embodiments, an isolated FN3 domain that binds CD71 comprises the amino acid sequence of SEQ ID NO: 1496. In some embodiments, an isolated FN3 domain that binds CD71 comprises the amino acid sequence of SEQ ID NO: 1497. In some embodiments, an isolated FN3 domain that binds CD71 comprises the amino acid sequence of SEQ ID NO: 1498. In some embodiments, an isolated FN3 domain that binds CD71 comprises the amino acid sequence of SEQ ID NO: 1499. In some embodiments, an isolated FN3 domain that binds CD71 comprises the amino acid sequence of SEQ ID NO: 1500. In some embodiments, an isolated FN3 domain that binds CD71 comprises the amino acid sequence of SEQ ID NO: 1501. In some embodiments, an isolated FN3 domain that binds CD71 comprises the amino acid sequence of SEQ ID NO: 1502. In some embodiments, an isolated FN3 domain that binds CD71 comprises the amino acid sequence of SEQ ID NO: 1503. In some embodiments, an isolated FN3 domain that binds CD71 comprises the amino acid sequence of SEQ ID NO: 1504. In some embodiments, an isolated FN3 domain that binds CD71 comprises the amino acid sequence of SEQ ID NO: 1505. In some embodiments, an isolated FN3 domain that binds CD71 comprises the amino acid sequence of SEQ ID NO: 1506. In some embodiments, an isolated FN3 domain that binds CD71 comprises the amino acid sequence of SEQ ID NO: 1507. In some embodiments, an isolated FN3 domain that binds CD71 comprises the amino acid sequence of SEQ ID NO: 1508. In some embodiments, an isolated FN3 domain that binds CD71 comprises the amino acid sequence of SEQ ID NO: 1509. In some embodiments, an isolated FN3 domain that binds CD71 comprises the amino acid sequence of SEQ ID NO: 1510. In some embodiments, an isolated FN3 domain that binds CD71 comprises the amino acid sequence of SEQ ID NO: 1511. In some embodiments, an isolated FN3 domain that binds CD71 comprises the amino acid sequence of SEQ ID NO: 1512. In some embodiments, an isolated FN3 domain that binds CD71 comprises the amino acid sequence of SEQ ID NO: 1513. In some embodiments, an isolated FN3 domain that binds CD71 comprises the amino acid sequence of SEQ ID NO: 1514. In some embodiments, an isolated FN3 domain that binds CD71 comprises the amino acid sequence of SEQ ID NO: 1515. In some embodiments, an isolated FN3 domain that binds CD71 comprises the amino acid sequence of SEQ ID NO: 1516. In some embodiments, an isolated FN3 domain that binds CD71 comprises the amino acid sequence of SEQ ID NO: 1517. In some embodiments, an isolated FN3 domain that binds CD71 comprises the amino acid sequence of SEQ ID NO: 1518. In some embodiments, an isolated FN3 domain that binds CD71 comprises the amino acid sequence of SEQ ID NO: 1519. In some embodiments, an isolated FN3 domain that binds CD71 comprises the amino acid sequence of SEQ ID NO: 1520. In some embodiments, an isolated FN3 domain that binds CD71 comprises the amino acid sequence of SEQ ID NO: 1521. In some embodiments, an isolated FN3 domain that binds CD71 comprises the amino acid sequence of SEQ ID NO: 1522. In some embodiments, an isolated FN3 domain that binds CD71 comprises the amino acid sequence of SEQ ID NO: 1523. In some embodiments, an isolated FN3 domain that binds CD71 comprises the amino acid sequence of SEQ ID NO: 1524. In some embodiments, an isolated FN3 domain that binds CD71 comprises the amino acid sequence of SEQ ID NO: 1525. In some embodiments, an isolated FN3 domain that binds CD71 comprises the amino acid sequence of SEQ ID NO: 1526. In some embodiments, an isolated FN3 domain that binds CD71 comprises the amino acid sequence of SEQ ID NO: 1527. In some embodiments, an isolated FN3 domain that binds CD71 comprises the amino acid sequence of SEQ ID NO: 1528. In some embodiments, an isolated FN3 domain that binds CD71 comprises the amino acid sequence of SEQ ID NO: 1529. In some embodiments, an isolated FN3 domain that binds CD71 comprises the amino acid sequence of SEQ ID NO: 1530. In some embodiments, an isolated FN3 domain that binds CD71 comprises the amino acid sequence of SEQ ID NO: 1531. In some embodiments, an isolated FN3 domain that binds CD71 comprises the amino acid sequence of SEQ ID NO: 1532. In some embodiments, an isolated FN3 domain that binds CD71 comprises the amino acid sequence of SEQ ID NO: 1533. In some embodiments, an isolated FN3 domain that binds CD71 comprises the amino acid sequence of SEQ ID NO: 1534. In some embodiments, an isolated FN3 domain that binds CD71 comprises the amino acid sequence of SEQ ID NO: 1535. In some embodiments, an isolated FN3 domain that binds CD71 comprises the amino acid sequence of SEQ ID NO: 1536. In some embodiments, an isolated FN3 domain that binds CD71 comprises the amino acid sequence of SEQ ID NO: 1537. In some embodiments, an isolated FN3 domain that binds CD71 comprises the amino acid sequence of SEQ ID NO: 1538. In some embodiments, an isolated FN3 domain that binds CD71 comprises the amino acid sequence of SEQ ID NO: 1539. In some embodiments, an isolated FN3 domain that binds CD71 comprises the amino acid sequence of SEQ ID NO: 1540. In some embodiments, an isolated FN3 domain that binds CD71 comprises the amino acid sequence of SEQ ID NO: 1541. In some embodiments, an isolated FN3 domain that binds CD71 comprises the amino acid sequence of SEQ ID NO: 1542. In some embodiments, an isolated FN3 domain that binds CD71 comprises the amino acid sequence of SEQ ID NO: 1543. In some embodiments, an isolated FN3 domain that binds CD71 comprises the amino acid sequence of SEQ ID NO: 1544. In some embodiments, an isolated FN3 domain that binds CD71 comprises the amino acid sequence of SEQ ID NO: 1545. In some embodiments, an isolated FN3 domain that binds CD71 comprises the amino acid sequence of SEQ ID NO: 1546. In some embodiments, an isolated FN3 domain that binds CD71 comprises the amino acid sequence of SEQ ID NO: 1547. In some embodiments, an isolated FN3 domain that binds CD71 comprises the amino acid sequence of SEQ ID NO: 1548. In some embodiments, an isolated FN3 domain that binds CD71 comprises the amino acid sequence of SEQ ID NO: 1549. In some embodiments, an isolated FN3 domain that binds CD71 comprises the amino acid sequence of SEQ ID NO: 1550. In some embodiments, an isolated FN3 domain that binds CD71 comprises the amino acid sequence of SEQ ID NO: 1551. In some embodiments, an isolated FN3 domain that binds CD71 comprises the amino acid sequence of SEQ ID NO: 1552. In some embodiments, an isolated FN3 domain that binds CD71 comprises the amino acid sequence of SEQ ID NO: 1553. In some embodiments, an isolated FN3 domain that binds CD71 comprises the amino acid sequence of SEQ ID NO: 1554. In some embodiments, an isolated FN3 domain that binds CD71 comprises the amino acid sequence of SEQ ID NO: 1555. In some embodiments, an isolated FN3 domain that binds CD71 comprises the amino acid sequence of SEQ ID NO: 1556. In some embodiments, an isolated FN3 domain that binds CD71 comprises the amino acid sequence of SEQ ID NO: 1557. In some embodiments, an isolated FN3 domain that binds CD71 comprises the amino acid sequence of SEQ ID NO: 1558. In some embodiments, an isolated FN3 domain that binds CD71 comprises the amino acid sequence of SEQ ID NO: 1559. In some embodiments, an isolated FN3 domain that binds CD71 comprises the amino acid sequence of SEQ ID NO: 1560. In some embodiments, an isolated FN3 domain that binds CD71 comprises the amino acid sequence of SEQ ID NO: 1561. In some embodiments, an isolated FN3 domain that binds CD71 comprises the amino acid sequence of SEQ ID NO: 1562. In some embodiments, an isolated FN3 domain that binds CD71 comprises the amino acid sequence of SEQ ID NO: 1563. In some embodiments, an isolated FN3 domain that binds CD71 comprises the amino acid sequence of SEQ ID NO: 1564. In some embodiments, an isolated FN3 domain that binds CD71 comprises the amino acid sequence of SEQ ID NO: 1565. In some embodiments, an isolated FN3 domain that binds CD71 comprises the amino acid sequence of SEQ ID NO: 1566. In some embodiments, an isolated FN3 domain that binds CD71 comprises the amino acid sequence of SEQ ID NO: 1567. In some embodiments, an isolated FN3 domain that binds CD71 comprises the amino acid sequence of SEQ ID NO: 1568. In some embodiments, an isolated FN3 domain that binds CD71 comprises the amino acid sequence of SEQ ID NO: 1569. In some embodiments, an isolated FN3 domain that binds CD71 comprises the amino acid sequence of SEQ ID NO: 1570. In some embodiments, an isolated FN3 domain that binds CD71 comprises the amino acid sequence of SEQ ID NO: 1571. In some embodiments, an isolated FN3 domain that binds CD71 comprises the amino acid sequence of SEQ ID NO: 1572. In some embodiments, an isolated FN3 domain that binds CD71 comprises the amino acid sequence of SEQ ID NO: 1573. In some embodiments, an isolated FN3 domain that binds CD71 comprises the amino acid sequence of SEQ ID NO: 1574. In some embodiments, an isolated FN3 domain that binds CD71 comprises the amino acid sequence of SEQ ID NO: 1575. In some embodiments, an isolated FN3 domain that binds CD71 comprises the amino acid sequence of SEQ ID NO: 1576. In some embodiments, an isolated FN3 domain that binds CD71 comprises the amino acid sequence of SEQ ID NO: 1577. In some embodiments, an isolated FN3 domain that binds CD71 comprises the amino acid sequence of SEQ ID NO: 1578. In some embodiments, an isolated FN3 domain that binds CD71 comprises the amino acid sequence of SEQ ID NO: 1579. In some embodiments, an isolated FN3 domain that binds CD71 comprises the amino acid sequence of SEQ ID NO: 1580. In some embodiments, an isolated FN3 domain that binds CD71 comprises the amino acid sequence of SEQ ID NO: 1581. In some embodiments, an isolated FN3 domain that binds CD71 comprises the amino acid sequence of SEQ ID NO: 1582. In some embodiments, an isolated FN3 domain that binds CD71 comprises the amino acid sequence of SEQ ID NO: 1583. In some embodiments, an isolated FN3 domain that binds CD71 comprises the amino acid sequence of SEQ ID NO: 1584. In some embodiments, an isolated FN3 domain that binds CD71 comprises the amino acid sequence of SEQ ID NO: 1585. In some embodiments, an isolated FN3 domain that binds CD71 comprises the amino acid sequence of SEQ ID NO: 1586. In some embodiments, an isolated FN3 domain that binds CD71 comprises the amino acid sequence of SEQ ID NO: 1587. In some embodiments, an isolated FN3 domain that binds CD71 comprises the amino acid sequence of SEQ ID NO: 1588. In some embodiments, an isolated FN3 domain that binds CD71 comprises the amino acid sequence of SEQ ID NO: 1589. In some embodiments, an isolated FN3 domain that binds CD71 comprises the amino acid sequence of SEQ ID NO: 1590. In some embodiments, an isolated FN3 domain that binds CD71 comprises the amino acid sequence of SEQ ID NO: 1591. In some embodiments, an isolated FN3 domain that binds CD71 comprises the amino acid sequence of SEQ ID NO: 1592. In some embodiments, an isolated FN3 domain that binds CD71 comprises the amino acid sequence of SEQ ID NO: 1593. In some embodiments, an isolated FN3 domain that binds CD71 comprises the amino acid sequence of SEQ ID NO: 1594. In some embodiments, an isolated FN3 domain that binds CD71 comprises the amino acid sequence of SEQ ID NO: 1595. In some embodiments, an isolated FN3 domain that binds CD71 comprises the amino acid sequence of SEQ ID NO: 1596. In some embodiments, an isolated FN3 domain that binds CD71 comprises the amino acid sequence of SEQ ID NO: 1597. In some embodiments, an isolated FN3 domain that binds CD71 comprises the amino acid sequence of SEQ ID NO: 1598. In some embodiments, an isolated FN3 domain that binds CD71 comprises the amino acid sequence of SEQ ID NO: 1599. In some embodiments, an isolated FN3 domain that binds CD71 comprises the amino acid sequence of SEQ ID NO: 1600. In some embodiments, an isolated FN3 domain that binds CD71 comprises the amino acid sequence of SEQ ID NO: 1601. In some embodiments, an isolated FN3 domain that binds CD71 comprises the amino acid sequence of SEQ ID NO: 1602. In some embodiments, an isolated FN3 domain that binds CD71 comprises the amino acid sequence of SEQ ID NO: 1603. In some embodiments, an isolated FN3 domain that binds CD71 comprises the amino acid sequence of SEQ ID NO: 1604. In some embodiments, an isolated FN3 domain that binds CD71 comprises the amino acid sequence of SEQ ID NO: 1605. In some embodiments, an isolated FN3 domain that binds CD71 comprises the amino acid sequence of SEQ ID NO: 1606. In some embodiments, an isolated FN3 domain that binds CD71 comprises the amino acid sequence of SEQ ID NO: 1607. In some embodiments, an isolated FN3 domain that binds CD71 comprises the amino acid sequence of SEQ ID NO: 1608. In some embodiments, an isolated FN3 domain that binds CD71 comprises the amino acid sequence of SEQ ID NO: 1609. In some embodiments, an isolated FN3 domain that binds CD71 comprises the amino acid sequence of SEQ ID NO: 1610. In some embodiments, an isolated FN3 domain that binds CD71 comprises the amino acid sequence of SEQ ID NO: 1611. In some embodiments, an isolated FN3 domain that binds CD71 comprises the amino acid sequence of SEQ ID NO: 1612. In some embodiments, an isolated FN3 domain that binds CD71 comprises the amino acid sequence of SEQ ID NO: 1613. In some embodiments, an isolated FN3 domain that binds CD71 comprises the amino acid sequence of SEQ ID NO: 1614. In some embodiments, an isolated FN3 domain that binds CD71 comprises the amino acid sequence of SEQ ID NO: 1615. In some embodiments, an isolated FN3 domain that binds CD71 comprises the amino acid sequence of SEQ ID NO: 1616. In some embodiments, an isolated FN3 domain that binds CD71 comprises the amino acid sequence of SEQ ID NO: 1617. In some embodiments, an isolated FN3 domain that binds CD71 comprises the amino acid sequence of SEQ ID NO: 1618. In some embodiments, an isolated FN3 domain that binds CD71 comprises the amino acid sequence of SEQ ID NO: 1619. In some embodiments, an isolated FN3 domain that binds CD71 comprises the amino acid sequence of SEQ ID NO: 1620. In some embodiments, an isolated FN3 domain that binds CD71 comprises the amino acid sequence of SEQ ID NO: 1621. In some embodiments, an isolated FN3 domain that binds CD71 comprises the amino acid sequence of SEQ ID NO: 1622. In some embodiments, an isolated FN3 domain that binds CD71 comprises the amino acid sequence of SEQ ID NO: 1623. In some embodiments, an isolated FN3 domain that binds CD71 comprises the amino acid sequence of SEQ ID NO: 1624. In some embodiments, an isolated FN3 domain that binds CD71 comprises the amino acid sequence of SEQ ID NO: 1625. In some embodiments, an isolated FN3 domain that binds CD71 comprises the amino acid sequence of SEQ ID NO: 1626. In some embodiments, an isolated FN3 domain that binds CD71 comprises the amino acid sequence of SEQ ID NO: 1627. In some embodiments, an isolated FN3 domain that binds CD71 comprises the amino acid sequence of SEQ ID NO: 1628. In some embodiments, an isolated FN3 domain that binds CD71 comprises the amino acid sequence of SEQ ID NO: 1629. In some embodiments, an isolated FN3 domain that binds CD71 comprises the amino acid sequence of SEQ ID NO: 1630. In some embodiments, an isolated FN3 domain that binds CD71 comprises the amino acid sequence of SEQ ID NO: 1631. In some embodiments, an isolated FN3 domain that binds CD71 comprises the amino acid sequence of SEQ ID NO: 1632. In some embodiments, an isolated FN3 domain that binds CD71 comprises the amino acid sequence of SEQ ID NO: 1633. In some embodiments, an isolated FN3 domain that binds CD71 comprises the amino acid sequence of SEQ ID NO: 1634. In some embodiments, an isolated FN3 domain that binds CD71 comprises the amino acid sequence of SEQ ID NO: 1635. In some embodiments, an isolated FN3 domain that binds CD71 comprises the amino acid sequence of SEQ ID NO: 1636. In some embodiments, an isolated FN3 domain that binds CD71 comprises the amino acid sequence of SEQ ID NO: 1637. In some embodiments, an isolated FN3 domain that binds CD71 comprises the amino acid sequence of SEQ ID NO: 1638. In some embodiments, an isolated FN3 domain that binds CD71 comprises the amino acid sequence of SEQ ID NO: 1639. In some embodiments, an isolated FN3 domain that binds CD71 comprises the amino acid sequence of SEQ ID NO: 1640. In some embodiments, an isolated FN3 domain that binds CD71 comprises the amino acid sequence of SEQ ID NO: 1641. In some embodiments, an isolated FN3 domain that binds CD71 comprises the amino acid sequence of SEQ ID NO: 1642. In some embodiments, an isolated FN3 domain that binds CD71 comprises the amino acid sequence of SEQ ID NO: 1643. In some embodiments, an isolated FN3 domain that binds CD71 comprises the amino acid sequence of SEQ ID NO: 1644. In some embodiments, an isolated FN3 domain that binds CD71 comprises the amino acid sequence of SEQ ID NO: 1645. In some embodiments, an isolated FN3 domain that binds CD71 comprises the amino acid sequence of SEQ ID NO: 1646. In some embodiments, an isolated FN3 domain that binds CD71 comprises the amino acid sequence of SEQ ID NO: 1647. In some embodiments, an isolated FN3 domain that binds CD71 comprises the amino acid sequence of SEQ ID NO: 1648. In some embodiments, an isolated FN3 domain that binds CD71 comprises the amino acid sequence of SEQ ID NO: 1649. In some embodiments, an isolated FN3 domain that binds CD71 comprises the amino acid sequence of SEQ ID NO: 1650. In some embodiments, an isolated FN3 domain that binds CD71 comprises the amino acid sequence of SEQ ID NO: 1651. In some embodiments, an isolated FN3 domain that binds CD71 comprises the amino acid sequence of SEQ ID NO: 1652. In some embodiments, an isolated FN3 domain that binds CD71 comprises the amino acid sequence of SEQ ID NO: 1653. In some embodiments, an isolated FN3 domain that binds CD71 comprises the amino acid sequence of SEQ ID NO: 1654. In some embodiments, an isolated FN3 domain that binds CD71 comprises the amino acid sequence of SEQ ID NO: 1655. In some embodiments, an isolated FN3 domain that binds CD71 comprises the amino acid sequence of SEQ ID NO: 1656. In some embodiments, an isolated FN3 domain that binds CD71 comprises the amino acid sequence of SEQ ID NO: 1657. In some embodiments, an isolated FN3 domain that binds CD71 comprises the amino acid sequence of SEQ ID NO: 1658. In some embodiments, an isolated FN3 domain that binds CD71 comprises the amino acid sequence of SEQ ID NO: 1659. In some embodiments, an isolated FN3 domain that binds CD71 comprises the amino acid sequence of SEQ ID NO: 1660. In some embodiments, an isolated FN3 domain that binds CD71 comprises the amino acid sequence of SEQ ID NO: 1661. In some embodiments, an isolated FN3 domain that binds CD71 comprises the amino acid sequence of SEQ ID NO: 1662. In some embodiments, an isolated FN3 domain that binds CD71 comprises the amino acid sequence of SEQ ID NO: 1663. In some embodiments, an isolated FN3 domain that binds CD71 comprises the amino acid sequence of SEQ ID NO: 1664. In some embodiments, an isolated FN3 domain that binds CD71 comprises the amino acid sequence of SEQ ID NO: 1665. In some embodiments, an isolated FN3 domain that binds CD71 comprises the amino acid sequence of SEQ ID NO: 1666. In some embodiments, an isolated FN3 domain that binds CD71 comprises the amino acid sequence of SEQ ID NO: 1667. In some embodiments, an isolated FN3 domain that binds CD71 comprises the amino acid sequence of SEQ ID NO: 1668. In some embodiments, an isolated FN3 domain that binds CD71 comprises the amino acid sequence of SEQ ID NO: 1669. In some embodiments, an isolated FN3 domain that binds CD71 comprises the amino acid sequence of SEQ ID NO: 1670. In some embodiments, an isolated FN3 domain that binds CD71 comprises the amino acid sequence of SEQ ID NO: 1671. In some embodiments, an isolated FN3 domain that binds CD71 comprises the amino acid sequence of SEQ ID NO: 1672. In some embodiments, an isolated FN3 domain that binds CD71 comprises the amino acid sequence of SEQ ID NO: 1673. In some embodiments, an isolated FN3 domain that binds CD71 comprises the amino acid sequence of SEQ ID NO: 1674. In some embodiments, an isolated FN3 domain that binds CD71 comprises the amino acid sequence of SEQ ID NO: 1675. In some embodiments, an isolated FN3 domain that binds CD71 comprises the amino acid sequence of SEQ ID NO: 1676. In some embodiments, an isolated FN3 domain that binds CD71 comprises the amino acid sequence of SEQ ID NO: 1677. In some embodiments, an isolated FN3 domain that binds CD71 comprises the amino acid sequence of SEQ ID NO: 1678. In some embodiments, an isolated FN3 domain that binds CD71 comprises the amino acid sequence of SEQ ID NO: 1679. In some embodiments, an isolated FN3 domain that binds CD71 comprises the amino acid sequence of SEQ ID NO: 1680. In some embodiments, an isolated FN3 domain that binds CD71 comprises the amino acid sequence of SEQ ID NO: 1681. In some embodiments, an isolated FN3 domain that binds CD71 comprises the amino acid sequence of SEQ ID NO: 1682. In some embodiments, an isolated FN3 domain that binds CD71 comprises the amino acid sequence of SEQ ID NO: 1683. In some embodiments, an isolated FN3 domain that binds CD71 comprises the amino acid sequence of SEQ ID NO: 1684. In some embodiments, an isolated FN3 domain that binds CD71 comprises the amino acid sequence of SEQ ID NO: 1685. In some embodiments, an isolated FN3 domain that binds CD71 comprises the amino acid sequence of SEQ ID NO: 1686. In some embodiments, an isolated FN3 domain that binds CD71 comprises the amino acid sequence of SEQ ID NO: 1687. In some embodiments, an isolated FN3 domain that binds CD71 comprises the amino acid sequence of SEQ ID NO: 1688. In some embodiments, an isolated FN3 domain that binds CD71 comprises the amino acid sequence of SEQ ID NO: 1689. In some embodiments, an isolated FN3 domain that binds CD71 comprises the amino acid sequence of SEQ ID NO: 1690. In some embodiments, an isolated FN3 domain that binds CD71 comprises the amino acid sequence of SEQ ID NO: 1691. In some embodiments, an isolated FN3 domain that binds CD71 comprises the amino acid sequence of SEQ ID NO: 1692. In some embodiments, an isolated FN3 domain that binds CD71 comprises the amino acid sequence of SEQ ID NO: 1693. In some embodiments, an isolated FN3 domain that binds CD71 comprises the amino acid sequence of SEQ ID NO: 1694. In some embodiments, an isolated FN3 domain that binds CD71 comprises the amino acid sequence of SEQ ID NO: 1695. In some embodiments, an isolated FN3 domain that binds CD71 comprises the amino acid sequence of SEQ ID NO: 1696. In some embodiments, an isolated FN3 domain that binds CD71 comprises the amino acid sequence of SEQ ID NO: 1697. In some embodiments, an isolated FN3 domain that binds CD71 comprises the amino acid sequence of SEQ ID NO: 1698. In some embodiments, an isolated FN3 domain that binds CD71 comprises the amino acid sequence of SEQ ID NO: 1699. In some embodiments, an isolated FN3 domain that binds CD71 comprises the amino acid sequence of SEQ ID NO: 1700. In some embodiments, an isolated FN3 domain that binds CD71 comprises the amino acid sequence of SEQ ID NO: 1701. In some embodiments, an isolated FN3 domain that binds CD71 comprises the amino acid sequence of SEQ ID NO: 1702. In some embodiments, an isolated FN3 domain that binds CD71 comprises the amino acid sequence of SEQ ID NO: 1703. In some embodiments, an isolated FN3 domain that binds CD71 comprises the amino acid sequence of SEQ ID NO: 1704. In some embodiments, an isolated FN3 domain that binds CD71 comprises the amino acid sequence of SEQ ID NO: 1705. In some embodiments, an isolated FN3 domain that binds CD71 comprises the amino acid sequence of SEQ ID NO: 1706. In some embodiments, an isolated FN3 domain that binds CD71 comprises the amino acid sequence of SEQ ID NO: 1707. In some embodiments, an isolated FN3 domain that binds CD71 comprises the amino acid sequence of SEQ ID NO: 1708. In some embodiments, an isolated FN3 domain that binds CD71 comprises the amino acid sequence of SEQ ID NO: 1709. In some embodiments, an isolated FN3 domain that binds CD71 comprises the amino acid sequence of SEQ ID NO: 1710. In some embodiments, an isolated FN3 domain that binds CD71 comprises the amino acid sequence of SEQ ID NO: 1711. In some embodiments, an isolated FN3 domain that binds CD71 comprises the amino acid sequence of SEQ ID NO: 1712. In some embodiments, an isolated FN3 domain that binds CD71 comprises the amino acid sequence of SEQ ID NO: 1713. In some embodiments, an isolated FN3 domain that binds CD71 comprises the amino acid sequence of SEQ ID NO: 1714. In some embodiments, an isolated FN3 domain that binds CD71 comprises the amino acid sequence of SEQ ID NO: 1715. In some embodiments, an isolated FN3 domain that binds CD71 comprises the amino acid sequence of SEQ ID NO: 1716. In some embodiments, an isolated FN3 domain that binds CD71 comprises the amino acid sequence of SEQ ID NO: 1717. In some embodiments, an isolated FN3 domain that binds CD71 comprises the amino acid sequence of SEQ ID NO: 1718. In some embodiments, an isolated FN3 domain that binds CD71 comprises the amino acid sequence of SEQ ID NO: 1719. In some embodiments, an isolated FN3 domain that binds CD71 comprises the amino acid sequence of SEQ ID NO: 1720. In some embodiments, an isolated FN3 domain that binds CD71 comprises the amino acid sequence of SEQ ID NO: 1721. In some embodiments, an isolated FN3 domain that binds CD71 comprises the amino acid sequence of SEQ ID NO: 1722. In some embodiments, an isolated FN3 domain that binds CD71 comprises the amino acid sequence of SEQ ID NO: 1723. In some embodiments, an isolated FN3 domain that binds CD71 comprises the amino acid sequence of SEQ ID NO: 1724. In some embodiments, an isolated FN3 domain that binds CD71 comprises the amino acid sequence of SEQ ID NO: 1725. In some embodiments, an isolated FN3 domain that binds CD71 comprises the amino acid sequence of SEQ ID NO: 1726. In some embodiments, an isolated FN3 domain that binds CD71 comprises the amino acid sequence of SEQ ID NO: 1727. In some embodiments, an isolated FN3 domain that binds CD71 comprises the amino acid sequence of SEQ ID NO: 1728. In some embodiments, an isolated FN3 domain that binds CD71 comprises the amino acid sequence of SEQ ID NO: 1729. In some embodiments, an isolated FN3 domain that binds CD71 comprises the amino acid sequence of SEQ ID NO: 1730. In some embodiments, an isolated FN3 domain that binds CD71 comprises the amino acid sequence of SEQ ID NO: 1731. In some embodiments, an isolated FN3 domain that binds CD71 comprises the amino acid sequence of SEQ ID NO: 1732. In some embodiments, an isolated FN3 domain that binds CD71 comprises the amino acid sequence of SEQ ID NO: 1733. In some embodiments, an isolated FN3 domain that binds CD71 comprises the amino acid sequence of SEQ ID NO: 1734. In some embodiments, an isolated FN3 domain that binds CD71 comprises the amino acid sequence of SEQ ID NO: 1735. In some embodiments, an isolated FN3 domain that binds CD71 comprises the amino acid sequence of SEQ ID NO: 1736. In some embodiments, an isolated FN3 domain that binds CD71 comprises the amino acid sequence of SEQ ID NO: 1737. In some embodiments, an isolated FN3 domain that binds CD71 comprises the amino acid sequence of SEQ ID NO: 1738. In some embodiments, an isolated FN3 domain that binds CD71 comprises the amino acid sequence of SEQ ID NO: 1739. In some embodiments, an isolated FN3 domain that binds CD71 comprises the amino acid sequence of SEQ ID NO: 1740. In some embodiments, an isolated FN3 domain that binds CD71 comprises the amino acid sequence of SEQ ID NO: 1741. In some embodiments, an isolated FN3 domain that binds CD71 comprises the amino acid sequence of SEQ ID NO: 1742. In some embodiments, an isolated FN3 domain that binds CD71 comprises the amino acid sequence of SEQ ID NO: 1743. In some embodiments, an isolated FN3 domain that binds CD71 comprises the amino acid sequence of SEQ ID NO: 1744. In some embodiments, an isolated FN3 domain that binds CD71 comprises the amino acid sequence of SEQ ID NO: 1745. In some embodiments, an isolated FN3 domain that binds CD71 comprises the amino acid sequence of SEQ ID NO: 1746. In some embodiments, an isolated FN3 domain that binds CD71 comprises the amino acid sequence of SEQ ID NO: 1747. In some embodiments, an isolated FN3 domain that binds CD71 comprises the amino acid sequence of SEQ ID NO: 1748. In some embodiments, an isolated FN3 domain that binds CD71 comprises the amino acid sequence of SEQ ID NO: 1749. In some embodiments, an isolated FN3 domain that binds CD71 comprises the amino acid sequence of SEQ ID NO: 1750. In some embodiments, an isolated FN3 domain that binds CD71 comprises the amino acid sequence of SEQ ID NO: 1751. In some embodiments, an isolated FN3 domain that binds CD71 comprises the amino acid sequence of SEQ ID NO: 1752. In some embodiments, an isolated FN3 domain that binds CD71 comprises the amino acid sequence of SEQ ID NO: 1753. In some embodiments, an isolated FN3 domain that binds CD71 comprises the amino acid sequence of SEQ ID NO: 1754. In some embodiments, an isolated FN3 domain that binds CD71 comprises the amino acid sequence of SEQ ID NO: 1755. In some embodiments, an isolated FN3 domain that binds CD71 comprises the amino acid sequence of SEQ ID NO: 1756. In some embodiments, an isolated FN3 domain that binds CD71 comprises the amino acid sequence of SEQ ID NO: 1757. In some embodiments, an isolated FN3 domain that binds CD71 comprises the amino acid sequence of SEQ ID NO: 1758. In some embodiments, an isolated FN3 domain that binds CD71 comprises the amino acid sequence of SEQ ID NO: 1759. In some embodiments, an isolated FN3 domain that binds CD71 comprises the amino acid sequence of SEQ ID NO: 1760. In some embodiments, an isolated FN3 domain that binds CD71 comprises the amino acid sequence of SEQ ID NO: 1761. In some embodiments, an isolated FN3 domain that binds CD71 comprises the amino acid sequence of SEQ ID NO: 1762. In some embodiments, an isolated FN3 domain that binds CD71 comprises the amino acid sequence of SEQ ID NO: 1763. In some embodiments, an isolated FN3 domain that binds CD71 comprises the amino acid sequence of SEQ ID NO: 1764. In some embodiments, an isolated FN3 domain that binds CD71 comprises the amino acid sequence of SEQ ID NO: 1765. In some embodiments, an isolated FN3 domain that binds CD71 comprises the amino acid sequence of SEQ ID NO: 1766. In some embodiments, an isolated FN3 domain that binds CD71 comprises the amino acid sequence of SEQ ID NO: 1767. In some embodiments, an isolated FN3 domain that binds CD71 comprises the amino acid sequence of SEQ ID NO: 1768. In some embodiments, an isolated FN3 domain that binds CD71 comprises the amino acid sequence of SEQ ID NO: 1769. In some embodiments, an isolated FN3 domain that binds CD71 comprises the amino acid sequence of SEQ ID NO: 1770. In some embodiments, an isolated FN3 domain that binds CD71 comprises the amino acid sequence of SEQ ID NO: 1771. In some embodiments, an isolated FN3 domain that binds CD71 comprises the amino acid sequence of SEQ ID NO: 1772. In some embodiments, an isolated FN3 domain that binds CD71 comprises the amino acid sequence of SEQ ID NO: 1773. In some embodiments, an isolated FN3 domain that binds CD71 comprises the amino acid sequence of SEQ ID NO: 1774. In some embodiments, an isolated FN3 domain that binds CD71 comprises the amino acid sequence of SEQ ID NO: 1775. In some embodiments, an isolated FN3 domain that binds CD71 comprises the amino acid sequence of SEQ ID NO: 1776. In some embodiments, an isolated FN3 domain that binds CD71 comprises the amino acid sequence of SEQ ID NO: 1777. In some embodiments, an isolated FN3 domain that binds CD71 comprises the amino acid sequence of SEQ ID NO: 1778. In some embodiments, an isolated FN3 domain that binds CD71 comprises the amino acid sequence of SEQ ID NO: 1779. In some embodiments, an isolated FN3 domain that binds CD71 comprises the amino acid sequence of SEQ ID NO: 1780. In some embodiments, an isolated FN3 domain that binds CD71 comprises the amino acid sequence of SEQ ID NO: 1781. In some embodiments, an isolated FN3 domain that binds CD71 comprises the amino acid sequence of SEQ ID NO: 1782. In some embodiments, an isolated FN3 domain that binds CD71 comprises the amino acid sequence of SEQ ID NO: 1783. In some embodiments, an isolated FN3 domain that binds CD71 comprises the amino acid sequence of SEQ ID NO: 1784. In some embodiments, an isolated FN3 domain that binds CD71 comprises the amino acid sequence of SEQ ID NO: 1785. In some embodiments, an isolated FN3 domain that binds CD71 comprises the amino acid sequence of SEQ ID NO: 1786. In some embodiments, an isolated FN3 domain that binds CD71 comprises the amino acid sequence of SEQ ID NO: 1787. In some embodiments, an isolated FN3 domain that binds CD71 comprises the amino acid sequence of SEQ ID NO: 1788. In some embodiments, an isolated FN3 domain that binds CD71 comprises the amino acid sequence of SEQ ID NO: 1789. In some embodiments, an isolated FN3 domain that binds CD71 comprises the amino acid sequence of SEQ ID NO: 1790. In some embodiments, an isolated FN3 domain that binds CD71 comprises the amino acid sequence of SEQ ID NO: 1791. In some embodiments, an isolated FN3 domain that binds CD71 comprises the amino acid sequence of SEQ ID NO: 1792. In some embodiments, an isolated FN3 domain that binds CD71 comprises the amino acid sequence of SEQ ID NO: 1793. In some embodiments, an isolated FN3 domain that binds CD71 comprises the amino acid sequence of SEQ ID NO: 1794. In some embodiments, an isolated FN3 domain that binds CD71 comprises the amino acid sequence of SEQ ID NO: 1795. In some embodiments, an isolated FN3 domain that binds CD71 comprises the amino acid sequence of SEQ ID NO: 1796. In some embodiments, an isolated FN3 domain that binds CD71 comprises the amino acid sequence of SEQ ID NO: 1797. In some embodiments, an isolated FN3 domain that binds CD71 comprises the amino acid sequence of SEQ ID NO: 1798. In some embodiments, an isolated FN3 domain that binds CD71 comprises the amino acid sequence of SEQ ID NO: 1799. In some embodiments, an isolated FN3 domain that binds CD71 comprises the amino acid sequence of SEQ ID NO: 1800. In some embodiments, an isolated FN3 domain that binds CD71 comprises the amino acid sequence of SEQ ID NO: 1801. In some embodiments, an isolated FN3 domain that binds CD71 comprises the amino acid sequence of SEQ ID NO: 1802. In some embodiments, an isolated FN3 domain that binds CD71 comprises the amino acid sequence of SEQ ID NO: 1803. In some embodiments, an isolated FN3 domain that binds CD71 comprises the amino acid sequence of SEQ ID NO: 1804. In some embodiments, an isolated FN3 domain that binds CD71 comprises the amino acid sequence of SEQ ID NO: 1805. In some embodiments, an isolated FN3 domain that binds CD71 comprises the amino acid sequence of SEQ ID NO: 1806. In some embodiments, an isolated FN3 domain that binds CD71 comprises the amino acid sequence of SEQ ID NO: 1807. In some embodiments, an isolated FN3 domain that binds CD71 comprises the amino acid sequence of SEQ ID NO: 1808. In some embodiments, an isolated FN3 domain that binds CD71 comprises the amino acid sequence of SEQ ID NO: 1809. In some embodiments, an isolated FN3 domain that binds CD71 comprises the amino acid sequence of SEQ ID NO: 1810. In some embodiments, an isolated FN3 domain that binds CD71 comprises the amino acid sequence of SEQ ID NO: 1811. In some embodiments, an isolated FN3 domain that binds CD71 comprises the amino acid sequence of SEQ ID NO: 1812. In some embodiments, an isolated FN3 domain that binds CD71 comprises the amino acid sequence of SEQ ID NO: 1813. In some embodiments, an isolated FN3 domain that binds CD71 comprises the amino acid sequence of SEQ ID NO: 1814. In some embodiments, an isolated FN3 domain that binds CD71 comprises the amino acid sequence of SEQ ID NO: 1815. In some embodiments, an isolated FN3 domain that binds CD71 comprises the amino acid sequence of SEQ ID NO: 1816. In some embodiments, an isolated FN3 domain that binds CD71 comprises the amino acid sequence of SEQ ID NO: 1817. In some embodiments, an isolated FN3 domain that binds CD71 comprises the amino acid sequence of SEQ ID NO: 1818. In some embodiments, an isolated FN3 domain that binds CD71 comprises the amino acid sequence of SEQ ID NO: 1819. In some embodiments, an isolated FN3 domain that binds CD71 comprises the amino acid sequence of SEQ ID NO: 1820. In some embodiments, an isolated FN3 domain that binds CD71 comprises the amino acid sequence of SEQ ID NO: 1821. In some embodiments, an isolated FN3 domain that binds CD71 comprises the amino acid sequence of SEQ ID NO: 1822. In some embodiments, an isolated FN3 domain that binds CD71 comprises the amino acid sequence of SEQ ID NO: 1823. In some embodiments, an isolated FN3 domain that binds CD71 comprises the amino acid sequence of SEQ ID NO: 1824. In some embodiments, an isolated FN3 domain that binds CD71 comprises the amino acid sequence of SEQ ID NO: 1825. In some embodiments, an isolated FN3 domain that binds CD71 comprises the amino acid sequence of SEQ ID NO: 1826. In some embodiments, an isolated FN3 domain that binds CD71 comprises the amino acid sequence of SEQ ID NO: 1827. In some embodiments, an isolated FN3 domain that binds CD71 comprises the amino acid sequence of SEQ ID NO: 1828. In some embodiments, an isolated FN3 domain that binds CD71 comprises the amino acid sequence of SEQ ID NO: 1829. In some embodiments, an isolated FN3 domain that binds CD71 comprises the amino acid sequence of SEQ ID NO: 1830. In some embodiments, an isolated FN3 domain that binds CD71 comprises the amino acid sequence of SEQ ID NO: 1831. In some embodiments, an isolated FN3 domain that binds CD71 comprises the amino acid sequence of SEQ ID NO: 1832. In some embodiments, an isolated FN3 domain that binds CD71 comprises the amino acid sequence of SEQ ID NO: 1833. In some embodiments, an isolated FN3 domain that binds CD71 comprises the amino acid sequence of SEQ ID NO: 1834. In some embodiments, an isolated FN3 domain that binds CD71 comprises the amino acid sequence of SEQ ID NO: 1835. In some embodiments, an isolated FN3 domain that binds CD71 comprises the amino acid sequence of SEQ ID NO: 1836. In some embodiments, an isolated FN3 domain that binds CD71 comprises the amino acid sequence of SEQ ID NO: 1837. In some embodiments, an isolated FN3 domain that binds CD71 comprises the amino acid sequence of SEQ ID NO: 1838. In some embodiments, an isolated FN3 domain that binds CD71 comprises the amino acid sequence of SEQ ID NO: 1839. In some embodiments, an isolated FN3 domain that binds CD71 comprises the amino acid sequence of SEQ ID NO: 1840. In some embodiments, an isolated FN3 domain that binds CD71 comprises the amino acid sequence of SEQ ID NO: 1841. In some embodiments, an isolated FN3 domain that binds CD71 comprises the amino acid sequence of SEQ ID NO: 1842. In some embodiments, an isolated FN3 domain that binds CD71 comprises the amino acid sequence of SEQ ID NO: 1843. In some embodiments, an isolated FN3 domain that binds CD71 comprises the amino acid sequence of SEQ ID NO: 1844. In some embodiments, an isolated FN3 domain that binds CD71 comprises the amino acid sequence of SEQ ID NO: 1845. In some embodiments, an isolated FN3 domain that binds CD71 comprises the amino acid sequence of SEQ ID NO: 1846. In some embodiments, an isolated FN3 domain that binds CD71 comprises the amino acid sequence of SEQ ID NO: 1847. In some embodiments, an isolated FN3 domain that binds CD71 comprises the amino acid sequence of SEQ ID NO: 1848. In some embodiments, an isolated FN3 domain that binds CD71 comprises the amino acid sequence of SEQ ID NO: 1849.

In some embodiments, the isolated FN3 domain that binds CD71 comprises an initiator methionine (Met) linked to the N-terminus of the molecule.

In some embodiments, the isolated FN3 domain that binds CD71 comprises an amino acid sequence that is 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to one of the amino acid sequences of SEQ ID NOs: 365-644, 663-672, or 1395-1849. Percent identity can be determined using the default parameters to align two sequences using BlastP available through the NCBI website. The sequences of the FN3 domains that bind to CD71 can be found, for example, in Table 9. These sequences are illustrated with a N-terminal methionine. The sequence of the domain can also be utilized without the N-terminal methionine. Simply for the avoidance of duplicating almost identical sequences, a table of such sequences is not being provided, but one of skill in the art could immediately envisage the sequences provided for herein without the N-terminal methionine and the disclosure should be understood and construed to include such sequences.

TABLE 9
CD71-binding FN3 Domain Sequences

| SEQ ID | |
|---|---|
| NO: | SEQUENCE |
| 365 | MLPAPKNLVVSRVTEDSARLSWTAPDAAFDSFPITYIEAVVLGEAIVLTVPGS |
| | ERSYDLTGLKPGTEYPVGISGVKGGHNSMPLSAIFTT |
| 366 | MLPAPKNLVVSEVTEDSARLSWQGVARAFDSFMINYSELFWMGEAIVLTVPGS |
| | ERSYDLTGLKPGTEYVVRIKGVKGGKGSWPLHAHFTT |
| 367 | MLPAPKNLVVSRVTEDSARLSWTAPDAAFDSFNIEYAETRWYGEAIVLTVPGS |
| | ERSYDLTGLKPGTEYVVPIDGVKGGIASKPLSAIFTT |
| 368 | MLPAPKNLVVSEVTEDSARLSWQGVARAFDSFLITYRDQIFAGEVIVLTVPGS |
| | ERSYDLTGLKPGTEYPVWIQGVKGGSPSAPLSAESTT |
| 369 | MLPAPKNLVVSEVTEDSARLSWQGVARAFDSFLITYREQIFAGEVIVLTVPGS |
| | ERSYDLTGLKPGTEYWVYIWGVKGGKPSFPLRAGFTT |
| 370 | MLPAPKNLVVSRVTEDSARLSWTAPDAAFDSFPIIYMETFSRGEAIVLTVPGS |
| | ERSYDLTGLKPGTEYRVPIGGVKGGSSSCPLSAIFTT |
| 371 | MLPAPKNLVVSDVTEDSARLSWQGVARAFDSFLITYREQIFAGEVIVLTVPGS |
| | ERSYDLTGLKPGTEYPVWIQGVKGGSPSAPLSAEFTT |
| 372 | MLPAPKNLVVSRVTEDSARLSWTAPDAAFDSFKIAYIETATRGEAIVLTVPGS |
| | ERSYDLTGLKPGTEYVVPIPGVKGGNTSSPLSAIFTT |
| 373 | MLPAPKNLVVSRVTEDSARLSWTAPDAAFDSFFIPYAEPSPTGEAIVLTVPGS |
| | ERSYDLTGLKPGTEYSVLIHGVKGGHLSDPLSAISTT |
| 374 | MLPAPKNLVVSRVTEDSARLSWTAPDAAFDSFLIAYPEDGFRGEAIVLTVPGS |
| | ERSYDLTGLKPGTEYPVPILGVKGGGGSGPLSAIFTT |
| 375 | MLPAPKNLVVSRVTEDSARLSWTAPDAAFDSFPIYYVENVVWGEAIVLTVPGS |
| | ERSYDLTGLKPGTEYWEVIIGVKGGQCSRPLSAIFTT |
| 376 | MLPAPKNLVVSRVTEDSARLSWQGVARAFDSFLITYREQIFAGEVIVLTVPGS |
| | ERSYDLTGLKPGTECPVWIQGVKGGSPSAPLSAEFTT |
| 377 | MLPAPKNLVVSRVTEDSARLSWTAPDAAFDSFGIAYREFRPSGEAIVLTVPGS |
| | ERSYDLIVETGYRNEVVICGVKGGPWSGPLSAIFTT |
| 378 | MLPAPKNLVVSRVTEDSARLSWTAPDAAFDSFPILYTECVYRGEAIVLTVPGS |
| | ERSYDLTGLKPGTEYHVPITGVKGGGGSWPLSAIFTT |
| 379 | MLPAPKNLVVSRVTEDSARLSWTAPDAAFDSFNIMYHEIIYVGEAIVLTVPGS |
| | ERSYDLTGLKPGTEYPVPIEGVKGGGISGPLSAIFTT |
| 380 | MLPAPKNLVVSRVTEDSARLSWTAPDAAFDSFAITYTEAALCGEAIVLTVPGS |
| | ERSYDLTGLKPGTEYPVPINGVKGGGTSGPLSAIFTT |
| 381 | MLPAPKNLVVARVTEDSARLSWTAPDAAIDSFPIDYSEYWWGGEAIVLTVPGS |
| | ERSYDLTGLKPGTEYPVLITGVKGGYRSGPLSAIFTT |
| 382 | MLPAPKNLVVSRVTEDSARLSWTAPDAAFDSFSIRYNEFIVAGEAIVLTVPGS |
| | ERSYDLTGLKPGTEYDVPIAGVKGGGASWPLSAIVTT |
| 383 | MLPAPKNLVVSRVTEDSARLSWTAPDAAFDSFWYLELQFAGEAIVLTVPGSER |
| | SYDLTGLKPGTEYNVPITGVKGGIISFPLSAIFTT |
| 384 | MLPAPKNLVVSRVTEDSARLSWTAPDAAFDSFAIWYHEWYGDGEAIVLTVPGS |
| | ERSYDLTGPKPGTEYRVRISGVKGGFESGPLSAIFTT |
| 385 | MLPAPKNLVVSRVTEDSARLSWTAPDAAFDSFMIRYQEGTRWGEAIVLTVPGS |
| | ERSYDLTGLKPGTEYIVMIAGVKGGQISLPLSAIFTT |
| 386 | MLPAPKNLVVSRVTEDSARLSWTAPDAAFDSFPIWYLEKSYQGEAIVLTVPGS |
| | ERSYDLTGLKPGTEYVVPIIGVKGGRDSCPLSAIFTT |
| 387 | MLPAPKNLVVSEVTEDSARLSWQGVARAFDSFLITYREQIFAGEVIVLTVPGS |
| | ERSYDLTGLKPGTEYPVWIQGVKGGSPSAPLSAEFTT |
| 388 | MLPAPKNLVVSRVTEDSARLSWTAPDAAFDSFRISYAETVRQGEAIVLTVPGS |
| | ERSYDLTVETGYRNWVMILGVKGGPGSLPLSAIFTT |
| 389 | MLPAPKNLVVSEVTEDSARLSWQGVVRAFDSFLITYREQIFAGEVIVLTVPGS |
| | ERSYDLTGLKPGTEYPVWIQGVKGGSPSAPLSAEFTT |
| 390 | MLPAPKNLVVSRVTEDSARLSWTAPDAAFDSFWIEYWEAVGFGEAIVLTVPGS |
| | ERSYDLTGLKPGTEYFVGIYGVKGGYLSAPLSAIFTT |
| 391 | MLPAPKNLVVSRVTEDSARLSWTAPDAAFDSFIIHYVEQQLIGEAIVLTVPGS |
| | ERSYDLTGLKPGTEYPVPITGVKGGACSWPLSAIFTT |
| 392 | MLPAPKNLVVSRVTEDSARLSWTAPDAAFDSFTIEYSEHPIDGEAIPLFVPGS |
| | ERSYDLTGLKPGTEYYVRIHGVKGGWFSHPLWAFFTT |
| 393 | MLPAPKNLVVSRVTEDSARLSWQGVARAFDSFLITYREQIFAGEVIVLTVPGS |
| | ERSYDLTGLKPGTEYGVTIAGVKGGWRSKPLNAESTT |
| 394 | MLPAPKNLVVSRVTEDSARLSWTAPDAAFDSFGIAYVESYWYGEAIVLTVPGS |
| | ERSYDLTGLKPGTEYNVPIYGVKGGDGSGPLSAIFTT |
| 395 | MLPAPKNLVVSRVTEDSARLSWTAPDAAFDSFYITYVELNLAGEAIVLTVPGS |
| | ERSYDLTGLKPGTEYPVPILGVKGGSLSQPLSAIFTT |
| 396 | MLPAPKNLVVSRVTEDSARLSWTAPDAAFDSFPISYIESIADGEAIVLTVPGS |
| | ERSYDLTGLKPGTEYWVAIVGVKGGPFSWSLSAIVTT |
| 397 | MLPAPKNLVVSEVTEDSARLSWQGVARAFDSFLITYREQIFAGEVIVPTVPGS |
| | ERSYDLTGLKPGTEYPVPIAGVKGGGPSAPLSAIFTT |
| 398 | MLPAPKNLVVSRVTEDSARLSWTTPDAAFDSFPIYYWEVTITGEAIYLSVPGS |
| | ERSYDLTGLKPGTEYPVDIPGVKGGAASPPLSAIFTT |
| 399 | MLPAPKNLVVSRVTEDSARLSWTAPDAAFDSFPILYLEHTVRSEAIVLTVPGS |
| | ERSYDLTDLKPGTEYCVPIDGVKGGLRSRPLSAIFTT |
| 400 | MLPAPKNLVVSRVTEDSARLSWTAPDAAFDSFPIPYTEPPDPGEAIVLTVPGS |
| | ERSYDLTGLKPGTEYLVTILGVKGGSMSVPLSAIFTT |
| 401 | MLPAPKNLVVSRVTEDSARLSWTAPDAAFDSFTIDYWENRCPGEAIVLTVPGS |
| | ERSYDLTGLKPGTEYCVWISGVKGGYSSWPLSAIFTT |
| 402 | MLPAPKNLVVSRVTEDSARLSWQGVARAFDSFLITYREQIFAGEVIVLTVPGS |
| | ERSYDLTGLKPGTEYPVWIQGVKGGHLSDPLSAIVTT |
| 403 | MLPAPKNLVVSRVTEDSARLSWTAPDAAFDSFLIPYAETSPSGEAIVLTVPGS |
| | ERSYDLTGLKPGTEYSVLIHGVKGGDYSEPLSAIFTT |
| 404 | MLPAPKNLVVSRVTEDSARLSWTAPDAAFDSFMIVYYEYTRFGEAIVLTVPGS |
| | ERSYDLTGLKPGTEYTVPIDGVKGGGRSSPLSAIFTT |
| 405 | MLPAPKNLVVSRVTEDSARLSWTAPDAAFDSFFIPYAEPSPTGEAIVLTVPGS |
| | ERSYDLTGLKPGTEYSVLIHGVKGGHLSDPLSAIVTT |
| 406 | MLPAPKNLVVSEVTEDSARLSWQGVARAFDSFLITYREQIFAGEVIVLTVPGS |
| | ERSYDLTGLKPGTEYPVWIQGVKGGSPSAPLSAEFTT |
| 407 | MLPAPKNLVVSRVTEDSARLSWTAPDAAFDSFIIPYAEVRPDGEAIVLTVPGS |
| | ERSYDLTGLKPGTEYSVLIHGVKGGKLSLPLSAIFTT |
| 408 | MLPAPKNLVVSRVTEDSARLSWTAPDAAFDSFGIVYLEMMVTGEAIVLTVPGS |
| | ERSYDLTGLKPGTEYDVPILGVKGGTRSVPLSAIFTT |
| 409 | MLPAPKNLVVSRVTEDSARLSWTAPDAAFDSFLIYYEEGYLEYYYSGEAIVLT |
| | VPGSERSYDLTGLKPGTEYYVGIVGVKGGGLSGPLSAISTT |
| 410 | MLPAPKNLVVSRVTEDSARLSWTAPDAAFDSEPIAYAEPRPDGEAIVLTVPGS |
| | ERSYDLTGLKPGTEYSVLIHGVKGGDWSLPLSAIFTT |
| 411 | MSLPAPKNLVVSRVTEDSARLSWTAPDAAFDSFTIHYREFQLSGEAIVLIVPG |
| | SERSYDLTGLKPGTEYDVPIEGVKGGPGSRPLSAIFTT |
| 412 | MLPAPKNLVVSEVTEDSARLSWQGVARAFDSFLITYREQIFAGEVIVLTVPGS |
| | ECSYDLTGLKPGTEYPVWIQGVKGGSPSAPLSAEFTT |
| 413 | MLPAPKNLVVSRVTEDSARLSWTAPDAAFDSFEIDYDELAIYGEAIVLTVPGS |
| | ERSYDLTGLKPGTEYGVRIPGVKGGMPSLPLSAIVTT |
| 414 | MLPAPENLVVSEVTEDSARLSWQGVARAFDSFLITYREQIFAGEVIVLTVPGS |
| | ERSYDLTGLKPGTEYPVWIQGVKGGSPSAPLSAESTT |
| 415 | MLPAPKNLVVSRVTEDSARLSWTAPDAAFDSFAIAYGEHIVIGEAIVLTVPGS |
| | ERSYDLTGLKPGTEYMVPIAGVKGGPISLPLSAIFTT |
| 416 | MLPAPKNLVVSRVTEDSARLSWTAPDAAFDSFFIPYAEPSPTGEAIVLTVPGS |
| | ERSYDLTGLKPGTEYSVLIHGVKGGHLSDPLSAIFTT |
| 417 | MLPAPKNLVVSRVTEDSARLSWTAPDAAFDSFSIGYVELVLLGEAIVLTVPGS |
| | ERSYDLTGLKPGTEYDVLIPGVKGGSLSRPLSAIFTT |
| 418 | MLPAPKNLVVSRVTEDSARLSWTAPDAAFDSFAIPYAELSRNGEAIVLTVPGS |
| | ERSYDLTGLKPGTEYTVLIHGVKGGCLSDPLSAIFTT |
| 419 | MLPAPKNLVVSRVTEDSARLSWTAPDAAFDSFHIEYLELSRHGEAIVLTVPGS |
| | ERSYDLTGLKPGTEYWVMIFGVKGGGPSKPLSAIFTT |
| 420 | MLPAPKNLVVSRVTEDSARLSWTAPDAAFDSFVYNEVHWIGEAIVLTVPGSER |
| | SYDLTGLKPGTEYFVGIYGVKGGHWSKPLSAIFTT |
| 421 | MLPAPKNLVVSRVTEDSARLSWTAPDAAFDSFEIDYDELAIYGEAIVLTVPGS |
| | ERSYDLTGLKPGTEYGVRIPGVKGGMPSLPLSAIVTT |
| 422 | MLPAPKNLVVSRVTEDSARLSWTAPDAAFDSFQIVYSELWIKGEAIVLTVPGS |
| | ERSYDLTGLKPGTEYQVPIPGVKGGRNSFPLSAIFTT |
| 423 | MLPAPKNLVVSRVTEDSARLSWTAPDAAFDSFKIRYTETRSIGEAIVLTVPGS |
| | ERSYDLTGLKPGTEYCVPIGGVKGGDSSWPLSAISTT |
| 424 | MLPAPKNLVVSRVTEDSARLSWTAPDAAFDSFCISYYERMGRGEAIVLTVPGS |
| | ERSYDLTGLKPGTEYMVYIFGVKGGLNSLPLSAIFTT |
| 425 | MLPAPKNLVVSRVTEDSARLSWTAPDAAFDSFYIVYAEPIPNGEAIVLTVPGS |
| | ERSYDLTGLKPGTEYSVLIHGVKGGRNSDPLSAIFTT |
| 426 | MLPAPKNLVVSRVTKDSARLSWTAPDAAFDSFPIAYAEPRPDGEAIVLTVPGS |
| | ERSYDLTGLKPGTEYSVLIHGVKGGLLSSPLSAIFTT |
| 427 | MLPAPKNLVVSRVTEDSARLSWTAPDAAFDSFTIDYDEPRSPGEAIVLTVPGS |
| | ERSYDLTGLKPGTEYRVFIWGIKGGDTSFPLSAIFTT |
| 428 | MLPAPKNLVVSRVTEDSARLSWTAPDAAFDSFTILYAEQAQFGEAIVLTVPGS |
| | ERSYDLTGLKPGTEYPITGVKGGTRSGPLSAISTT |
| 429 | MLPAPKNLVVSRVTEDSARLSWTAPDAAFDSFIIPYAEVRPDGEAIVLTVPGS |
| | ERSYDLTGLKPGTEYSVLIHGVKGGHLSDPLSAISTT |
| 430 | MLPAPKNLVVSRVTEDSARLSWTAPDAAFDSFHIAYEETATSGEAIYLRVPGS |
| | ERSYDLTGLKPGTEYGVEIEGVKGGARSRPLYADFTT |
| 431 | MLPAPKNLVVSRVTEDSARLSWQGVARAFDSFLITYREQIFAGEVIVLTVPGS |
| | ERSYDLTGLKPGTEYPVWIQGVKGGDLSNPLSAIFTT |
| 432 | MLPAPKNLVVSRVTEDSARLSWTAPDAAFDSFPISYLELSLYGEAIVLTVPGS |
| | ERSYDLTGLKPGTEYPVGIAGVKGGVVSRPLSAIFTT |
| 433 | MLPAPKNLVVSRVTEDSARLSWTAPDAAFDSFTIGYREWYWYGEAIVLTVPGS |
| | ERSYDLTGLKPGTEYNVPISGVKGGLDSFPLSAIFTT |
| 434 | MLPAPKNLVVSEVTEDSARLSWQGVARAFDSFLITYREQIFAGEVIVLTVPGS |
| | ERSYDLTGLKPGTEYPVWIQGVKGGSPSAPLSAESTT |
| 435 | MLPAPKNLVVSRVTEDSARLSWTAPDAAFDSFSITYLEWWNLGEAIVLTVPGS |
| | ERSYDLTGLKPGTEYMVTIPGVKGGMSSYPLSAIFTT |
| 436 | MLPAPKNLVVSRVTEDSARLSWTAPDAAFDSFTISYGEEALIGEAIYLRVPGS |
| | ERSYDLTGLKPGTEYYVHIEGVKGGSWSQPLAAAFTT |
| 437 | MLPAPKNLVVSRVTEDSARLSWTAPDAAFDSFTIEYYENIGIGEAIVLTVPGS |
| | ERSYDLTGLKPGTEYSVPIVGVKGGPYSHPLSAIFTT |
| 438 | MLPAPKNLVVSRVTEDSARLSWTAPDAAFDSFPIAYAEPRPDGEAIVLTVPGS |
| | ERSYDLTGLKPGTEYSVLIHGVKGGLLSSPLSAIFTT |
| 439 | MLPAPKNLVVSRVTEDSARLSWTAPDAAFDSFGIGYYEHKRFGEAIQLSVPGS |
| | ERSYDLTGLKPGTEYEVDIEGVKGGVLSWPLFAEFTT |
| 440 | MLPAPKNLVVSRVTEDSARLSWTAPDAAFDSFVIEYTERFWSGEAIVLTVPGS |
| | ERSYDLTGLKPGTEYSVPIDGVKGGQCSTPLSAIFTT |
| 441 | MLPAPKNLVVSRVTEDSARLSWTAPDAAFDSFWIDYEEEGVIGEAIYLHVPGS |
| | ERSYDLTGLKPGTEYVVKIHGVKGGHPSHPLVAVETT |
| 442 | MLPAPKNLVVSRVTEDSARLSWQGVARAFDSFLITYVELRHLGEAIVLTVPGS |
| | ERSYDLTGLKPGTEYPVWIQGVKGGSPSAPLSAEFTT |
| 443 | MLPAPKNLVVSRVTEDSARLSWTAPDAAFDSFLIPYAETSPSGEAIVLTVPGS |
| | ERSYDLTGLKPGTEYSVLIHGVKGGDYSSPLSAIFTT |
| 444 | MLPAPKNLVVSRVTEDSARLSWTAPDAAFDSFPIAYAEPRPDGEAIVLTVPGS |
| | ERSYDLTGLKPGTEYSVLIHGVKGGLLSSPLSAISTT |
| 445 | MLPAPKNLVVSRVTEDSARLSWQGVARAFDSFSILYLELTPKGEAIVLTVPGS |
| | ERSYDLTGLKPGTEYPVWIQGVKGGSPSAPLSAEFTT |
| 446 | MLPAPKNLVVSRVTEDSARLSWTAPDAAFDSFIIEYFEPIPIGEAIVLTVPGS |
| | ERSYDLTGLKPGTEYAVNIYGVKGGYLSHPLSAIFTT |
| 447 | MLPAPKNLVVSEVTEDSARLSWQGVARAFDSFLITYREQIFAGEVIVLTVPGS |
| | ECSYDLTGLKPGTEYPVWIQGVKGGSPSAPLSAEFTT |
| 448 | MLPAPKNLVVSRVTEDSARLSWTAPDAAFDSFAIEYTEFLYSGEAIVLTVPGS |
| | ERSYDLTGLKPGTEYGVPINGVKGGFVSPPLSAIVTT |
| 449 | MLPAPKNLVVSRVTEDSARLSWTAPDAAFDSFPIKYREVLRCGEAIVLTVPGS |
| | ERSYDLTGLKPGTEYTVPITGVKGGFGSSPLSAIFTT |
| 450 | MLPAPENLVVSRVTEDSARLSWTAPDAAFDSFWIEYYEGVIQGEAIVLTVPGS |
| | ERSYDLTGLKPGTEYFVAIWGVKGGKWSVPLSAIFTT |
| 451 | MLPAPKNLVVSRVTEDSARLSWQGVARAFDSFLITYREQIFAGEVIVLTVPGS |
| | ERSYDLTGLKPGTEYPVWIQGVKGGSPSAEFTT |
| 452 | MLPAPKNLVVSRVTEDSARLSWTAPDAAFDSFQIHYWETQGFGEAIVLTVPGS |
| | ERSYDLTGLKPGTEYPVLIPGVKGGPSSLPLSAIFTT |
| 453 | MLPAPKNLVVSRVTEDSARLSWTAPDAAFDSFFIPYAEPSPTGEAIVLTVPGS |
| | ERSYDLTGLKPGTEYSVLIHGVKGGHLSDPLSAIFTT |
| 454 | MLPAPKNLVVSRVTEDSARLSWTAPDAAFDSFAIEYYEPVPAGEAIYLDVPGS |
| | ERSYDLTGLKPGTEYDVTIYGVKGGYYSHPLFASFTT |
| 455 | MLPAPKNLVVSEVTEDSARLSWQGVARAFDSFLITYREQIFAGEVIVLTVPGS |
| | ERSYDLTGLKPGTEYPVWIQGVKGGSPSAPLSAIFTT |
| 456 | MLPAPKNLVVSEVTEDSARLSWQGVARAFDSFLITYREQIFAGEVIVLTVPGS |
| | ERSYDLTGLKPGTEYPVWIQGVKGGSPSAPLSAEFTT |
| 457 | MSLPAPKNLVVSRVTEDSARLSWTAPDAAFDSFPIAYLEVFYEGEAIVLTVPG |
| | SERSYDLTGLKPGTEYQVPIEGVKGGAMSLPLSAIFTT |
| 458 | MLPAPKNLVVSRVTEDSARLSWTAPDAAFDSFHIWYEEETTIGEAIYLHVPGS |
| | ERSYDLTGLKPGTEYEVHITGVKGGPYSRPLFANFTT |
| 459 | MLPAPKNLVVSRVTEDSARLSWTAPDAAFDSFGIAYDEWPEFGEAIVLTVPGS |
| | ERSYDLTGLKPDTEYIVEIYGVKGGWFSWPLSAIFTT |
| 460 | MLPAPKNLVVSRVTEDSARLSWTAPDAAFDSFFIPYAEPSPTGEAIVLTVPGS |
| | ERSYDLTGLKPGTEYSVLIHGVKGGHLSDPLSVIFTT |
| 461 | MLPAPKNLVVSRVTEDSARLSWTAPDAAFDSFYIWYEEVMYLGEAIVLTVPGS |
| | ERSYDLTGLKPGTEYNVPIPGVKGGHSSPPLSAIFTT |
| 462 | MLPAPKNLVVSRVTEDSARLSWTAPDAAFDSFHILYEELFLVGEAIVLTVPGS |
| | ERSYDLTGLKPGTEYKVPISGVKGGPVSRPLSAIFTT |
| 463 | MLPAPKNLVVSRVTEDSARLSWQGVARAFDSFLITYREQIFAGEVIVLTVPGS |
| | ERSYDLTGLKPGTEYPVWIQGVKGGSPSAPLSAEFTT |
| 464 | MLPAPKNLVVSRVTEDSARLSWTAPDAAFDSFHIVYHEPRPSGEAIWLHVPGS |
| | ERSYDLTGLKPGTEYEVGIVSVKGGDLSVPLVAFFTT |
| 465 | MLPAPKNLVVSRVTEDSARLSWTAPDAAFDSFYIAYAEPRPDGEAISLLVPGS |
| | ERSYDLTGLKPGTEYSVLIHGVKGGLLSSPLYAVETT |
| 466 | MLPAPKNLVVSRVTEDSARLSWTAPDAAFDSFYIAYAEPRPDGEAIFLVVPGS |
| | ERSYDLTGLKPGTEYSVLIHGVKGGLLSSPLHANFTT |
| 467 | MLPAPKNLVVSRVTEDSARLSWTAPDAAFDSFYIAYAEPRPDGEAILLDVPGS |
| | ERSYDLTGLKPGTEYSVLIHGVKGGLLSSPLYASFTT |
| 468 | MSLPAPKNLVVSRVTEDSARLSWTAPDAAFDSFYIAYAEPRPDGEAISLYVPG |
| | SERSYDLTGLKPGTEYSVLIHGVKGGLLSSPLSAISTT |
| 469 | MLPAPKNLVVSRVTEDSARLSWTAPDAAFDSFFIGYLEPQPPGEAIQLRVPGS |
| | ERSYDLTGLKPGTEYNVTIQGVKGGFPSMPLSAIFTT |
| 470 | MLPAPKNLVVSRVTEDSARLSWTAPDAAFDSFHISYEEDYTFGEAIYLRVPGS |
| | ERSYDLTGLKPGTEYRVVIGGVKGGWFSEPLLAAFTT |
| 471 | MLPAPKNLVVSRVTEDSARLSWTAPDAAFDSFFIGYLEPQPPGEAIYLTVPGS |
| | ERSYDLTGLKPGTEYNVTIQGVKGGFPSYPLDASFTT |
| 472 | MLPAPKNLVVSRVTEDSARLSWTAPDAAFDSFFIGYLEPQPPGEAIDLGVPGS |
| | ERSYDLTGLKPGTEYNVTIQGVKGGFPSMPLDPLEAYFTT |
| 473 | MLPAPKNLVVSRVTEDSARLSWTAPDAAFDSFYIAYAEPRPDGEAILLLVPGS |
| | ERSYDLTGLKPGTEYSVLIHGVKGGLLSSPLSAIFTT |
| 474 | MLPAPKNLVVSRVTEDSARLSWTAPDAAFDSFYIAYAEPRPDGEAINLQVPGS |
| | ERSYDLTGLKPGTEYSVLIHGVKGGLLSSPLVAFFTT |
| 475 | MLPAPKNLVVSRVTEDSARLSWTTPDAAFDSFFIGYLEPQPPGEAISLQVPGS |
| | ERSYDLTGLKPGTEYNVTIQGVKGGFPSSPLFAVETT |
| 476 | MLPAPKNLVVSRVTEDSARLSWTAPDAAFDSFYIAYAEPRPDGEAIELHVPGS |
| | ERSYDLTGLKPGTEYSVLIHGVKGGLLSSPLFTT |
| 477 | MLPAPKNLVVSRVTEDSARLSWTAPDAAFDSFYIAYAEPRPDGEAIQLVVPGS |
| | ERSYDLTGLKPGTEYSVLIHGVKGGLLSSPLSAIFTT |
| 478 | MLPAPKNLVVSRVTEDSARLSWTAPDAAFDSFYIAYAEPRPDGEAITLDVPGS |
| | ERSYDLTGLKPGTEYSVLIHGVKGGLLSSPLSAIFTT |
| 479 | MLPAPKNLVVSRVTEDSARLSWTAPDAAFDSFYIAYAEPRPDGEAIWLVVPGS |
| | ERSYDLTGLKPGTEYSVLIHGVKGGLLSSPLVASFTT |
| 480 | MLPAPKNLVVSRVTEDSARLSWTAPDAAFDSFYIAYAEPRPDGEAINLDVPGS |
| | ERSYDLTGLKPGTEYSVLIHGVKGGLLSSPLVAEFTT |
| 481 | MLPAPKNLVVSRVTEDSARLSWTAPDAAFDSFYIAYAEPRPDGEAIHLSVPGS |
| | ERSYDLTGLKPGTEYSVLIHGVKGGLLSSPLVAIFTT |
| 482 | MLPAPKNLVVSRVTEDSARLSWTAPDAAFDSFFIGYLEPQPPGEAIALWVPGS |
| | ERSYDLTGLKPGTEYNVTIQGVKGGFPSMPLSAIFTT |
| 483 | MLPAPKNLVVSRVTEDSARLSWTAPDAAFDSFYIAYAEPRPDGEAIILVVPGS |
| | ERSYDLTGLKPGTEYSVLIHGVKGGLLSSPLSAHFTT |
| 484 | MLPAPKNLVVSRVTEDSARLSWTAPDAAFDSFFIGYLEPQPPGEAIQLWVPGS |
| | ERSYDLTGLKPGTEYNVTIQGVKGGFPSHPLGAVETT |
| 485 | MLPAPKNLVVSRVTEDSARLSWTAPDAAFDSFYIAYAEPRPDGEAIQLHVPGS |
| | ERSYDLTGLKPGTEYSVLIHGVKGGLLSSPLLASFTT |
| 486 | MLPAPKNLVVSRVTEDSARLSWTAPDAAFDSFYIAYAEPRPDGEAIALHVPGS |
| | ERSYDLTGLKPGTEYSVLIHGVKGGLLSSPLVAFFTT |
| 487 | MLPAPKNLVVSRVTEDSARLSWTAPDAAFDSFFIGYLEPQPPGEAIHLHVPGS |
| | ERSYDLTGLKPGTEYNVTIQGVKGGFPSIPLHANFTT |
| 488 | MLPAPKNLVVSRVTEDSARLSWTAPDAAFDSFYIAYAEPRPDGEAIFLGVPGS |
| | ERSYDLTGLKPGTEYSVLIHGVKGGLLSSPLSAIFTT |
| 489 | MLPAPKNLVVSRVTEDSARLSWTAPDAAFDSFYIAYAEPRPDGEAIVLRVPGS |
| | ERSYDLTGLKPGTEYSVLIHGVKGGLLSSPLIASFTT |
| 490 | MLPAPKNLVVSRVTEDSARLSWTAPDAAFDSFYIAYAEPRPDGEAINLWVPGS |
| | ERSYDLTGLKPGTEYSVLIHGVKGGLLSSPLDASFTT |
| 491 | MLPAPKNLVVSRVTEDSARLSWTAPDAAFDSFVIEYFEWTLNGEAIVLTVPGS |
| | ERSYDLTGLKPGTEYSVQIYGVKGGCLSRPLSAIFTT |
| 492 | MLPAPKNLVVSRVTEDSARLSWTAPDAAFDSFYIAYAEPRPDGEAIHLWVPGS |
| | ERSYDLTGLKPGTEYSVLIHGVKGGLLSSPLIAHFTT |
| 493 | MLPAPKNLVVSRVTEDSARLSWTAPDAAFDSFFIPYAEPSPTGEAIVLTVPGS |
| | ERSYDLTGLKPGTEYSVLIHGVKGGLLSSPLSAHFTT |
| 494 | MLPAPKNLVVSRVTEDSARLSWTAPDAAFDSFYIAYAEPRPDGEAIYLYVPGS |
| | ERSYDLTGLKPGTEYSVLIHGVKGGLLSSPLDAFFTT |
| 495 | MLPAPKNLVVSRVTEDSARLSWTAPDAAFDSFYIAYAEPRPDGEAIGLQVPGS |
| | ERSYDLTGLKPGTEYSVLIHGVKGGLLSSPLSAIFTT |
| 496 | MLPAPKNLVVSRVTEDSARLSWTAPDAAFDSFYIAYAEPRPDGEAIQLAVPGS |
| | ERSYDLTGLKPGTEYSVLIHGVKGGLLSSPLHAFFTT |
| 497 | MLPAPKNLVVSRVTEDSARLSWTAPDAAFDSFFIGYLEPQPPGEAIWLHVPGS |
| | ERSYDLTGLKPGTEYNVTIQGVKGGFPSIPLIAIFTT |
| 498 | MLPAPKNLVVSRVTEDSARLSWTAPDAAFDSFYIAYAEPRPDGEAIVLDVPGS |
| | ERSYDLTGLKPGTEYSVLIHGVKGGLLSSPLVAEFTT |
| 499 | MLPTPKNLVVSRVTEDSARLSWTAPDAAFDSFYIAYAEPRPDGEAIQLRVPGS |
| | ERSYDLTGLKPGTEYSVLIHGVKGGLLSSPLHASFTT |
| 500 | MLPAPKNLVVSRVTEDSARLSWTAPDAAFDSFFIGYLEPQPPGEAIQLGVPGS |
| | ERSYDLTGLKPGTEYNVTIQGVKGGFPSHPLNANFTT |
| 501 | MLPAPKNLVVSRVTEDSARLSWTAPDAAFDSFFIGYLEPQPPGEAIQLEVPGS |
| | ERSYDLTGLKPGTEYNVTIQGVKGGFPSMPLSAIFTT |
| 502 | MLPAPKNLVVSRVTEDSARLSWTAPDAAFDSFYIAYAEPRPDGEAIFLGVPGS |
| | ERSYDLTGLKPGTEYSVLIHGVKGGLLSSPLIAFFTT |
| 503 | MLPAPKNLVVSRVTEDSARLSWTAPDAAFDSFFIGYLEPQPPGEAIGLQVPGS |
| | ERSYDLTGLKPGTEYNVTIQGVKGGFPSHPLKAQFTT |
| 504 | MLPAPKNLVVSRVTEDSARLSWTAPDAAFDSFYIAYAEPRPDGEAILLFVPGS |
| | ERSYDLTGLKPGTEYSVLIHGVKGGLLSSPLVAHFTT |
| 505 | MLPAPKNLVVSRVTEDSARLSWTAPDAAFDSFYIAYAEPRPDGEAIGLYVPGS |
| | ERSYDLTGLKPGTEYSVLIHGVKGGLLSSPLGAFFTT |
| 506 | MLPAPKNLVVSRVTEDSARLSWTAPDAAFDSFYIAYAEPRPDGEAILLQVPGS |
| | ERSYDLTGLKPGTEYSVLIHGVKGGLLSSPLTAIFTT |
| 507 | MLPAPKNLVVSRVTEDSARLSWTAPDAAFDSFYIAYAEPRPDGEAITLHVPGS |
| | ERSYDLTGLKPGTEYSVLIHGVKGGLLSSPLSAIFTT |
| 508 | MLPAPKNLVVSRVTEDSARLSWTAPDAAFDSFYIAYAEPRPDGEAIVLEVPGS |
| | ERSYDLTGLKPGTEYSVLIHGVKGGLLSSPLDAHFTT |
| 509 | MLPAPKNLVVSRVTEDSARLSWTAPDAAFDSFYIAYAEPRPDGEAIALHVPGS |
| | ERSYDLTGLKPGTEYSVLIHGVKGGLLSSPLRAVETT |
| 510 | MLPAPKNLVVSRVTEDSARLSWTAPDAAFDSFYIAYAEPRPDGEAIQLWVPGS |
| | ERSYDLTGLKPGTEYSVLIHGVKGGLLSSPLSAIFTT |
| 511 | MLPAPKNLVVSRVTEDSARLSRTAPDAAFDSFYIAYAEPRPDGEAIVLTVPGS |
| | ERSYDLTGLKPGTEYSVLIHGVKGGLLSSPLSAIFTT |
| 512 | MLPAPKNLVVSRVTEDSARLSWTAPDAAFDSFFIGYLEPQPPGEAIQLWVPGS |
| | ERSYDLTGLKPGTEYNVTIQGVKGGFPSRPLQAHFTT |
| 513 | MLPAPKNLVVSRVTEDSARLSWTAPDAAFDSFYIAYAEPRPDGEAITLDVPGS |
| | ERSYDLTGLKPGTEYSVLIHGVKGGLLSSPLYAFFTT |
| 514 | MLPAPKNLVVSRVTEDSARLSWTAPDAAFDSFYIAYAEPRPDGEAIALHVPGS |
| | ERSYDLTGLKPGTEYSVLIHGVKGGLLSSPLSAIFTT |
| 515 | MLPAPKNLVVSRVTEDSARLSWTAPDAAFDSFYIAYAEPRPDGEAIGLWVPGS |
| | ERSYDLTGLKPGTEYSVLIHGVKGGLLSSPLIAHFTT |
| 516 | MLPAPKNLVVSRVTEDSARLSWTAPDAAFDSFYIAYAEPRPDGEAIWLVVPGS |
| | ERSYDLTGLKPGTEYSVLIHGVKGGLLSSPLHARFTT |
| 517 | MLPAPKNLVVSRVTEDSARLSWTAPDAAFDSFYIAYAEPRPDGEAIFLQVPGS |
| | ERSYDLTGLKPGTEYSVLIHGVKGGLLSSPLAAVFTT |
| 518 | MLPAPKNLVVSRVTEDSARLSWTAPDAAFDSFYIAYAEPRPDGEAILLHVPGS |
| | ERSYDLTGLKPGTEYSVLIHGVKGGLLSSPLSAISTT |
| 519 | MLPAPKNLVVSRVTEDSARLSWTAPDAAFDSFYIAYAEPRPDGEAIILQVPGS |
| | ERSYDLTGLKPGTEYSVLIHGVKGGLLSSPLSAVETT |
| 520 | MLPAPKNLVVSRVTEDSARLSWTAPDAAFDSFYIAYAEPRPDGEAIYLKVPGS |
| | ERSYDLTGLKPGTEYSVLIHGVKGGLLSSPLVAHFTT |
| 521 | MLPAPKNLVVSRVTEDSARLSWTAPDAAFDSFYIAYAEPRPDGEAIVLTVPGS |
| | ERSYDLTGLKPGTEYSVLIHGVKGGLLSSPLLAYFTT |
| 522 | MLPAPKNLVVSRVTEDSARLSWTAPDAAFDSFYIAYAEPRPDGEAIILHVPGS |
| | ERSYDLTGLKPGTEYSVLIHGVKGGLLSSPLEAKFTT |
| 523 | MLPAPKNLVVSRVTEDSARLSWTAPDAAFDSFYIAYAEPRPDGEAIKLEVPGS |
| | ERSYDLTGLKPGTEYSVLIHGVKGGLLSSPLAIFTT |
| 524 | MLPAPKNLVVSRVTEDSARLSWTAPDAAFDSFFIGYLEPQPPGEAIYLEVPGS |
| | ERSYDLTGLKPGTEYNVTIQGVKGGFPSFPLKAAFTT |
| 525 | MLPAPKNLVVSRVTEDSARLSWTAPDAAFDSFYIAYAEPRPDGEAIILRVPGS |
| | ERSYDLTGLKPGTEYSVLIHGVKGGLLSSPLVAIFTT |
| 526 | MLPAPKNLVVSRVTEDSARLSWTAPDAAFDSFYIAYAEPRPDGEAIVLQVPGS |
| | ERSYDLTGLKPGTEYSVLIHGVKGGLLSSPLAAWFTT |
| 527 | MLPAPKNLVVSRVTEDSARLSWTAPDAAFDSFYIAYAEPRPDGEAIFLQVPGS |
| | ERSYDLTGLKPGTEYSVLIHGVKGGLLSSPLNAFFTT |
| 528 | MLPAPKNLVVSRVTEDSARLSWTAPDAAFDSFFIGYLEPQPPGEAIILGVPGS |
| | ERSYDLTGLKPGTEYSVLIHGVKGGLLSSPLHAYSTT |
| 529 | MLPAPKNLVVSRVTEDSARLSWTAPDAAFDSFYIAYAEPRPDGEAILLDVPGS |
| | ERSYDLTGLKPGTEYSVLIHGVKGGLLSSPLSAIFTT |
| 530 | MLPAPKNLVVSRVTEDSARLSWTAPDAAFDSFYIAYAEPRPDGEAILLLVPGS |
| | ERSYDLTGLKPGTEYSVLIHGVKGGLLSSPLSAVFTT |
| 531 | MLPAPKNLVVSRVTEDSARLSWTAPDAAFDSFYIAYAEPRPDGEAIHLLVPGS |
| | ERSYDLTGLKPGTEYSVLIHGVKGGLLSSPLLAHFTT |
| 532 | MLPAPKNLVVSRVTEDSARLSWTAPDAAFDSFYIAYAEPRPDGEAIQLWVPGS |
| | ERSYDLTGLKPGTEYSVLIHGVKGGLLSSPLDAYFTT |
| 533 | MLPAPKNLVVSRVTEDSARLSWTAPDAAFDSFFIGYLEPQPPGEAIVLTVPGS |
| | ERSYDLTGLKPGTEYNVTIQGVKGGFPSMPLASFTT |
| 534 | MLPAPKNLVVSRVTEDSARLSWTAPDAAFDSFHIVYHEPRPSGEAIHLQVPGS |
| | ERSYDLTGLKPGTEYNVTIQGVKGGFPSYPLSAFFTT |
| 535 | MLPAPKNLVVSRVTEDSARLSWTAPDAAFDSFFIGYLEPQPPGEAIQLWVPGS |
| | ERSYDLTGLKPGTEYNVTIQGVKGGFPSMPLSAIFTT |
| 536 | MLPAPKNLVVSRVTEDSARLSWTAPDAAFDSFRISYCETFYHGEAIVLTVPGS |
| | ERSYDLTGLKPGTEYSVLIHGVKGGLLSSPLIAKFTT |
| 537 | MLPAPKNLVVSRVTEDSARLSWTAPDAAFDSFYIAYAEPRPDGEAIWLKVPGS |
| | ERSYDLTGLKPGTEYSVLIHGVKGGLLSSPLQANFTT |
| 538 | MLPAPKNLVVSRVTEDSARLSWTAPDAAFDSFYIAYAEPRPDGEAIWLKVPGS |
| | ERSYDLTGLKPGTEYSVLIHGVKGGLLSSPLQANFTT |
| 539 | MLPAPKNLVVSRVTEDSARLSWTAPDAAFDSFYIAYAEPRPDGEAIQLQVPGS |
| | ERSYDLTGLKPGTEYSVLIHGVKGGLLSSPLSAIVTT |
| 540 | MLPAPKNLVVSRVTEDSARLSWTAPDAAFDSFPIAYAEPRPDGEAIVLTVPGS |
| | ERSYDLTGLKPGTEYSVLIHGVKGGLLSSPLSAFFTT |
| 541 | MLPAPKNLVVSRVTEDSARLSWTAPDAAFDSFYIAYAEPRPDGEAIALLVPGS |
| | ERSYDLTGLKPGTEYSVLIHGVKGGLLSSPLVAQFTT |
| 542 | MLPAPKNLVVSRVTEDSARLSWTAPDAAFDSFYIAYAEPRPDGEAIILHVPGS |
| | ERSYDLTGLKPGTEYSVLIHGVKGGLLSSPLEAKFTT |
| 543 | MLPAPKNLVVSRVTEDSARLSWTAPDAAFDSFYIAYAEPRPDGEAIDLHVPGS |
| | ERSYDLTGLKPGTEYSVLIHGVKGGLLSSPLHALFTT |
| 544 | MLPAPKNLVVSRVTEDSARLSWTAPDAAFDSFYIAYAEPRPDGEAILLDVPGS |
| | ERSYDLTGLKPGTEYSVLIHGVKGGFPSMPLSAIFTT |
| 545 | MLPAPKNLVVSRVTEDSARLSWTAPDAAFDSFFIGYLEPQPPGEAIDLAVPGS |
| | ERSYDLTGLKPGTEYSVLIHGVKGGLLSSPLSFTT |
| 546 | MLPAPKNLVVSRVTEDSARLSWTAPDAAFDSFYIAYAEPRPDGEAIYLGVPGS |
| | ERSYDLTGLKPGTEYSVLIHGVKGGLLSSPLRAKFTT |
| 547 | MLPAPKNLVVSRVTEDSARLSWTAPDAAFDSFYIAYAEPRPDGEAIQLGVPGS |
| | ERSYDLTGLKPGTEYSVLIHGVKGGLLSSPLSAIFTT |
| 548 | MLPAPKNLVVSRVTEDSARLSWTAPDAAFDSFFIGYLEPQPPGEAISLLVPDS |
| | ERSYDLTGLKPGTEYNVTIQGVKGGFPSMPLKFTT |
| 549 | MLPAPKNLVVSRVTEDSARLSWTAPDAAFDSFYIAYAEPRPDGEAIGLGVPGS |
| | ERSYDLTGLKPGTEYSVLIHGVKGGLLSSPLDASFTT |
| 550 | MLPAPKNLVVSRVTEDSARLSWTAPDAAFDSFYIAYAEPRPDGEAIQLTVPGS |
| | ERSYDLTGPKPGTEYWVLIQGVKGGGSSVPLVAYFTT |
| 551 | MLPAPKNLVVSRVTEDSARLSWTAPDAAFDSFYIAYAEPRPDGEAISLDVPGS |
| | ERSYDLTGLKPGTEYSVLIHGVKGGLLSSPLEASFTT |
| 552 | MLPAPKNLVVSRVTEDSARLSWTAPDAAFDSFFIGYLEPQPPGEAIILAVPGS |
| | ERSYDLTGLKPGTEYNVTIQGVKGGFPSLPLVASFTT |
| 553 | MLPAPKNLVVSRVTEDSARLSWTAPDAAFDSFDIGYTEYGGYGEAIYLSVPGS |
| | ERSYDLTGLKPGTEYWVLIQGVKGGGSSVPLSAIFTT |
| 554 | MLPAPKNLVVSRVTEDSARLSWTAPDAAFDSFYIAYAEPRPDGEAISLSVPGS |
| | ERSYDLTGLKPGTEYSVLIHGVKGGLLSSPLIANFTT |
| 555 | MLPAPKNLVVSRVTEDSARLSWTAPDAAFDSFYIAYAEPRPDGEAIALLVPGS |
| | ERSYDLTGLKPGTEYSVLIHGVKGGLLSSPLSAIVTT |
| 556 | MLPAPKNLVVSRVTEDSARLSWTAPDAAFDSFYIAYAEPRPDGEAIILDVPGS |
| | ERSYDLTGLKPGTEYSVLIHGVKGGLLSSPLSSIFTT |
| 557 | MLPAPKNLVVSRVTEDSARLSWTAPDAAFDSFYIAYAEPRPDGEAIVLWVPGS |
| | ERSYDLTGLKPGTEYSVLIHGVKGGLLSSPLRASFTT |
| 558 | MLPAPKNLVVSRVTEDSARLSWTAPDAAFDSFYIAYAEPRPDGEAIKLDVPGS |
| | ERSYDLTGLKPGTEYSVLIHGVKGGLLSSPLVAFFTT |
| 559 | MLPAPKNLVVSRVTEDSARLSWTAPDAAFDSFYIAYAEPRPDGEAIILEVPGS |
| | ERSYDLTGLKPGTEYSVLIHGVKGGLLSSPLVAYFTT |
| 560 | MLPAPKNLVVSRVTEDSARLSWTAPDAAFDSFYIAYAEPRPDGEAIHLWVPGS |
| | ERSYDLTGLKPGTEYSVLIHGVKGGLLSSPLHADFTT |
| 561 | MLPAPKNLVVSRVTEDSARLSWTAPDAAFDSFYIAYAEPRPDGEAIWLEVPGS |
| | ERSYDLTGLKPGTEYSVLIHGVKGGLLSSPLVADFTT |
| 562 | MLPAPKNLVVSRVTEDSARLSWTAPDAAFDSFYIAYAEPRPDGEAISLWVPGS |
| | ERSYDLTGLKPGTEYSVLIHGVKGGLLSSPLLAHFTT |
| 563 | MLPAPKNLVVSRVTEDSARLSWTAPDAAFDSFDIGYTEYGGYGEAILHVPGSE |
| | RSYDLTGLKPGTEYWVLIQGVKGGGSSVPLSAIFTT |
| 564 | MLPAPKNLVVSRVTEDSARLSWTAPDAAFDSFFIGYLEPQPPGEAIVLLVPGS |
| | ERSYDLTGLKPGTEYNVTIQGVKGGFPSVPLAAFFTT |
| 565 | MLPAPKNLVVSRVTEDSARLSWTAPDAAFDSFFIGYLEPQPPGEAILLWVPGS |
| | ERSYDLTGLKPGTEYNVTIQGVKGGFPSQFTT |
| 566 | MLPAPKNLVVSRVTEDSARLSWTAPDAAFDSFFIGYLEPQPPGEAILLGVPGS |
| | ERSYDLTGLKPGTEYNVTIQGVKGGFPSMPLHPLVALFTT |
| 567 | MLPAPKNLVVSRVTEDSARLSWTAPDAAFDSFYIAYAEPRPDGEAIGLDVPGS |
| | ERSYDLTGLKPGTEYSVLIHGVKGGLLSSPLSAIFTT |
| 568 | MLPAPKNLVVSRVTEDSARLSWTAPDAAFDSFYIAYAEPRPDGEAIHLSVPGS |
| | ERSYDLTGLKPGTEYSVLIHGVKGGLLSSPLAAYFTT |
| 569 | MLPAPKNLVVSRVTEDSARLSWTAPDAAFDSFFIGYLEPQPPGEAIVLAVPGS |
| | ERSYDLTGLKPGTEYNVTIQGVKGGFPSYPLVAAFTT |
| 570 | MLPAPKNLVVSRVTEDSARLSWTAPDAAFDSFYIAYAEPRPDGEAILLQVPGS |
| | CRSYDLTGLKPGTEYSVLIHGVKGGLLSSPLTAIFTT |
| 571 | MLPAPKNLVVSRVTEDSARLSWTAPDAAFDSFFIGYLEPQPPGEAINLQVPGS |
| | ERSYDLTGLKPGTEYNVTIQGVKGGFPSFPLSAVFTT |
| 572 | MLPAPKNLVVSRVTEDSARLSWTAPDAAFDSFYIAYAEPRPDGEAIQLHVPGS |
| | ERSYDLTGLKPGTEYSVLIHGVKGGLLSSPLVAIFTT |
| 573 | MLPAPKNLVVSRVTEDSARLSWTAPDAAFDSFYIAYAEPRPDGEAIWLAVPGS |
| | ERSYDLTGLKPGTEYSVLIHGVKGGLLSSPLHAQFTT |
| 574 | MLPAPKNLVVSRVTEDSARLSWTAPDAAFDSFYIAYAEPRPDGEAILLGVPGS |
| | ERSYDLTGLKPGTEYSVLIHGVKGGLLSSPLFTT |
| 575 | MLPAPKNLVVSRVTEDSARLSWTAPDAAFDSFYIAYAEPRPDGEAIGLQVPGS |
| | ERSYDLTGLKPGTEYSVLIHGVKGGLLSSPLCAEFTT |
| 576 | MLPAPKNLVVSRVTEDSARLSWTAPDAAFDSFYIAYAEPRPDGEAIVLWVPGS |
| | ERSYDLTGLKPGTEYSVLIHGVKGGLLSSPLIAEFTT |
| 577 | MLPAPKNLVVSRVTEDSARLSWTAPDAAFDSFYIAYAEPRPDGEAISLSVPGS |
| | ERSYDLTGLKPGTEYSVLIHGVKGGLLSSPPKFTT |
| 578 | MLPAPKNLVVSRVTEDSARLSWTAPDAAFDSFYIAYAEPRPDGEAIILEVPGS |
| | ERSYDLTGLKPGTEYSVLIHGVKGGLLSSPLRAVFTT |
| 579 | MLPAPKNLVVSRVTEDSARLSWTAPDAAFDSFYIAYAEPRPDGEAIHLVVPGS |
| | ERSYDLTGLKPGTEYSVLIHGVKGGLLSSPLSAIFTT |
| 580 | MLPAPKNLVVSRVTEDSARLSWTAPDAAFDSFYIAYAEPRPDGEAISLKVPGS |
| | ERSYDLTGLKPGTEYSVLIHGVKGGLLSSPLEAIFTT |
| 581 | MLPAPKNPVVSRVTEDSARLSWTAPDAAFDSFYIAYAEPRPDGEAIHLLVPGS |
| | ERSYDLTGLKPGTEYSVLIHGVKGGLLSSPLKRLSPPVVTITITMAVCRKPVA |
| | ENLSQTLS |
| 582 | MLPAPKNLVVSRVTEDSARLSWTAPDAAFDSFFIGYLEPQPPGEAIFLDVPGS |
| | ERSYDLTGLKPGTEYNVTIQGVKGGFPSPLTAFFTT |
| 583 | MLPAPKNLVVSRVTEDSARLSWTAPDAAFDSFFIGYLEPQPPGEAIVLDVPGS |
| | ERSYDLTGLKPGTEYNVTIQGVKGGFPSHPLAAAFTT |
| 584 | MLPAPKNLVVSRVTEDSARLSWTAPDAAFDSFFIGYLEPQPPGEAIGLAVPGS |
| | ERSYDLTGLKPGTEYNVTIQGVKGGFPSVPLQANFTT |
| 585 | MLPAPKNLVVSRVTEDSARLSWTAPDAAFDSFYIAYAEPRPDGEAILLRVPGS |
| | ERSYDLTGLKPGTEYSVLIHGVKGGLLSSPLVAEFTT |
| 586 | MLPAPKNLVVSRVTEDSARLSWTAPDAAFDSFYIAYAEPRPDGEAILLQVPGS |
| | ERSYDLTGLKPGTEYSVLIHGVKGGLLSSPLSASFTT |
| 587 | MLPAPKNLVVSRVTEDSARLSWTAPDAAFDSFYIAYAEPRPDGEAIGLHVPGS |
| | ERSYDLTGLKPGTEYSVLIHGVKGGLLSSPLTASFTT |
| 588 | MLPAPKNLVVSRVTEDSARLSWTAPDAAFDSFYIAYAEPRPDGEAIGLRVPGS |
| | ERSYDLTGLKPGTEYSVLIHGVKGGLLSSPLSAIFTT |
| 589 | MLPAPKNLVVSRVTEDSARLSWTAPDAAFDSFYIAYAEPRPDGEAIVLRVPGS |
| | ERSYDLTGLKPGTEYSVLIHGVKGGLLSSPLAASFTT |
| 590 | MLPAPKNLVVSRVTEDSARLSWTAPDAAFDSFYIAYAEPRPDGEAIQLLVPGS |
| | ERSYDLTGLKPGTEYSVLIHGVKGGLLSSPLVAHFTT |
| 591 | MLPAPKNLVVSRVTEDSARLSWTAPDAAFDSFYIAYAEPRPDGEAIWLLVPGS |
| | ERSYDLTGLKPGTEYSVLIHGVKGGLLSSPLVAFFTT |
| 592 | MLPAPKNLVVSRVTEDSARLSWTAPDAAFDSFFIGYLEPQPPGEAIHLYVPGS |
| | ERSYDLTGLKPGTEYNVTIQGVKGGFPSDPLDAVFTT |
| 593 | MLPAPKNLVVSRVTEDSARLSWTAPDAAFDSFFIGYLEPQPPGEAIYLDVPGS |
| | ERSYDLTGLKPGTEYNVTIQGVKGGFPSTFTT |
| 594 | MLPAPKNLVVSRVTEDSARLSWTAPDAAFDSFYIAYAEPRPDGEAIQLFVPGS |
| | ERSYDLTGLKPGTEYSVLIHGVKGGLLSSPLKAYFTT |
| 595 | MLPAPKNLVVSRVTEDSARLSWTAPDAAFDSFYIAYAEPRPDGEAIVLVVPGS |
| | ERSYDLTGLKPGTEYSVLIHGVKGGLLSSPLSAIFTT |
| 596 | MLPAPKNLVVSRVTEDSARLSWTAPDAAFDSFFIGYLEPQPPGEAIQLTVPGS |
| | ERSYDLTGLKPGTEYNVTIQGVKGGFPSLPLSADFTT |
| 597 | MLPAPKNLVVSRVTEDSARLSWTAPDAAFDSFYIAYAEPRPDGEAILLQVPGS |
| | ERSYDLTGLKPGTEYSVLIHGVKGGLLSSPLDAEFTT |
| 598 | MLPAPKNLVVSRVTEDSARLSWTAPDAAFDSFYIAYAEPRPDGEAIVLAVPGS |
| | ERSYDLTGLKPGTEYSVLIHGVKGGLLSSPLYASFTT |
| 599 | MLPAPKNLVVSRVTEDSARLSWTTPDAAFDSFYIAYAEPRPDGEAIRLQVPGS |
| | ERSYDLTGLKPGTEYSVLIHGVKGGLLSSPLGFTT |
| 600 | MLPAPKNLVVSRVTEDSARLSWTAPDAAFDSFYIAYAEPRPDGEAIQLVVPGS |
| | ERSYDLTGLKPGTEYSVLIHGVKGGLLSSPLYAIFTT |
| 601 | MLPAPKNLVVSRVTEDSARLSWTAPDAAFDSFYIAYAEPRPDGEAISLSVPGS |
| | ERSYDLTGLKPGTEYSVLIHGVKGGLLSSPLHAKFTT |
| 602 | MLPAPKNLVVSRVTEDSARLSWTAPDAAFDSFFIGYLEPQPPGEAIHLGVPGS |
| | ERSYDLTGLKPGTEYNVTIQGVKGGFPSIPLFASFTT |
| 603 | MLPAPKNLVVSRVTEDSARLSWTAPDAAFDSFYIAYAEPRPDGEAIQLLVPGS |
| | ERSYDLTGLKPGTEYSVLIHGVKGGLLSSPLYAAFTT |
| 604 | MLPAPKNLVVSRVTEDSARLSWTAPDAAFDSFFIGYLEPQPPGEAIHLAVPGS |
| | ERSYDLTGLKPGTEYNVTIQGVKGGFPSVPLAAVETT |
| 605 | MLPAPKNLVVSRVTEDSARLSWTAPDAAFDSFYIAYAEPRPDGEAISLQVPGS |
| | ERSYDLTGLKPGTEYSVLIHGVKGGLLSSPLGAHFTT |
| 606 | MLPAPKNLVVSRVTEDSARLSWTAPDAAFDSFYIAYAEPRPDGEAIALWVPGS |
| | ERSYDLTGLKPGTEYSVLIHGVKGGLLSSPLVASFTT |
| 607 | MLPAPKNLVVSRVTEDSARLSWTAPDAAFDSFYIAYAEPRPDGEAIQLHVPGS |
| | ERSYDLTGLKPGTEYSVLIHGVKGGLLSSPLYAFFTT |
| 608 | MLPAPKNLVVSRVTEDSARLSWTAPDAAFDSFYIAYAEPRPDGEAILLHVPGS |
| | ERSYDLTGLKPGTEYSVLIHGVKGGLLSSPLRASFTT |
| 609 | MLPAPKNLVVSRVTEDSARLSWTAPDAAFDSFYIAYAEPRPDGEAIWLGVPGS |
| | ERSYDLTGLKPGTEYSVLIHGVKGGLLSSPLHATFTT |
| 610 | MLPAPKNLVVSRVTEDSARLSWTAPDAAFDSFYIAYAEPRPDGEAIVLEVPGS |
| | ERSYDLTGLKPGTEYSVLIHGVKGGLLSSPLHANFTT |
| 611 | MLPAPKNLVVSRVTEDSARLSWTAPDAAFDSFYIAYAEPRPDGEAILLRVPGS |
| | ERSYDLTGLKPGTEYSVLIHGVKGGLLSSPLYAKFTT |
| 612 | MLPAPKNLVVSRVTEDSARLSWTAPDAAFDSFFIGYLEPQPPGEAIGLWVPGS |
| | ERSYDLTGLKPGTEYNVTIQGVKGGFPSDPLQAVETT |
| 613 | MLPAPKNLVVSRVTEDSARLSWTAPDAAFDSFYIAYAEPRPDGEAIVLHVPGS |
| | ERSYDLTGLKPGTEYSVLIHGVKGGLLSSPLDAFFTT |
| 614 | MLPAPKNLVVSRVTEDSARLSWTAPDAAFDSFYIAYAEPRPDGEAIILHVPGS |
| | ERSYDLTGLKPGTEYSVLIHGVKGGLLSSPLDAYFTT |
| 615 | MLPAPKNLVVSRVTEDSARLSWTAPDAAFDSFYIAYAEPRPDGEAILLAVPGS |
| | ERSYDLTGLKPGTEYSVLIHGVKGGLLSSPLSAKFTT |
| 616 | MLPAPKNLVVSRVTEDSARLSWTAPDAAFDSFFIGYLEPQPPGEAILLFVPGS |
| | ERSYDLTGLKPGTEYNVTIQGVKGGFPSTPLSASFTT |
| 617 | MLPAPKNLVVSRVTEDSARLSWTAPDAAFDSFYIAYAEPRPDGEAIQLTVPGS |
| | ERSYDLTGLKPGTEYSVLIHGVKGGLLSSPLHAYFTT |
| 618 | MLPAPKNLVVSRVTEDSARLSWTAPDAAFDSFYIAYAEPRPDGEAIQLGVPGS |
| | ERSYDLTGLKPGTEYSVLIHGVKGGLLSSPLRAYFTT |
| 619 | MLPAPKNLVVSRVTEDSARLSWTAPDAAFDSFYIAYAEPRPDGEAILLEVPGS |
| | ERSYDLTGLKPGTEYSVLIHGVKGGLLSSPLVAFFTT |
| 620 | MLPAPKNLVVSRVTEDSARLSWTAPDAAFDSFYIAYAEPRPDGEAIQLGVPGS |
| | ERSYDLTGLKPGTEYSVLIHGVKGGLLSSPLLAVFTT |
| 621 | MLPAPKNLVVSRVTEDSARLSWTAPDAAFDSFYIAYAEPRPDGEAIHLRVPGS |
| | ERSYDLTGLKPGTEYNVTIQGVKGGFPSMPLSAIFTT |
| 622 | MLPAPKNLVVSRVTEDSARLSWTAPDAAFDSFYIAYAEPRPDGEAILLQVPGS |
| | ERSYDLTGLKPGTEYSVLIHGVKGGLLSSPLIAKFTT |
| 623 | MLPAPKNLVVSRVTEDSARLSWTAPDAAFDSFYIAYAEPRPDGEAIVLHVPGS |
| | ERSYDLTGLKPGTEYSVLIHGVKGGLLSSPLQAIFTT |
| 624 | MLPAPKNLVVSRVTEDSARLSWTAPDAAFDSFYIAYAEPRPDGEAIALVVPGS |
| | ERSYDLTGLKPGTEYSVLIHGVKGGLLSSPLAANFTT |
| 625 | MLPAPKNLVVSRVTEDSARLSWTAPDAAFDSFYIAYAEPRPDGEAINLSVPGS |
| | ERSYDLTGLKPGTEYSVLIHGVKGGLLSSPLDAYFTT |
| 626 | MLPAPKNLVVSRVTEDSARLSWTAPDAAFDSFYIAYAEPRPDGEAIVLEVPGS |
| | ERSYDLTGLKPGTEYSVLIHGVKGGLLSSPLTASFTT |
| 627 | MLPAPKNLVVSRVTEDSARLSWTAPDAAFDSFYIAYAEPRPDGEAIRLQVPGS |
| | ERSYDLTGLKPGTEYSVLIHGVKGGLLSSPLGASFTT |
| 628 | MLPAPKNLVVSRVTEDSARLSWTAPDAAFDSFYIAYAEPRPDGEAIGLWVPGS |
| | ERSYDLTGLKPGTEYSVLIHGVKGGLLSSPLVAYFTT |
| 629 | MLPAPKNLVVSRVTEDSARLSWTAPDAAFDSFYIAYAEPRPDGEAIYLEVPGS |
| | ERSYDLTGLKPGTEYSVLIHGVKGGLLSSPLFTT |
| 630 | MLPAPKNLVVSRVTEDSARLSWTAPDAAFDSFYIAYAEPRPDGEAIWLDVPGS |
| | ERSYDLTGLKPGTEYSVLIHGVKGGLLSSPLDAYFTT |
| 631 | MLPAPKNLVVSRVTEDSARLSWTAPDAAFDSFAIEYCETKMCGEAIVLTVPGS |
| | ERSYDLTGLKPGTEYRVPIPGVKGGTASLPLSAIFTT |
| 632 | MLPAPKNLVVSRVTEDSARLSWTAPDAAFDSFFIYYIESYPAGEAIVLTVPGS |
| | ERSYDLTGLKPGTEYWVGIDGVKGGRWSTPLSAIFTT |
| 633 | MLPAPKNLVVSRVTEDSARLSWTAPDAAFDSFFIYYIESYPAGEAIVLTVPGS |
| | CRSYDLTGLKPGTEYWVGIDGVKGGRWSTPLSAIFTT |
| 634 | MLPAPKNLVVSRVTEDSARLSWTAPDAAFDSFAIVYHEPRPDGEAIVLTVPGS |
| | CRSYDLTGLKPGTEYEVVILGVKGGVHSYPLSAIFTTAPAPAPAPAPLPAPKN |
| | LVVSRVTEDSARLSWTAPDAAFDSFAIVYHEPRPDGEAIVLTVPGSERSYDLT |
| | GLKPGTEYEVVILGVKGGVHSYPLSAIFTT |
| 635 | MLPAPKNLVVSRVTEDSARLSWTAPDAAFDSFAIVYHEPRPDGEAIVLTVPGS |
| | CRSYDLTGLKPGTEYEVVILGVKGGVHSYPLSAIFTTGGGGSGGGGSGGGGSG |
| | GGGSLPAPKNLVVSRVTEDSARLSWTAPDAAFDSFAIVYHEPRPDGEAIVLIV |
| | PGSERSYDLTGLKPGTEYEVVILGVKGGVHSYPLSAIFTT |
| 636 | MLPAPKNLVVSRVTEDSARLSWTAPDAAFDSFAIVYHEPRPDGEAIVLTVPGS |
| | CRSYDLTGLKPGTEYEVVILGVKGGVHSYPLSAIFTTEAAAKEAAAKEAAAKE |
| | AAAKLPAPKNLVVSRVTEDSARLSWTAPDAAFDSFAIVYHEPRPDGEAIVLTV |
| | PGSERSYDLTGLKPGTEYEVVILGVKGGVHSYPLSAIFTT |
| 637 | MLPAPKNLVVSRVTEDSARLSWTAPDAAFDSFAIVYHEPRPDGEAIVLTVPGS |
| | CRSYDLTGLKPGTEYEVVILGVKGGVHSYPLSAIFTTEAAAKLPAPKNLVVSR |
| | VTEDSARLSWTAPDAAFDSFAIVYHEPRPDGEAIVLTVPGSERSYDLIGLKPG |
| | TEYEVVILGVKGGVHSYPLSAIFTT |
| 638 | MLPAPKNLVVSRVTEDSARLSWTAPDAAFDSFKIEYFEYVGYGEAIVLTVPGS |
| | CRSYDLTGLKPGTEYYVAIYGVKGGWYSRPLSAIFTTAPAPAPAPAPLPAPKN |
| | LVVSRVTEDSARLSWTAPDAAFDSFKIEYFEYVGYGEAIVLTVPGSERSYDLT |
| | GLKPGTEYYVAIYGVKGGWYSRPLSAIFTT |
| 639 | MLPAPKNLVVSRVTEDSARLSWTAPDAAFDSFKIEYFEYVGYGEAIVLTVPGS |
| | CRSYDLTGLKPGTEYYVAIYGVKGGWYSRPLSAIFTTGGGGSGGGGSGGGGSG |
| | GGGSLPAPKNLVVSRVTEDSARLSWTAPDAAFDSFKIEYFEYVGYGEAIVLIV |
| | PGSERSYDLTGLKPGTEYYVAIYGVKGGWYSRPLSAIFTT |
| 640 | MLPAPKNLVVSRVTEDSARLSWTAPDAAFDSFKIEYFEYVGYGEAIVLTVPGS |
| | CRSYDLTGLKPGTEYYVAIYGVKGGWYSRPLSAIFTTEAAAKEAAAKEAAAKE |
| | AAAKLPAPKNLVVSRVTEDSARLSWTAPDAAFDSFKIEYFEYVGYGEAIVLTV |
| | PGSERSYDLTGLKPGTEYYVAIYGVKGGWYSRPLSAIFTT |
| 641 | MLPAPKNLVVSRVTEDSARLSWTAPDAAFDSFKIEYFEYVGYGEAIVLTVPGS |
| | CRSYDLTGLKPGTEYYVAIYGVKGGWYSRPLSAIFTTEAAAKLPAPKNLVVSR |
| | VTEDSARLSWTAPDAAFDSFKIEYFEYVGYGEAIVLTVPGSERSYDLIGLKPG |
| | TEYYVAIYGVKGGWYSRPLSAIFTT |
| 642 | MLPAPKNLVVSRVTEDSARLSWTAPDAAFDSFAIVYHEPRPDGEAIVLTVPGS |
| | CRSYDLTGLKPGTEYEVVILGVKGGVHSYPLSAIFTT |
| 643 | MLPAPKNLVVSRVTEDSARLSWTAPDAAFDSFAIVYHEPRPDGEAIVLTVPGS |
| | ERSYDLTGLKPGTEYEVVILGVKGGVHSYPLSAIFTT |
| 644 | MLPAPKNLVVSRVTEDSARLSWTAPDAAFDSFFIGYLEPQPPGEAIHLGVPGS |
| | CRSYDLTGLKPGTEYNVTIQGVKGGFPSIPLFASFTT |
| 663 | MLPAPKNLVVSRVTEDSARLSWTAPDAAFDSFFIGYLEPQPPGEAIVLTVPGS |
| | ERSYDLTGLKPGTEYNVTIQGVKGGFPSMPLSAIFTT |
| 664 | MLPAPKNLVVSRVTEDSARLSWTAPDAAFDSFIIPYAEVRPDGEAIVLTVPGS |
| | ERSYDLTGLKPGTEYSVLIHGVKGGKLSLPLSAIFTT |
| 665 | MLPAPKNLVVSRVTEDSARLSWTAPDAAFDSFYIAYAEPRPDGEAIVLTVPGS |
| | ERSYDLTGLKPGTEYSVLIHGVKGGLLSSPLSAIFTT |
| 666 | MLPAPKNLVVSRVTEDSARLSWTAPDAAFDSFYIVYAEPIPNGEAIVLTVPGS |
| | ERSYDLTGLKPGTEYSVLIHGVKGGRNSDPLSAIFTT |
| 667 | MLPAPKNLVVSRVTEDSARLSWTAPDAAFDSFDIGYTEYGGYGEAIVLTVPGS |
| | ERSYDLTGLKPGTEYWVLIQGVKGGGSSVPLSAIFTT |
| 668 | MLPAPKNLVVSRVTEDSARLSWTAPDAAFDSFHIVYHEPRPSGEAIVLTVPGS |
| | ERSYDLTGLKPGTEYEVGIVSVKGGDLSVPLSAIFTT |
| 669 | MLPAPKNLVVSRVTEDSARLSWTAPDAAFDSFAIVYHEPRPDGEAIVLTVPGS |
| | ERSYDLTGLKPGTEYEVVILGVKGGVHSYPLSAIFTT |
| 670 | MLPAPKNLVVSRVTEDSARLSWTAPDAAFDSFLIPYAETSPSGEAIVLTVPGS |
| | ERSYDLTGLKPGTEYSVLIHGVKGGDYSSPLSAIFTT |
| 671 | MLPAPKNLVVSRVTEDSARLSWTAPDAAFDSFPIAYAEPRPDGEAIVLTVPGS |
| | ERSYDLTGLKPGTEYSVLIHGVKGGLLSSPLSAISTT |
| 672 | MLPAPKNLVVSRVTEDSARLSWTAPDAAFDSFYIAYAEPRPDGEAILLQVPGS |
| | ERSYDLTGLKPGTEYSVLIHGVKGGLLSSPLTAIFTT |
| 1395 | MLPAPKNLVVSRVTEDSARLSWTAPDAAFDSFYIAYAEPRPDGEAILLLVPGS |
| | ERSYDLTGLKPGTEYSVLIHGVKGGLLSSPLVASFTT |
| 1396 | MLPAPKNLVVSRVTEDSARLSWTAPDAAFDSFGIGYWERRWYGEAIVLTVPGS |
| | ERSYDLTGLKPGTEYEVTIRGVKGGGYSGPLSAIFTT |
| 1397 | MLPAPKNLVVSRVTEDSARLSWTAPDAAFDSFDIGYTEYGGYGEAIILQVPGS |
| | ERSYDLTGLKPGTEYWVLIQGVKGGGSSVPLVAYFTT |
| 1398 | MLPAPKNLVVSRVTEDSARLSWTAPDAAFDSFYIAYAEPRPDGEAIILGVPGS |
| | ERSYDLTGLKPGTEYSVLIHGVKGGLLSSPLSAYSTT |
| 1399 | MLPAPKNLVVSRVTEDSARLSWTAPDAAFDSFFIGYLEPQPPGEAIHLIVPGS |
| | ERSYDLTGQKPGTEYNVTIQGVKGGFPSDPLVASFTT |
| 1400 | MLPAPKNLVVSRVTEDSARLSWTAPDAAFDSFHIVYHEPRPSGEAIWLWVPGS |
| | ERSYDLTGLKPGTEYEVGIVSVKGGDLSVPLSAIFTT |
| 1401 | MLPAPKNLVVSRVTEDSARLSWTAPDAVFDSFYIAYAEPRPDGEAIGLYVPGS |
| | ERSYDLTGLKPGTEYSVLIHGVKGGLLSSPLTASFTT |
| 1402 | MLPAPKNLVVSRVTEDSARLSWTAPDAAFDSFFIGYLEPQPPGEAIGLQVPGS |
| | ERSYDLTGLKPGTEYNVTIQGVKGGFPSMPLSAIFTT |
| 1403 | MLPAPKNLVVSRVTEDSARLSWTAPDAAFDSFYIAYAEPRPDGEAILLQVPGS |
| | ERSYDLTGLKPGTEYSVLIHGVKGGLLSSPLVAKFTT |
| 1404 | MLPAPKNLVVSRVTEDSARLSWTAPDAAFDSFFIGYLEPQPPGEAIILHVPGS |
| | ERSYDLTGLKPGTEYNVTIQGVKGGFPSLPLNANFTT |
| 1405 | MLPAPKNLVVSRVTEDSARLSWTAPDAAFDSFHIVYHEPRPSGEAIYLEVPGS |
| | ERSYDLTGLKPGTEYEVGIVSVKGGDLSVPLRAHFTT |
| 1406 | MLPAPKNLVVSRVTEDSARLSWTAPDAAFDSFFIGYLEPQPPGEAISLWVPGS |
| | ERSYDLTGLKPGTEYNVTIQGVKGGFPSIPLWAIFTT |
| 1407 | MLPAPKNLVVSRVTEDSARLSWTAPDAAFDSFFIGYLEPQPPGEAIDLWVPGS |
| | ERSYDLTGLKPGTEYNVTIQGVKGGFPSMPLSAIFTT |
| 1408 | MLPAPKNLVVSRVTEDSARLSWTAPDAAFDSFFIGYLEPQPPGEAIYLLVPGS |
| | ERSYDLTGLKPGTEYNVTIQGVKGGFPSDPLSAIFTT |
| 1409 | MLPAPKNLVVSRVTEDSARLSWTAPDAAFDSFHIVYHEPRPSGEAIWLFVPGS |
| | ERSYDLTGLKPGTEYEVGIVSVKGGDLSVPLSAIFTT |
| 1410 | MLPAPKNLVVSRVTEDSARLSWTAPDAAFDSFFIGYLEPQPPGEAIDLYVPGS |
| | ERSYDLTGLKPGIEYNVTIQGVKGGFPSLPLQAHFTT |
| 1411 | MLPAPKNLVVSRVTEDSARLSWTAPDAAFDSFYIAYAEPRPDGEAILLLVPGS |
| | ERSYDLTGLKPGTEYSVLIHGVKGGLLSSPLIAGFTT |
| 1412 | MLPAPKNLVVSRVTEDSARLSWTAPDAAFDSFFIGYLEPQPPGEAIWLLVPGS |
| | ERSYDLTGLKPGTEYNVTIQGVKGGFPSTPLSAIFTT |
| 1413 | MLPAPKNLVVSRVTEDSARLSWTAPDAAFDSFYIAYAEPRPDGEAITLWVPGS |
| | ERSYDLTGLKPGTEYSVTIHGVKGGLLSSPLSAIFTT |
| 1414 | MSLPAPKNLVVSRVTEDSARLSWTAPDAAFDSFFIGYLEPQPPGEAIELYVPG |
| | SERSYDLTGLKPGTEYNVTIQGVKGGFPSDPLVAFFTT |
| 1415 | MSLPAPKNLVVSRVTEDSARLSWTAPDAAFDSFEIAYQELSLWGEAIVLTVPG |
| | SERSYDLTGLKPGTEYWVQIGGVKGGEWSAPLSAIFTT |
| 1416 | MSLPAPKNLVVSRVTEDSARLSWTAPDAAFDSFFIGYLEPQPPGEAITLYVPG |
| | SERSYDLTGLKPGTEYNVTIQGVKGGFPSAPLSAIFTT |
| 1417 | MSLPAPKNLVVSRVTEDSARPSWTAPDAAFDSFEIGYFENLYLGEAIVLTVPG |
| | SERSYDLTGLKPGTEYYVHIGGVKGGIWSSPLSAYFTT |
| 1418 | MSLPAPKNLVVSRVTEDSARLSWTAPDAAFDSFAIRYWELRATGEAIPLSVPG |
| | SERSYDLTGLKPGTEYHVAISGVKGGKSSYPLRASFTT |
| 1419 | MSLPAPKNLVVSRVIEDSARLSWTAPDAAFDSFEIGYFENLYLGEAIVLTVPG |
| | SERSYDLTGLKPGTEYWVQIGGVKGGNWSAPLSAIFTT |
| 1420 | MSLPAPKNLVVSRVTEDSARLSWTAPDAAFDSFYIAYAEPRPDGEAIILLVPG |
| | SERSYDLTGLKPGTEYSVLIHGVKGGLLSSPLHAHFTT |
| 1421 | MSLPAPKNLVVSRVTEDSARLSWTAPDAVFDSFEIAYQELSLWGEAIVLTVPG |
| | SERSYDLTGLKPGTEYYVHIGGVKGGIWSSPLSAYFTT |
| 1422 | MSLPAPKNLVVSRVTEDSARLSWTAPDAAFDSFFIGYLEPQPPGEAITLLVPG |
| | SERSYDLTGLKPGTEYNVTIQGVKGGFPSGPLQASFTT |
| 1423 | MSSLPAPKNLVVSRVTEDSARLSWTAPDAAFDSFEIGYFENLYLGEAIVLTVP |
| | GSERSYDLTGLKPGTEYWVQIGGVKGGNWSAPLSAIFTT |
| 1424 | MGGSLPAPKNLVVSRVTEDSARLSWTAPDAAFDSFEIGYFENLYLGEAIVLIV |
| | PGSERSYDLTGLKPGTEYWVQIGGVKGGNWSAPLSAIFTT |
| 1425 | MSLPAPKNLVVSRVTEDSARLSWTAPDAAFDSFEIAYFERTWFGEAIVLTVPG |
| | SERSYDLTGLKPGTEYWVQIGGVKGGDWSAPLSAIFTT |
| 1426 | MSLPAPKNLVVSRVTEDSARLSWTAPDAAFDSFEIAYFENDWAGEAIVLTVPG |
| | SERSYDLTGLKPGTEYVVFIGGVKGGDFSPPLSAIFTT |
| 1427 | MSLPAPKNLVVSRVTEDSARLSWTAPDAAFDSFEIGYFENLYLGEAIVLTVPG |
| | SERSYDLTGLKPGTEYWVQIGGVKGVNWSAPLSAIFTT |
| 1428 | MSLPAPKNLVVSRVTEDSARLSWTAPDAAFDSFKIKYAEYDRYGEAIALFVPG |
| | SERSYDLTGLKPGTEYFVHIDGVKGGTDSQPLVASFTT |
| 1429 | MSLPAPKNLVVSRVTEDSARLSWTAPDAAFDSEDINYWENESGGEAIALFVPG |
| | SERSYDLTGLKPGTEYLVTIAGVKGGWWSKPLYATFTT |
| 1430 | MSLPAPKNLVVSRVTEDSARLSWTAPDAAFDSFFIGYLEPQSPGEAIALYVPG |
| | SERSYDLTGLKPGTEYNVTIQGVKGGFPSEPLIANFTT |
| 1431 | MSLPAPKNLVVSRVTEDSARLSWTAPDAAFDSFEIDYVEFIFTGEAIGLIVPG |
| | SERSYDLTGLKPGTEYWVTIAGVKGGEWSTPLQAFLTT |
| 1432 | MSLPAPKNLVVSRVTEDSARLSWTAPDAAFDSFFIGYLEPQPPGEAITLYVPG |
| | SERSYDLTGLKPGTEYNVTIQGVKGGFPSDPLVAHFTT |
| 1433 | MSLPAPKNLVVSRVTEDSARLSWTAPDAAFDSFFIGYLEPQPPGEVIHLFVPG |
| | SERSYDLTGLKPGTEYNVTIQGVKGGFPSMPLSAIFTT |
| 1434 | MSLPAPKNLVVSRVTEDSARLSWTAPDAAFDSFEIGYFENLYLGEAIVLTVPG |
| | SERSYDLTGLKPGTEYWVQIGGVKGGAYSTPLSAIFTT |
| 1435 | MSLPAPKNLVVSRVTEDSARLSWTAPDAAFDSFFIGYLEPQPPGEAIHLYVPG |
| | SERSYDLTGLKPGTEYNVTIQGVKGGFPSMPLSAIFTT |
| 1436 | MSLPAPKNLVVSRVTEDSARLSWTAPDAAFDSFEIGYFENLYLGEAIVLTVPG |
| | SERSYDLTGLKPGTEYWVQIGGVKGGNWSAPLSAYFTT |
| 1437 | MSLPAPKNLVVSRVTEDSARLSWTASDAAFDSFHIWYFEKANEGEAIPLVVPG |
| | SERSYDLTGLKPGTEYDVDIGGVKGGAWSIPLGARFTT |
| 1438 | MSLPAPKNLVVSRVTEDSARLSWTAPDAAFDSFAIQYVEVIGSGEAIELIVPG |
| | SERSYDLTGLKPGTEYHVYIDGVKGGKDSKPLYAGFTT |
| 1439 | MSLPAPKNLVVSRVTEDSARLSWTAPDAAFDSFEIGYFENLYLGEAIVLTVPG |
| | SERSYDLTSLKPGTEYWVQIGGVKGGNWSAPLSATFTT |
| 1440 | MSLPAPKNLVVSRVTEDSARLSWTAPDAAFDSFFIGYLEPQPPGEAIALLVPG |
| | SERSYDLTGLKPGTEYNVTIQGVKGGFPSHPLSAKFTT |
| 1441 | MSLPAPKNLVVSRVTEDSARLSWTAPDAAFDSFDIGYFENTYEGEAIVLTVPG |
| | SERSYDLTGLKPGTEYVVWIGGVKGGSYSSPLSAIFTT |
| 1442 | MSLPAPKNLVVSRVTEDSARLSWTAPDAAFDSFFIGYLEPQPPGEAIELFVPG |
| | SERSYDLTGLKPGTEYNVTIQGVKGGFPSGPLIASFTT |
| 1443 | MSLPAPKNLVVSRVTEDSARLSWTAPDAAFDSFEIVYQENSAYGEAIVLTVPG |
| | SERSYDLTGLKPGTEYWVQIGGVKGGEWSAPLSAIFTT |
| 1444 | MSLPAPKNLVVSRVTEDSARLSWTAPDAAFDSFDIGYFELRYYGEAIVLTVPG |
| | SERSYDLTGLKPGTEYWVSIGGVKGGTYSAPLSAIFTT |
| 1445 | MSLPAPKNLVVSRVTEDSARLSWTAPDAAFDSFDIGYFENSYAGEAIVLTVPG |
| | SERSYDLIGLKPGTEYYVSIAGVKGGRFSPPLSAIFTT |
| 1446 | MSLPAPKNLVVSRITEDSARLSWTAPDAAFDSFEIAYFETCRTGEAIVLTVPG |
| | SERSYDLTGLKPGTEYRVWIGGVKGGVWSRPLSAIVTT |
| 1447 | MSLPAPKNLVVSRVTEDSARLSWTAPDAAFDSFDIGYFEATYYGEAIVLIVPG |
| | SERSYDLTGLKPGTEYWVQIGGVKGGNWSAPLSAIFTT |
| 1448 | MSLPAPKNLVVSRVTEDSARLSWTAPDAAFDSFYIAYAEPRPDGEAIVLAVPG |
| | SERSYDLTGLKPGTEYSVLIHGVKGGLLSSPLGADFTT |
| 1449 | MSLPAPKNLVVSRVTEDSARLSWTAPDAAFDSFEIGYFENLYLGEAIVLTVPG |
| | SERSYDLTGLKPGTEYWVQIGGVKGGNWSAPLSAIFTT |
| 1450 | MSLPAPENLVVSRVTEDSARLSWTAPDAAFDSFEIGYFENLYLGEAIVLTVPG |
| | SERSYDLTGLKPGTEYWVQIGGVKGGVWSRPLSAIFTT |
| 1451 | MSLPAPKNLVVSRVTEDSARLSWTAPDAAFDSFEIGYFEMSWTGEAIVLTVPG |
| | SERSYDLTGLKPGTEYVVFIGGVKGGNWSAPLSAIFTT |
| 1452 | MSLPAPKNLVVSRVTEDSARLSWTAPDAAFDSFEIGYGEFTYYGEAIVLTVPG |
| | SERSYDLTGLKPGTEYWVQIGGVKGGNWSNPLSAIFTT |
| 1453 | MSLPAPKNLVVSRVTEDSARLSWTAPDAAFDSFEIAYFERTWEGEAIVLTVPG |
| | SERSYDLTGLKPGTEYWVQIGGVKGGDWSKPLSAIFTT |
| 1454 | MSLPAPKNLVVSRVTEDSARLSWTAPDAAFDSFEIVYQENSAYGEAIVLIVPG |
| | SERSYDLTGLKPGTEYWVQIGGVKGGNWSAPLSAISTT |
| 1455 | MSLPAPKNLVVSRVTEDSARLSWTAPDAAFDSFEIGYFENLYLGEAIVLTVPG |
| | SERSYDLTGLKPGTEYWVQIGGVKGGNWSVPLSAIFTT |
| 1456 | MSLPAPKNLVVSRVTEDSARLSWTAPDAAFDSFFIGYLEPQPPGEAIVLTVPG |
| | SERSYDLTGLKPGTEYNVTIQGVKGGFPSEPLVAHFTT |
| 1457 | MSLPAPKNLVVSRVTEDSARLSWTALDAAFDSFEISYWEHTENGEAIILFVPG |
| | SERSYDLTGLKPGTEYYVTIGGVKGGAWSPPLWAEFTT |
| 1458 | MSLPAPKNLVVSRVTEDSARLSWTAPDAAFDSFFIGYLEPQPPGEVIHLYVPG |
| | SERSYDLTGLKPGTEYVVFIGGVKGGVWSVPLSAIFTT |
| 1459 | MSLPAPKNLVVSRVTEDSARLSWTAPDAAFDSFFIGYLEPQPPGEAIHLYVPG |
| | SERSYDLTGLKPGTEYWVQIGGVKGGNWSAPLSAIFTT |
| 1460 | MSLPAPKNLVVSRVTEDSARLSWTAPDAAFDSFYIAYAEPRPDGEAILLEVPG |
| | SERSYDLAGLKPGTEYSVLIHGVKGGLLSSPLGAEFTT |
| 1461 | MSLPAPKNLVVSRVTEDSARLSWTAPDAAFDSFEIGYFENLYLGEAIVLTVPG |
| | SERSYDLTGLKPGTEYWVQIGGIKGGNWSAPLSAIFTT |
| 1462 | MSLPAPKNLVVSRVTEDSARLSWTAPDAAFDSFFIGYLEPQPPGEAIVLTVPG |
| | SERSYDLTGLKPGTEYNVTIQGVKGGFPSVPLSAAFTT |
| 1463 | MSLPAPKNLVVSRVTEDSARLSWTAPDAAFDSFIIGYLEPQPPDEAIALYVPG |
| | SERSYDLTGLKPGTEYNVTIQGVKGGFPSLPLVATFTT |
| 1464 | MSLPAPKNLVVSRVTEDSARLSWTAPDAAFDSFEIGYFENLYLGEAIVLTVPG |
| | SERSYDLTGLKPGTEYWVQIGGVKGGNWSAPLSATFTT |
| 1465 | MSLPAPKNLVVSRVTEDSARLSWTAPDAAFDSFEIGYFENLYLGEAIVLIVPG |
| | SERSYDLTGLKPGTEYWVQIGGVKGGNWSTPLSAIFTT |
| 1466 | MSLPAPKNLVVSRVTEDSARLSWTAPDAAFDSFEIGYFENLYLGEAIVLIVPG |
| | SERSYDLTGLKPGTEYWVQIGGVKGGEWSSPLSAIFTT |
| 1467 | MSRPAPKNLVVSRVTEDSARLSWTAPGAAFDSFEIAYFERTWEGEAIVLTVPS |
| | SERSYDLTGLKPGTEYWVQIGGVKGGDWSKPLSAIFTT |
| 1468 | MSLPAPKNLVVSRVTEDSARLSWTAPDAAFDSFEIAYQELSLWGEAIVLTVPG |
| | SERSYDLTGLKPGTEYYVHIGGVKGGIWSSPLVAHFTT |
| 1469 | MSLPAPKNLVVSRVTEDSARLSWTAPNAAFDSFEIGYFENLYLGEAIVLTVPG |
| | SERSYDLTGLKPGTEYWVQIGGVKGGNWSAPLSAIFTT |
| 1470 | MSLPAPKNLVVSRVTEDSARLSWTAPDAAFDSFEIAYQELSLWGEAIVLIVPG |
| | SERSYDLTGLKPGTEYVVFIGGVKGGVWSVPLSAIFTT |
| 1471 | MSLPAPKNLVVSRVTEDSARLSWTAPDAAFDSFFIGYLEPQPPGEAIHLFVPG |
| | SERSYDLTGLKPGTEYWVQIGGVKGGNWSAPLSAIFTT |
| 1472 | MSLPAPKNLVVSRVTEDSARLSWTAPDAAFDSFEIGYFENLYLGEAIVLIVPG |
| | SERSYDLTSLKPGTEYWVQIGGVKGGNWSAPLSAIFTT |
| 1473 | MGSLPAPKNLVVSRVTEDSARLSWTAPDAAFDSFFIGYLEPQPPGEAISLYVP |
| | GSERSYDLTGLKPGTEYNVTIQGVKGGFPSDPLVAHFTT |
| 1474 | MSLPAPKNLVVSRVTEDSARLSWTAPDAAFDSFFIGYLEPQPPGEAITLLVPG |
| | SERSYDLTGLKPGTEYNVTIQGVKGGFPSHPLVATTTT |
| 1475 | MSLPAPKNLVVSRVTEDSARLSWTAPDAAFDSFFIGYLEPQPPGEAISLYVPG |
| | SERSYDLTGLKPGTEYNVTIQGVKGGFPSEPLIANFTT |
| 1476 | MSLPAPKNLVVSRVTEDSARLSWTAPDAAFDSFEIGYFENDYFGEAIVLTVPG |
| | SERSYDLTGLKPGTEYWVQIGGVKGGNWSAPLSAIFTT |
| 1477 | MSLPAPENLVVSRVTEDSARLSWTAPDAAFDSFEIGYWENAYKGEAIVLTVPG |
| | SERSYDLTGLKPGTEYVVFIGGVKGGVWSVPLSVIFTT |
| 1478 | MSLPAPKNLVISRVTEDSARLSWTAPDAAFDSFEIVYAEPVRFGEAIVLTVPG |
| | SERSYDLTGLKPGTEYWVQIGGVKGGEWSSPLSAIFTT |
| 1479 | MSLPAPKNLVVSRVTEDSARPSWTAPDAAFDSFEIAYFERTWFGEAIVLTVPG |
| | SERSYDLTGLKPGTEYWVQIGGVKGGDWSKPLSAIFTT |
| 1480 | MGSLPAPKNLVVSRVTEDSARLSWTAPDAAFDSFEISYWEHTENGEAIILFVP |
| | GSERSYDLTGLKPGTEYWVQIGGVKGGNWSAPLSAIFTT |
| 1481 | MSLPAPKNLVVSRVTEDSARLSWTAPDAAFDSFEIGYFENPYLGEAIVLIVPG |
| | SERSYDLTGLKPGTEYWVQIGGVKGGNWSAPLSAIFTT |
| 1482 | MSLPAPKNLVVSRVTEDSARLSWTAPDAAFDSFEIGYWENAYKGEAIVLTVPG |
| | SERSYDLTGLKPGTEYRVWIGGVKGGVWSRPLSAIFTT |
| 1483 | MSLPAPKNLVVSRVTEDSARLSWTAPDAAFDSFEIGYWENAYKGEAIVLTVPG |
| | SERSYDLTGLKPGTEYVVFIGGVKGGHGSQPLSAIFTT |
| 1484 | MSLPAPKNLVVSRVTEDSARLSWTAPDAAFDSFEIGYFEHTYYGEAIVLTVPG |
| | SERSYDLTGLKPGTEYWVQIGGVKGGNWSAPLSAIFTT |
| 1485 | MSLPAPKNLVISRVTEDSARLSWTAPDAAFDSFEIGYFENLYLGEAIVLTVPG |
| | SERSYDLTGLKPGTEYWVQIGGVKGGNWSAPLSAISTT |
| 1486 | MSLPAPKNLVVSRVTEDSARLSWTAPDAAFDSFEIGYFENLYLGEAIVLTVPG |
| | SERSYDLAGLKPGTEYWVQIGGVKGGNWSAPLSAIFTT |
| 1487 | MSLPAPKNLVVSRVTEDSARLSWTAPDAAFDSFEIGYFENLYLGEAIVLTVPG |
| | SERSYDLTGLKPGTEYWVQIGGVKGGWGSNPLSAIFTT |
| 1488 | MSLPAPKNLVVSRVTEDSARLSWTAPDAAFDSFEIGYFENLYLGEAIVLTVPG |
| | SERSYDLTGLKPGTEYWVQIGGVKGGNWSEPLSAIFTT |
| 1489 | MSLPAPKNLVVSRVTEDSARLSWTAPDAAFDSFEIGYFENLYLGEAIVLTVPG |
| | SERSYDLTGLKPGTEYPVWIGGVKGGTWSVPLSTIFTT |
| 1490 | MSLPAPKNLIVSRVTEDSARLSWTAPDAAFDSFEIGYFENLYLGEAIVLTVPG |
| | SERSYDLTGLKPGTEYWVQIGGVKGSNWSAPLSAIFTT |
| 1491 | MSLPAPKNLVVSRVTEDSARLSWTAPDAAFDSFEIGYFENLYLGEAIVLTVPG |
| | SERSYDLTGLKPGTEYWVQIGGVKGGNWSAPLSAIFTT |
| 1492 | MSLPAPKNLVVSRVTEDSARLSWTAPDAAFDSFFIGYLEPQPPGEAISLLVPG |
| | SERSYDLTGLKPGTEYNVTIQGVKGGFPSEPLHAIFTT |
| 1493 | MSLPAPKNLVVSRVTEDSARLSWTAPDAAFDSFLIGYTEYSVYGEAIVLTVPG |
| | SERSYDLTGLKPGTEYTVWIMGVKGGIKSTPLSAISTT |
| 1494 | MSLPAPKNLVVSRVTEDSARLSWTAPDAAFDSFEIGYFENLYLGEAIVLTVPG |
| | YERSYDLTGLKPGTEYWVQIGGVKGGNWSAPLSAIFTT |
| 1495 | MSLPAPKNLVVSRVTEDSARLSWTAPDAAFDSFEIGYFENLYLGEAIVLTVPG |
| | SERSYDLTGLKPGTEYWVQIGGVKGSNWSAPLSAIFTT |
| 1496 | MSLPAPKNLVVSRVTEDSARLSWTAPDAAFDSFEIAYFEGTFHGEAIVLFVPG |
| | SERSYDLTGLKPGTEYAVWIGGVKGGDYSRPLSAAFTT |
| 1497 | MSLPAPKNLVVSRVTEDSARLSWTAPDAAFDSFEIGYFENLYLGEAIVLIVPG |
| | SERSYDLTGLKPGTEYYVHIGGVKGGIWSSPLSAIFTT |
| 1498 | MSLPAPKNLVVSRVTEDFARLSWTAPDAAFDSFEIAYWEQSYTGEAIVLTVPG |
| | SERSYDLTGLKPGTEYFVHIGGVKGGVWSTPLSAIFTT |
| 1499 | MSLPAPKNLVVSRVTEDSARLSWTAPDAAFDSFLIQYDEWPTYGEAIVLIVPG |
| | SERSYDLTGLKPGTEYLVEIVGVKGGNLSGPLSAIFTT |
| 1500 | MSLPAPKNLVVSRVTEDSARLSWTAPDAAFDSFEIGYFENLYLGEAIHLFVPG |
| | SERSYDLTGLKPGTEYNVTIQGVKGGFPSEPLIANFTT |
| 1501 | MSLPAPKNLVASRVTEDSARLSWTAPDAAFDSFEIGYFENLYLGEAIVLIVPG |
| | SERSYDLTGLKPGTEYWVQIGGVKGGNWSAPLSAIFTT |
| 1502 | MSLPAPKNLVVSRVTEDSARLSWTAPDAAFDSFEIGYWENAYKGEAIVLTVPG |
| | SERSYDLTGLKPGTEYVVFIGGVKGGVWSVPLSAISTT |
| 1503 | MSLPAPKNLVVSRVTEDSARLSWTAPDAAFDSFFIGYFENLYLGEAIVLTVPG |
| | SERSYDLTGLKPGTEYWVQIGGVKGGNWSAPLSAIFTT |
| 1504 | MSLPAPKNLVVSRVTEDSARLSWTAPDAAFDSFEIAYFERTWFGEAIVLTVPG |
| | SERSYDLTGLKPGTEYWVQIGGVKGGDWSRPLSAIFTT |
| 1505 | MSLRRRKTWLFLALPKTLRVCLGPRRTRRSTLSKSATSKTCTWVKRSF*PFRV |
| | LNVLTT*PV*NRVPNTRLRSPVLKVVRCLTRCLRSSPPVG |
| 1506 | MSLPAPKNLVVSRVTEDSARLSWTAPDAAFDSFEIVYAEPVRFGEAIVLIVPG |
| | SERSYDLTGLKPGTEYWVQIGGVKGGEWSSLLSAIFTT |
| 1507 | MSLPAPKNLVVSRVTEDSARLSWTAPDAAFDSFEIGYFENLYLGEAIVLIVPG |
| | SERYYDLTGLKPGTEYWVQIGGVKGGNWSAPLSAIFTT |
| 1508 | MSLPAPKNLVVSRVTEDSARLSWTAPDAAFDSFEIGYFENLYLGEAIVLTVPG |
| | SERSYDLTGLKPGTEYWVQIGGAKGGNWSAPLSAIFTT |
| 1509 | MSLPAPKNLVVSRVTEDSARLSWTAPDAAFDSFEIDYGEYSQAGEAIGLLVPG |
| | SERSYDLTGLKPGTEYWVQIGGVKGGNWSAPLSAIFTT |
| 1510 | MSLPAPKNLVVSRVTEDSARLSWTAPDAAFDSLEIGYFENLYLGEAIVLTVPG |
| | SERSYDLTGLKPGTEYWVQIGGVKGGNWSAPLSAIFTT |
| 1511 | MSLPAPKNLVVSRVTEDSARLSWTAPDAAFDSFEIAYFEQTYTGEAIVLIVPG |
| | SERSYDLTGLKPGTEYWVQIGGVKGGTWSAPLSAISTT |
| 1512 | MSLPAPKNLVVSRVTEDSARLSWTAPDAAFDSFEIAYFEQTYTGEAIVLIVPG |
| | SERSYDLTGLKPGTEYWVAIGGVKGGLWSLPLSAIFTT |
| 1513 | MSLPAPKNLVVSRVTEDSARLSWTAPDAAFDSFEIAYHEDDFYGEAIALLVPG |
| | SERSYDLTGLKPGTEYIVHIGGVKGGFFSSPLYAWFTT |
| 1514 | MSLPAPKNLVVSRVTEDSARLSWTAPDAAFDSFAIMYGERGNPGEAIVLTVPG |
| | SERSYDLIGLKPGTEYAVWIYGVKGGNYSYPLSAIFTT |
| 1515 | MSLPAPKNLVVSRVTEDSARLSWTAPDAAFDSFEIAYWEQSYTGEAIVLTVPG |
| | SERSYDLTGLKPGTEYWVQIGGVKGGNWSAPLSAIFTT |
| 1516 | MSLPAPKNLVVSRVTEDSARLSWTAPDAAFDSFFIGYLEPRPPGEAIHLYVPG |
| | SERSYDLTGLKPGTEYNITIQGVKGGFPSIPLIASFTT |
| 1517 | MSLPAPKNLVVSRVTEDSARLSWTAPDAAFDSFFIGYLEPQPPGEAITLYVPG |
| | SERSYDLTGLKPGTEYNVTIQGVKGGFPSGPLIASFTT |
| 1518 | MSLPAPKNLVVSRVTEDSARLSWTAPDAAFDSFEIAYFERTWFGEAIVLTVPG |
| | SERSYDLTGLKPGTEYWVAIGGVKGGLWSLPLSVIFTT |
| 1519 | MSLPAPKNLVVSRVTEDSARLSWTAPDAAFDSFEIGYWENAYKGEAIVLTVPG |
| | SERSYDLTGLKPGTEYVVFIGGVKGGGWSGPLSAIFTT |
| 1520 | MSLPAPKNLVVSRVTEDSARLSWTAPDAAFDSFEIGYFENLYLGEAIVLTVPG |
| | SERSYDLTGLKPGTEYWVQIGGVKGGNWSAPLSAIFTT |
| 1521 | MSLPAPKNLVISRVTEDSARLSWTAPDAAFDSFEIGYFENLYLGEAIVLTVPG |
| | SERSYDLTGLKPGTEYWVAIGGVKGGLWSLPLSAIFTT |
| 1522 | MSLPAPKNLVVSRVTEDSARLSWTAPDAAFDSFEIAYAELSTGEAIVLTVPGS |
| | ERSYDLTGLKPGTEYYVHIGGVKGGSWSIPLSAIFTT |
| 1523 | MSLPAPKNLVVSRVTEDSARLSWTAPDAAFDSFEIGYWENAYKGEAIVLTVPG |
| | SERSYDLTGLKPGTEYVVFIGGVKGGVWSKPLSVIFTT |
| 1524 | MSLPAPKNLVVSRVTEDSARLSWTAPDAAFDSFSIMYAEQKVNGEAIVLIVPG |
| | SERSYDLTGLKPGTEYLVLIWGVKGGGRSLPLSAIFTT |
| 1525 | MSLPAPKNLIVSRVTEDSARLSWTAPDAAFDSLEIGYFENLYLGEAIVLTVPG |
| | SERSYDLTGLKPGTEYWVQIGGVKGGNWSAPLSAIFTT |
| 1526 | MSLPAPKNLVVSRVTEDSARLSWTAPDAAFDSFFIGYLEPQPPGEAIHLFVPG |
| | SERSYDLTGLKPGTEYNVTIQGVKGGFPSEPLIANSTT |
| 1527 | MSLPAPKNLVVSRVTEDSARLSWTAPDAAFDSFEISYWEHTENGEAIILFVPG |
| | SERSYDLTGLKPGTEYVVFIGGVKGGVWSVPLSAIFTT |
| 1528 | MSLPAPKNLVVSRVTEDSARLSWTAPDAAFDSFFIGYLEPQPPGEAIHLSVPG |
| | SERSYDLTGLKPGTEYWVQIGGVKGGNWSAPLSAIFTT |
| 1529 | MSLPAPKNLVVSRVTEDSARLSWTAPDAAFDSFFIGYLEPQPPGEAIALYVPG |
| | SERSYDLTGLKPGTEYNVTIQGVKGGFPSEPLVAHFTT |
| 1530 | MSLPAPKNLVVSRVTEDSARLSWTAPDAAFDSFEIVYAEPVRFGEAIVLIVPG |
| | SERSYDLTGLKPGTEYWVQIGGVKGGNWSAPLSAIFTT |
| 1531 | MSLPAPKNLVVSRVTEDSARLSWTEPDAAFDSFEIGYFENLYLGEAIVLTVPG |
| | SERSYDLTGLKPGTEYWVQIGGVKGGNWSAPLSAIFTT |
| 1532 | MSLPAPKNLVVSRVTEDSARLSWTAPDAAFDSFEIAYFERTWFGEAIVLTVPG |
| | SERSYDLTGLKPGTEYWVQIGGVKGGDWSKPLSAIFTT |
| 1533 | MSLPAPKNLVVSRVTEDSARLSWTAPDAAFDSFEIGYFENDYFGEAIVLTVPG |
| | SERSYDLTGLKPGTEYVVFIGGVKGGKWSAPLSAIFTT |
| 1534 | MSLPAPKNLVVSRVTEDSARLSWTAPDAAFDSFEIVYQENSAYGEAIVLTVPG |
| | SERSYDLTGLKPGTEYWVQIGGVKGGNWSAPLSAIFTT |
| 1535 | MSLPAPKNLVVSRVTEDSARLSWTAPDAAFDSFHIWYFEKANEGEAIPLVVPG |
| | SERSYDLTGLKPGTEYDVDIGGVKGGAWSIPLGARFTT |
| 1536 | MSLPAPKNLVVSRVTEDSARLSWTAPDAAFDSFMIAYDEFIVWGEAVVLTVPG |
| | SERSYDLTGLKPGTEYLVEILGVKGGTISGPLSAIFTT |
| 1537 | MSLPAPKNLVVSRVTEDSARLSWTAPDAAFDSFEIGYFENLYLGEAIVLTVPG |
| | SERSYDLTGLKPGTKYWVQIGGVKGGNWSAPLSAIFTT |
| 1538 | MSLPAPKNLVVSRVTEDSARLSWTAPDAAFDSFFIGYLEPQPPGEAIHLYVPG |
| | SERSYDLTGLKPGTEYNVTIQGVKGGEWSAPLSAIFTT |
| 1539 | MSLPAPKNLVVSRVTEDSARLSWTAPDAAFDSFEIGYFENLYLGEAIVLTVPG |
| | SERSYDLTGLKPGTEYVVFIGGVKGGEYSIPLSAIFTT |
| 1540 | MSLPAPKNLVVSRVTEDSARLSWTAPDAAFDSFEISYWEHTENGEAIILFVPG |
| | SERSYDLTGLKPGTEYYVTIGGVKGGAWSPPLWAHFTT |
| 1541 | MSLPAPKNLVVSRVTEDSARLSWTAPDAAFDSFEIAYQELSLWGEAIVLIVPG |
| | SERSYDLTGLKPGTEYYVHIGGVKGGIWSSPLSAYFTT |
| 1542 | MSLPAPKNLVVSRVTEDSARLSWTAPDAAFDSFEIGYFENLYLGEAIVLTVPG |
| | SERSYDLTGLKPGTEYWVQIGGVKGGEWSAPLSAIFTT |
| 1543 | MSLPAPKNLVVSRVTEDSARLSWTAPDAAFDSFEIGYFENLYLGEAIVLTVPG |
| | SERSYDLTGLKPGTEYVVFIGGVKGGDFSPPLSAIFTT |
| 1544 | MSLPAPKNLVVSRVTEDSARLSWTAPDAAFDSFEIGYWENAYRGEAIVLTVPG |
| | SERSYDLTGLKPGTEYVVFIGGVKGGVWSVPLSAIFTT |
| 1545 | MSLPAPKNLVVSRVTEDSARLSWTAPDAAFDSFEIGYFENLYLGEAIVLIVPG |
| | SERSYDLTGLKPGTEYWVQIGGVNGGNWSAPLSAIFTT |
| 1546 | MSLPAPKNLVVSRVTEDSARLSWTAPDAAFDSFEIGYFENLYLGEAIVLTVPG |
| | SERSYDLTGLKPGTEYWVQIGGVKGGNWSAPLVATFTT |
| 1547 | MSLPAPKNLVVSRVTEDSARLSWTAPDAAFDSFEIGYFENLYLGEAIVLTVPG |
| | SERSYDLTGLKPGTEYWVQIGGVKGGNWSAPLSAIFTT |
| 1548 | MSLPAPKNLVVSRVTEDSARLSWTAPDAAFDSFDINYWENESGGEAIALFVPG |
| | SERSYDLTGLKPGTEYWVSIGGVKGGRFSEPLYARFTT |
| 1549 | MSLPAPKNLVVSRVTEDSARLSWTAPDAAFDSFEIGYFENLYLGEAIVLWVPG |
| | SERSYDLTGLKPGTEYWVQIGGVKGGNWSAPLSAIFTT |
| 1550 | MSLPAPKNLVVSRVTEDSARLSWTTPDAAFDSFEIAYFEIAWLGEAIVLTVPG |
| | SERSYDLTGLKPGTEYWVQIGGVKGGAYSTPLSAIFTT |
| 1551 | MSLPVPKNLVVSRVTEDSARLSWTAPDAAFDSFEIGYFENLYLGEAIVLTVPG |
| | SERSYDLTGLKPGTEYWVQIGGVKGGNWSAPLSAIFTT |
| 1552 | MSLPAPKNLVVSHVTEDSARLSWTAPDAAFDSFEIGYWENAYKGEAIVLTVPG |
| | SERSYDLTGLKPGTEYVVFIGGVKGGVWSVPLSAIFTT |
| 1553 | MSLPAPKNLVVSRVTEDSARLSWTAPDAAFDSFFIGYLEPQPPGEAIVLGVPG |
| | SERSYDLTGLKPGTEYNVTIQGVKGGFPSDPLVASFTT |
| 1554 | MSLPAPKNLVVSRVTEDSARLSWTAPDAAFDSFYIAYAEPRPDGEAILLEVPG |
| | SERSYDLTGLKPGTEYSVLIHGVKGGLLSSPLSATFTT |
| 1555 | MSLPAPKNLVVSRITEDSARLSWTAPDAAFDSFEIGYFENLYLGEAIVLTVPG |
| | SERSYDLTGLKPGTEYWVQIGGVKGGNWSAPLSAIFTT |
| 1556 | MSLPAPKNLVVSRVTEDSARLSWTAPDAAFDSFYIAYAEPRPDGEAILLWVPG |
| | SERSYDLTGLKPGTEYSVLIHGVKGGLLSSPLHAYFTT |
| 1557 | MSLPAPKNLVVSRVTEDSARLSWTAPDAAFDSFFIGYLEPQPPGEVIHLFVPG |
| | SERSYDLTGLKPGTEYNVTIQGVKGGFPSDPLVASFTT |
| 1558 | MSLPAPKNLVVSRVTEDSARLSWTAPDAAFDSFEIGYFENLYLGEAIVLTVPG |
| | SERSYDLTGLKPGTEYWVQIGGVKGGNWSSPLSAIFTT |
| 1559 | MSLPAPKNLVVSRVTEDSARLSWTAPDAAFDSFFIGYLEPQPPGEAIALYVPG |
| | SERSYDLTGLKPGTEYNVTIQGVKGGFPSEPLVVHFTT |
| 1560 | MSLPAPKNLVVSRVTEDSARLSWTAPDAAFDSFFIGYLEPQPPGEAISLYVPG |
| | SERSYDLTGLKPGTEYNVTIQGVKGGFPSDPLVAHFTT |
| 1561 | MSLPAPKNLVVSRVTEDSARLSWTAPDAAFDSFFIGYLEPQPPGEAIYLLVPG |
| | SERSYDLIGLKPGTEYNVTIQGVKGGFPSLPLVATFTT |
| 1562 | MSLPAPKNLVVSRVTEDSARLSWTAPDAAFDSFEISYWEHTENGEAIILFVPG |
| | SERSYDLIGLKPGTEYYVTIGGVKGGAWSPPLWAEFTT |
| 1563 | MSLPAPKNLVVSRVTEDSARLSWTAPDAAFDSFEIGYFENLYLGEAIVLTVPG |
| | SERSYDPTGLKPGTEYWVQIGGVKGGNWSAPLSAIFTT |
| 1564 | MSLPAPKNLVVSRVTEDSARLSWTAPDAAFDSFEIGYFENDYFGEAIVLTVPG |
| | SERSYDLTGLKPGTEYVVFIGGVKGGNWSAPLSAIFTT |
| 1565 | MSLPAPKNLVVSRVTEDSARLSWTAPDAAFDSFFIGYLEPQPPGEAIHLYVPG |
| | SERSYDLTGLKPGTEYNVTIQGVKGGFPSEPLIANSTT |
| 1566 | MSLPAPKNLVVSRITEDSARLSWTAPDAAFDSFEIGYWENAYKGEAIVLTVPG |
| | SERSYDLTGLKPGTEYVVFIGGVKGGVWSVPLSAIFTT |
| 1567 | MSLPAPKNLVVSRVTEDSARLSWTAPDAAFDSFEIAYGEAAYDGEAIALLVPG |
| | SERSYDLTGLKPGTEYWVQIGGVKGGNWSAPLSAIFTT |
| 1568 | MLPAPKNLVVSRVTEDSARLSWTAPDAAFDSFEISYWEHTENGEAIILFVPGS |
| | ERSYDLTGLKPGTEYYVTIGGVKGGAWSPPLWAEFTT |
| 1569 | MSLPAPKNLVVSRVTEDSARLSWTAPDAAFDSFEIVYWEQVGVGEAIVLTVPG |
| | SERSYDLTGLKPGTEYWVQIGGVKGGGFSEPLSAIFTT |
| 1570 | MSLPAPKNLVVSRVTEDSARLSWTAPDAAFDSFEIGYFENLYLGEAIVLTVPG |
| | SERSYDLTGLKPGTEYWVQIGGVKGGNWSAPLVAEFTT |
| 1571 | MSLPAPKNLVVSRVTEDSARLSWTAPDAAFDSFEIAYFELDYVGEAIVLTVPG |
| | SERSYDLTGLKPGTEYWVQIGGVKGGTWSGPLSAIFTI |
| 1572 | MSLPAPKNLVVSRVTEDSARLSWTAPDAAFDSFEIGYFELTYYGEAIVLTVPG |
| | SERSYDLTGLKPGTEYWVQIGGVKGGGFSEPLSAIFTT |
| 1573 | MSLPAPKNLVVSRVTEDSARLSWTAPDAAFDSFEIGYFENLYLGEAIVLIVPG |
| | SERSHDLTGLKPGTEYWVQIGGVKGGNWSAPLSAIFTT |
| 1574 | MSLPAPKNLVVSRVTEDSARLSWTAPDAAFDSFFIGYLEPQPPGEAIHLYVPG |
| | SERSYDLTGLKPGTEYNVTIQGVKGGNWSAPLSAIFTT |
| 1575 | MSLPAPKNLVVSRVTEDSARLSWTALDAAFDSFEIGYFENLYLGEAIVLTVPG |
| | SERSYDLTGLKPGTEYWVQIGGVKGGNWSAPLSAIFTT |
| 1576 | MSLPAPKNLVVSRVTEDSARLSWTAPDAAFDSFFIGYLEPQPPGEAIALYVPG |
| | SERSYDLTGLKPGTEYNVTIQGVKGGFPSEPLIANFTT |
| 1577 | MSLPAPKNLVVSRVTEDSARLSWTAPDAAFDSFDIGYFELTWYGEAIVLIVPG |
| | SERSYDLTGLKPGTEYWVSIGGVKGGRFSEPLYARFTT |
| 1578 | MSLPAPKNLVVSRVTEDSARLSWTAPDAAFDSFEIAYQELSLWGEAIVLTVPG |
| | SERSYDLTGLKPGTEYYVHIGGVKGGIWSSPLSAYSTT |
| 1579 | MSLPAPKNLVVSRVTEDSARLSWTAPDAAFDSFEIGYWENAYKGEAIVLTVPG |
| | SERSYDLTGLKPGTEYMVFIGGVKGGVWSVPLSAIFTT |
| 1580 | MSLPAPKNLVVSRVTEDSARLSWTATDAAFDSFEIGYWENAYKGEAIVLIVPG |
| | SERSYDLTGLKPGTEYVVFIGGVKGGVWSVPLSAIFTT |
| 1581 | MSLPAPKNLVVSRVTEDSARLSWTAPDAAFDSFEIVYAEPVRFGEAIVLIVPG |
| | SERSYDLTGLKPGTEYWVQIGGVKGGEWSSPLSAIFTT |
| 1582 | MSLPAPKNLVVSRVTEDSARLSWTAPDAAFDSFEIGYFENLYLGEAIVLTVPG |
| | SERSYDLTGLKPGTEYWVQIGGVKGGNWSAPLSAIFTT |
| 1583 | MSLPAPKNLVVSRITEDSARLSWTAPDAAFDSFEIGYFENLYLGEAIVLTVPG |
| | SERSYDLTGLKPGTEYWVQIGGVKGGNWSAPLSAIFTT |
| 1584 | MSLPAPKNLVVSRVTEDSARLSWTAPDAAFDSFEIAYFEIAWLGEAIVLTVPG |
| | SERSYDLTGLKPGTEYWVQIGGVKGGAYSTPLSAIFTT |
| 1585 | MSLPAPKNLVVSRVTEDSARLSWTAPDAAFDSFFIGYLEPQPPGEAIHLYVPG |
| | SERSYDLIGLKPGTEYNVTIQGVKGGFPSEPLVATFTT |
| 1586 | MSLPAPKNLVVSRITEDSARLSWTAPDAAFDSFEIGYFENLYLGEAIVLTVPG |
| | SERSYDLTGLKPGTEYWVQIGGVKGGNWSAPLSAIFTT |
| 1587 | MSLPAPKNLVVSRVTEDSARLSWTAPDAAFDSFEIGYWENAYKGEAIVLIVPG |
| | SERSYDLTGLKPGTEYVVFIGGVKGGVWSVSLSAIFTT |
| 1588 | MSLPAPKNLVVSRVTEDSARLSWTAPDAAFDSFFIGYLEPQPPGEAIDLYVPG |
| | SERSYDLTGLKPGTEYNVTIQGVKGGFPSHPLVATFTT |
| 1589 | MSLPAPKNLVVSRVTEDSARLSWTAPDAAFDSFFIGYLEPQPLGEAIHLFVPG |
| | SERSYDLTGLKPGTEYNVTIQGVKGGFPSEPLIASFTT |
| 1590 | MSLPAPKNLVVSRVTEDSARLSWTAPDAAFDSFFIGYLEPQPPGEAIDLYVPG |
| | SERSYDLTGLKPGTEYNVTIQGVKGGFPSEPLVATFTT |
| 1591 | MSLPAPKNLVVSRVTEDSARLSWTAPDAAFDSFDIGYFELTWYGEAIVLIVPG |
| | SERSYDLTGLKPGTEYWVSIGGVKGGIWSSPLSAIFTT |
| 1592 | MSLPAPKNLVVSRVTEDSARLSWTAPDAAFDSFEISYFENTWYGEAIVLIVPG |
| | SERSYDLTGLKPGTEYSVRIFGVKGGNFSFPLSAIFTT |
| 1593 | MSLPAPKNLVVSRVTEDSARLSWTAPDAAFDSFEIGYFENLYLGEAIVLTVPG |
| | SERSYDLTGLKPGTEYWVQIGGVKGDNWSAPLSAIFTT |
| 1594 | MSLPAPKNLVVSRVTEDSARLSWTAPDAAFDSFEIAYFENLYLGEAIVLTVPG |
| | SERSYDLTGLKPGTEYWVQIGGVKGGNWSAPLSAIFTT |
| 1595 | MSLPAPKNLVVSRVTEDSARLSWTAPDAAFDSFEIGYFEATPNGEAIVLTVPG |
| | SERSYDLTGLKPGTEYKVFIGGVKGGRWSKPLSAIFTT |
| 1596 | MSLPAPKNLVVSRVTEDSARLSWTTPDAAFDSFEIGYFENLYLGEAIVLTVPG |
| | SERSYDLTGLKPGTEYWVQIGGVKGGNWSAPLSAIFTT |
| 1597 | MSLPAPKNLVVSRVTEDSARLSWTAPDAAFDSF*ISYKESFTYGEAIVLTVPG |
| | SERSYDLTGLKPGTEYLVEIVGVKGGNLSGPLSAIFTT |
| 1598 | MSLPAPKNLVVSRVTEDSARLSWTAPDAAFDSFWIEYWEGWKSGEAIVLTVPG |
| | SERSYDLTGLKPGTEYRVHIWGVKGGIVSWPLSAIFTT |
| 1599 | MSLPAPKNLVVSRVTEDSARLSWTAPDAAFDSFEIGYFENLYLGEAIVLTVPG |
| | SERSYDLTGLKPGTEYVVFIGGVKGGVWSVPLSAIFTT |
| 1600 | MSLPAPKNLVVSRVTEDSARLSWTAPDAAFDSFEIAYFEQTYTGEAIVLTVPG |
| | SERSYDLTGLKPGTEYWVQIGGVKGGNWSAPLSAIFTT |
| 1601 | MSLPAPKNLVVSRVTEDSARLSWTAPDAAFDSFEIGHFENLYLGEAIVLTVPG |
| | SERSYDLTGLKPGIEYWVQIGGVKGGVWSVSLSAIFTT |
| 1602 | MSLPAPKNLVVSRVTEDSARLSWTAPDAAFNSFEIGYFENLYLGEAIVLTVPG |
| | SERSYDLTGLKPGTEYWVQIGGVKGGTFSAPLSAIFTT |
| 1603 | MSLPAPKNLVVSRVTEDSARMSWTTPDAAFDSFEIGYFENLYLGEAIVLTVPG |
| | SERSYDLTGLKPGTEYWVQIGGVKGGNWSAPLSAIFTT |
| 1604 | MSLPAPENLVVSRVTEDSARLSWTAPDAAFDSFEIGYFENLYLGEAIVLIVPG |
| | SERSYDLTGLKPGTEYWVQIGGVKGGNWSAPLSAIFTT |
| 1605 | MSLPAPKNLVVSRVTEDSARLSWTAPDAAFDSFEIDYGEYSQAGEAIGLLVPG |
| | SERSYDLTGLKPGTEYAVWIGGVKGGFFSTPLEADFTT |
| 1606 | MSLPAPKNLVVSRVTEDSARLSWTAPDAAFDSFEIGYWENAYKGEAIVLTVPG |
| | SERSYDLTGLKPGTEYVVFIGGVKGGSWSNPLSAIFTT |
| 1607 | MSLPAPKNLVVSRVTEDSARLSWTAPDAAFDSFEIGYFENLYLGEAIVLTIPG |
| | SERSYDLTGLKPGTEYWVQIGGVKGGNWSAPLSAIFTT |
| 1608 | MSLPAPKNLVVSRVTEDSARLSWTAPDAAFDSFEIAYFENDWAGEAIVLTIPG |
| | SERSYDLTGLKPGTEYWVQIGGVKGGNWSAPLSAIFTT |
| 1609 | MGSLPAPKNLVVSRVTEDSARLSWTAPDAAFDSFEIGYFENLYLGEAIVLTVP |
| | GSERSYDLTGLKPGTEYWVQIGGVKGGNWSAPLSAIFTT |
| 1610 | MSLPAPKNLVVSRVTEDSARLSWTAPDAAFDSFEIGYFENLYLGEAIVLIVPG |
| | SERSYDLTGLKPGTEYWVQIGGVKGGNWSAPLSAISTT |
| 1611 | MSLPAPKNLVISRVTEDSARLSWTAPDAAFDSFEIGYFENLYLGEAIVLIVPG |
| | SERSYDLTGLKPGTEYWVQIGGVKGGNWSAPLSAIFTT |
| 1612 | MSLPAPKNLVVSRVTEDSARLSWTAPDAAFDSFEIGYFENLYLGEAIVLIVPG |
| | SERSYDLTGLKPGTEYWVQIGGVKGGNWSAPLSAIFTT |
| 1613 | MSLPAPKNLVVSRVTEDSARLSWTAPDAAFDSFEIGYFENLYLGEAIVLTVPG |
| | SERSYDLTGLKPGTEYRVVILGVKGGW*SGPLSAIFTT |
| 1614 | MSLPAPKNLVVSRVTEDSARLSWTAPGAAFDSFEIGYFENLYLGEAIVLTVPG |
| | SERSYDLTGLKPGTEYWVQIGGVKGGVWSVPLSAIFTT |
| 1615 | MSLPVPKNLVVSRVTEDSARLSWTAPDAAFDSFEIGYWENAYKGEAIVLIVPG |
| | SERSYDLTGLKPGTEYWVQIGGVKGGNWSAPLSAIFTT |
| 1616 | MSLPAPKNLVVSRVTEDSARLSWTAPDAAFDSFEIGYFENLYLGEAIVLIVPG |
| | SERSYDLTGLKPGTEYWVQIGGVKGGNWSAPLSAIFTT |
| 1617 | MSLPAPKNLVVSRVTEDSARLSWTAPDAAFDSFEIGYFEITRYGEAIILFVPG |
| | SERSYDLTGLKPGTEYWVQIGGVKGGNWSAPLSAIFTT |
| 1618 | MSLPAPKNLVVSRVTEDSARLSWTAPDAAFDSFEIGYFENLYLGEAIVLTVPG |
| | SERSYDLTGLKPGTEYWVQIGGVKGGNWSGPLSAIFTT |
| 1619 | MSLPAPKNLVVSRITEDSARLSWTAPDAAFDSFEIGYFENLYLGEAIVLTVPG |
| | SERSYDLTGLKPGTEYWVQIGGVKGGNWSAPLSAISTT |
| 1620 | MSLPAPKNLVVSRVTEDSARLSWTAPDAAFDSFEIGYFENLYLGEAIVLTVPG |
| | SERSYDLTGLKPGTEYWVQIGGVKGGNWSAPLSEIFTT |
| 1621 | MSLPAPKNLVVSRVTEDSARLSWTAPDAAFDSFEIGYWENAYKGEAIVLTVPG |
| | SERSYDLTGLKPGTEYWVQIGGVKGGVWSTPLSAIFTT |
| 1622 | MSLPAPKNLVVSRVTEDSARLSWTAPDAAFDSFEIGYWENAYKGEAIVLTVPG |
| | SERSYDLTGLKPGTEYWVQIGGVKGGNWSAPLSAIFTT |
| 1623 | MSLPAPKNLVVSRVTEDSARLSWTAPDAAFDSFEIGYFGNLYLGEAIVLTVPG |
| | SERSYDLTGLKPGTEYWVQIGGVKGGNWSAPLSAIFTT |
| 1624 | MSLPAPKNLVVSRVTEDSARLSWTAPDAAFDSFDIGYFEATYYGEAIVLTVPG |
| | SERSYDLIGLKPGTEYRVWIGGVKGGYYSNPLSAIFTT |
| 1625 | MSLPAPKNLVVSRVTEDSARLSWTAPDAAFDSFEIGYFENLYLGEAIVLIVPG |
| | SERSYDLTGLKPGTEYWVQIGGVKGGNWSAPLSAIVTT |
| 1626 | MSLPAPKNLVVSRVTEDSARLSWTAPDAAFDSFFIGYLEPQPPGEAIHLYVPG |
| | SERSYDLTGLKPGTEYNVTIQGVKVGFPSEPLIANFTT |
| 1627 | MSLPAPKNLVVSRVTEDSARLSWTAPDAAFDSFEIGYWEGPGGGEAIILVVPG |
| | SERSYDLTGLKPGTEYYVQIGGVKGGDWSTPLWATFTT |
| 1628 | MSLPAPENLVVSRVTEDSARLSWTAPDAAFDSFFIGYLEPQPPGEAIALYVPG |
| | SERSYDLTGLKPGTEYNVTIQGVKGGFPSLPLVATFTT |
| 1629 | MSLPAPKNLVVSRVTEDSARLSWTAPDAAFDSFEIAYQELSLWGEAIVLTVPG |
| | SERSYDLTGLKPGTEYWVQIGGVKGGIWSSPLVASFTT |
| 1630 | MSLPAPKNLVVSRVTEDSARLSWTAPDAAFDSFFIGYLEPQPPGEAIDLYVPG |
| | SERSYDLTGLKPGTEYNVTIQGVKGGFPSLPLVATFTT |
| 1631 | MSLPAPKNLVVSRVTEDSARLSWTAPDAAFDSFNIMYHEHYDNGEAIVLTVPG |
| | SERSYDLTGLKPGTEYSVVINGVKGGPHSAPLSAIFTT |
| 1632 | MSLPAPKNLVVSRVTEDSARLSWTAPDAAFDSFFIGYLEPQPPGEAIHLYVPG |
| | SERSYDLTGLKPGTEYNVTIQGVKGGFPSIPLIASFTT |
| 1633 | MSLPAPKNLVVSRVTEDSARLSWTAPDAAFDSFEIGYFENLYLGEAIVLTVPG |
| | SERSYDLTGLKPGTEYCVYICGVKGGRDSMPLSAIFTT |
| 1634 | MSLPAPKNLVVSRVTEDSARLSWTAPDAAFDSFEIGYWEMSYYGEAIVLIVPG |
| | SERSYDLTGLKPGTEYGVSIGGVKGGRWSLPLSAIFTT |
| 1635 | MSLPAPKNLVVSRVTEDSSRLSWTAPDAAFDSFEIGYFENLYLGEAIVLTVPG |
| | SERSYDLTGLKPGTEYWVQIGGVKGGNWSAPLSAIFTT |
| 1636 | MSLPAPKNLVVSRVTEDSARLSWTAPDAAFDSFFIGYLEPQPPGEAIELYVPG |
| | SERSYDLTGLKPGTEYNVTIQGVKGGFPSLPLVAIFTT |
| 1637 | MSLPAPKNLVVSRVTEDSARLSWTAPDAAFDSFFIGYLEPQPPGEAIVLTVPG |
| | SERSYDLTGLKPGTEYNVTIQGVKGGFPSEPLIANFTT |
| 1638 | MSLPAPKNLVVSRVTEDSARLSWTAPDAAFDSFFIGYLEPQPPGEAIALWVPG |
| | SERSYDLTGLKPGTEYNVTIQGVKGGFPSEPLVATFTT |
| 1639 | MSLPAPKNLFVSRVTEDSARLSWTAPDAAFDSFEIGYWENAYKGEAIVLIVPG |
| | SERSYDLTGLKPGTEYWVQIGGVKGGNWSAPLSAIFTT |
| 1640 | MSLPAPKNLVVSRVTEDSAHLSWTAPDAAFDSFEIGYFENDYFGEAIVLIVPG |
| | SERSYDLTGLKPGTEYVVFIGGVKGGKWSAPLSAIFTT |
| 1641 | MLPAPKNLVVSRVTEDSARLSWTAPDAAFDSFFIGYLEPQPPGEAISLFVPGS |
| | ERSYDLTGLKPGTEYNVTIQGVKGGFPSDPLNATENT |
| 1642 | MLPAPKNLVVSRVTEDSARLSWTAPDAAFDSFFIGYLEPQPPGEAIGLFVPGS |
| | ERSYDLTGLKPGTEYNVTIQGVKGGFPSEPLKAAFTT |
| 1643 | MLPAPKNLVVSRVTEDSARLSWTAPDAAFDSFEISYFENTWYGEAIVLTVPGS |
| | ERSYDLTGLKPGTEYVVWIGGVKGGLWSKPLSAIFTT |
| 1644 | MLPAPKNLVVSRVTEDSARLSWTAPDAAFDSFFIGYLEPQPPGEAIELYVPGS |
| | ERSYDLTGLKPGTEYNVTIQGVKGGFPSEPLIAHFTT |
| 1645 | MLPAPKNLVVSRVTEDSARLSWTAPDAAFDSFFIGYLEPQPPGEAISLFVPGS |
| | ERSYDLTGLKPGTEYNVTIQGVKGGFPSMPLVAQFTT |
| 1646 | MLPAPKNLVVSRVTEDSARLSWTAPDAAFDSFFIGYLEPQPPGEAITLAVPGS |
| | ERSYDLTGLKPGTEYNVTIQGVKGGFPSNPLLAAFTT |
| 1647 | MLPAPKNLVVSRVTEDSARLSWTAPDAAFDSFEIAYQELSLWGEAIVLTVPGS |
| | ERSYDLTGLKPGTEYYVHIGGVKGGIWSSPLSAIFTT |
| 1648 | MLPAPKNLVVSRVTEDSARLSWTAPDAAFDSFFIGYLEPQPPGEAIGLFVPGS |
| | ERSYDLTGLKPGTEYNVTIQGVKGGFPSNPLEARFTT |
| 1649 | MLPAPKNLVVSRVTEDSARLSWTAPDAAFDSFFIGYLEPQPPGEAIQLWVPGS |
| | ERSYDLTGLKPGTEYNVTIQGVKGGFPSLPLVATFTT |
| 1650 | MLPAPKNLVVSRVTEDSARLSWTAPDAAFDSFFIGYLEPQPPGEAIHLYVPGS |
| | ERSYDLTGLKPGTEYNVTIQGVKGGFPSHPLYADFTT |
| 1651 | MLPAPKNLVVSRITEDSARLSWTAPDAAFDSFFIGYLEPQPPGEAITLLVPAS |
| | ERSYDLTGLKPGTEYNVTIQGVKGGFPSHPLQAQFTT |
| 1652 | MLPAPKNLVVSRVTEDSARLSWTAPDAAFDSFFIGYLEPQPPGEAIGLFVPGS |
| | ERSYDLTGLKPGTEYNVTIQGVKGGFPSDPLVAYFTT |
| 1653 | MLPAPKNLVVSRVTEDSARLSWTAPDAAFDSFEIGYWENAYKGEAIVLTVPGS |
| | ERSYDLTGLKPGTEYVVFIGGVKGGVWSVPLSAIFTT |
| 1654 | MLPAPKNLVVSRVTEDSARLSWTAPDAAFDSFLIPYAETSPSGEAIVLTVPGS |
| | ERSYDLTGLKPGTEYSVLIHGVKGGDYSSPLSAIFTT |
| 1655 | MLPAPKNLVVSRVTEDSARLSWTAPDAAFDSFFIGYLEPQPPGEAISLYVPGS |
| | ERSYDLTGLKPGTEYNVTIQGVKGGFPSHPLLAVFTT |
| 1656 | MLPAPKNLVVSRVTEDSARLSWTAPDAAFDSFFIGYLEPQPPGEAIQLFVPGS |
| | ERSYDLTGLKPGTEYNVTIQGVKGGFPSIPLSALFTT |
| 1657 | MLPAPKNLVVSRVTEDSARLSWTAPDAAFDSFEIAYWENNENGEAIILIVPGS |
| | ERSYDLTGLKPGTEYVVFISGVKGGTWSYPLVAQFTT |
| 1658 | MLPAPKNLVVSRVTEDSARLSWTAPDAAFDSFFIGYLEPQPPGEAIDLWVPGS |
| | ERSYDLTGLKPGTEYNVTIQGVKGGFPSLPLSAIFTT |
| 1659 | MLPAPNNLVVSRVTEDSARLSWTAPDAAFDSFFIGYLEPQPPGEAIHLFVPGS |
| | ERSYDLTGLKPGTEYNVTIQGVKGGFPSGPLHATFTT |
| 1660 | MLPAPKNLVVSRVTEDSARLSWTAPDAAFDSFFIGYLEPQPPGEAIVLWVPGS |
| | ERSYDLTGLKPGTEYNVTIQGVKGGFPSHPLVAKFTT |
| 1661 | MLPAPKNLVVSRVTEDSARLSWTAPDAAFDSFFIGYLEPQPPGEAIDLWVPGS |
| | ERSYDLTGLKPGTEYNVTIQGVKGGFPSNPLKAQFTT |
| 1662 | MLPAPKNLVVSRVTEDSARLSWTAPDAAFDSFYIAYAEPRPDGEAILLVVPGS |
| | ERSYDLTGLKPGTEYSVLIHGVKGGLLSSPLVAFFTT |
| 1663 | MLPAPKNLVVSRVTEDSARLSWTAPDAAFDSFFIGYLEPQPPGEAITLTVPGS |
| | ERSYDLTGLKPGTEYNVTIQGVKGGFPSGPLIASFTT |
| 1664 | MLPAPKNLVVSRVTEDSARLSWTAPDAAFDSFFIGYLEPQPPGEAIVLWVPGS |
| | ERSYDLTGLKPGTEYNVTIQGVKGGFPSHPLVAKSTT |
| 1665 | MLPAPKNLVVSRVTEDSARLSWTAPDAAFDSFYIAYAEPRPDGEAIVLVVPGS |
| | ERSYDLTGLKPGTEYSVLIHGVKGGLLSSPLSAHFTT |
| 1666 | MLPAPKNLVVSRVTEDSARLSWTAPDAAFDSFDIDYVEFGWTGEAIALLVPGS |
| | ERSYDLTGLKPGTEYWVWIGGVKGGDYSPPLNAYFTT |
| 1667 | MLPAPKNLVVSRVTEDSARLSWTAPDAAFDSFFIGYLEPQPPGEAINLLVPGS |
| | ERSYDLTGLKPGTEYNVTIQGVKGGFPSLPLLAEFTT |
| 1668 | MLPAPKNLVVSRVTEDSARLSWTAPDAAFDSFFIGYLEPQPPGEAISLFVPGS |
| | ERSYDLTGLKPGTEYNVTIQGVKGGFPSDPLVASFTT |
| 1669 | MLPAPKNLVVSRVTEDSARLSWTAPDAAFDSFYIAYAEPRPDGEAIQLSVPGS |
| | ERSYDLTGLKPGTEYSVLIHGVKGGLLSSPLHASFTT |
| 1670 | MLPAPKNLVVSRVTEDSARLSWTAPDAAFDSFFIGYLEPQPPGEAIDLTVPGS |
| | ERSYDLTGLKPGTEYNVTIQGVKGGFPSMPLSAIFTT |
| 1671 | MLPAPKNLVVSRVTEDSARLSWTAPDAAFDSFFIGYLEPQPPGEAIALLVPGS |
| | ERSYDLTGLKPGTEYNVTIQGVKGGFPSNPLEAKFTT |
| 1672 | MLPAPKNLVVSRVTEDSARLSWTAPDAAFDSFFIGYLEPQPPGEAIQLWVPGS |
| | ERSYDLTGLKPGTEYNVTIQGVKGGFPSTPLQALFTT |
| 1673 | MLPAPKNLVVSRVTEDSARLSWTAPDAAFDSFFIGYLEPQPPGEAIDLWVPGS |
| | ERSYDLTGLKPGTEYNVTIQGVKGGFPSTPLIATVTT |
| 1674 | MLPAPKNLVVSRVTEDSARLSWTAPDAAFDSFYIEYWEGYRSGEAIVLTVPGS |
| | ERSYDLTGLKPGTEYRVHIWGVKGGAVSYPLSAIFTT |
| 1675 | MLPAPKNLVVSRVTEDSARLSWTAPDAAFDSFFIGYLEPQPPGEAIGLFVPGS |
| | ERSYDLTGLKPGTEYNVTIQGVKGGFPSMPLSAIFTT |
| 1676 | MLPAPKNLVVSRVTEDSARLSWTAPDAAFDSFEIAYQELSLWGEAIVLTVPGS |
| | ERSYDLTGLKPGTEYYVHIGGVKGGIWSRPLSAIFTT |
| 1677 | MLPAPKNLVVSRVTEDSARLSWTAPDAAFDSFFIGYLEPQPPGEAIELYVPGS |
| | ERSYDLTGLKPGTEYNVTIQGVKGGFPSDPLEADFTT |
| 1678 | MLPAPKNLVVSRVTEDSARLSWTAPDAAFDSFFIGYLEPQPPGEAIDLWVPGS |
| | ERSYDLTGLKPGTEYNVTIQGVKGGFPSLPLDAVETT |
| 1679 | MLPAPKNLVVSRVTEDSARLSWTAPDAAFDSFEIDYAENRHHGEAIALLVPGS |
| | ERSYDLTGLKPGTEYIVFIGGVKGGRWSQPLVASFTT |
| 1680 | MLPAPKNLVVSRVTEDSARLSWTAPDAAFDSFFIGYLEPQPPGEAIDLYVPGS |
| | ERSYDLTGLKPGTEYNVTIQGVKGGFPSHPLQAHFTT |
| 1681 | MLPAPKNLVVSRVTEDSARLSWTAPDAAFDSFYIAYAEPRPDGEAITLLVPGS |
| | ERSYDLTGLKPGTEYSVLIHGVKGGLLSSPLSAIFTT |
| 1682 | MLPAPKNLVVSRVTEDSARLSWTAPDAAFDSFFIGYLEPQPPGEAIHLVVPGS |
| | ERSYDLTGLKPGTEYNVTIQGVKGGFPSDPLYAWFTT |
| 1683 | MLPAPKNLVVSRVTEDSARLSWTAPDAAFDSFEIAYFEYCLNGEAIVLTVPGS |
| | ERSYDLTGLKPGTEYWVQIGGVKGGTWSWPLSAIFTT |
| 1684 | MLPAPKNLVVSRVTEDSARLSWTAPDAAFDSFFIGYLEPQPPGEAIHLLVPGS |
| | ERSYDLTGLKPGTEYNVTIQGVKGGFPSMPLSAIVTT |
| 1685 | MLPAPKNLVVSRVTEDSARLSWTAPDAAFDSFEIGYFERAWFGEAIVLTVPGS |
| | ERSYDLTGLKPGTEYAVFIGGVKGGSYSYPLSAIFTT |
| 1686 | MLPAPKNLIVSRVTEDSARLSWTAPDAAFDSFFIGYLEPQPPGEAISLYVPGS |
| | ERSYDLTGLKPGTEYNVTIQGVKGGFPSDPLQAIFTT |
| 1687 | MLPAPKNLVVSRVTEDSARLSWTAPDAAFDSFFIGYLEPQPPGEAIHLGVPGS |
| | ERSYDLTGLKPGTEYNVTIQGVKGGFPSLPLVATFTT |
| 1688 | MLPAPKNLVVSRVTEDSARLSWTAPDAAFDSFFIGYLEPQPPGEAIGLFVPGS |
| | ERSYDLTGLKPGTEYNVTIQGVKGGFPSLPLQAHFTT |
| 1689 | MLPAPKNLVVSRVTEDSARLSWTAPDAAFDSFFIGYLEPQPPGEAIYLLVPGS |
| | ERSYDLTGLKPGTEYNVTIQGVKGGFPSTPLEASFTT |
| 1690 | MLPAPKNLVVSRVTEDSARLSWTAPDTAFDSFFIGYLEPQPPGEAIGLFVPGS |
| | ERSYDLTGLKPGTEYNVTIQGVKGGFPSDPLSAFFTT |
| 1691 | MLPAPKNLVVSRVTEDSARLSWTAPDAAFDSFFIGYLEPQPPGEAIYLTVPGS |
| | ERSYDLTGLKPGTEYNVTIQGVKGGFPSMPLSAIFTT |
| 1692 | MLPAPKNLVVSRVTEDSARLSWTAPDAAFDSFIILYGEAQYDGEAIVLTVPGS |
| | ERSYDLTGLKPGTEYPVDIYGVKGGPYSWPLSAIFTT |
| 1693 | MLPAPKNLVVSRVTEDSARLSWTAPDAAFDSFFIGYLEPQPPGEAIYLTVPGS |
| | ERSYDLTGLKPGTEYNVTIQGVKGGFPSKPLTANFTT |
| 1694 | MLPAPKNLVVSRVTEDSARLSWTAPDAAFDSFFIGYLEPQPPGEAITLYVPGS |
| | ERSYDLTGLKPGTEYNVTIQGVKGGFPSTPLTATFTT |
| 1695 | MLPAPKNLVVSRVTEDSARLSWTAPDAAFDSFFIGYLEPQPPGEAIHLFVPGS |
| | ERSYDLTGLKPGTEYNVTIQGVKGGFPSGPLHATFTT |
| 1696 | MLPAPKNLVVSRVTEDSARLSWTAPDAAFDSFFIGYLEPQPPGEAIALWVPGS |
| | ERSYDLTGLKPGTEYNITIQGVKGGFPSMPLVANFTT |
| 1697 | MLPAPKNLVVSRVTEDSARLSWTAPDAAFDSFGIYYWEYTGGEAIVLTVPGSE |
| | RSYDLTGLKPGTEYVVRILGVKGGAYSTPLSAIFTT |
| 1698 | MLPAPKNLVVSRVTEDSARLSWTAPDAAFDSFYIAYAEPRPDGEAIVLDVPGS |
| | ERSYDLTGLKPGTEYSVLIHGVKGGLLSSPLVADETT |
| 1699 | MLPAPKNLVVSRVTEDSARLSWTAPDAAFDSFFIGYLEPQPPGEAIHLLVPGS |
| | ERSYDLTGLKPGTEYNVTIQGVKGGFPSEPLLAIVTT |
| 1700 | MLPAPKNLVVSRVTEDSARLSWTAPDAAFDSFFIGYLEPQPPGEAIHLWVPGS |
| | ERSYDLTGLKPGTEYNVTIQGVKGGFPSLPLSAFFTT |
| 1701 | MLPAPKNLVVSRVTEDSARLSWTAPDAAFDSFFIDYDESLDSGEAIVLTVPGS |
| | ERSYDLTGLKPGTEYNVTIQGVKGGFPSSPLEAAFTT |
| 1702 | MLPAPKNLVVSRVTEDSARLSWTAPDAAFDSFEIAYFETCRTGEAIVLTVPGS |
| | ERSYDLTGLKPGTEYRVWIGGVKGGVWSRPLSAIFTT |
| 1703 | MLPAPKNLVVSRVTEDSARLSWTAPDAAFDSFFIGYLEPQPPGEAIGLWVPGS |
| | ERSYDLTGLKPGTEYNVTIQGVKGGFPSMPLSAIFTT |
| 1704 | MLPAPKNLVVSRVTEDSARLSWTAPDAAFDSFFIGYLEPQPPGEAIHLFVPGS |
| | ERSYDLTGLKPGTEYNVTIQGVKGGFPSNPLSAQFTT |
| 1705 | MLPAPKNLVVSRVTEDSARLSWTAPDAAFDSFFIGYLEPQPPGEAIALWVPGS |
| | ERSYDLTGLKPGTEYNVTIQGVKGGFPSQPLHAHFTT |
| 1706 | MLPAPKNLVVSRVTEDSARLSWTAPDAAFDSFFIGYLEPQPPGEAISLFVPGS |
| | ERSYDLTGLKPGTEYNVTIQGVKGGFPSVPLSATFTT |
| 1707 | MLPAPKNLVVSRVTEDSARLSWTAPDAAFDSFFIGYLEPQPPGEAIGLLVPGS |
| | ERSYDLTGLKPGTEYNVTIQGVKGGFPSHPLLAVETT |
| 1708 | MLPAPKNLVVSRVTEDSARLSWTAPDAAFDSFFIGYLEPQPPGEAIYLRVPGS |
| | ERSYDLTGLKPGTEYNVTIQGVKGGFPSAPLHAHFTT |
| 1709 | MLPAPKNLVVSRVTEDSARLSWTAPDAAFDSFFIGYLEPQPPGEAIHLYVPGS |
| | ERSYDLTGLKPGTEYNVTIQGVKGGFPSHPLIANFTT |
| 1710 | MLPAPKNLVVSRVTEDSARLSWTAPDAAFDSFFIGYLEPQPPGEAIHLQVPGS |
| | ERSYDLTGLKPGTEYNVTIQGVKGGFPSMPLSAIFTT |
| 1711 | MLPAPKNLVVSRVTEDSARLSWTAPDAAFDSFFIGYLEPQPPGEAIALYVPGS |
| | ERSYDLTGLKPGTEYNVTIQGVKGGFPSLPLVATFTT |
| 1712 | MLPAPKNLVVSRVTEDSARLSWTAPDAAFDSFFIGYLEPQPPGEAIQLFVPGS |
| | ERSYDLTGLKPGTEYNVTIQGVKGGFPSDPLVASFTT |
| 1713 | MLPAPKNLVVSRVTEDSARLSWTAPDAAFDSFEIAYFENSYYGEAIVLTVPGS |
| | ERSYDLTGLKPGTEYAVYIGGVKGGSWSNPLSAISTT |
| 1714 | MLPAPKNLVVSRVTEDSARLSWTAPDAAFDSFFIGYLEPQPPGEAIALLVPGS |
| | ERSYDLTGLKPGTEYNVTIQGVKGGFPSSPLNAYFTT |
| 1715 | MLPAPKNLVVSRVTEDSARLSWTAPDAAFDSFFIGYLEPQPPGEAIHLQVPGS |
| | ERSYDLTGLKPGTEYNVTIHGVKGGIPSMPLSAKFTT |
| 1716 | MLPAPKNLVVSRVTEDSARLSWTAPDAAFDSFFIGYLEPQPPGEAIDLWVPGS |
| | ERSYDLTGLKPGTEYNVTIQGVKGGFPSMPLEAVFTT |
| 1717 | MLPAPKNLVVSRVTEDSARLSWTAPDAAFDSFFIGYLEPQPPGEAISLLVPGS |
| | ERSYDLTGLKPGTEYNVTIQGVKGGFPSHPLDASFTT |
| 1718 | MLPAPKNLVVSRVTEDSARLSWTAPDAAFDSFFIGYLEPQPPGEAIALYVPGS |
| | ERSYDLTGLKPGTEYNVTIQGVKGGFPSEPLVATFTT |
| 1719 | MLPAPKNLVVSRVTEDSARLSWTAPDAAFDSFFIGYLEPQPPGEAIHLIVPGS |
| | ERSYDLTGLKPGTEYNVTIQGVKGGFPSDPLDASFTT |
| 1720 | MLPAPKNLVVSRVTEDSARLSWTAPDAAFDSFFIGYLEPQPPGEAIGLLVPGS |
| | ERSYDLTGLKPGTEYNVTIQGVKGGFPSNPLSAIFTT |
| 1721 | MLPAPKNLVVSRVTEDSARLSWTAPDAAFDSFFIGYLEPQPPGEAIFLLVPGS |
| | ERSYDLTGLKPGTEYNVTIQGVKGGFPSMPLSAIFTT |
| 1722 | MLPAPKNLVVSRVTEDSARLSWTAPDAAFDSFFIGYLEPQPPGEAIHLRVPGS |
| | ERSYDLTGLKPGTEYNVTIQGVKGGFPSDPLIAVETT |
| 1723 | MLPAPKNLVVSRVTEDSARLSWTAPDAAFDSFFIGYLEPQPPGEAISLYVPGS |
| | ERSYDLTGLKPGTEYNVTIQGVKGGFPSNPLIAQFTT |
| 1724 | MLPAPKNLVVSRVTEDSARLSWTAPDAAFDSFEIGYFERSFYGEAIILFVPGS |
| | ERSYDLTGLKPGTEYAVWIGGVKGGVWSRPLSAIFTT |
| 1725 | MLPAPKNLVVSRVTEDSARLSWTAPDAAFDSFFIGYLEPQPPGEAIDLWVPGS |
| | ERSYDLTGLKPGTEYNVTIQGVKGGFPSYPLSASFTT |
| 1726 | MLPAPKNLVVSRVTEDSARLSWTAPDAAFDSFFIGYLEPQPPGEAIVLFVPGS |
| | ERSYDLTGLKPGTEYNVTIQGVKGGFPSNPLSAISTT |
| 1727 | MLPAPKNLVVSRVTEDSARLSWTAPDAAFDSFFIGYLEPQPPGEAIALYVPGS |
| | ERSYDLTGLKPGTEYNVTIQGVKGGFPSGPLEAYFTT |
| 1728 | MLPAPKNLVVSRVTEDSARLSWTAPDAAFDSFFIGYLEPQPPGEAIALYVPGS |
| | ERSYDLTGLKPGTEYNVTIQGVKGGFPSLPLEANFTT |
| 1729 | MLPAPKNLVVSRVTEDSARLSWTAPDAAFDSFFIGYLEPQPPGEAIGLQVPGS |
| | ERSYDLTGLKPGTEYNVTIQGVKGGFPSDPLQAIFTT |
| 1730 | MLPAPKNLVVSRVTEDSARLSWTAPDAAFDSFFIGYLEPQPPGEAIALWVPGS |
| | ERSYDLTGLKPGTEYNVTIQGVKGGFPSHPLAAVETT |
| 1731 | MLPAPKNLVVSRVTEDSARLSWTAPDAAFDSFFIGYLEPQPPGEAIHLLVPGS |
| | ERSYDLTGLKPGTEYNVTIQGVKGGFPSEPLWAKFTT |
| 1732 | MLPAPKNLVVSRVTEDSARLSWTAPDAAFDSFFIGYLEPQPPGEAISLWVPGS |
| | ERSYDLTGLKPGTEYNVTIQGVKGGFPSTPLSAKFTT |
| 1733 | MLPAPKNLVVSRVTEDSARLSWTAPDAAFDSFFIGYLEPQPPGEAIDLWVPGS |
| | ERSYDLTGLKPGTEYNVTIQGVKGGFPSYPLLAIFTT |
| 1734 | MLPAPKNLVVSRVTEDSARLSWTAPDAAFDSFFIGYLEPQPPGEAIHLFVPGS |
| | ERSYDLTGLKPGTEYNVTIQGVKGGFPSVPLNAYFTT |
| 1735 | MLPAPKNLVVSRVTEDSARLSWTAPDAAFDSFEIVYAEWTQHGEAIVLTVPGS |
| | ERSYDLTGLKPGTEYYVHIGGVKGGKWSPPLYAIFTT |
| 1736 | MLPAPKNLVVSRVTEDSARLSWTAPDAAFDSFFIGYLEPQPPGEAILLWVPGS |
| | ERSYDLTGLKPGTEYNVTIQGVKGGFPSDPLIANFTT |
| 1737 | MLPAPKNLVVSRVTEDSARLSWTAPDAAFDSFEIWYAEWRQVGEAIVLTVPGS |
| | ERSYDLTGLKPGTEYNVDIHGVKGGKVSWPLSAISTT |
| 1738 | MLPAPKNLVVSRVTEDSARLSWTAPDAAFDSFFIGYLEPQPPGEAIELYVPGS |
| | ERSYDLTGLKPGTEYNVTIQGVKGGFPSDPLHAAFTT |
| 1739 | MLPAPKNLVVSRVTEDSARLSWTAPDAAFDSFEIDYVEFIFTGEAIGLIVPGS |
| | ERSYDLTGLKPGTKYWVTIAGVKGGEWSTPLQAFFTT |
| 1740 | MLPAPKNLVVSRVTEDSARLSWTAPDAAFDSFFIGYLEPQPPGEAIHLFVPGS |
| | ERSYDLTGLKPGTEYNVTIQGVKGGFPSDPLVASFTT |
| 1741 | MLPAPKNLVVSRVTEDSARLSWTAPDAAFDSFFIGYLEPQPPGEAIQLFVPGS |
| | ERSYDLTGLKPGTEYNVTIQGVKGGFPSLPLIAYFTT |
| 1742 | MLPAPKNLVVSRVTEDSARLSWTAPDAAFDSFFIGYLEPQPPGEAIYLYVPGS |
| | ERSYDLTGLKPGTEYNVTIQGVKGGFPSYPLTAQFTT |
| 1743 | MLPAPKNLVVSRVTEDSARLSWTAPDAAFDSFFIGYLEPQPPGEAIDLYVPGS |
| | ERSYDLTGLKPGTEYNVTIQGVKGGFPSMPLYARFTT |
| 1744 | MLPAPKNLVVSRVTEDSARLSWTAPDAAFDSFYIAYQELSLWGEAIVLTVPGS |
| | ERSYDLTGLKPGTEYNVTIQGVKGGFPSLPLAAQFTT |
| 1745 | MLPAPKNLVVSRVTEDSARLSWTAPDAAFDSFFIGYLEPQPPGEAISLFVPGS |
| | ERSYDLTGLKPGTEYNVTIQGVKGGFPSEPLAAKFTT |
| 1746 | MLPAPKNLVVSRVTEDSARLSWTAPDAAFDSFFIGYLEPQPPGEAIGLLVPGS |
| | ERSYDLTGLKPGTEYNVTIQGVKGGFPSIPLVADFTT |
| 1747 | MLPAPKNLVVSRVTEDSARLSWTAPDAAFDSFFIGYLEPQPPGEAIHLQVPGS |
| | ERSYDLTGLKPGTEYNVTIQGVKGGFPSIPLVAQFTT |
| 1748 | MLPAPKNLVVSRVTEDSARLSWTAQDAAFDSFFIGYLEPQPPGEAIALLVPGS |
| | ERSYDLTGLKPGTEYNVTIQGVKGGFPSHPLSASFTT |
| 1749 | MLPAPKNLVVSRVTEDSARLSWTAPDAAFDSFFIGYLEPQPPGEAINLTVPGS |
| | ERSYDLTGLKPGTEYNVTIQGVKGGFPSYPLQASFTT |
| 1750 | MLPAPKNLVVSRVTEDSARLSWTAPDAAFDSFTISYPEEAKHGEAIVLTVPGS |
| | ERSYDLTGLKPGTEYGVPINGVKGGVSSLPLSAIFTT |
| 1751 | MLPAPKNLVVSRVTEDSARLSWTAPDAAFDSFFIGYLEPQPPGEAIALYVPGS |
| | ERSYDLTGLKPGTEYNVTIQGVKGGFPSHPLQAHFTT |
| 1752 | MLPAPKNLVVSRVTEDSARLSWTAPDAAFDSFFIGYLEPQPPGEAIVLLVPGS |
| | ERSYDLTGLKPGTEYNVTIQGVKGGFPSAPLVATFTT |
| 1753 | MLPAPKNLVVSRVTEDSARLSWTAPDAAFDSFLDLLPSNWDIAGEAIVLTVPG |
| | SERSYDLTGLKPGTEYHVNILGVKGGKESLPLVANFTT |
| 1754 | MLPAPKNLVVSRVTEDSARLSWTAPDAAFDSFFIRYLEPQPPGEAIHLSVPGS |
| | ERSYDLTGLKPGTEYNVTIQGVKGGFPSMPLYAIFTT |
| 1755 | MLPAPKNLVVSRVTEDSARLSWTAPDAAFDSFFIGYLEPQPPGEAISLYVPGS |
| | ERSYDQTGLKPGTEYNVTIQGVKGGFPSDPLSARFTT |
| 1756 | MLPAPKNLVVSRVTEDSARLSWTAPDAAFDSFFIGYLEPQPPGEAIGLFVPGS |
| | ERSYDLTGLKPGTEYNVTIQGVKGGFPSMPLSPIFTT |
| 1757 | MLPAPKNLVVSRVTEDSARLSWTAPDAAFDSFFIGYLEPQPPGEAIHLQVPGS |
| | ERSYDLTGLKPGTEYNVTIQGVKGGFPSVPLHARFTT |
| 1758 | MLPAPKNLVVSRVTEDSARLSWTAPDAAFDSFLILYIEQDHRGEAIVLTVPGS |
| | ERSYDLTGLKPGTEYWVHITGVKGGYYSAPLSAIFTT |
| 1759 | MLPAPKNLVVSRVTEDSARLSWTAPDAAFDSFFIGYLEPQPPGEAIQLFVPGS |
| | ERSYDLTGLKPGTEYNVTIQGVKGGFPSTPLVASFTT |
| 1760 | MLPAPKNLVVSRVTEDSARLSWTAPDAAFDSFFIGYLEPQPPGEAIDLYVPGS |
| | ERSYDLTGLKPGTEYNVTIQGVKGGFPSEPLWADFTT |
| 1761 | MLPAPKNLVVSRVTEDSARLSWTAPDAAFDSFYIAYAEPRPDGEAIWLLVPGS |
| | ERSYDLTGLKPGTEYSVLIHGVKGGLLSSPLSADFTT |
| 1762 | MLPAPKNLVVSRVTEDSARLSWTAPDAAFDSFFIGYLEPQPPGEAIQLLVPGS |
| | ERSYDLTGLKPGTEYNVTIQGVKGGFPSDPLVAYSTT |
| 1763 | MLPAPKNLVVSRVTEDSARLSWTAPDAAFDSFLIQYDEEGVWGEAIVLTVPGS |
| | ERSYDLTGLKPGTEYSVGIGGVKGGWSSVPLSAIFTT |
| 1764 | MLPAPKNLVVSRVTEDSARLSWTAPDAAFDSFFIGYLEPQPPGEAIHLYVPGS |
| | ERSYDLTGLKPGTEYNVTIQGVKGGFPSLPLVATFTT |
| 1765 | MLPAPKNLVVSRVTEDSARLSWTAPDAAFDSFFIGYLEPQPPGEAITLLVPGS |
| | ERSYDLTGLKPGTEYNVTIQGVKGGFPSMPLSAIFTT |
| 1766 | MLPAPKNLVVSRVTEDSARLSWTAPDAAFDSFTIWYYESPTGGEAIVLTVPGS |
| | ERSYDLTGLKPGTEYMVFIQGVKGGCFSTPLYAIFTT |
| 1767 | MLPAPKNLVVSRVTEDSARLSWTAPDAAFDSFFIGYLEPQPPGEAIELYVPGS |
| | ERSYDLTGLKPGTEYNVTIQGVKGGFPSLPLVATFTT |
| 1768 | MLPAPKNLVVSRVTEDSARLSWTAPDAAFDSFFIGYLEPQPPGEAIDLWVPGS |
| | ERSYDLTGLKPGTEYNVTIQGVKGGFPSLPLSASFTT |
| 1769 | MLPAPKNLVVSRVTEDSARLSWTAPDAAFDSFFIGYLEPQPPGEAIGLAVPGS |
| | ERSYDLTGLKPGTEYNVTIQGVKGGFPSEPLIADFTT |
| 1770 | MLPAPKNLVVSRVTEDSARLSWTAPDAAFDSFFIGYLEPQPPGEAITLYVPGS |
| | ERSYDLTGLKPGTEYNVTIQGVKGGFPSIPLSAEFTT |
| 1771 | MLPAPKNLVVSRVTEDSARLSWTAPDAAFDSFFIGYLEPQPPGEAIHLQVPGS |
| | ERSYDLTGLKPGTEYNVTIQGVKGGFPSIPLIAVETT |
| 1772 | MLPAPKNLVVSRVTEDSARLSWTAPDAAFDSFEIAYFETCRTGEAIVLTVPGS |
| | ERSYDLTGLKPGTEYGVSIYGVKGGAHSGPLSAIFTT |
| 1773 | MLPAPKNLVVSRVTEDSARLSWTAPDAAFDSFFIGYLEPQPPGEAIHLYVPGS |
| | ERSYDLTGLKPGTEYNVTIQGVKGGFPSVPLIAKFTT |
| 1774 | MLPAPKNLVVSRVTEDSARLSWTAPDAAFDSFFIGYLEPQPPGEAINLWVPGS |
| | ERSYDLTGLKPGTEYNVTIQGVKGGFPSTPLFAGFTT |
| 1775 | MLPAPKNLVVSRVTEDSARLSWTAPDAAFDSFEIDYAEDIREGEAIALVVPGS |
| | ERSYDLTGLKPGTEYWVSIGGVKGGTWSRPLFAPFTT |
| 1776 | MLPAPKNLVVSRVTEDSARLSWTAPDAAFDSFFIGYLEPQPPGEAITLYVPGS |
| | ERSYDMTGLKPGTEYNVTIQGVKGGFPSLPLQAHFTT |
| 1777 | MLPAPKNLIVSRVTEDSARLSWTAPDAAFDSFFIGYLEPQPPGEAIHLQVPGS |
| | ERSYDLTGLKPGTEYNVTIQGVKGGFPSNPLHAEFTT |
| 1778 | MLPAPKNLVVSRVTEDSARLSWTAPDAAFDSFFIGYLEPQPPGEAITLVVPGS |
| | ERSYDLTGLKPGTEYNVTIQGVKGGFPSDPLVASFTT |
| 1779 | MLPAPKNLVVSRVTEDSARLSWTAPDAAFDSFEIAYQELSLWGEAIVLTVPGS |
| | ERSYDLIGLKPGTEYYVHIGGVKGGIWSSPLSAISTT |
| 1780 | MLPAPKNLVVSRVTEDSARLSWTAPDAAFDSFFIGYLEPQPPGEAIHLWVPGS |
| | ERSYDLTGLKPGTEYNVTIQGVKGGFPSLPLLAKFTT |
| 1781 | MLPAPENLVVSRVTEDSARLSWTALDAAFDSFFIGYLEPQPPGEAISLLVPGS |
| | ERSYDLTGLKPGTEYNVTIQGVKGGIASEPLSAAFTT |
| 1782 | MLPAPKNLVVSRVTEDSARLSWTAPDAAFDSFFIGYLEPQPPGEAIGLRVPGS |
| | ERSYDLTGLKPGTEYNVTIQGVKGGFPSEPLIADFTT |
| 1783 | MLPAPKNLVVSRVTEDSARLSWTAPDAAFDSFIIQYGEYLRWGEAIVLTVPGS |
| | ERSYDLTGLKPGTEYQVEIYGVKGGPLSKPLSAIFTT |
| 1784 | MLAPKNLVVSRVTEDSARLSWTAPDAAFDSFFIGYLEPQPPGEAISLLVPGSE |
| | RSYDLTGLKPGTEYNVTIQGVKGGFPSSPLLAGFTT |
| 1785 | MLPAPKNLVVSRVTEDSARLSWTAPDAAFDSFFIGYLEPQPPGEAISLQVPGS |
| | ERSYDLTGLKPGTEYNVTIQGVKGGFPSAPLVANFTT |
| 1786 | MLPAPKNLVVSRVTEDSARLSWTAPDAAFDSFFIGYLEPQPSGEAIHLIVPGS |
| | ERSYDLTGLKPGTEYNVTIQGVKGGFPSDPLVASFTT |
| 1787 | MLPAPKNLVVSRVTEDSARLSWTAPDAAFDSFFIGYLEPQPPGEAIVLWVPGS |
| | ERSYDLTGLKPGTEYNVTIQGVKGGFPSHPLVAKFTT |
| 1788 | MLPAPKNLVVSRVTEDSARLSWTAPDAAFDSFFIGYLEPQPPGEAILLAVPGS |
| | ERSYDLTGLKPGTEYNVTIQGVKGGFPSLPLVAHFTT |
| 1789 | MLPAPKNLVVSRVTEDSARLSWTTPDAAFDSFFIGYLEPQPPGEAISLYVPGS |
| | ERSYDLTGLKPGTEYNVTIQGVKGGFPSMPLSAIFTT |
| 1790 | MLPAPKNLVVSRVTEDSARLSWTAPDAAFDSFFINYREDKWIGEAIVLTVPGS |
| | ERSYDLTGLKPGTEYSVPIDGVKGGAASPPLSAIFTT |
| 1791 | MLPAPKNLVVSRVTEDSARLSWTAPDAAFDSFFIGYLEPQPPGEAIILWVPGS |
| | ERSYDLTGLKPGTEYNVTIQGVKGGFPSVPLIAQFTT |
| 1792 | MLPAPKNLVVSRVTEDSARLSWTAPDAAFDSFFIGYLEPQPPGEAITLLVPGS |
| | ERSYDLTGLKPGTEYNVTIQGVKGGFPSKPLYAYFTT |
| 1793 | MLPAPKNLVVSRVTEDSARLSWTAPDAAFDSFFIGYLEPQPPGEAIILWVPGS |
| | ERSYDLTGLKPGTEYNVTIQGVKGGFPSIPLIAVETT |
| 1794 | MLPAPKNLVVSRVTEDSARLSWTAPDAAFDSFFIGYLEPQPPGEAISLYVPGS |
| | ERSYDLTGLKPGTEYNVTIQGVKGGFPSSPLFADFTT |
| 1795 | MLPAPKNLVVSRVTEDSARLSWTAPDAAFDSFFIGYLEPQPPGEAIDLLVPGS |
| | ERSYDLTGLKPGTEYNVTIQGVKGGFPSMPLSAIFTT |
| 1796 | MLPAPKNLVVSRVTEDSARLSWTAPDAAFDSFFIGYLEPQPPGEAISLLVPGS |
| | ERSYDLTGLKPGTEYNVTIQGVKGGFPSVPLSAIFTT |
| 1797 | MLPAPKNLVVSRVTEDSARLSWTAPDAAFDSFFIGYLEPQPPGEAIGLHVPGS |
| | ERSYDLTGLKPGTEYNVTIQGVKGGFPSIPLEAKFTT |
| 1798 | MLPAPKNLVVSRVTEDSARLSWTAPDAAFDSFEISYFETSWHGEAIVLIVLGS |
| | ERSYDLTGLKPGTEYRVYIGGVKGGSWSQPLSAIFTT |
| 1799 | MLPAPKNLVVSRVTEDSARLSWTAPDAAFDSFFIGYLEPQPPGEAIGLVVPGS |
| | ERSYDLTGLKPGTEYNVTIQGVKGGFPSHPLGAIFTT |
| 1800 | MLPAPKNLVVSRVTEDSARLSWTALDAAFDSFFIGYLEPQPPGEAIHLFVPGS |
| | ERSYDLTGLKPGTEYNVTIQGVKGGFPSEPLIANFTT |
| 1801 | MLPAPKNLVVSRVTEDSARLSWTAPDAAFDSFEIGYWENAYKGEAIVLTVPGS |
| | ERSYDLTGLKPGTEYVVFIGGVKGGVWSVPLSAISTT |
| 1802 | MLPAPKNLVVSRVTEDSARLSWTAPDAAFDSFFIGYLEPQPPGEAIHLLVPGS |
| | ERSYDLTGLKPGTEYNVTIQGVKGGFPSMPLSAIFTT |
| 1803 | MLPAPKNLVVSRVTEDSARLSWTAPDAAFDSFFIGYLEPQPPGEAITLSVPGS |
| | ERSYDLTGLKPGTEYNVTIQGVKGGFPSEPLIAIFTT |
| 1804 | MLPAPKNLVVSRVTEDSARLSWTAPDAAFDSFEIGYWENAYKGEAIVLTVPGS |
| | ERSYDLTGLKPGTEYEVFIGGVKGGVWSVPLSAIFTT |
| 1805 | MLPAPKNLVVSRVTEDSARLSWTAPDAAFDSFFIAYGESPESGEAIVLTVPGS |
| | ERSYDLTGLKPGTEYLVWIAGVKGGYYSDPLSAIFTT |
| 1806 | MLPAPKNLVVSRVTEDSARLSWTAPDAAFDSFFIGYLEPQPPGEAIDLFVPGS |
| | ERSYDLTGLKPGTEYNVTIQGVKGGFPSLPLVASFTT |
| 1807 | MLPAPKNLVVSRVTEDSARLSWTAPDAAFDSFFIGYLEPQPPGEAIDLWVPGS |
| | ERSYDLTGLKPGTEYNVTIQGVKGGFPSDPLDAHFTT |
| 1808 | MLPAPKNLVVSRVTEDSARLSWTAPDAAFDSFFIGYLEPQPPGEAISLYVPGS |
| | ERSYDLTGLKPGTEYNVTIQGVKGGFPSLPLAAQFTT |
| 1809 | MLPAPKNLVVSRVTEDSARLSWTAPDAAFDSFFIGYLEPQPPGEAIHLFVPGS |
| | ERSYDLTGLKPGTEYNVTIQGVKGGFPSEPLIANFTT |
| 1810 | MLPAPKNLVVSRVTEDSARLSWTAPDAAFDSFFIGYLEPQPPGEAIALWVPGS |
| | ERSYDLTGLKPGTEYNVTIQGVKGGFPSTPLIAQFTT |
| 1811 | MLPAPKNLVVSRVTEDSARLSWTAPDAAFDSFFIGYLEPQPPGEAISLWVPGS |
| | ERSYDLTGLKPGTEYNVTIQGVKGGFPSSPLVAYFTT |
| 1812 | MLPAPKNLVVSRVTEDSARLSWTAPDAAFDSFFIGYLEPQPPGEAIALVVPGS |
| | ERSYDLTGLKPGTEYNVTIQGVKGGFPSMPLFAYFTT |
| 1813 | MLPAPKNLVVSRVTEDSARLSWTAPDAAFDSFFIGYLEPQPPGEAISLWVPGS |
| | ERSYDLTGLKPGTEYNVTIQGVKGGFPSDPLGAHFTT |
| 1814 | MLPAPKNLVVSRVTEDSARLSWTAPDAAFDSFPIAYPEHLPPGEAIVLTVPGS |
| | ERSYDLTGLKPGTEYPVNIRGVKGGFVSFPLSAIFTT |
| 1815 | MLPAPKNLVVSRVTEDSARLSWTAPDAAFDSFFIGYLEPQPPGEAIVLWVPGS |
| | ERSYDLTGLKPGTEYNVTIQGVKGGFPSHPLVAKFTT |
| 1816 | MLPAPKNLVVSRVTEDSARLSWTAPDAAFDSFFIGYLEPQPPGEAIVLWVPGS |
| | ERSYDLTGLKPGTEYNVTIQGVKGGFPSHPLVAHFTT |
| 1817 | MLPAPKNLVVSRVTEDSARLSWTAPDAAFDSFFIGYLEPQPPGEAITLYVPGS |
| | ERSYDLNGLKPGTEYNVTIQGVKGGFPSMPLSAIFTT |
| 1818 | MLPAPKNLVVSRVTEDSARLSWTAPDAAFDSFFIGYLEPQPPGEAIDLLVPGS |
| | ERSYDLTGLKPGTEYNVTIQGVKGGFPSEPLTAEFTT |
| 1819 | MLPAPKNLVVSRVTEDSARLSWTAPDAAFDSFFIGYLEPQPPGEAIHLWVPGS |
| | ERSYDLTGLKPGTEYNVTIQGVKGGFPSMPLSAIFTT |
| 1820 | MLPAPKNLVVSRVTEDSARLSWTAPDAAFDSFFIGYLEPQPPGEAIALYVPGS |
| | ERSYDLTGLKPGTEYNVTIQGVKGGFPSEPLVATFTT |
| 1821 | MLPAPKNLVVSRVTEDSARLSWTAPDAAFDSFFIGYLEPQPPGEAISLIVPGS |
| | ERSYDLTGLKPGTEYNVTIQGVKGGFPSDPLIAIFTT |
| 1822 | MLPAPKNLVVSRVTEDSARLSWTAPDAAFDSFFIGYLEPQPPGEAIHLYVPGS |
| | ERSYDLTGLKPGTEYNVTIQGVKGGFPSEPLIANFTT |
| 1823 | MLPAPKNLVVSRVTEDSARLSWTAPDAAFDSFFIGYLEPQPPGEAITLLVPGS |
| | ERSYDLTGLKPGTEYNVTIQGVKGGFPSIPLIAIFTT |
| 1824 | MLPAPKNLVVSRVTEDSARLSWTAPDAAFDSFFIGYLEPQPPGEAIDLYVPGS |
| | ERSYDLTGLKPGTEYNVTIQGVKGGFPSLPLQAHFTT |
| 1825 | MLPAPKNLVVSRVTEDSARLSWTAPDAAFDSFFIGYLEPQPPGEAIDLLVPGS |
| | ERSYDLTGLKPGTEYNVTIQGVKGGFPSEPLFAHFTT |
| 1826 | MLPAPKNLVVSRVTEDSARLSWTAPDAAFDSFEIGYFEYSYYGEAIVLTVPGS |
| | ERSYDLIGLKPGTEYRVYIGGVKGGGWSRPLSAIFTT |
| 1827 | MLPAPKNLVVSRVTEDSARLSWTAPDAAFDSFDINYDELSGEGEAIALFVPGS |
| | ERSYDLTGLKPGTEYWVSIGGVKGGRFSEPLYARFTT |
| 1828 | MLPAPKNLVVSRVTEDSARLSWTAPDAAFDSFFIGYLEPQPPGEAINLFVPGS |
| | ERSYDLTGLKPGTEYNVTIQGVKGGFPSMPLSAIFTT |
| 1829 | MLPAPKNLVVSRVTEDSARLSWTAPDAAFDSFFIGYLEPQPPGEAIHLLVPGS |
| | ERSYDLTGLKPGTEYNVTIQGVKGGFPSGPLQASFTT |
| 1830 | MLPAPKNLVVSRVTEDSARLSWTAPDAAFDSFFIGYLEPQPPGEAIHLQVPGS |
| | ERSYDLTGLKPGTEYNVTIQGVKGGFPSDPLVAKFTT |
| 1831 | MLPAPKNLVVSRVTEDSARLSWTAPDAAFDSFDIGYTETVSFGEAIVLTVPGS |
| | ERSYDLTGLKPGTEYIVKILGVKGGFASFPLSAIFTT |
| 1832 | MLPAPKNLVVSRVTEDSARLSWTAPDAAFDSFEIGYFENLYLGEAIVLTVPGS |
| | ERSYDLTGLKPGTEYWVQIGGVKGGNWSAPLSAIFTT |
| 1833 | MLPAPKNLVVSRVTEDSARLSWTAPDAAFDSFFIGYLEPQPPGEAIDLYVPGS |
| | ERSYDLTGLKPGTEYNVTIQGVKGGFPSHPLVAVETT |
| 1834 | MLPAPKNLVVSRVTEDSARLSWTAPDAAFDSFFIGYLEPQPPGEAIHLFVPGS |
| | ERSYDLTGLKPGTEYNVTIQGVKGGFPSDPLHAAFTT |
| 1835 | MLPAPKNLVVSRVTEDSARLSWTAPDAAFDSFYIAYAEPRPDGEAIILVVPGS |
| | ERSYDLTGLKPGTEYSVLIHGVKGGLLSSPLDAGETT |
| 1836 | MLPAPKNLVVSRVTEDSARLSWTAPDAAFDSFFIGYLEPQPPGEAIDLWVPGS |
| | ERSYDLTGLKPGTEYNVTIQGVKGGFPSMPLDAYFTT |
| 1837 | MLPAPKNLVVSRVTEDSARLSWTAPDAAFDSFFIGYLEPQPPGEAIELFVPGS |
| | ERSYDLTGLKPGTEYNVTIQGVKGGFPSMPLSAIFTT |
| 1838 | MLPAPKNLVVSRVTEDSARLSWTAPDAAFDSFFIGYLEPQPPGEAISLYVPGS |
| | ERSYDLIGLKPGTEYNVTIQGVKGGFPSIPLVAVETT |
| 1839 | MLPAPKNLVVSRVTEDSARLSWTAPDAAFDSFFIGYLEPQPPGEAIALLVPGS |
| | ERSYDLTGLKPGTEYNVTIQGVKGGFPSHPLSASFTT |
| 1840 | MLPAPKNLVVSRVTEDSARLSWTAPDAAFDSFFIGYLEPQPPGEAISLVVPGS |
| | ERSYDLTGLKPGTEYNVTIQGVKGGFPSNPLIATFTT |
| 1841 | MLPAPKNLVVSRVTEDSARLSWTAPDAAFDSFEIAYIEVNIQGEAIVLTVPGS |
| | ERSYDLTGLKPGTEYYVHIGGVKGGPSSSPLSAIFTT |
| 1842 | MLPAPKNLVVSRVTEDSARLSWTAPDAAFDSFFIGYLEPQPPGEAIHLIVPGS |
| | ERSYDLIGLKPGTEYNVTIQGVKGGFPSDPLVASFTT |
| 1843 | MLPAPKNLVVSRVTEDSARLSWTAPDAAFDSFFIGYLEPQPPGEAIYLFVPGS |
| | ERSYDLTGLKPGTEYNVTIQGVKGGFPSMPLSAIFTT |
| 1844 | MLPAPKNLVVSRVTEDSARLSWTAPDAAFDSFFIGYLEPQPPGEAISLWVPGS |
| | ERSYDLTGLKPGTEYNVTIQGVKGGFPSMPLSAIFTT |
| 1845 | MLPAPKNLVVSRVTEDSARLSWTAPDAAFDSFFIGYLEPQPPGEAIHLFVPGS |
| | ERSYDLTGLKPGTEYNVTIQGVKGGFPSIPLYARFTT |
| 1846 | MLPAPKNLVVSRVTEDSARLSWTAPDAAFDSFEIAYFETCRTGEAIVLTVPGS |
| | ERSYDLTGLKPGTEYWVQIGGVKGGNWSAPLSAIFTT |
| 1847 | MLPAPKNLVVSRVTEDSARLSWTAPDAAFDSFFIGYLEPQPPGEAIHLFVPGS |
| | ERSYDLTGLKPGTEYNVTIQGVKGGFPSMPLSAIFTT |
| 1848 | MLPAPKNLVVSRVTEDSARLSWTAPDAAFDSFFIGYLEPQPPGEAIELYVPGS |
| | CRSYDLTGLKPGTEYNVTIQGVKGGFPSLPLVATFTT |
| 1849 | MLPAPKNLVVSRVTEDSARLSWTAPDAAFDSFFIGYLEPQPPGEAIHLYVPGS |
| | CRSYDLTGLKPGTEYNVTIQGVKGGFPSVPLIAKFTT |

Without being bound to any particular theory, in some embodiments, the FN3 domains that are linked to the nucleic acid molecule may be used in the targeted delivery of the therapeutic agent to cells that express the binding partner of the one or more FN3 domains, and lead intracellular accumulation of the nucleic acid molecule therein. This can allow the siRNA molecule to properly interact with the cell machinery to inhibit the expression of the target gene, improve efficacy, and also avoid, in some embodiments, toxicity that may arise with untargeted administration of the same siRNA molecule.

The FN3 domains described herein that bind to their specific target protein may be generated as monomers, dimers, or multimers, for example, as a means to increase the valency and thus the avidity of target molecule binding, or to generate bi- or multispecific scaffolds simultaneously binding two or more different target molecules. The dimers and multimers may be generated by linking monospecific, bi- or multispecific protein scaffolds, for example, by the inclusion of an amino acid linker, for example a linker containing poly-glycine, glycine and serine, or alanine and proline.

Thus, as provided herein, the different FN3 domains that are linked to the siRNA molecule can also be conjugated or linked to another FN3 domain that binds to a different target. The linker can be a flexible linker. The linker can be a short peptide sequence, such as those described herein. For example, the linker can be a G/S or G/A linker and the like. As provided herein, the linker can be, for example, a linker as shown in Table 10.

TABLE 10

| Exemplary Peptide Linker Sequences | |
|---|---|
| SEQ ID NO | SEQUENCE |
| 645 | (GS)₂ |
| 646 | (GGGS)₂ |
| 647 | (GGGGS)₁₋₅ |
| 648 | (GGGGS)₅ |
| 649 | (GGGGA)₁₋₅ |
| 650 | (AP)₁₋₂₀ |
| 651 | (AP)₂₋₂₀ |
| 652 | (AP)₂ |
| 653 | (AP)₅ |
| 654 | (AP)₁₀ |
| 655 | (AP)₂₀ |
| 656 | A(EAAAK)₅AAA |
| 657 | (EAAAK)₁₋₅ |
| 658 | EAAAKEAAAKEAAAKEAAAK |
| 659 | GGGGSGGGGSGGGGSGGGGS |
| 660 | APAPAPAPAP |
| 661 | EAAAK |

In some embodiments, the FN3 domain comprising two FN3 domains connected by a linker have the amino acid sequence of SEQ ID NO: 645. In some embodiments, the FN3 domain comprising two FN3 domains connected by a linker have the amino acid sequence of SEQ ID NO: 646. In some embodiments, the FN3 domain comprising two FN3 domains connected by a linker have the amino acid sequence of SEQ ID NO: 647, In some embodiments, the FN3 domain comprising two FN3 domains connected by a linker have the amino acid sequence of SEQ ID NO: 648, In some embodiments, the FN3 domain comprising two FN3 domains connected by a linker have the amino acid sequence of SEQ ID NO: 649. In some embodiments, the FN3 domain comprising two FN3 domains connected by a linker have the amino acid sequence of SEQ ID NO: 650. In some embodiments, the FN3 domain comprising two FN3 domains connected by a linker have the amino acid sequence of SEQ ID NO: 651. In some embodiments, the FN3 domain comprising two FN3 domains connected by a linker have the amino acid sequence of SEQ ID NO: 652. In some embodiments, the FN3 domain comprising two FN3 domains connected by a linker have the amino acid sequence of SEQ ID NO: 653. In some embodiments, the FN3 domain comprising two FN3 domains connected by a linker have the amino acid sequence of SEQ ID NO: 654. In some embodiments, the FN3 domain comprising two FN3 domains connected by a linker have the amino acid sequence of SEQ ID NO: 655. In some embodiments, the FN3 domain comprising two FN3 domains connected by a linker have the amino acid sequence of SEQ ID NO: 656. In some embodiments, the FN3 domain comprising two FN3 domains connected by a linker have the amino acid sequence of SEQ ID NO: 657. In some embodiments, the FN3 domain comprising two FN3 domains connected by a linker have the amino acid sequence of SEQ ID NO: 658. In some embodiments, the FN3 domain comprising two FN3 domains connected by a linker have the amino acid sequence of SEQ ID NO: 659. In some embodiments, the FN3 domain comprising two FN3 domains connected by a linker have the amino acid sequence of SEQ ID NO: 660. In some embodiments, the FN3 domain comprising two FN3 domains connected by a linker have the amino acid sequence of SEQ ID NO: 661. In some embodiments, the FN3 domain comprising two FN3 domains connected by a linker have the amino acid sequence of one of SEQ ID Nos: 645-661.

The dimers and multimers may be linked to each other in a N- to C-direction. The use of naturally occurring as well as synthetic peptide linkers to connect polypeptides into novel linked fusion polypeptides is well known in the literature (Hallewell et al., J Biol Chem 264, 5260-5268, 1989; Alfthan et al., Protein Eng. 8, 725-731, 1995; Robinson & Sauer, Biochemistry 35, 109-116, 1996; U.S. Pat. No. 5,856,456). The linkers described in this paragraph may be also be used to link the domains provided in the formula provided herein and above.

Half-Life Extending Moieties

The FN3 domains may also, in some embodiments, incorporate other subunits for example via covalent interaction. In some embodiments, the FN3 domains further comprise a half-life extending moiety. Exemplary half-life extending moieties are albumin, albumin variants, albumin-binding proteins and/or domains, an aliphatic chain or chains that thing to serum proteins, transferrin and fragments and analogues thereof, and Fc regions. Amino acid sequences of the human Fc regions are well known, and include IgG1, IgG2, IgG3, IgG4, IgM, IgA and IgE Fc regions. In some embodiments, the FN3 domain binds to albumin, albumin variants, albumin-binding proteins and/or domains, and fragments and analogues thereof, extending the half-life of the entire molecule.

In some embodiments, the albumin binding domain comprises the amino acid sequence of SEQ ID NOs: 5, 6, 7, 8, 9, 10, 11, 12, 13, 14, 15, 16, 17, 18, 19, 20, 21, 22, or 23. In some embodiments, the albumin binding domain (protein) is isolated. In some embodiments, the albumin binding domain comprises an amino acid sequence that is at least, or is, 85%, 86%, 87%, 88%, 89%, 90%, 91%, 92%, 93%, 94%, 95%, 96%, 97%, 98%, or 99% identical to the amino acid sequence of SEQ ID NO: 5, 6, 7, 8, 9, 10, 11, 12, 13, 14, 15, 16, 17, 18, 19, 20, 21, 22, or 23. In some embodiments, the albumin binding domain comprises an amino acid sequence that is at least, or is, 85%, 86%, 87%, 88%, 89%, 90%, 901%, 92%, 93%, 94%, 95%, 96%, 97%, 98%, or 99% identical to the amino acid sequence of SEQ ID NO: 5, 6, 7, 8, 9, 10, 11, 12, 13, 14, 15, 16, 17, 18, 19, 20, 21, 22, or 23 provided that the protein has a substitution that corresponds to position 10 of SEQ ID NO: 5, 6, 7, 8, 9, 10, 11, 12, 13, 14, 15, 16, 17, 18, 19, 20, 21, 22, or 23. In some embodiments, the substitution is A10V. In some embodiments, the substitution is A10G, A10L, A10I, A10T, or A10S. In some embodiments, the substitution at position 10 is any naturally occurring amino acid. In some embodiments, the isolated albumin binding domain comprises an amino acid sequence that has 1, 2, 3, 4, 5, 6, 7, 8, 9, 10, 11, 12, 13, or 14 substitutions when compared to the amino acid sequence of SEQ ID NOs: 5, 6, 7, 8, 9, 10, 11, 12, 13, 14, 15, 16, 17, 18, 19, 20, 21, 22, or 23. In some embodiments, the substitution is at a position that corresponds to position 10 of SEQ ID NOs: 5, 6, 7, 8, 9, 10, 11, 12, 13, 14, 15, 16, 17, 18, 19, 20, 21, 22, or 23. In some embodiments, FN3 domains provided comprises a cysteine residue in at least one residue position corresponding to residue positions 6, 11, 22, 25, 26, 52, 53, 61, 88 or positions 6, 8, 10, 11, 14, 15, 16, 20, 30, 34, 38, 40, 41, 45, 47, 48, 53, 54, 59, 60, 62, 64, 70, 88, 89, or90 of SEQ ID NO: 5, 6, 7, 8, 9, 10, 11, 12, 13, 14, 15, 16, 17, 18, 19, 20, 21, 22, or 23, or at a C-terminus. Although the positions are listed in a series, each position can also be chosen individually. In some embodiments, the cysteine is at a position that corresponds to position 6, 53, or 88. In some embodiments, additional examples of albumin binding domains can be found in U.S. Pat. No. 10,925,932, which hereby incorporated by reference.

All or a portion of an antibody constant region may be attached to the FN3 domain to impart antibody-like properties, especially those properties associated with the Fc region, such as Fc effector functions such as C1q binding, complement dependent cytotoxicity (CDC), Fc receptor binding, antibody-dependent cell-mediated cytotoxicity (ADCC), phagocytosis, downregulation of cell surface receptors (e.g., B cell receptor; BCR), and may be further modified by modifying residues in the Fc responsible for these activities (for review; see Strohl, Curr Opin Biotechnol. 20, 685-691, 2009).

Additional moieties may be incorporated into the FN3 domains such as polyethylene glycol (PEG) molecules, such as PEG5000 or PEG20,000, fatty acids and fatty acid esters of different chain lengths, for example laurate, myristate, stearate, arachidate, behenate, oleate, arachidonate, octanedioic acid, tetradecanedioic acid, octadecanedioic acid, docosanedioic acid, and the like, polylysine, octane, carbohydrates (dextran, cellulose, oligo- or polysaccharides) for desired properties. These moieties may be direct fusions with the protein scaffold coding sequences and may be generated by standard cloning and expression techniques. Alternatively, well known chemical coupling methods may be used to attach the moieties to recombinantly produced molecules disclosed herein.

A PEG moiety may for example be added to the FN3 domain t by incorporating a cysteine residue to the C-terminus of the molecule, or engineering cysteines into residue positions that face away from the binding face of the molecule, and attaching a PEG group to the cysteine using well known methods.

FN3 domains incorporating additional moieties may be compared for functionality by several well-known assays. For example, altered properties due to incorporation of Fc domains and/or Fc domain variants may be assayed in Fc receptor binding assays using soluble forms of the receptors, such as the FcγRI, FcγRII, FcγRIII or FcRn receptors, or using well known cell-based assays measuring for example ADCC or CDC, or evaluating pharmacokinetic properties of the molecules disclosed herein in in vivo models.

The compositions provided herein can be prepared by preparing the FN3 proteins and the nucleic acid molecules and linking them together. The techniques for linking the proteins to a nucleic acid molecule are known and any method can be used. For example, in some embodiments, the nucleic acid molecule is modified with a linker, such as the linker provided herein, and then the protein is mixed with the nucleic acid molecule comprising the linker to form the composition. For example, in some embodiments, an FN3 domain is conjugated to an siRNA via a cysteine using thiol-maleimide chemistry. In some embodiments, a cysteine-containing FN3 domain can be reduced in, for example, phosphate buffered saline (or any other appropriate buffer) with a reducing agent (e.g., tris(2-carboxyethyl) phosphine (TCEP)) to yield a free thiol. Then, in some embodiments, the free thiol containing FN3 domain is mixed with a maleimide linked-modified siRNA duplex and incubated under conditions to form the linked complex. In some embodiments, the mixture is incubated for 0-5 hr, or about 1, 2, 3, 4 or 5 hr at room temperature (RT). The reaction can be, for example, quenched with N-ethyl maleimide. In some embodiments, the conjugates can be purified using affinity chromatography and ion exchange. Other methods can also be used and this is simply one non-limiting embodiment.

Methods of making FN3 proteins are known and any method can be used to produce the proteins. Examples are provided in the references incorporated by reference herein.

In some embodiments, the FN3 domain specifically binding CD71 comprises the amino acid sequence of SEQ ID NOs: 365-644 or 663-672, wherein a histidine tag has been appended to the N-terminal or C-terminal end of the polypeptide for ease of purification. In some embodiments, the histidine tag (His-tag) comprises six histidine residues (SEQ ID NO: 662). In further embodiments, the His-tag to connected to the FN3 domain by at least one glycine residue or about 2 to about 4 glycine residues. Accordingly, after purification of the FN3 domain and cleavage of the His-tag from the polypeptide one or more glycine may be left on the N-terminus or C-terminus. In some embodiments, if the His-tag is removed from the N-terminus all of the glycines are removed. In some embodiments, if the His-tag is removed from the C-terminus one or more of the glycines are retained.

In some embodiments, the FN3 domain specifically binding CD71 comprises the amino acid sequence of SEQ ID NOs: 365-644 or 663-672, wherein the N-terminal methionine is retained after purification of the FN3 domain. In some embodiments, the FN3 domain specifically binding CD71 comprises the amino acid sequence of SEQ ID NOs: 365-644 or 663-672, wherein the N-terminal methionine is not retained after purification of the FN3 domain.

For example, as described herein, in some embodiments, the amino acid sequence of SEQ ID NO: 570 without the methionine would be as follows:

(SEQ ID NO: 2310)
LPAPKNLVVSRVTEDSARLSWTAPDAAFDSFYIAYAEPR
PDGEAILLQVPGSCRSYDLTGLKPGTEYSVLIHGVKGGL
LSSPLTAIFTT

As provided for herein, the FN3 domains can be linked to a siRNA molecule. Although certain FN3 domains are illustrated with the methionine, it should be understood that the FN3 domain can be linked to the siRNA without the N-terminal methionine. Additionally, one of skill in the art would appreciate that the numbering for the cysteine residue location, which is provided for herein would be shifted to one residue lower without the N-terminal methionine being present.

For example, the amino acid sequence of the FN3 domain can be such as those provided for herein, including but not limited to an amino acid sequence that is at least 90%, 915, 92%, 93%, 94%, 95%, 95%, 96%, 97%, 98%, 99%, or is identical to the amino acid sequence of SEQ ID NO: 570 or SEQ ID NO: 2310, can be linked to a siRNA pair as provided for herein. In some embodiments, siRNA pair comprises a sense strand and an antisense strand. In some embodiments, the sense strand comprises the nucleotide sequence of SEQ ID NO: 2110, or a modified version thereof, and the antisense strand comprises the nucleotide sequence of SEQ ID NO: 2220, or a modified version thereof. In some embodiments, the sense strand comprises the nucleotide sequence of SEQ ID NO: 2113, or a modified version thereof, and the antisense strand comprises the nucleotide sequence of SEQ ID NO: 2223, or a modified version thereof. In some embodiments, the sense strand comprises the nucleotide sequence of SEQ ID NO: 2111, or a modified version thereof, and the antisense strand comprises the nucleotide sequence of SEQ ID NO: 2221, or a modified version thereof. In some embodiments, the sense strand comprises the nucleotide sequence of SEQ ID NO: 1890 and the antisense strand comprises the nucleotide sequence of SEQ ID NO: 2290. In some embodiments, the sense strand comprises the nucleotide sequence of SEQ ID NO: 1893 and the antisense strand comprises the nucleotide sequence of SEQ ID NO: 2293. In some embodiments, the sense strand comprises the nucleotide sequence of SEQ ID NO: 1941 and the antisense strand comprises the nucleotide sequence of SEQ ID NO: 2051. In some embodiments, the sense strand comprises the nucleotide sequence of SEQ ID NO: 1942 and the antisense strand comprises the nucleotide sequence of SEQ ID NO: 2052. In some embodiments, the sense strand comprises the nucleotide sequence of SEQ ID NO: 1944 and the antisense strand comprises the nucleotide sequence of SEQ ID NO: 2054. In some embodiments, the sense strand and the antisense strand pair (siRNA pair) are as provided for herein.

Kits

In some embodiments, a kit comprising the compositions described herein are provided. The kit may be used for therapeutic uses and as a diagnostic kit. In some embodiments, the kit comprises the FN3 domain conjugated to the nucleic acid molecule.

Uses of the Conjugates

The compositions provided for herein may be used to diagnose, monitor, modulate, treat, alleviate, help prevent the incidence of, or reduce the symptoms of human disease or specific pathologies in cells, tissues, organs, fluid, or, generally, a host.

In some embodiments, the FN3 domain can facilitate delivery to activated lymphocytes, dendritic cells, or other immune cells for treatment of immunological diseases. Thus, in some embodiments, the FN3 domain that binds to CD71 is directed to immune cells. In some embodiments, the FN3 domain that binds to CD71 is directed to B cells. In some embodiments, the FN3 domain that binds to CD71 is directed to T cells. In some embodiments, the FN3 domain that binds to CD71 is directed to dendritic cells. In some embodiments, the FN3 domain that binds to CD71 is directed to monocytes. In some embodiments, the FN3 domain that binds to CD71 does not have an anti-proliferative effect on immune cells. For example, in some embodiments, the FN3 domain that binds to CD71 does not have an anti-proliferative effect on B cells, T cells, dendritic cells, monocytes, or any combination thereof.

In some embodiments, methods of treating an autoimmune disease in a subject in need thereof are provided. In some embodiments, the methods comprise administering to the subject a polypeptide or the pharmaceutical composition that binds to CD71. In some embodiments, that the polypeptide is a FN3 domain that binds to CD71. In some embodiments, the polypeptide comprises an amino acid sequence such as SEQ ID NOs: 361-644 or 663-672, or a polypeptide as provided herein that is linked to or conjugated to a therapeutic agent. In some embodiments, a method of treating an autoimmune disease in a subject, the method comprising administering to the subject an FN3 domain that binds CD71 and the FN3 domain is conjugated to a therapeutic agent (e.g., cytotoxic agent, an oligonucleotide, such as an siRNA, ASO, and the like, an FN3 domain that binds to another target, and the like).

In some embodiments, the autoimmune disease is selected from the group consisting of rheumatoid arthritis, Hashimoto's autoimmune thyroiditis, celiac disease, diabetes mellitus type 1, vitiligo, rheumatic fever, pernicious anemia/atrophic gastritis, alopecia areata, immune thrombocytopenic purpura, psoriasis, inflammatory bowel disease, systemic lupus erythematosus, pemphigus, Sjogren's syndrome, myositis, lupus nephritis, neuroinflammatory diseases such as multiple sclerosis, or prevention of solid organ transplant rejection.

In some embodiments, methods of reducing the expression of a target gene in a cell are provided. In some embodiments, the methods comprise delivering to the cell with a composition or a pharmaceutical composition as provided herein. In some embodiments, the cell is ex vivo. In some embodiments, the cell is in vivo. In some embodiments, the target gene is CD40. The target gene, however, can be any target gene as the evidence provided herein demonstrates that siRNA molecules can be delivered efficiently when conjugated to an FN3 domain. In some embodiments, the siRNA targeting CD40 is linked to an FN3 domain. In some embodiments, the FN3 polypeptide (domain) is one that binds to CD71. In some embodiments, the FN3 polypeptide is as provided for herein or as provided for in PCT Application No. PCT/US20/55509, U.S. application Ser. No. 17/070,337, PCT Application No. PCT/US20/55470, or U.S. application Ser. No. 17/070,020, each of which is hereby incorporated by reference in its entirety. In some embodiments, the siRNA is not conjugated to an FN3 domain. In some embodiments, a method of reducing the expression of a target gene results in a reduction of about 99%, 90-99%, 50-90%, or 10-50% in the expression of the target gene.

In some embodiments, a method of reducing the expression of CD40 is provided. In some embodiments, the reduced expression is the expression (amount) of CD40 mRNA. In some embodiments, a method of reducing the expression of CD40 results in a reduction of about 99%, 90-99%, 50-90%, or 10-50% in the expression of CD40. In some embodiments, the reduced expression is the expression (amount) of CD40 protein. In some embodiments, the reduced protein is CD40 protein. In some embodiments, reduction of CD40 protein occurs in immune cells. In some embodiments, reduction of CD40 protein occurs in B cells. In some embodiments, reduction of CD40 protein occurs in T cells. In some embodiments, reduction of CD40 protein occurs in dendritic cells. In some embodiments, the method comprises delivering to a cell an siRNA molecule as provided herein that targets CD40. In some embodiments, the siRNA is conjugated to an FN3 domain. In some embodiments, the FN3 domain is an FN3 domain that binds to CD71. In some embodiments, the FN3 domain is as provided for herein. In some embodiments, the FN3 domain is a dimer of two FN3 domains that bind to CD71. In some embodiments, the FN3 domains are the same. In some embodiments, the two FN3 domains are different, i.e., bind to different regions or amino acid residues of CD71, i.e. a different epitope. In some embodiments, the method comprises administering to a subject (patient) a CD40 targeting siRNA molecule, such as those provided herein. In some embodiments, the CD40 targeting siRNA molecule administered to the subject is conjugated or linked to an FN3 domain. In some embodiments, the FN3 domain is an FN3 domain that binds to CD71. In some embodiments, the FN3 domain is as provided for herein. In some embodiments, the FN3 domain is a dimer of two FN3 domains that bind to CD71. In some embodiments, the FN3 domains are the same. In some embodiments, the two FN3 domains are different, i.e., bind to different regions or amino acid residues of CD71, i.e. a different epitope. In some embodiments, the CD71 binding domain is a polypeptide as provided for herein.

In some embodiments, methods of delivering an siRNA molecule to a cell in a subject are provided. In some embodiments, the methods comprise administering to the subject a pharmaceutical composition comprising a composition as provided for herein. In some embodiments, the cell is a CD71 positive cell. The term “positive cell” in reference to a protein refers to a cell that expresses the protein. In some embodiments, the protein is expressed on the cell surface. In some embodiments, the cell is a tumor cell, a liver cell, an immune cell, a heart cell, a muscle cell, a cell of the CNS, or a cell inside the blood brain barrier. In some embodiments, the cell is an immune cell. In some embodiments, the cell is a B cell. In some embodiments, the cell is a T cell. In some embodiments, the cell is a dendritic cell. In some embodiments, the siRNA molecule downregulates expression of a target gene in the cell. In some embodiments, the target gene is CD40.

In some embodiments, methods of reducing one or more serum cytokines in a subject are provided. In some embodiments, the method comprises administering an siRNA molecule. In some embodiments, the siRNA molecule downregulates expression of a target gene in a cell. In some embodiments, the target gene is CD40. In some embodiments, the one or more cells is a CD71 positive cell. In some embodiments, the one or more cells is an immune cell. In some embodiments, the cell is a B cell. In some embodiments, the cell is a dendritic cell. In some embodiments, the cell is a T cell. In some embodiments, the one or more serum cytokines comprises IFN-γ, IL-6, TNF-α, IL-12, IP-10, and/or RANTES, or any combination thereof. In some embodiments, the one or more serum cytokines comprises IFN-γ. In some embodiments, the one or more serum cytokines comprises IL-6. In some embodiments, the one or more serum cytokines comprises TNF-α. In some embodiments, the one or more serum cytokines comprises IL-12. In some embodiments, the one or more serum cytokines comprises IP-10. In some embodiments, the one or more serum cytokines comprises RANTES.

In some embodiments, methods of reducing or inhibiting cell migration are provided. In some embodiments, the methods comprise contacting a cell with an siRNA molecule. In some embodiments, the siRNA molecule downregulates expression of a target gene in the cell. In some embodiments, the target gene is CD40. In some embodiments, the cell is a CD71 positive cell. In some embodiments, the cell is an immune cell. In some embodiments, the cell is a B cell. In some embodiments, the cell is a dendritic cell. In some embodiments, the methods comprise reducing or inhibiting cell migration from blood to tissue. In some embodiments, the methods comprise reducing or inhibiting cell migration from blood to lymphoid organ tissue. In some embodiments, the methods comprise selectively reducing or inhibiting migration of B cells and/or dendritic cells, and not reducing or inhibiting migration of T cells.

In some embodiments, methods of inhibiting margination are provided. In some embodiments, the methods comprise contacting a cell with an siRNA molecule. In some embodiments, the siRNA molecule downregulates expression of a target gene in the cell. In some embodiments, the target gene is CD40. In some embodiments, the cell is a CD71 positive cell. In some embodiments, the cell is an immune cell. In some embodiments, the cell is a B cell. In some embodiments, the cell is a dendritic cell. In some embodiments, the methods comprise reducing or inhibiting cell margination from the interior of a blood vessel towards to the blood vessel wall. In some embodiments, the methods comprise selectively reducing or inhibiting margination of B cells and/or dendritic cells, and not reducing or inhibiting margination of T cells.

In some embodiments, the compositions or pharmaceutical compositions provided herein may be administered alone or in combination with other therapeutics, that is, simultaneously or sequentially.

“Treat” or “treatment” refers to the therapeutic treatment and prophylactic measures, wherein the object is to prevent or slow down (lessen) an undesired physiological change or disorder, such as the development or spread of cancer. In some embodiments, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

A “therapeutically effective amount” refers to an amount effective, at dosages and for periods of time necessary, to achieve a desired therapeutic result. A therapeutically effective amount of the compositions provided herein may vary according to factors such as the disease state, age, sex, and weight of the individual. Exemplary indicators of an effective amount is improved well-being of the patient, decrease or shrinkage of the size of a tumor, arrested or slowed growth of a tumor, and/or absence of metastasis of cancer cells to other locations in the body.

Administration/Pharmaceutical Compositions

In some embodiments, pharmaceutical compositions comprising the compositions provided herein and a pharmaceutically acceptable carrier, are provided. For therapeutic use, the compositions may be prepared as pharmaceutical compositions containing an effective amount of the domain or molecule as an active ingredient in a pharmaceutically acceptable carrier. “Carrier” refers to a diluent, adjuvant, excipient, or vehicle with which the active compound is administered. Such vehicles can be liquids, such as water and oils, including those of petroleum, animal, vegetable or synthetic origin, such as peanut oil, soybean oil, mineral oil, sesame oil, and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the molecules disclosed herein in such pharmaceutical formulation can vary widely, i.e., from less than about 0.5%, usually at least about 1% to as much as 15 or 20% by weight and will be selected primarily based on required dose, fluid volumes, viscosities, etc., according to the particular mode of administration selected. Suitable vehicles and formulations, inclusive of other human proteins, e.g., human serum albumin, are described, for example, in Remington: The Science and Practice of Pharmacy, 21^{st} Edition, Troy, D. B. ed., Lipincott Williams and Wilkins, Philadelphia, P A 2006, Part 5, Pharmaceutical Manufacturing, pp. 691-1092 (see especially pp. 958-989).

The mode of administration for therapeutic use of the compositions disclosed herein may be any suitable route that delivers the agent to the host, such as parenteral administration, e.g., intradermal, intramuscular, intraperitoneal, intravenous or subcutaneous, pulmonary; transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by for example intrarticular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

Pharmaceutical compositions can be supplied as a kit comprising a container that comprises the pharmaceutical composition as described herein. A pharmaceutical composition can be provided, for example, in the form of an injectable solution for single or multiple doses, or as a sterile powder that will be reconstituted before injection. Alternatively, such a kit can include a dry-powder disperser, liquid aerosol generator, or nebulizer for administration of a pharmaceutical composition. Such a kit can further comprise written information on indications and usage of the pharmaceutical composition.

### ENUMERATED EMBODIMENTS

Embodiments provided herein also include, but are not limited to, the following:

1. A composition comprising an siRNA molecule comprising a sense strand and antisense strand that targets CD40 gene, such as those provided herein.

2. The composition of embodiment 1, wherein the siRNA molecule does not contain any modified nucleobases.

3. The composition of embodiment 1 or 2, wherein the siRNA molecule further comprises a linker covalently attached to the sense strand or the antisense strand.

4. The composition of embodiment 3, wherein the linker is attached to a 5′ end or a 3′ end of the sense strand or the antisense strand.

5. The composition of any one of embodiments 1-4, wherein the siRNA molecule further comprises a vinyl phosphonate modification on the sense strand or the antisense strand.

6. The composition of embodiment 5, wherein the vinyl phosphonate modification is attached to a 5′ end or a 3′ end of the sense strand or the antisense strand.

7. The composition of any one of embodiments 1-6, wherein the sense strand comprises a nucleic acid sequence selected from any of SEQ ID NOs: 46-178, 312-331, 352-356, 673-805, and 939-958.

8. The composition of any one of embodiments 1-7, wherein the antisense strand comprises a nucleic acid sequence selected from any of SEQ ID NOs: 179-311, 332-351, 356-359, 806-938, and 959-978.

9. The composition of any one of the preceding embodiments, wherein the siRNA molecule comprises the siRNA pair of A1, B1, C1, D1, E1, F1, G1, H1, I1, J1, K1, L1, M1, N1, O1, P1, Q1, R1, S1, T1, U1, V1, W1, X1, Y1, Z1, A2, B2, C2, D2, E2, F2, G2, H2, I2, J2, K2, L2, M2, N2, O2, P2, Q2, R2, S2, T2, U2, V2, W2, X2, Y2, Z2, A3, B3, C3, D3, E3, F3, G3, H3, I3, J3, K3, L3, M3, N3, O3, P3, Q3, R3, S3, T3, U3, V3, W3, X3, Y3, Z3, A4, B4, C4, D4, E4, F4, G4, H4, I4, J4, K4, L4, M4, N4, O4, P4, Q4, R4, S4, T4, U4, V4, W4, X4, Y4, Z4, A5, B5, C5, D5, E5, F5, G5, H5, I5, J5, K5, L5, M5, N5, O5, P5, Q5, R5, S5, T5, U5, V5, W5, X5, Y5, Z5, A6, B6, C6, D6, E6, F6, G6, H6, I6, J6, K6, L6, M6, N6, O6, P6, Q6, R6, S6, T6, U6, V6, W6, B7, C7, P8, Q8, R8, S8, T8, U8, V8, W8, X8, Y8, Z8, A9, B9, C9, D9, E9, F9, G9, H9, I9, J9, K9, L9, M9, N9, O9, P9, Q9, R9, S9, T9, U9, V9, W9, X9, Y9, Z9, A10, B10, F10, G10, H10, I10, J10, K10, L10, M10, N10, O10, P10, Q10, R10, S10, T10, U10, V10, W10, X10, Y10, Z10, A11, B11, C11, D11, E11, F11, G11, H11, I11, J11, K11, L11, M11, N11, O11, P11, Q11, R11, or as set forth in Table 3A, Table 3B, Table 4A, Table 4B, Table 5A, or Table 5B.

10. The composition of any one of embodiments 1-6, wherein the sense strand consists essentially of 19 or 20 nucleotides.

11. The composition of any one of embodiments 1-6, wherein the antisense strand consists essentially of 21 nucleotides.

12. The composition of any one of the preceding embodiments, wherein the composition comprises an siRNA pair as provided for in Table 3A, Table 3B, Table 4A, Table 4B, Table 5A, or Table 5B, with a linker and/or vinyl phosphonate modification as provided for herein.

13. The composition of any one of the preceding embodiments, wherein the siRNA molecule has the formula as illustrated in Formula III:

(III)
N₁N₂N₃N₄N₅N₆N₇N₈N₉N₁₀N₁₁N₁₂N₁₃
N₁₄N₁₅N₁₆N₁₇N₁₈N₁₉
Sense-strand (SS)
N₂₁N₂₀N₁₉N₁₈N₁₇N₁₆N₁₅N₁₄N₁₃N₁₂
N₁₁N₁₀N₉N₈N₇N₆N₅N₄N₃N₂N₁
Antisense-strand (AS),

wherein each nucleotide represented by N, is independently, A, U, C, or G or a modified nucleotide base, such as those provided for herein.

14. The composition of embodiment 13, wherein the sense strand comprises a 2′O-methyl modified nucleotide with a phosphorothioate (PS)-modified backbone at N₁ and N₂, a 2′-fluoro modified nucleotide at N₃, N₇, N₈, N₉, N₁₂, and N₁₇, and a 2′O-methyl modified nucleotide at N₄, N₅, N₆, N₁₀, N₁₁, N₁₃, N₁₄, N₁₅, N₁₆, N₁₈, and N₁₉.

15. The composition of embodiment 13, wherein the antisense strand comprises a vinyl phosphonate moiety with a phosphorothioate (PS) modified backbone attached at N₁, a 2′fluoro-modified nucleotide with a PS-modified backbone at N₂, a 2′O-methyl modified nucleotide at N₃, N₄, N₅, N₆, N₇, N₈, N₉, N₁₀, N₁₁, N₁₂, N₁₃, Nis, N₁₆, N₁₇, Nis, and N₁₉, a 2′fluoro-modified nucleotide at N₁₄, and a 2′O-methyl modified nucleotide with a PS-modified backbone at N₂₀ and N₂₁.

16. The composition of embodiment 13, wherein the antisense strand comprises a vinyl phosphonate moiety is attached to N₁.

17. The composition of any one of embodiments 13-16, wherein the siRNA molecule is conjugated to a linker as shown in the following formula:

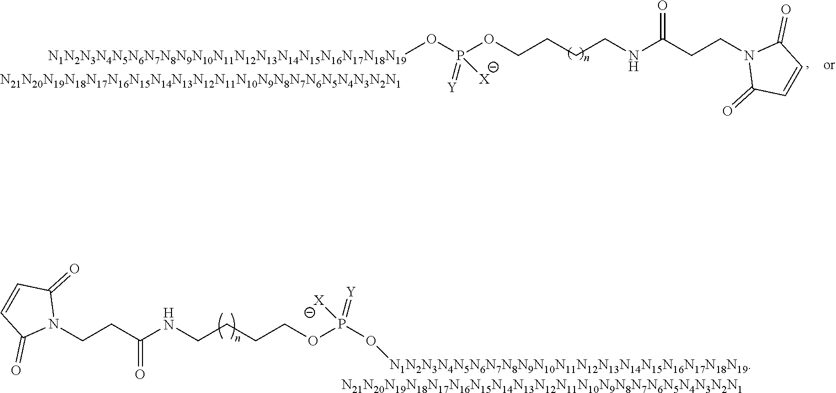

18. The composition of any one of the preceding embodiments, wherein the siRNA molecule has the formula as illustrated in Formula III:

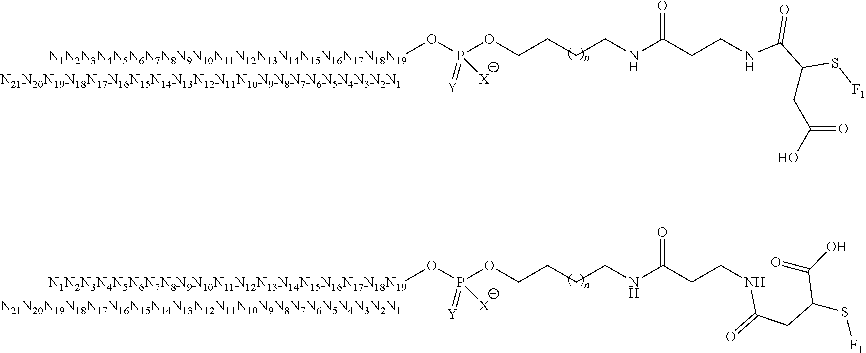

wherein F₁ is a polypeptide comprising at least one FN3 domain.

19. The composition of embodiment 18, wherein F₁ comprises polypeptide having a formula of (X₁)ₙ—(X₂)_{q}—(X₃)_{y}, wherein X₁ is a first FN3 domain; X₂ is a second FN3 domain; X₃ is a third FN3 domain or half-life extender molecule; wherein n, q, and y are each independently 0 or 1, provided that at least one of n, q, and y is 1.

20. A composition comprising one or more FN3 domains conjugated to an siRNA molecule comprising a sense strand and an antisense strand that targets CD40, such as those provided for herein.

21. The composition of embodiment 20, wherein the siRNA molecule does not contain any modified nucleobases.

22. The composition of embodiment 20 or 21, wherein the siRNA molecule further comprises a linker.

23. The composition of embodiment 22, wherein the linker is covalently attached to the sense strand or the antisense strand.

24. The composition of embodiment 22 or 23, wherein the linker is attached to a 5′ end or a 3′ end of the sense strand or the antisense strand.

25. The composition of any one of embodiments 20-24, wherein the siRNA molecule further comprises a vinyl phosphonate modification on the sense strand or the antisense strand.

26. The composition of 25, wherein the vinyl phosphonate modification is attached to a 5′ end or a 3′ end of the sense strand or the antisense strand.

27. The composition of any one of embodiments 20-26, wherein the sense strand comprises a nucleic acid sequence selected from any of SEQ ID NOs: 46-178, 312-331, 352-356, 673-805, and 939-958.

28. The composition of any one of embodiments 20-26, wherein the antisense strand comprises a nucleic acid sequence selected from any of SEQ ID NOs: 179-311, 332-351, 356-359, 806-938, and 959-978.

29. The composition of any one of embodiments 20-26, wherein the siRNA molecule comprises the siRNA pair of A1, B1, C1, D1, E1, F1, G1, H1, I1, J1, K1, L1, M1, N1, O1, P1, Q1, R1, S1, T1, U1, V1, W1, X1, Y1, Z1, A2, B2, C2, D2, E2, F2, G2, H2, I2, J2, K2, L2, M2, N2, O2, P2, Q2, R2, S2, T2, U2, V2, W2, X2, Y2, Z2, A3, B3, C3, D3, E3, F3, G3, H3, I3, J3, K3, L3, M3, N3, O3, P3, Q3, R3, S3, T3, U3, V3, W3, X3, Y3, Z3, A4, B4, C4, D4, E4, F4, G4, H4, I4, J4, K4, L4, M4, N4, O4, P4, Q4, R4, S4, T4, U4, V4, W4, X4, Y4, Z4, A5, B5, C5, D5, E5, F5, G5, H5, I5, J5, K5, L5, M5, N5, O5, P5, Q5, R5, S5, T5, U5, V5, W5, X5, Y5, Z5, A6, B6, C6, D6, E6, F6, G6, H6, I6, J6, K6, L6, M6, N6, O6, P6, Q6, R6, S6, T6, U6, V6, W6, B7, C7, P8, Q8, R8, S8, T8, U8, V8, W8, X8, Y8, Z8, A9, B9, C9, D9, E9, F9, G9, H9, I9, J9, K9, L9, M9, N9, O9, P9, Q9, R9, S9, T9, U9, V9, W9, X9, Y9, Z9, A10, B10, F10, G10, H10, I10, J10, K10, L10, M10, N10, O10, P10, Q10, R10, S10, T10, U10, V10, W10, X10, Y10, Z10, A11, B11, C11, D11, E11, F11, G11, H11, I11, J11, K11, L11, M11, N11, O11, P11, Q11, R11, or as set forth in Table 3A, Table 3B, Table 4A, Table 4B, Table 5A, or Table 5B.

30. The composition of any one of embodiments 20-26, wherein the siRNA molecule comprises the siRNA pair as provided for in Table 3A, Table 3B, Table 4A, Table 4B, Table 5A, or Table 5B, with a linker and/or vinyl phosphonate modification as set forth herein.

31. The composition of any one of embodiments 20-30, wherein the one or more FN3 domains comprises an FN3 domain conjugated to the siRNA molecule through a cysteine in the FN3 domain.

32. The composition of embodiment 31, wherein the cysteine is at a position as described herein.

33. The composition of embodiment 31 or 32, wherein the cysteine in the FN3 domain is at a position that corresponds to residue 6, 8, 10, 11, 14, 15, 16, 20, 30, 34, 38, 40, 41, 45, 47, 48, 53, 54, 59, 60, 62, 64, 70, 88, 89, 90, 91, or 93 of the FN3 domain based on SEQ ID NO: 2311.

34. The composition of embodiment 33, wherein the cysteine is located at a position that corresponds to residue 6, 53, or 88.

35. The composition of any one of embodiments 20-34, wherein the one or more FN3 domains comprises an FN3 domain that binds to CD71.

36. The composition of any one of embodiments 20-35, wherein the one or more FN3 domains comprises an FN3 domain comprising an amino acid sequence that is at least 87%%, 88%, 89%, 90%, 91%, 92%, 93%, 94%, 95%, 96%, 97%, 98%, or 99% identical to or is identical to a sequence selected from any of SEQ ID NOs: 360-644, 663-672, and 1395-1849.

37. The composition of embodiment 36, wherein the FN3 domain comprises an amino acid sequence that is at least 87% identical to a sequence selected from SEQ ID NOs: 360-644, 663-672, and 1395-1849.

38. The composition of embodiment 36, wherein the FN3 domain comprises an amino acid sequence that is at least 88% identical to a sequence selected from SEQ ID NOs: 360-644, 663-672, and 1395-1849.

39. The composition of embodiment 36, wherein the FN3 domain comprises an amino acid sequence that is at least 89% identical to a sequence selected from SEQ ID NOs: 360-644, 663-672, and 1395-1849.

40. The composition of embodiment 36, wherein the FN3 domain comprises an amino acid sequence that is at least 90% identical to a sequence selected from SEQ ID NOs: 360-644, 663-672, and 1395-1849.

41. The composition of embodiment 36, wherein the FN3 domain comprises an amino acid sequence that is at least 91% identical to a sequence selected from SEQ ID NOs: 360-644, 663-672, and 1395-1849.

42. The composition of embodiment 36, wherein the FN3 domain comprises an amino acid sequence that is at least 92% identical to a sequence selected from SEQ ID NOs: 360-644, 663-672, and 1395-1849.

43. The composition of embodiment 36, wherein the FN3 domain comprises an amino acid sequence that is at least 93% identical to a sequence selected from SEQ ID NOs: 360-644, 663-672, and 1395-1849.

44. The composition of embodiment 36, wherein the FN3 domain comprises an amino acid sequence that is at least 93% identical to a sequence selected from SEQ ID NOs: 360-644, 663-672, and 1395-1849.

45. The composition of embodiment 36, wherein the FN3 domain comprises an amino acid sequence that is at least 94% identical to a sequence selected from SEQ ID NOs: 360-644, 663-672, and 1395-1849.

46. The composition of embodiment 36, wherein the FN3 domain comprises an amino acid sequence that is at least 94% identical to a sequence selected from SEQ ID NOs: 360-644, 663-672, and 1395-1849.

47. The composition of embodiment 36, wherein the FN3 domain comprises an amino acid sequence that is at least 95% identical to a sequence selected from SEQ ID NOs: 360-644, 663-672, and 1395-1849.

48. The composition of embodiment 36, wherein the FN3 domain comprises an amino acid sequence that is at least 96% identical to a sequence selected from SEQ ID NOs: 360-644, 663-672, and 1395-1849.

49. The composition of embodiment 36, wherein the FN3 domain comprises an amino acid sequence that is at least 97% identical to a sequence selected from SEQ ID NOs: 360-644, 663-672, and 1395-1849.

50. The composition of embodiment 36, wherein the FN3 domain comprises an amino acid sequence that is at least 98% identical to a sequence selected from SEQ ID NOs: 360-644, 663-672, and 1395-1849.

51. The composition of embodiment 36, wherein the FN3 domain comprises an amino acid sequence that is at least 99% identical to a sequence selected from SEQ ID NOs: 360-644, 663-672, and 1395-1849.

52. The composition of embodiment 36, wherein the FN3 domain comprises an amino acid sequence that is identical to a sequence selected from SEQ ID NOs: 360-644, 663-672, and 1395-1849.

53. The composition of any one of embodiments 20-52, wherein the one or more FN3 domains comprises at least two FN3 domains linked by a peptide linker.

54. The composition of embodiment 53, wherein the peptide linker comprises an amino acid sequence selected from any of SEQ ID NOs: 645-661.

55. The composition of embodiment 53 or 54, wherein the one or more FN3 domains comprises a first FN3 domain and a second FN3 domain.

56. The composition of embodiment 55, wherein the first FN3 domain comprises an amino acid sequence that is at least 87%%, 88%, 89%, 90%, 91%, 92%, 93%, 94%, 95%, 96%, 97%, 98%, or 99% identical to or is identical to a sequence selected from any of SEQ ID NOs: 360-644, 663-672, and 1395-1849.

57. The composition of embodiment 55 or 66, wherein the first FN3 domain binds to CD71.

58. The composition of any one of embodiments 55-57, wherein the second FN3 domain binds to a different target than the first FN3 domain.

59. The composition of embodiment 58, wherein the second FN3 domain binds to albumin and comprises an amino acid sequence selected from any of SEQ ID NOs: 5-23, or a binding fragment thereof.

60. The composition of any one of embodiments 55-57, wherein the second FN3 domain binds the same target as the first FN3 domain.

61. The composition of any one of embodiments 20-60, further comprising a third FN3 domain.

62. The composition of embodiment 61, wherein the third FN3 domain binds to CD71 or albumin.

63. The composition of embodiment 62, wherein the FN3 domain that binds CD71 has an amino acid sequence as provided herein, including but not limited to any of SEQ ID NOs: 360-644, 663-672, and 1395-1849, or a binding fragment thereof.

64. The composition of embodiment 63, wherein the FN3 domain that binds CD71 has a cysteine substitution as provided herein.

65. The composition of embodiment 62, wherein the FN3 that binds albumin has an amino acid sequence as provided herein, including but not limited to any of SEQ ID NOs: 5-23, or a binding fragment thereof.

66. The composition of embodiment 65, wherein the FN3 domain that binds CD71 has a cysteine substitution as provided herein.

67. A composition having a formula of:

(X₁)ₙ—(X₂)_{q}—(X₃)_{y}-L-X₄;

C—(X₁)ₙ—(X₂)_{q}-L-X₄—(X₃)_{y};

(X₁)ₙ—(X₂)_{q}-L-X₄—(X₃)_{y}—C;

C—(X₁)ₙ—(X₂)_{q}-L-X₄-L-(X₃)_{y};

or (X₁)ₙ—(X₂)_{q}-L-X₄-L-(X₃)_{y}—C,

wherein:
X₁ is a first FN3 domain;
X₂ is a second FN3 domain;
X₃ is a third FN3 domain or a half-life extender molecule;
L is a linker;
X₄ is a nucleic acid molecule, such as an siRNA that targets CD40, such as those provided herein;
C is a polymer, such as PEG, albumin binding protein, or an aliphatic chain that binds to serum proteins; and/or
n, q, and y are each independently 0 or 1.

68. The composition of embodiment 67, wherein X₁, X₂, and X₃ bind to the same or different target proteins.

69. The composition of embodiment 67 or 68, wherein y is 0.

70. The composition of embodiment 67 or 68, wherein n is 1, q is 0, and y is 0.

71. The composition of embodiment 67 or 68, wherein n is 1, q is 1, and y is 0.

72. The composition of embodiment 67 or 68, wherein n is 1, q is 1, and y is 1.

73. The composition of any one of embodiments 67-72, wherein X₃ increases the half-life of the molecule as a whole as compared to a molecule without X₃.

74. The composition of any one of embodiments 67-73, wherein X₃ is a third FN3 domain that binds to albumin.

75. The composition of any one of embodiments 67-74, wherein the linker is a linker as provided herein.

76. The composition of any one of embodiments 67-75, wherein the FN3 domains are connected by a peptide linker.

77. The composition of embodiment 76, wherein the peptide linker comprise an amino acid sequence selected from any of SEQ ID NOs: 645-661 and combinations thereof.

78. The composition of any one of embodiments 67-77, wherein the first, second, and/or third FN3 domain comprises an amino acid sequence as provided herein.

79. The composition of any one of embodiments 67-78, wherein X₄ is an siRNA molecule that targets CD40.

80. The composition of embodiment 79, wherein the siRNA molecule is an siRNA molecule as provided herein.

81. The composition of embodiment 79 or 80, wherein the siRNA molecule reduces mRNA expression of CD40.

82. The composition of any one of embodiments 79-81, wherein the siRNA molecule specifically reduces mRNA expression of CD40.

83. The composition of any one of embodiments 79-82, wherein the siRNA molecule reduces mRNA expression of CD40 and does not significantly reduce expression of other mRNAs.

84. The composition of any one of embodiments 79-83, wherein the siRNA molecule reduces mRNA expression of CD40 and does not reduce expression of other mRNAs by more than 50% in an assay described herein at a concentration of no more than 200 nm as described herein.

85. The composition of any one of embodiments 79-84, wherein the siRNA molecule reduces mRNA expression of CD40 and reduces concentration of CD40 protein.

86. The composition of embodiment 85, wherein the siRNA molecule reduces concentration of CD40 protein in a cell.

87. The composition of embodiment 86, wherein the cell is an immune cell.

88. The composition of embodiment 87, wherein the immune cell is a B cell, a T cell, or a dendritic cell.

89. The composition of embodiment 88, wherein the immune cell is a B cell.

90. The composition of embodiment 88, wherein the immune cell is a T cell.

91. The composition of embodiment 88, wherein the immune cell is a dendritic cell.

92. The composition of any one of embodiments 79-91, wherein the siRNA molecule comprises an siRNA pair as provided in the following formula:

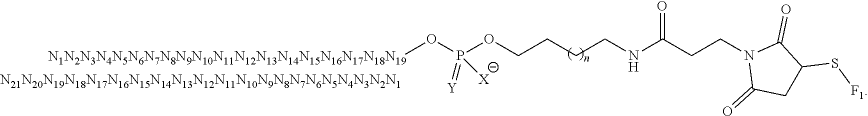

93. The composition of embodiment 92, wherein the antisense strand comprises a vinyl phosphonate modification at N₁.

94. The composition of embodiment 92, wherein maleimide is hydrolyzed to form the following mixture of compounds, or one or both of each compound, or exclusively one of the following compounds:

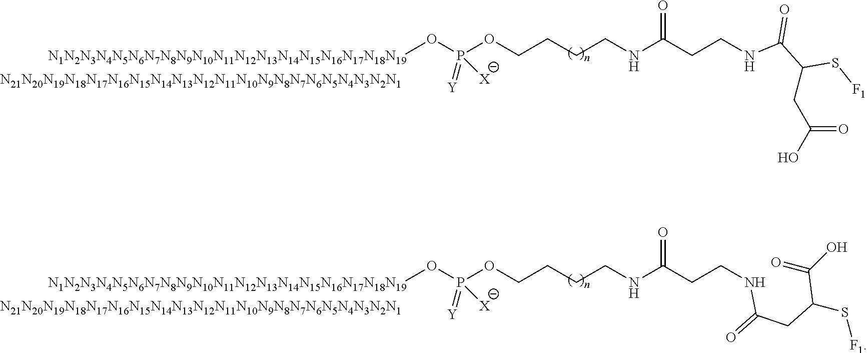

95. The composition of any one of embodiments 67-94, wherein the siRNA molecule comprises an siRNA pair as provided herein or an siRNA pair as provided for in Table 3A, Table 3B, Table 4A, Table 4B, Table 5A, or Table 5B.

96. A composition having a formula A₁-B₁, wherein A₁ has a formula of (C)ₙ-(L₁)ₜ-X_{S} and
B₁ has a formula of X_{AS}-(L₂)_{q}-(F1)_{y},
   wherein:
C is a polymer, such as PEG, albumin binding protein, or an aliphatic chain that binds to serum proteins;
L₁ and L₂ are each, independently, a linker;
X_{S} is a 5′ to 3′ oligonucleotide sense strand of a double stranded siRNA molecule;
X_{AS} is a 3′ to 5′ oligonucleotide antisense strand of a double stranded siRNA molecule;
F₁ is a polypeptide comprising at least one FN3 domain;
n, t, q, and y are each independently 0 or 1; and/or
X_{S} and X_{AS} form a double stranded oligonucleotide molecule to form the composition/complex that targets CD40.

97. A composition having a formula A₁-B₁, wherein A₁ has a formula of (F₁)˜-(L₁)ₜ-X_{S} and B₁ has a formula of X_{AS}-(L₂)_{q}-(C)_{y}, wherein:
C is a polymer, such as PEG, albumin binding protein, or an aliphatic chain that binds to serum proteins;
L₁ and L₂ are each, independently, a linker;
X_{S} is a 5′ to 3′ oligonucleotide sense strand of a double stranded siRNA molecule;
X_{AS} is a 3′ to 5′ oligonucleotide antisense strand of a double stranded siRNA molecule;
F₁ is a polypeptide comprising at least one FN3 domain;
n, t, q, and y are each independently 0 or 1; and/or
X_{S} and X_{AS} form a double stranded oligonucleotide molecule to form the composition/complex that targets CD40.

98. The composition of embodiment 96 or 97, wherein L₁ has a formula of:

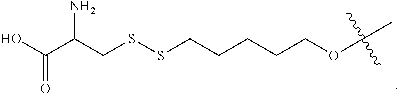 [SMILES: *OCCCCCSSCC(N)C(=O)O]

99. The composition of embodiment 96 or 97, wherein L₂ has a formula of:

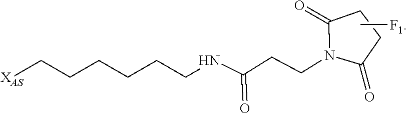

100. The composition of embodiment 96 or 97, wherein A₁-B₁ has a formula of:

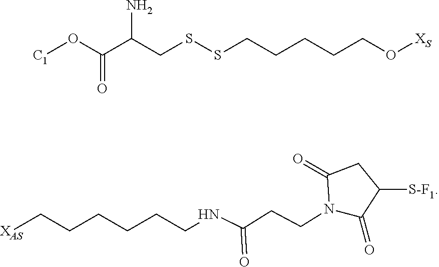

101. The composition of embodiment 96 or 97, wherein A₁-B₁ has a formula of:

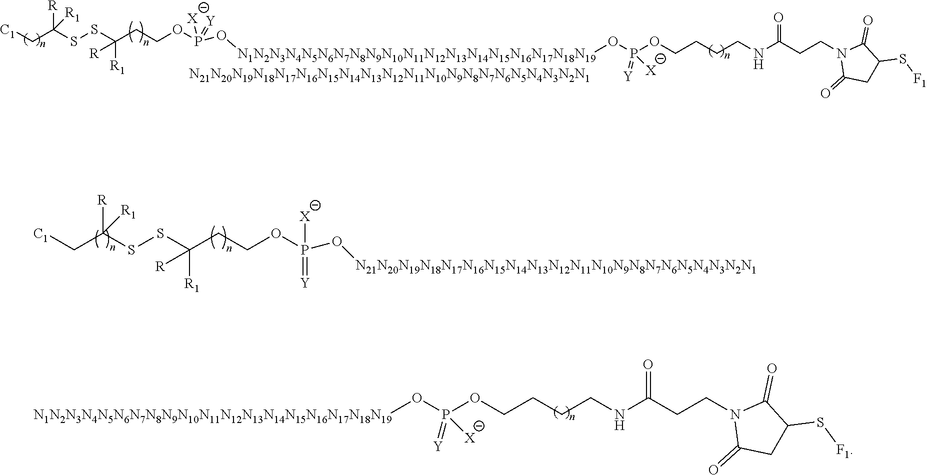

102. The composition of embodiment 96 or 97, wherein F₁ comprises polypeptide having a formula of (X₁)ₙ—(X₂)_{q}—(X₃)_{y}, wherein X₁ is a first FN3 domain; X₂ is a second FN3 domain; X₃ is a third FN3 domain or a half-life extender molecule; wherein n, q, and y are each independently 0 or 1, provided that at least one of n, q, and y is 1.

103. The composition of embodiment 96 or 97, wherein X₁ is a CD71 binding FN3 domain.

104. The composition of embodiment 96 or 97, wherein X₂ is a CD71 binding FN3 domain.

105. The composition of embodiment 96 or 97, wherein X₃ is an FN3 domain that binds to human serum albumin.

106. The composition of embodiment 96 or 97, wherein X₃ is an Fc domain without effector function that extends the half-life of a protein.

107. The composition of any one of embodiments 96-106, wherein X_{S} comprises a nucleic acid sequence selected from any of SEQ ID NOs: 46-178, 312-331, 352-356, 673-805, and 939-958.

108. The composition of any one of embodiments 96-106, wherein X_{AS} comprises a nucleic acid sequence selected from any of SEQ ID NOs: 179-311, 332-351, 356-359, 806-938, and 959-978.

109. The composition of any one of embodiments 96-106, wherein X_{S} and X_{AS} form an siRNA pair selected from any of A1, B1, C1, D1, E1, F1, G1, H1, I1, J1, K1, L1, M1, N1, O1, P1, Q1, R1, S1, T1, U1, V1, W1, X₁, Y1, Z1, A2, B2, C2, D2, E2, F2, G2, H2, I2, J2, K2, L2, M2, N2, O2, P2, Q2, R2, S2, T2, U2, V2, W2, X2, Y2, Z2, A3, B3, C3, D3, E3, F3, G3, H3, I3, J3, K3, L3, M3, N3, O3, P3, Q3, R3, S3, T3, U3, V3, W3, X3, Y3, Z3, A4, B4, C4, D4, E4, F4, G4, H4, I4, J4, K4, L4, M4, N4, O4, P4, Q4, R4, S4, T4, U4, V4, W4, X₄, Y4, Z4, A5, B5, C5, D5, E5, F5, G5, H5, I5, J5, K5, L5, M5, N5, O5, P5, Q5, R5, S5, T5, U5, V5, W5, X5, Y5, Z5, A6, B6, C6, D6, E6, F6, G6, H6, I6, J6, K6, L6, M6, N6, O6, P6, Q6, R6, S6, T6, U6, V6, W6, B7, C7, P8, Q8, R8, S8, T8, U8, V8, W8, X8, Y8, Z8, A9, B9, C9, D9, E9, F9, G9, H9, I9, J9, K9, L9, M9, N9, O9, P9, Q9, R9, S9, T9, U9, V9, W9, X9, Y9, Z9, A10, B10, F10, G10, H10, I10, J10, K10, L10, M10, N10, O10, P10, Q10, R10, S10, T10, U10, V10, W10, X10, Y10, Z10, A11, B11, C11, D11, E11, F11, G11, H11, I11, J11, K11, L11, M11, N11, O11, P11, Q11, R11, or as set forth in Table 3A, Table 3B, Table 4A, Table 4B, Table 5A, or Table 5B.

110. The composition of any one of embodiments 96-106, wherein F₁ comprises an amino acid sequence that is at least 87%%, 88%, 89%, 90%, 91%, 92%, 93%, 94%, 95%, 96%, 97%, 98%, or 99% identical to or is identical to a sequence selected from any of SEQ ID NOs: 360-644, 663-672, and 1395-1849.

111. The composition of embodiment 110, wherein F₁ comprises an amino acid sequence that is at least 87% identical to a sequence selected from any of SEQ ID NOs: 360-644, 663-672, and 1395-1849.

112. The composition of embodiment 110, wherein F₁ comprises an amino acid sequence that is at least 88% identical to a sequence selected from any of SEQ ID NOs: 360-644, 663-672, and 1395-1849.

113. The composition of embodiment 110, wherein F₁ comprises an amino acid sequence that is at least 89% identical to a sequence selected from SEQ ID NOs: 360-644, 663-672, and 1395-1849.

114. The composition of embodiment 110, wherein F₁ comprises an amino acid sequence that is at least 90% identical to a sequence selected from SEQ ID NOs: 360-644, 663-672, and 1395-1849.

115. The composition of embodiment 110, wherein F₁ comprises an amino acid sequence that is at least 91% identical to a sequence selected from SEQ ID NOs: 360-644, 663-672, and 1395-1849.

116. The composition of embodiment 110, wherein F₁ comprises an amino acid sequence that is at least 92% identical to a sequence selected from SEQ ID NOs: 360-644, 663-672, and 1395-1849.

117. The composition of embodiment 110, wherein F₁ comprises an amino acid sequence that is at least 93% identical to a sequence selected from SEQ ID NOs: 360-644, 663-672, and 1395-1849.

118. The composition of embodiment 110, wherein F₁ comprises an amino acid sequence that is at least 93% identical to a sequence selected from SEQ ID NOs: 360-644, 663-672, and 1395-1849.

119. The composition of embodiment 110, wherein F₁ comprises an amino acid sequence that is at least 94% identical to a sequence selected from SEQ ID NOs: 360-644, 663-672, and 1395-1849.

120. The composition of embodiment 110, wherein F₁ comprises an amino acid sequence that is at least 94% identical to a sequence selected from of SEQ ID NOs: 360-644, 663-672, and 1395-1849.

121. The composition of embodiment 110, wherein F₁ comprises an amino acid sequence that is at least 95% identical to a sequence selected from SEQ ID NOs: 360-644, 663-672, and 1395-1849.

122. The composition of embodiment 110, wherein the FN3 domain comprises an amino acid sequence that is at least 96% identical to a sequence selected from SEQ ID NOs: 360-644, 663-672, and 1395-1849.

123. The composition of embodiment 110, wherein F₁ comprises an amino acid sequence that is at least 97% identical to a sequence selected from SEQ ID NOs: 360-644, 663-672, and 1395-1849.

124. The composition of embodiment 110, wherein F₁ comprises an amino acid sequence that is at least 98% identical to a sequence selected from SEQ ID NOs: 360-644, 663-672, and 1395-1849.

125. The composition of embodiment 110, wherein F₁ comprises an amino acid sequence that is at least 99% identical to a sequence selected from SEQ ID NOs: 360-644, 663-672, and 1395-1849.

126. The composition of embodiment 110, wherein F₁ comprises an amino acid sequence that is identical to a sequence selected from SEQ ID NOs: 360-644, 663-672, and 1395-1849.

127. The composition of any one of embodiments 96-106, wherein F₁ comprises a polypeptide that binds to albumin.

128. A pharmaceutical composition comprising a composition of any one of embodiments 1-127.

129. A kit comprising a composition of any of embodiments 1-127.

130. A method of treating immunological diseases in a subject in need thereof, the method comprising administering to the subject composition of any of embodiments 1-127 or any composition provided herein.

131. A use of a composition as provided herein or of any of embodiments 1-127 in the preparation of a pharmaceutical composition or medicament for treating immunological diseases, such as autoimmune diseases provided herein.

132. Use of a composition as provided herein or any of embodiments 1-127 for treating immunological disease.

133. The use of embodiment 132, wherein the immunological disease is rheumatoid arthritis, Hashimoto's autoimmune thyroiditis, celiac disease, diabetes mellitus type 1, vitiligo, rheumatic fever, pernicious anemia/atrophic gastritis, alopecia areata, immune thrombocytopenic purpura, psoriasis, inflammatory bowel disease, systemic lupus erythematosus, pemphigus, Sjogren's syndrome, inflammatory myositis, lupus nephritis, Pemphigus vulgaris, multiple sclerosis, or prevention of solid organ transplant rejection.

134. A method of reducing the expression of a target gene in a cell, such as an immune cell, the method comprising contacting the immune cell with a composition of any of embodiments 1-127 or a composition as provided herein.

135. The method of embodiment 134, wherein the target gene is CD40.

136. A method of delivering an siRNA molecule to a cell, such as an immune cell, in a subject, the method comprising administering to the subject a pharmaceutical composition comprising a composition of any of embodiments 1-127.

137. The method of embodiment 136, wherein the cell is a CD71 positive cell.

138. The method of embodiment 136 or 137, wherein the cell is an immune cell.

139. The method of embodiment 138, wherein the immune cell is a B cell, a T cell, or a dendritic cell.

140. The method of embodiment 139, wherein the immune cell is a B cell.

141. The method of embodiment 139, wherein the immune cell is a T cell.

142. The method of embodiment 139, wherein the immune cell is a dendritic cell.

143. The method of any one of embodiments 136-142, wherein the siRNA molecule downregulates expression of a target gene in the cell.

144. The method of embodiment 143, wherein the downregulation of expression of the target gene results in a reduction of about 99%, 90-99%, 50-90%, or 10-50%.

145. The method of any one of embodiments 136-144, wherein the target gene is CD40.

146. A method of delivering an siRNA molecule that targets CD40 to a CD71 positive immune cell in a subject, the method comprising administering to the subject a pharmaceutical composition comprising a composition of any of embodiments 1-127, wherein the siRNA molecule downregulates expression of CD40 in the CD71 positive immune cell.

147. A method of reducing one or more cytokines in a population of CD71 positive immune cells, the method comprising contacting the population of CD71 positive immune cells with the composition of any one of embodiments 1-127.

148. A method of reducing one or more cytokines in a subject, the method comprising administering to the subject the composition of any one of embodiments 1-127.

149. A method of reducing one or more cytokines in a subject in need thereof, the method comprising administering to the subject in need thereof the composition of any one of embodiments 1-127.

150. The method of embodiment 147, wherein the one or more cytokines is selected from IFN-γ, IL-6, TNF-α, IL-12, IP-10, and/or RANTES, or any combination thereof.

151. The method of embodiment 147 or 148, wherein the one or more CD71 positive immune cells comprises a B cell, a T cell, a dendritic cell, or a combination thereof.

152. A method of reducing or inhibiting migration of a population of cells from blood to tissue, the method comprising contacting a CD40 targeting siRNA molecule, such as those described herein, to the population of cells.

153. The method of embodiment 152, wherein the population of cells comprises CD40 expressing cells.

154. The method of embodiment 152 or 153, wherein the population of cells comprises dendritic cells, B cells, or a combination thereof.

155. The method of any one of embodiments 152-154, wherein the tissue is lymphoid organ tissue.

### EXAMPLES

The following examples are illustrative of the embodiments disclosed herein. These examples are provided for the purpose of illustration only and the embodiments should in no way be construed as being limited to these examples, but rather should be construed to encompass any and all variations which become evidence as a result of the teaching provided herein. Those of skill in the art will readily recognize a variety of non-critical parameters that could be changed or modified to yield essentially similar results.

### Example 1: Cd40 siRNA Sequence Identification and Characterization

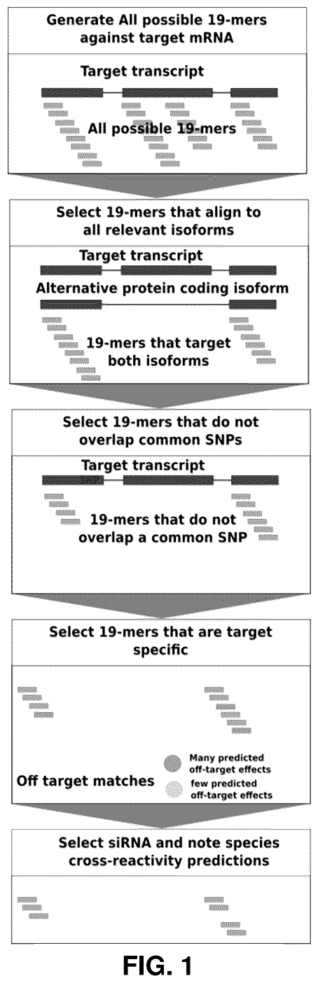

siRNA in silico screening: In silico siRNA screening was performed to identify an siRNA complementary to human CD40 mRNA. A flow chart depicting the steps and properties assessed during the screening is shown in FIG. 1. All possible 19-mer antisense sequences were generated from primary the human CD40 mRNA isoform sequence (NM_001250.6) and each 19-mer was assessed for complementarity against other relevant human CD40 isoforms. In silico cross-reactivity was further assessed in the transcriptomes of mouse, rat, and Cynomolgus macaque. A subset of well annotated transcripts from each model organism was assessed to determine cross reactivity (Table 17). Off-target effects were assessed by mapping both strands to the human RefSeq release 204 transcriptome using BOWTIE and identifying locations where the nucleotides in position 2-18 have alignments of 14, 15, 16, and 17 nucleotides respectively.

TABLE 17

| Species | Transcripts considered for activity |
|---|---|
| Human | NM_001250.6 |
| | NM_001302753.2 |
| | NM_001322421.2 |
| | NM_001362758.2 |
| | NM_152854.4 |
| | NM_001322422.2 |
| | XM_017028135.1 |
| | XM_017028136.1 |
| | XM_005260619.3 |
| | ENST00000372285.8 |
| | ENST00000372276.7 |
| | ENST00000620709.4 |
| Mouse | NM_011611.2 |
| | NM_170704.2 |
| | NM_170703.2 |
| | NM_170702.2 |
| | XM_006499155.5 |
| | XM_006499154.4 |
| | ENSMUST00000017799.11 |
| | ENSMUST00000073707.8 |
| Cynomolgus | XM_005569217.2 |
| Macaque | XM_005569218.2 |
| | XM_005569221.2 |
| | XM_005569219.2 |
| | XM_005569220.2 |
| | ENSMFAT00000070425.1 |
| | ENSMFAT00000070436.1 |
| Rat | NM_134360.1 |
| | XM_006235511.2 |
| | XM_008762464.1 |
| | XM_008762463.2 |
| | XM_008762465.1 |
| | ENSRNOT00000055148.4 |

Human siRNA target sites were assessed for common human polymorphisms (MAF>1%) NCBI dbSNP Build 2.0 153. Sequences that targeted a common allele were discarded. Next, siRNA off-target genes were assessed for sense and antisense strands in all relevant model organisms. siRNA molecules that did not overlap any common human genetic polymorphisms were considered candidates for synthesis. These candidates were prioritized for screening based on human off-target predictions. Those sequences containing relatively low number of off-target alignments to the transcriptome were selected for synthesis. Antisense position 1 was then replaced with a 5′ U (along with a corresponding 3′ sense A) and a 3′ UU was engineered into the antisense.

Oligonucleotide synthesis: Synthesis of oligonucleotides was performed on Mermade® 12 synthesizer using standard phosphoramidite chemistry on 500 Å controlled pore glass (CPG) with phosphoramidites at a 0.1 M concentration in acetonitrile. Iodine in THE/pyridine/water (0.02M) used as the oxidizing agent with 0.6 M ETT (5-ethylthiotetrazole) as the activation agent. N, N-dimethyl-N′-(3-thioxo-3H-1,2,4-dithiazol-5-yl)methanimidamide (DDTT), 0.09 M in pyridine, was used as the sulfurizing reagent for the introduction of phosphorothioate (PS) bonds. 3% (v/v) dichloroacetic acid in dichloromethane was used the deblocking solution. All single strands without maleimide were purified by ion-exchange chromatography with 20 mM phosphate at pH 8.5 as Buffer A and 20 mM phosphate pH 8.5 and 1 M sodium bromide as Buffer B. After purification, the oligonucleotide fractions were pooled, concentrated, and desalted. Desalted samples were then lyophilized to dryness and stored at −20° C.

Deprotection of antisense strands: After synthesis, the support was washed with acetonitrile (ACN) and dried in the column under vacuum and transferred into a 1 mL screw cap that could be tightly sealed and shaken with a solution of 5% diethylamine in aqueous ammonia at 65° C. for 5 h. Cleavage and deprotection of crude oligo was checked by liquid chromatography-mass spectrometry (LC-MS) and was subsequently purified by IEX-HPLC.

Synthesis, deprotection and annealing of maleimide-containing oligonucleotides: Maleimide containing oligos were made using either a 3′ amino-modified CPG solid support or a 5′ amino modifier phosphoramidite. The support was transferred into a 1 mL vial that could be tightly sealed and incubated with 50/50 v/v 40% aqueous methyl amine and aqueous ammonia (AMA) at room temperature for 2 h or 65° C. for 10 minutes to cleave and deprotect. The single strand was purified by ion-exchange chromatography and desalted the same conditions as the antisense before maleimide addition.

Approximately 20 mg/mL of the amine-modified sense strand in 0.05 M phosphate buffer at pH 7.1 was made to which 10 equivalents of the maleimide N-hydroxysuccinimide (NHS) ester, dissolved in ACN was added. The NHS ester solution was added to the aqueous oligonucleotide solution and shaken for 3 h at room temperature. The now maleimide conjugated oligonucleotides were purified by reverse-phase chromatography using 20 mM triethyl ammonium acetate with 80% acetonitrile in Buffer B as mobile phase.

After purification, the oligonucleotide fractions were pooled, concentrated, and desalted. To avoid hydrolysis of maleimide, duplexing of the sense and antisense strands was performed via freeze-drying using equimolar amounts of each desalted single strand.

FN3-siRNA conjugation and purification: FN3 domain-siRNA conjugates were prepared by conjugation of cysteine modified CD71-binding FN3 domains to maleimide containing siRNAs via cysteine-specific chemistry. For FN3-maleimide conjugation, cysteine-containing CD71-binding FN3 domains in PBS at 50-200 μM were reduced with 10 mM tris(2-carboxyethyl)phosphine (TCEP) at room temperature (30 mins) to yield a free thiol. To remove the TCEP, the FN3 protein was precipitated with saturated ammonium sulfate solution and then mixed with maleimide-modified siRNA duplex dissolved in water immediately prior at a molar ratio of ˜1.5:1 FN3:siRNA. After 1 hour incubation at RT or 37° C., reaction was quenched with N-ethyl maleimide (1 mM final NEM concentration in the reaction mixture).

To avoid loss of payload via retro-Michael reaction, maleimide ring hydrolysis is performed. Pooled fractions from conjugate are dialyzed into 25 mM TRIS pH 8.9 buffer. In this buffer the reaction is placed in an incubator shaker at 37° C. for 72 hrs. Reaction monitored for completion by LC-MS.

FN3 domain-siRNA conjugates were purified in two steps using IMAC chromatography (HisTrap HP) to remove unreacted siRNA linker, and anion exchange chromatography-Capto-DEAE; to remove unreacted FN3 proteins. FN3 domain-siRNA conjugates were characterized by PAGE, analytical size exclusion chromatography and LC/MS. Concentration of conjugate was calculated based on absorbance of conjugate solution at 260 using a Nanodrop.

In vitro screening 1: In vitro screen using single concentration to select first set of hits. Raji cells were electroporated with Neon 10 μl tips in the Neon Transfection System (Life Technologies) with settings 1300 V pulse voltage, 30 ms pulse width, and 1 pulse number. A single concentration of 20 nM of siRNA was used for electroporation. After electroporation, the cells were plated in RPMI-1640+10% heat-inactivated certified FBS+1% glutamax. The cells were incubated at 37° C. for 24 hours prior to lysis. Cells to Ct was used for qPCR. PGK1 was used as the endogenous control and RQ values were normalized to “electroporation of water only” control. SEQ ID NO of the siRNA sense strand used, relative CD40 expression, and KD % are shown in Tables 11, 12, and 13.

TABLE 11

| SEQ ID NO | Relative CD40 Expression | % KD |
|---|---|---|
| Cells Alone | 1.149254212 | −15% |
| Water | 1.003990555 | 0% |
| 46 | 0.909680702 | 9% |
| 47 | 0.743403529 | 26% |
| 48 | 0.703704739 | 30% |
| 49 | 0.947740379 | 5% |
| 50 | 0.864740935 | 14% |
| 51 | 0.62619472 | 37% |
| 52 | 0.768353208 | 23% |
| 53 | 0.987012714 | 1% |
| 54 | 0.869341663 | 13% |
| 55 | 0.841853463 | 16% |
| 56 | 0.817380579 | 18% |
| 57 | 0.552310134 | 45% |
| 58 | 1.082485112 | −8% |
| 59 | 1.042719552 | −4% |
| 60 | 0.899848414 | 10% |
| 61 | 0.880316424 | 12% |
| 62 | 0.664870191 | 34% |
| 63 | 0.729761005 | 27% |
| 64 | 0.902885713 | 10% |
| 65 | 0.729691208 | 27% |
| 66 | 0.590870903 | 41% |
| 67 | 0.798724017 | 20% |
| 68 | 0.774238883 | 23% |
| 69 | 0.743035586 | 26% |
| 70 | 0.983048203 | 2% |
| 71 | 0.993623025 | 1% |
| 72 | 0.875978927 | 12% |
| 73 | 1.135949571 | −14% |
| 74 | 1.053504041 | −5% |
| 75 | 1.008676715 | −1% |
| 76 | 0.948329523 | 5% |
| 77 | 1.091930907 | −9% |
| 78 | 1.034526434 | −3% |
| 79 | 0.924380748 | 8% |
| 80 | 1.121298524 | −12% |
| 81 | 0.984561964 | 2% |
| 82 | 0.978611164 | 2% |
| 83 | 0.881130218 | 12% |
| 84 | 1.320259339 | −32% |
| 85 | 0.928711683 | 7% |
| 86 | 0.990115269 | 1% |
| 87 | 1.024379909 | −2% |

TABLE 12

| SEQ ID NO | Relative CD40 Expression | % KD |
|---|---|---|
| Cells Alone | 1.343776 | −34% |
| Water | 1 | 0% |
| 101 | 0.831747016 | 17% |
| 102 | 0.81733404 | 18% |
| 103 | 0.563286873 | 44% |
| 104 | 0.680236801 | 32% |
| 105 | 0.748766708 | 25% |
| 106 | 1.210384159 | −21% |
| 107 | 0.626908315 | 37% |
| 108 | 0.824963716 | 18% |
| 109 | 0.586207223 | 41% |
| 110 | 0.538112572 | 46% |
| 111 | 0.498946757 | 50% |
| 112 | 0.704325487 | 30% |
| 113 | 0.812404254 | 19% |
| 114 | 0.514618913 | 49% |
| 115 | 0.531157496 | 47% |
| 116 | 1.121295754 | −12% |
| 117 | 1.045805998 | −5% |
| 118 | 0.617456337 | 38% |
| 119 | 0.988976941 | 1% |
| 120 | 0.937158154 | 6% |
| 121 | 0.625369433 | 37% |
| 122 | 0.661367656 | 34% |
| 123 | 0.443969715 | 56% |
| 124 | 0.558153316 | 44% |
| 125 | 0.745917202 | 25% |
| 126 | 0.71088051 | 29% |
| 127 | 0.776290399 | 22% |
| 128 | 0.350835076 | 65% |
| 129 | 0.396576832 | 60% |
| 130 | 0.394009796 | 61% |
| 131 | 0.565565057 | 43% |
| 132 | 0.666630372 | 33% |
| 133 | 0.860769602 | 14% |
| 134 | 0.457934389 | 54% |
| 135 | 0.801985609 | 20% |
| 136 | 1.089838975 | −9% |
| 137 | 1.068546521 | −7% |
| 138 | 0.751258525 | 25% |
| 139 | 0.714892672 | 29% |
| 140 | 0.543018731 | 46% |
| 141 | 0.470724331 | 53% |
| 142 | 0.437388094 | 56% |
| 143 | 0.49984402 | 50% |
| 144 | 0.474237371 | 53% |
| 145 | 0.331310913 | 67% |
| 146 | 0.504627385 | 50% |
| 147 | 0.814147169 | 19% |
| 148 | 0.872137532 | 13% |
| 149 | 0.927470102 | 7% |
| 150 | 0.767530331 | 23% |
| 151 | 0.702624434 | 30% |
| 152 | 0.865857788 | 13% |
| 153 | 0.972791933 | 3% |
| 154 | 0.947240401 | 5% |

TABLE 13

| SEQ ID NO | Relative CD40 Expression | % KD |
|---|---|---|
| Cells Alone | 1.122351745 | −12% |
| Water | 1 | 0% |
| 88 | 0.950617657 | 5% |
| 89 | 1.071355823 | −7% |
| 90 | 1.21709319 | −22% |
| 91 | 1.08442193 | −8% |
| 92 | 0.965247485 | 3% |
| 93 | 0.883124196 | 12% |
| 94 | 0.697111663 | 30% |
| 95 | 0.686988025 | 31% |
| 96 | 0.929588911 | 7% |
| 97 | 0.516791125 | 48% |
| 98 | 0.843864392 | 16% |
| 99 | 0.953386611 | 5% |
| 100 | 1.065865765 | −7% |
| 155 | 1.035884581 | −4% |
| 156 | 1.229445655 | −23% |
| 157 | 0.619988851 | 38% |
| 158 | 0.781808906 | 22% |
| 159 | 0.952489595 | 5% |
| 160 | 0.631758163 | 37% |
| 161 | 0.715872508 | 28% |
| 162 | 0.796020481 | 20% |
| 163 | 1.180848838 | −18% |
| 164 | 0.966813286 | 3% |
| 165 | 1.292063155 | −29% |
| 166 | 0.883030797 | 12% |
| 167 | 0.859540302 | 14% |
| 168 | 0.870443073 | 13% |
| 169 | 0.625400025 | 37% |
| 170 | 0.54956429 | 45% |
| 171 | 0.84474175 | 16% |
| 172 | 0.906642904 | 9% |
| 173 | 0.620974058 | 38% |
| 174 | 0.961299584 | 4% |
| 175 | 1.076118437 | −8% |
| 176 | 0.860515866 | 14% |
| 177 | 1.049361244 | −5% |
| 178 | 0.64558077 | 35% |

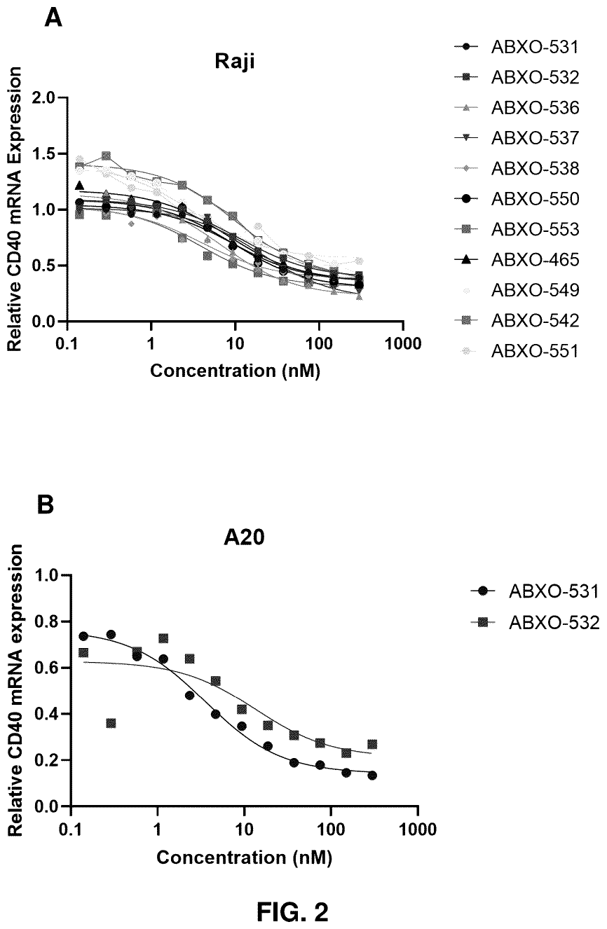

In vitro screening 2: Raji or A20 cells were electroporated with Neon 10 μl tips in the Neon Transfection System (Life Technologies) with settings 1300 V pulse voltage, 30 ms pulse width, and 1 pulse number for Raji or 1680 V pulse voltage, 20 ms pulse width, and 1 pulse for A20. A titration of siRNA was done for electroporation (2-fold dilutions were made starting at 300 nM). After electroporation, the cells were plated in RPMI-1640+10% heat-inactivated certified FBS+1% glutamax. The cells were incubated at 37° C. for 24 hours prior to lysis. Cells to Ct was used for qPCR. For Raji, PGK1, HPRT1, and UBE2D2 were used as endogenous controls and RQ values were normalized to “electroporation of water” control. For A20, B₂M, HPRT1, and PGK1 were used as endogenous controls and RQ values were normalized to “electroporation of water only” control. SEQ ID NO of the siRNA sense strand used, the KD % at 300 nM and the EC50 values are shown in Table 14 and Table 15 (Raji cells), and Table 16 and Table 17 (A20 cells). Titration curves corresponding to the sequences shown in Table 15 and Table 17 are shown in FIG. 2A and FIG. 2B, respectively.

TABLE 14

| SEQ ID NO | % KD at 300 nM | EC50 |
|---|---|---|
| 57 | 70% | 14.5 |
| 123 | 64% | 7.12 |
| 124 | 56% | 19.69 |
| 128 | 45% | 4.988 |
| 129 | 76% | 9.914 |
| 130 | 69% | 10.97 |
| 134 | 72% | 24.68 |
| 141 | 57% | 31.56 |
| 142 | 62% | 20.76 |
| 143 | 61% | 12.13 |
| 145 | 70% | 3.95 |

TABLE 15

| SEQ ID NO | % KD at 300 nM | EC50 |
|---|---|---|
| 328 | 65% | 8.194 |
| 332 | 61% | 9.342 |
| 333 | 59% | 11.48 |
| 314 | 78% | 6.852 |
| 315 | 73% | 20.39 |
| 316 | 61% | 4.03 |
| 329 | 61% | 10.29 |
| 330 | 57% | 4.835 |
| 317 | 67% | 9.59 |
| 331 | 46% | 3.077 |
| 318 | 67% | 3.91 |

TABLE 16

| SEQ ID NO | % KD at 300 nM | EC50 |
|---|---|---|
| 123 | 83% | 6.267 |
| 124 | 67% | 5.248 |

TABLE 17

| SEQ ID NO | % KD at 300 nM | EC50 |
|---|---|---|
| 332 | 87% | 3.557 |
| 333 | 73% | 13.45 |

### Example 2: Reduction of CD40 Expression by Administration of CD71-Binding FN3 Domain-CD40 siRNA Composition (Prophetic)

CD71-binding FN3 domains as provided herein are conjugated to CD40 siRNAs as provided herein and assayed to determine superior binding to immune cells, siRNA uptake, and reduction of CD40 expression in the target cells. Select compositions are formulated as pharmaceutical compositions as provided herein and administered to animal model having an autoimmune disease. CD40 expression is reduced in the targeted immune cells and the autoimmune disease is treated.

### Example 3: In Vitro Testing of siRNAs and Conjugates

Single dose and dose response curves using transfection reagent in SKBR3 and Cal27 cells: SKBR3 or Cal27 cells were reverse-transfected with a transfection reagent at 20,000 cells per well, using methods known to those of ordinary skill in the art. Briefly, siRNAs shown in Table 18 and the transfection reagent were diluted separately in reduced serum medium before combining at a 1:1 ratio. The mixture of transfection reagent and siRNAs was pipetted into 96 well flat-bottom plates and topped off with cells that were resuspended in antibiotic-free media containing RPMI 1640 and 10% fetal bovine serum. The cells were incubated at 37° C. for 24 hours and then cells were lysed for quantitative polymerase chain reaction (qPCR) analysis in SKBR3 cells. Bulk lysis reagents were used to lyse cells and RNA was isolated for measurement. PGK1 was used as the endogenous gene control. Relative quantification (RQ) values were normalized to the “transfection with transfection reagent only” control. Knockdown (KD) was also measured for the SKBR3 cell group. EC50 and Emax were determined for both SKBR3 and Cal27 cell groups. Table 18 shows relative CD40 mRNA expression, % KD at 10 nM, EC50 (pM), and Emax (%) are shown in each siRNA tested in SKBR3 cells and EC50 (pM) and Emax (%) for each siRNA tested in Cal27 cells. Relative CD40 mRNA expression was reduced in SKBR3 cells in vitro by about 77% to about 84% following treatment of the siRNAs listed in Table 18. Potency and maximal response were reduced after treatment of SKBR3 cells as compared to after treatment of Cal27 cells.

TABLE 18

| | | | Relative | | | | | |
|---|---|---|---|---|---|---|---|---|
| | Sense | Antisense | CD40 | | | | | |
| | Strand | Strand | mRNA | % KD at | EC50 | Emax | EC50 | Emax |
| | SEQ | SEQ | expression | 10 nM | (pM) | (%) | (pM) | (%) |
| siRNA ID | ID NO | ID NO | (SKBR3) | (SKBR3) | (SKBR3) | (SKBR3) | (Cal27) | (Cal27) |
| ABXO-1049 | 1890 | 2290 | 0.222 | 78 | 18.0 | 72.0 | 43.2 | 80 |
| ABXO-1050 | 1891 | 2291 | 0.166 | 83 | 6.0 | 74.0 | 23.8 | 83 |
| ABXO-1052 | 1893 | 2293 | 0.189 | 81 | 21.0 | 73.0 | 39.5 | 86 |
| ABXO-1059 | 1900 | 2010 | 0.202 | 80 | 36.0 | 79.0 | 64 | 86 |
| ABXO-537 | 315 | 335 | 0.163 | 84 | 19.0 | 75.0 | 114 | 85 |

Single dose and dose response curves using electroporation in A20 and C2C12 cells: A20 cells were electroporated at 50,000 cells per well with 10 μL tips at 1680 V pulse voltage, 20 ms pulse width, and 1 pulse. siRNAs in Table 19 were diluted in water for electroporation; water only was used as a control. After electroporation, the cells were plated in antibiotic-free medium containing RPMI 1640 and 10% fetal bovine serum. The cells were incubated at 37° C. for 24 hours prior to lysis. Bulk lysis reagents were used to lyse cells and RNA was isolated for measurement. B2M was used as the endogenous gene control. RQ values were normalized to control.

C2C12 cells were reverse transfected with a transfection reagent at 20,000 cells per well. Briefly, siRNAs and the transfection reagent were diluted separately in reduced serum medium before combining at a 1:1 ratio into a mixture. The mixture was pipetted into 96 well flat-bottom plates and topped off with cells that were resuspended in antibiotic-free medium containing RPMI 1640 and 10% fetal bovine serum. The cells were incubated at 37° C. for 24 hours prior to lysis. Cells to Ct was used for qPCR. B2M was used as the endogenous gene control. RQ values were normalized to the “transfection with transfection reagent only” control. Relative CD40 mRNA expression, % KD at 5 nM, EC50 (pM), and Emax (%) are shown in Table 19 for each conjugate tested in A20 cells; EC50 (pM) and Emax (%) are also shown for each siRNA tested in C2C12 cells. Relative CD40 mRNA expression was reduced in A20 cells in vitro following administration of some of the siRNAs listed below, in Table 19, namely. For example, relative CD40 mRNA expression was reduced in A20 cells in vitro by about 27.8% following administration of siRNA ABXO-1036.

TABLE 19

| | Relative CD40 | | | | | |
|---|---|---|---|---|---|---|
| | mRNA | | EC50 | | EC50 | Emax |
| | expression | % KD at | (nM) | Emax (%) | (pM) | (%) |
| siRNA ID | (A20) | 5 nM (A20) | (A20) | (A20) | (C2C12) | (C2C12) |
| ABXO-1022 | 0.764 | 24 | 6.1 | 83 | 62 | 94 |
| ABXO-1025 | 0.825 | 18 | 13 | 79 | 73 | 90 |
| ABXO-1036 | 0.722 | 28 | 0.63 | 73 | 15 | 94 |
| ABXO-1002 | 0.786 | 21 | 92.5 | 74 | N/A | N/A |
| ABXO-1003 | 0.827 | 17 | 161.3 | 70 | N/A | N/A |
| ABXO-1004 | 1.183 | 0 | N/A | N/A | N/A | N/A |
| ABXO-1005 | 0.885 | 12 | N/A | N/A | N/A | N/A |
| ABXO-1006 | 1.037 | 0 | N/A | N/A | N/A | N/A |
| ABXO-1007 | 1.095 | 0 | N/A | N/A | N/A | N/A |
| ABXO-1009 | 1.165 | 0 | N/A | N/A | N/A | N/A |
| ABXO-1010 | 0.88 | 12 | N/A | N/A | N/A | N/A |
| ABXO-1011 | 1.191 | 0 | N/A | N/A | N/A | N/A |
| ABXO-1012 | 1.12 | 0 | N/A | N/A | N/A | N/A |
| ABXO-1013 | 1.038 | 0 | N/A | N/A | N/A | N/A |
| ABXO-1014 | 1.06 | 0 | N/A | N/A | N/A | N/A |
| ABXO-1015 | 1.23 | 0 | N/A | N/A | N/A | N/A |
| ABXO-1016 | 1.07 | 0 | N/A | N/A | N/A | N/A |
| ABXO-1017 | 1.08 | 0 | N/A | N/A | N/A | N/A |
| ABXO-1018 | 0.919 | 8 | N/A | N/A | N/A | N/A |
| ABXO-1019 | 1.11 | 0 | N/A | N/A | N/A | N/A |
| ABXO-1028 | 0.947 | 5 | N/A | N/A | N/A | N/A |
| ABXO-1029 | 0.89 | 11 | N/A | N/A | N/A | N/A |
| ABXO-1030 | 1.14 | 0 | N/A | N/A | N/A | N/A |
| ABXO-1031 | 0.98 | 2 | N/A | N/A | N/A | N/A |
| ABXO-1032 | 1.19 | 0 | N/A | N/A | N/A | N/A |

Testing conjugates in dendritic cells (DCs) and SKBR3 cells: Dendritic cells (500,000 cells per well) and SKBR3 cells (50,000 cells per well) were treated with diluted conjugates and an activating agent for four days at 37° C. prior to cell lysis for qPCR. The media is composed of RPMI+10% fetal bovine serum+1% penicillin and streptomycin. For dendritic cells, RNA was extracted after cell lysis. For SKBR3 cells, bulk lysis reagents were used to lyse cells and RNA was isolated for measurement. For SKBR3 cells, PGK1 was used as the endogenous control and for dendritic cells, PGK1 and UBE2D2 were used as the endogenous control. Relative quantification (RQ) values were normalized to the “activated cells alone” control. Table 20 shows EC50 (in nM) and Emax (%) of the ABXC-83 and ABXC-225 conjugates tested in dendritic and SKBR3 cells, demonstrating potency and efficacy of the conjugates as compared to activated controls.

TABLE 20

| | | siRNA | siRNA | | | |
|---|---|---|---|---|---|---|
| | | Sense | Antisense | | | |
| Conjugate | Centyrin | Strand | Strand | | EC50 | Emax |
| ID | SEQ ID NO | SEQ ID NO | SEQ ID NO | Cell Type | (nM) | (%) |
| ABXC-83 | 570 | 354 | 358 | Dendritic Cell | 129 | 65 |
| ABXC-83 | 570 | 354 | 358 | Dendritic Cell | 14.9 | 49 |
| ABXC-83 | 570 | 354 | 358 | Dendritic Cell | 130 | 49 |
| ABXC-225 | 1849 | 354 | 358 | Dendritic Cell | 4.7 | 60 |
| ABXC-225 | 1849 | 354 | 358 | Dendritic Cell | 1.4 | 65 |
| ABXC-83 | 570 | 354 | 358 | SKBR3 | 4.7 | 65 |

### Example 4: In Vitro Administration of CD71-Binding FN3 Domains and CD40-Targeting siRNA Conjugates in Donated Human Dendritic Cells

Treatment with an exemplary composition comprising a CD71 FN3-CD40 siRNA conjugate, ABXC-79 (SEQ ID NOs: 1848, 354, 358), reduced expression of CD40 mRNA and reduced production of IL-12 in human dendritic cells activated and exposed to CD40 ligand. Dendritic cells (500,000 cells per well) were treated with 1 μM of conjugate and an activating agent for four days, followed by an additional activating agent for one day at 37° C. prior to collection of supernatants for electrochemiluminescence (ECL) immunoassay and cell lysis for qPCR. For ECL, a multiplex assay kit was used to quantify cytokines in the supernatants. The cell culture media used was composed of RPMI+10% fetal bovine serum+1% penicillin and streptomycin. For qPCR, RNA was extracted after cell lysis, and PGK1 and UBE2D2 were used as the endogenous controls in qPCR. Relative quantification (RQ) values were normalized to the “activated cells alone” control.

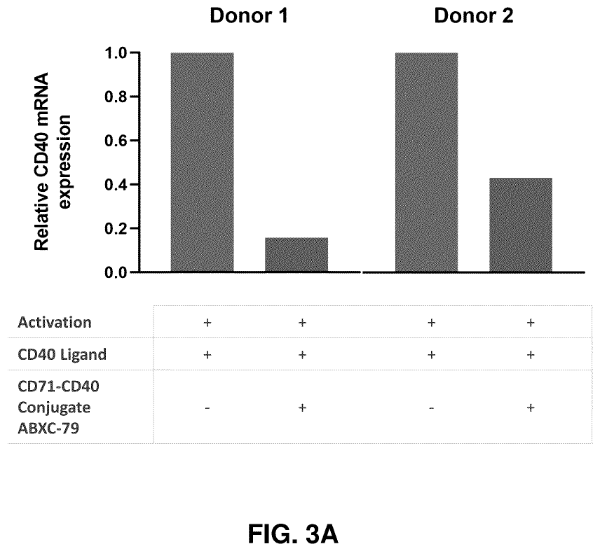

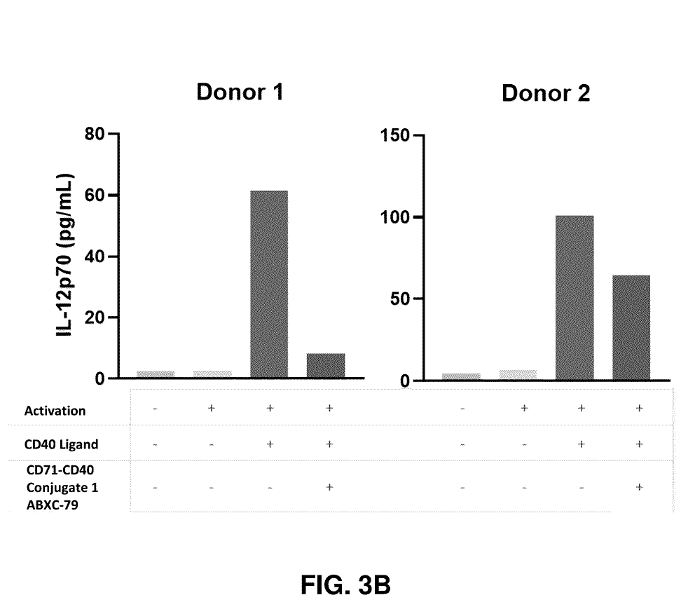

As shown in FIG. 3A, relative CD40 mRNA expression after treatment with was reduced by over 80% in cells from Donor 1, and reduced by about 60% in cells from Donor 2. As shown in FIG. 3B, IL-12 production (as measured in pg/mL) was reduced by about 80% in cells from Donor 1, and reduced by about 40% in cells from Donor 2. These results demonstrate functional activity of the CD71 FN3-CD40 siRNA conjugate in human dendritic cells.

### Example 5: In Vivo Administration in Rodent Models of Mouse Surrogates of CD71-Binding FN3 Domains and CD40-Targeting siRNA Conjugates

Male CD-1 mice under different experimental conditions (n=5 for each group) were treated on day −1, treated on day 0, and terminated on day 1, after which serum cytokine levels were quantified. There were three treatment groups, two of which were also activated on day 0. Group 1 was treated by administration of two mouse surrogates of CD71 FN3-CD40 siRNA conjugates, and activated prior to termination. Group 2 was treated by administration of buffered saline (“HBS”) and activated prior to termination. Group 3 (“Naïve”) was not treated and not activated prior to termination. Additionally, two other control groups were treated with siRNAs that were not specific for CD40 or a capped siRNA that is inactive against CD40. Administration of the mouse surrogate conjugates reduced serum cytokines and prevents margination of dendritic cells and B cells in vivo. Serum levels (as measured in pg/mL) of IFN-γ, IL-6, TNF-α, IL-12, IP-10, and RANTES were reduced following administration of the mouse surrogate conjugates.

Degree of reduction in CD40 expression was determined by administering an anti-CD40 agonist antibody, followed by measuring the amount of dendritic cells, B cells, and T cells via flow cytometric analysis. All treated control groups showed a reduction in numbers of dendritic cells and B cells after administration of the anti-CD40 agonist. By contrast, the experimental groups did not demonstrate the same level of reduction in dendritic cells and B cells, which indicates the ability of the exemplary conjugates in reducing CD40 expression in those cell types. CD40 negative T cells were not affected in any group, which demonstrates the specificity of the exemplary conjugates. Thus, these results demonstrate that exemplary conjugates can inhibit CD40 expression in dendritic cells and B cells, which, without being bound to any particular theory, indicates that that the migration of CD40-bearing dendritic cells and B cells was prevented or reduced.

These surprising results, and embodiments provided for herein, demonstrate that an siRNA molecule targeting CD40 can successfully be targeted to a subset of immune cells utilizing a FN3 domain that targets a cell surface protein, such as CD71, and that such constructs can selectively reduce CD40 expression in dendritic cells and B cells, and therefore, inhibit migration and margination of such cells, without affecting CD40 negative T cells. Accordingly, these compositions can be used to treat diseases mediated by dendritic and B cells and/or CD40 expression and activity, such as autoimmune diseases.

### Example 6: In Vitro Administration in Human Dendritic Cells of CD71-Binding FN3 Domains and CD40-Targeting siRNA Conjugates Reduced CD40 mRNA Expression and CD40 Protein Expression

Treatment with exemplary conjugates of CD71-binding FN3 domains and CD40-targeting siRNA, ABXC-285 (SEQ ID NOs: 570, 1941, 2051), ABXC-286 (SEQ ID NOs: 570, 1942, 2052), and ABXC-287 (SEQ ID NOs: 570, 1944, 2054), reduced expression of CD40 mRNA and reduced expression of CD40 protein in donated human dendritic cells.

Dendritic cells (500,000 cells per well) were treated with 1 μM of conjugate and an activating agent for four days in culture. The cell culture medium used was composed of RPMI, 10% fetal bovine serum, and 1% penicillin and streptomycin. On the fourth day, RNA was extracted after cell lysis and CD40 mRNA expression was quantified via qPCR. PGK1 and UBE2D2 were used as endogenous controls. Relative quantification (RQ) values were normalized to “activation only” control cells. Dendritic cells (500,000 cells per well) from the same donors underwent the same four-day treatment with conjugate and activating agent, as described above, and then remained in culture for 6-13 days after treatment. CD40 protein expression was measured via flow cytometry staining at 6, 8, 11, and 13 days after treatment. Relative expression values were normalized to “activation only” control cells.

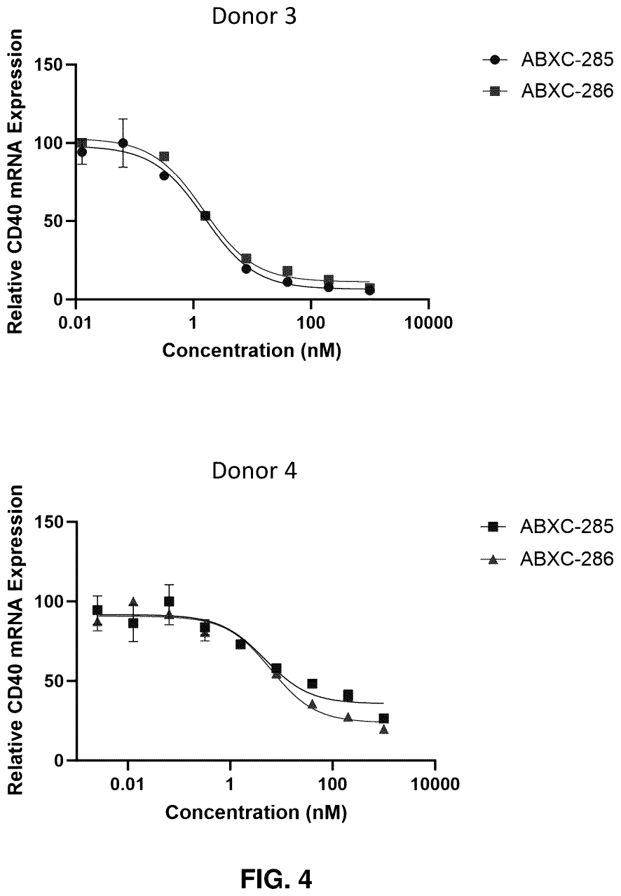

Relative CD40 mRNA expression after treatment with conjugates was reduced by over 70% across two donors. As shown in FIG. 4 and in Table 21, below, relative CD40 mRNA expression was reduced by over 90% in cells from Donor 3, and by over 70% in cells from Donor 4.

TABLE 21
CD40 mRNA Knockdown

| | | siRNA | siRNA | | | | |
|---|---|---|---|---|---|---|---|
| | | Sense | Antisense | Donor 3 Dendritic Cells | | Donor 4 Dendritic Cells | |
| Conjugate | Centyrin | Strand | Strand | EC50 | % Knockdown | EC50 | % Knockdown |
| ID | SEQ ID NO | SEQ ID NO | SEQ ID NO | (nM) | at 1 μM | (nM) | at 1 μM |
| ABXC-285 | 570 | 1941 | 2051 | <10 | >90 | <10 | >70 |
| ABXC-286 | 570 | 1942 | 2052 | <10 | >90 | <10 | >75 |

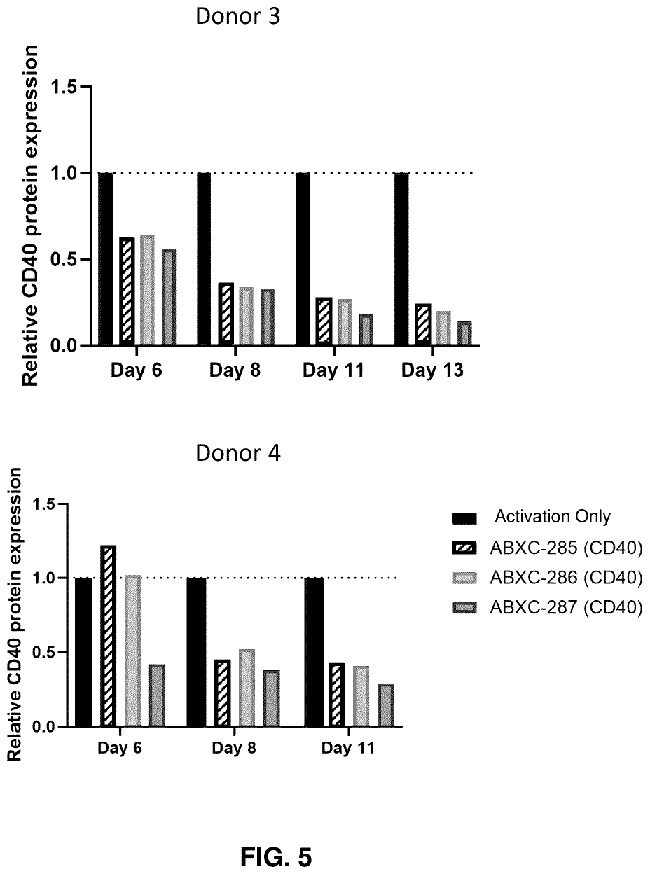

Relative CD40 protein surface expression within one week after treatment with conjugates was reduced by over 50% in dendritic cells across two donors. As shown in FIG. 5, dendritic cells from Donor 3 showed a reduction in relative CD40 protein expression of about 40% by day 6 after treatment. By day 13, relative CD40 protein expression was reduced by over 70% in cells from Donor 3. Dendritic cells from Donor 4 showed a reduction in relative CD40 protein expression of about 50% by day 8 after treatment. By day 11, relative CD40 protein expression was reduced by over 60% in cells from Donor 4.

These results demonstrate the functional activity of CD71-binding FN3 domains and CD40-targeting siRNA conjugates in human dendritic cells in vitro.

### Example 7: In Vitro Administration in Human Dendritic Cells of CD71-Binding FN3 Domain and CD40-Targeting siRNA Conjugates Reduced Cytokine Production

When activated, CD40-expressing dendritic cells produce and release cytokines. Treatment with exemplary conjugates of CD71-binding FN3 domains and CD40-targeting siRNA, ABXC-326 (SEQ ID NOs: 570, 1890, 2290) and ABXC-328 (SEQ ID NOs: 570, 1893, 2293), reduced cytokine production in human dendritic cells activated and exposed to CD40 ligand in vitro.

Dendritic cells (500,000 cells per well) were treated with 1 μM of conjugate and an activating agent for seven days, followed by exposure to CD40 ligand for one day. The cell culture medium used was composed of RPMI, 10% fetal bovine serum, and 1% penicillin and streptomycin. Supernatants were collected and a multiplex assay kit was used to quantify amounts (in pg/mL) of cytokines via electrochemiluminescence (ECL) immunoassay.

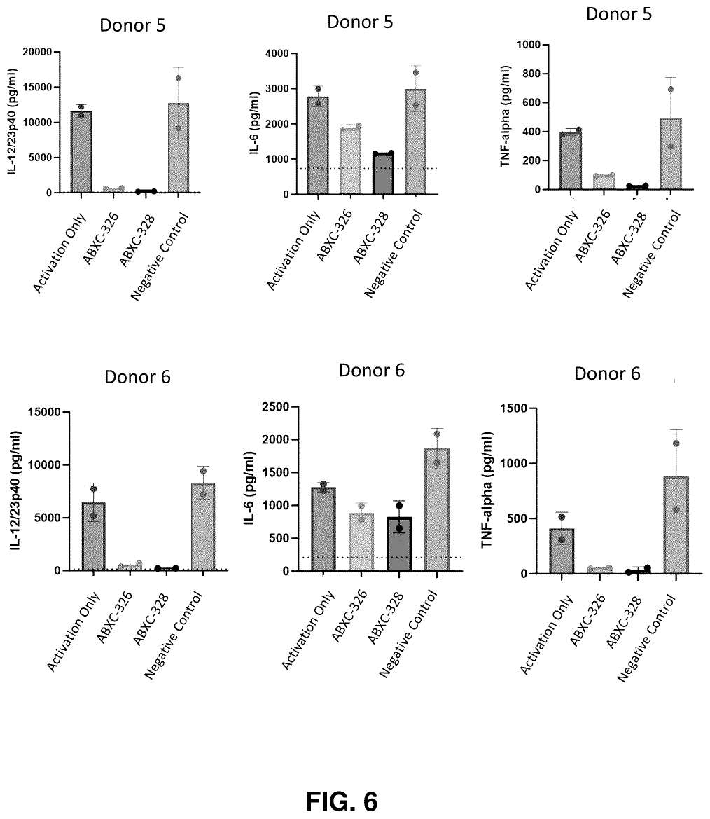

Production of cytokines after conjugate treatment was reduced in dendritic cells across two donors. As shown in FIG. 6, IL-12 production was reduced by over 90% in dendritic cells from Donor 5 and Donor 6. TNF-α production was reduced by over 70% in cells from Donor 5, and reduced by over 90% in cells from Donor 6. IL-6 production was reduced by over 30% in cells from Donor 5 and Donor 6.

These results demonstrate functional activity of the CD71-binding FN3 domains and CD40-targeting siRNA conjugates in human dendritic cells in vitro.

### Example 8: In Vivo Administration in Mice of Mouse Surrogates of CD71-Binding FN3 Domain and CD40-Targeting siRNA Conjugates Reduced Serum Cytokine Levels

Treatment with mouse surrogates of CD71-binding FN3 domain and CD40-targeting siRNA conjugates reduced serum levels of cytokines associated with dendritic cell-mediated immune responses in mice in vivo.

Six groups, each consisting of five male CD1 mice, were exposed to differing conditions over the course of three days (day 0, day 1, day 2) before termination. Two groups were treated with two different mouse surrogates of conjugates comprising CD71-binding FN3 domains and CD40-targeting siRNA (“mouse surrogate 1” and “mouse surrogate 2”). One group was treated with a conjugate of CD71 Centyrins and an siRNA not targeting CD40 (“negative control”). One group was treated with CD71 Centyrins alone, not conjugated to any siRNA (“Centyrin only”). One group was treated with hepes buffered saline only (“vehicle”). The final group received no treatment throughout the experiment (“naïve”).

On day 0, an anti-CD40 agonist monoclonal antibody was administered to all groups except the naïve group, to activate CD40-expressing cells in vivo. Two hours after administration of the anti-CD40 agonist, all groups except for the naïve group received treatment with vehicle or drug. On day 1, all groups except the naïve group received treatment again with vehicle or drug in addition to anti-CD40 agonist, after which serum cytokine levels were measured (in pg/mL) for all groups. The following cytokines were measured due to their association with dendritic cell-mediated immune responses: IFN-γ, IL-6, TNF-α, IL-12p40, IP-10, and RANTES. On day 2, all subjects were terminated.

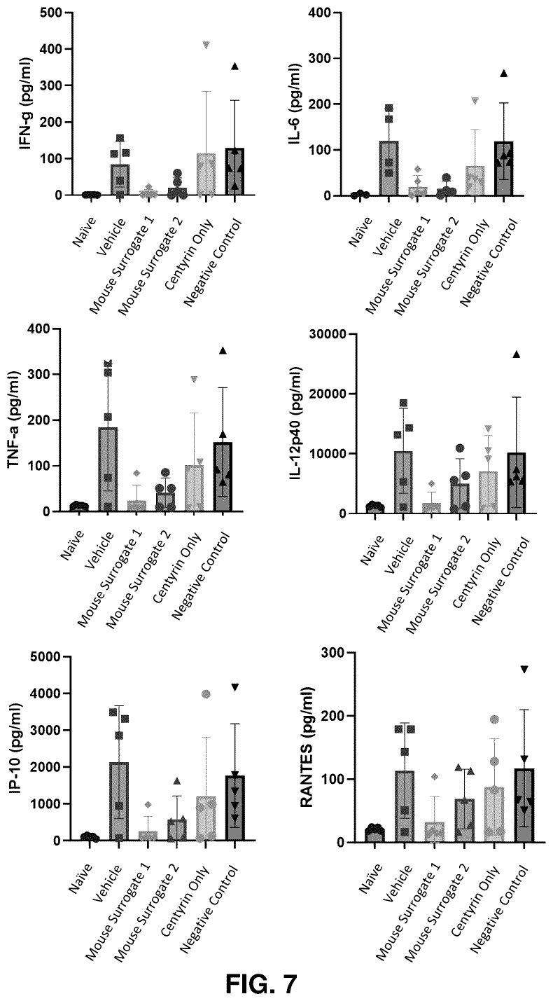

As shown in FIG. 7, mice in the naïve group, which were neither activated nor treated, showed very little amounts of any of the measured cytokines. By contrast, mice activated with an anti-CD40 agonist antibody and treated with vehicle, Centyrin only, or a negative control conjugate all displayed elevated levels of all measured cytokines relative to the naïve group, consistent with what one would expect in mice with activated CD40-expressing dendritic cells (not being bound by any particular theory). However, mice treated with mouse surrogates of CD71-CD40 conjugates experienced an almost total knockdown of IFN-γ and IL-6, comparable to levels in the naïve group. Surrogate-treated mice also showed very low levels of TNF-α, IL-12p40, IP-10, and RANTES comparable to, or statistically no different from, the naïve group. These mouse surrogates would correlate with what would be expected to be observed in human cells.

These surprising results, and embodiments provided for herein, demonstrate that an siRNA molecule targeting CD40 can successfully be targeted to a subset of immune cells utilizing a FN3 domain that targets a cell surface protein, such as CD71, and that such constructs can selectively reduce production of cytokines associated with an immune response. Accordingly, these compositions can be used to treat diseases mediated by dendritic and B cells and/or CD40 expression and activity, such as autoimmune diseases.

### Example 9: In Vivo Administration of Mouse Surrogates of CD71-Binding FN3 Domain and CD40-Targeting siRNA Conjugates Reduced Serum Cytokine Levels in a Rodent Model of CNS Autoimmune Disease and Inflammation

Experimental autoimmune encephalomyelitis (EAE) disease is a mouse model of autoimmune and inflammatory diseases in the central nervous system (CNS), such as, but not limited to, multiple sclerosis and amyotrophic lateral sclerosis. This experiment demonstrates the results of treating induced EAE mice with mouse surrogates of CD71-binding FN3 domain and CD40-targeting siRNA conjugates.

Four groups, each consisting of five female C57BL/6 mice, were exposed to differing conditions over the course of 12 days, before termination and before clinical symptoms began to manifest. One group was treated with a mouse surrogate of an exemplary CD71-CD40 conjugate as described herein, one group was treated with an anti-CD40 ligand clone (“positive control”), and one group was treated with hepes buffered saline (“vehicle”). The final group consisted of healthy mice that were neither treated nor induced to a diseased state (“naïve”).

On day 0, all groups except the naïve group were treated with drug or vehicle. On day 1, the treated groups were induced to have EAE by administration of MOG₃₅₋₅₅ in complete Freund's adjuvant, and received additional doses of drug or vehicle on days 1, 4, and 7. To quantify cytokine levels in the EAE model, serum was collected and analyzed via electrochemiluminescence (ECL) immunoassay to quantify IP-10, IL-12p70, IL-6, TNF-α, IFN-γ, and RANTES. To quantify immune cell frequency in the EAE model, spinal cord tissue and draining lymph node (dLN) tissue were collected for flow cytometry staining with the following antibodies: TCR-B, CD4, CD8, CD19, CD1 Ic, CD45, CD86, CD69, CD40, CXCR3, and CCR6.

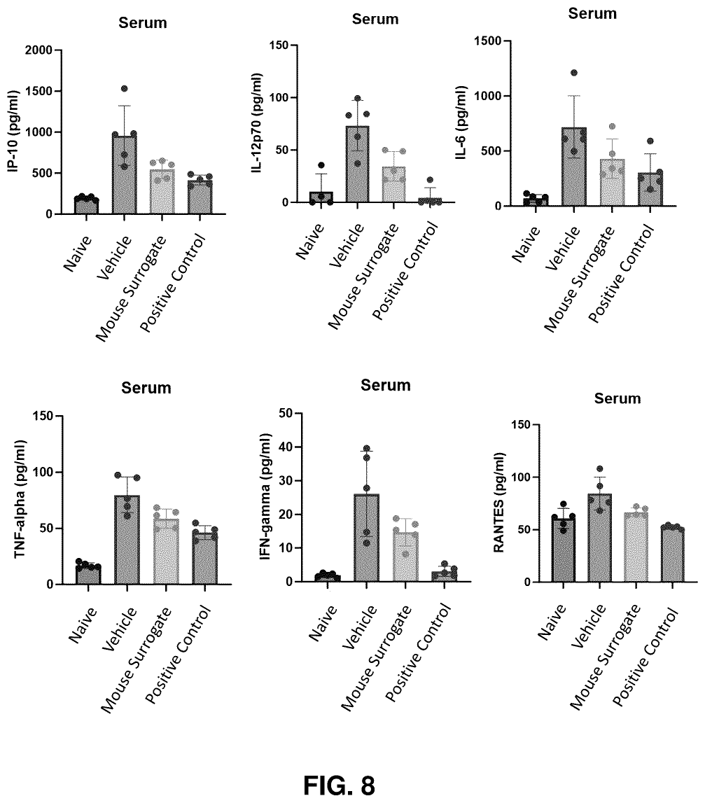

As shown in FIG. 8, EAE mice treated only with vehicle showed the highest serum levels of all measured cytokines. EAE mice treated with mouse surrogate showed relatively lower serum levels of all measured cytokines as compared to EAE mice treated with vehicle. By contrast, EAE mice treated with positive control showed serum levels of IP-10, IL-6, TNF-α, RANTES comparable to EAE mice treated with mouse surrogate, but showed knockdown of IL-12p70 and IFN-γ. These results indicate that treatment with a mouse surrogate of a CD71-CD40 conjugate suppresses cytokine induction in the serum in an EAE model.

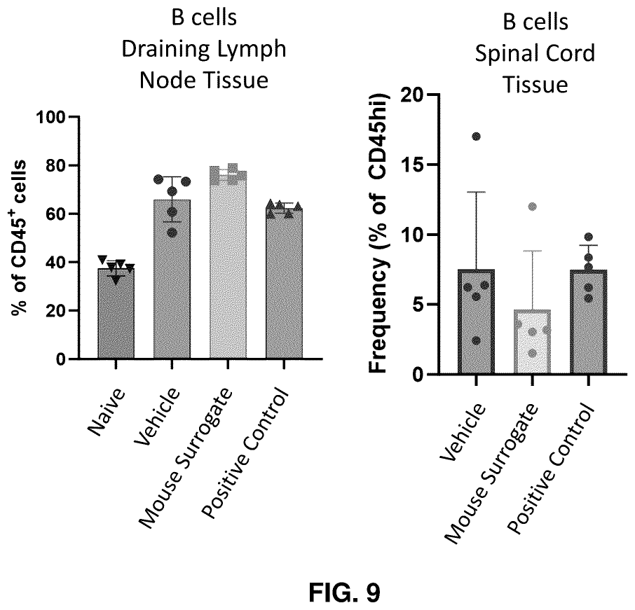

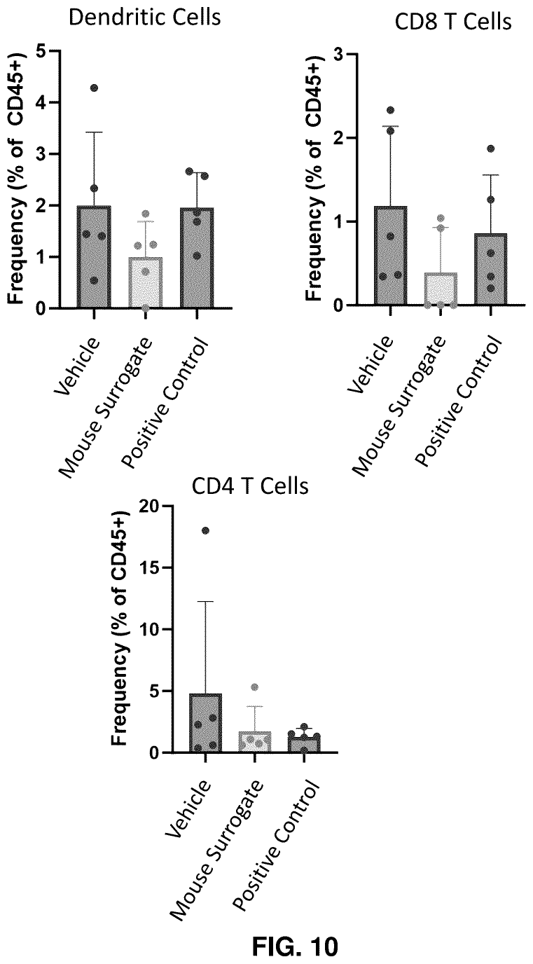

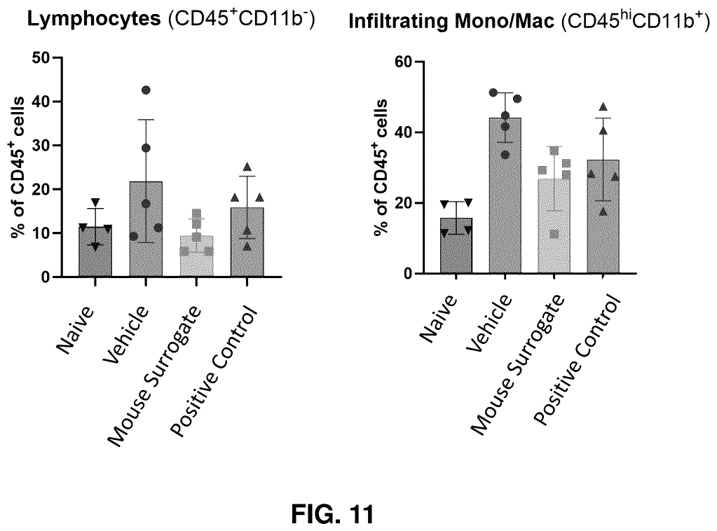

As shown in FIG. 9, all EAE mice showed a relatively higher proportion of B cells in dLN tissue as compared to spinal cord tissue. However, this difference was most pronounced in EAE mice treated with mouse surrogate. EAE mice treated with mouse surrogate showed the highest percentage of B cells in dLN tissue and the lowest percentage of B cells in spinal cord tissue as compared to EAE mice treated with vehicle or positive control. As shown in FIG. 10, EAE mice treated with mouse surrogate showed relatively lower proportions of dendritic cells, CD8 T cells, and CD4 T cells as compared to EAE mice treated with vehicle. Further, as shown in FIG. 11, EAE mice treated with mouse surrogate showed relatively lower proportions of lymphocytes, monocytes and macrophages in spinal cord tissue as compared to EAE mice treated with vehicle or positive control. These results indicate that treatment with a mouse surrogate of a CD71-CD40 conjugate suppressed B cell, dendritic cell, and T cell infiltration into the spinal cord. These mouse surrogates would correlate with what would be expected to be observed in other mammals, such as humans.

These surprising results, and embodiments provided for herein, demonstrate that an siRNA molecule targeting CD40 can successfully be targeted to a subset of immune cells utilizing a FN3 domain that targets a cell surface protein, such as CD71, and that such constructs can selectively reduce production of cytokines and suppress infiltration of immune cells into the central nervous system in an animal model of autoimmune disease. Accordingly, these compositions can be used to treat diseases mediated by dendritic and B cells and/or CD40 expression and activity, such as autoimmune diseases of the central nervous system, including but not limited to those provided for herein, and, for example, multiple sclerosis.

### Example 10: In Vitro Administration in SKBR3 Cells of CD40-Targeting siRNA Molecules Downregulates CD40 Expression with Limited Off-Target Interactions

This experiment demonstrates the results of administering CD40-targeting siRNAs to cells in vitro and measuring CD40 expression as a result. Two treatment groups of SKBR3 cells were transfected with two exemplary CD40-targeting siRNAs, ABXO-1049 (SEQ ID NOs: 1890, 2290) and ABXO-1052 (SEQ ID NOs: 1893, 2293), with lipofectamine at a concentration of 10 nM for 24 hours, with a control group of SKBR3 cells were treated only with lipofectamine, with 6 replicates per group. The concentration of 10 nM was selected because it is well past the concentration at which eMax is achieved for both siRNAs. mRNA was polyA+ selected from cells and subjected to unstranded 2×150 bp paired end sequencing to an average depth of >20 million reads.

The quality of the RNA-seq library was confirmed using FastQC. Libraries were pseudoaligned to the GrCH38.p13 human transcriptome using Kallisto. A read pseudoalignment rate average of 88.6% was achieved, demonstrating the good quality of the data. Differential expression was then assessed using DESeq2 comparing cells in each treatment group to cells in the control group. Any non-protein coding genes and genes express at less than 10 reads in 75% or greater of the libraries being compared were filtered out from downstream analyses. Significance of differential expression signal was calculated using the DESeq2 log 2 (fold-change) (LFC) threshold parameter, with which a 0.585 LFC baseline for statistical testing was applied. LFC shrinkage was estimated using AshR.

CD40 mRNA expression was substantially and significantly knocked down. CD40 expression in each of the two treatment groups was downregulated to 32% and 22% of the control group. CD40 was the most downregulated protein coding gene for all genes that were differentially expressed in both treatment groups.

In silico prediction of potential off-target effects was performed using BLAST and Bowtie aligners. The sense and antisense sequences of the exemplary siRNAs were aligned against internal databases built from the GRCh38 transcriptome. Of all the predicted potential off-target effects identified by these aligners, only one significantly downregulated off-target effect was identified in each siRNA.

In total, the first treatment group showed eight significant off-target effects detectable in these sequencing data, and second treatment group showed two significant off-target effects detectable. In the case of both siRNAs, these off-target signals were attributable to the antisense sequences.

Together, these data demonstrate that the exemplary CD40-targeting siRNAs are highly specific to CD40, with limited off-target interactions.

### Example 11: In Vitro Administration in Human Dendritic Cells of CD71-Binding FN3 Domains and CD40-Targeting siRNA Conjugates Reduced CD40 Protein Expression

Treatment with exemplary conjugates of CD71-binding FN3 domains and CD40-targeting siRNA, as detailed below in Table 22, knocked down expression of CD40 protein in donated human dendritic cells.

Dendritic cells (500,000 cells per well) were treated with 1 μM of conjugate and an activating agent for 11 days in culture. The cell culture medium used was composed of RPMI, 10% fetal bovine serum, and 1% penicillin and streptomycin. On day 11, CD40 protein expression was measured via flow cytometry using a CD40 antibody clone. Relative protein expression values were normalized to “activation only” control cells.

As shown in Table 22, below, expression of CD40 protein was knocked down by 50-82% in dendritic cells from three different human donors following administration of CD71-CD40 conjugates, relative to CD40 protein expression in activated control cells.

TABLE 22
CD40 Protein Knockdown

| | | siRNA | siRNA | | |
|---|---|---|---|---|---|
| | | Sense | Antisense | | % CD40 |
| | Centyrin | Strand | Strand | Dendritic | Protein |
| | SEQ | SEQ | SEQ | Cell | Knock- |
| Conjugate ID | ID NO | ID NO | ID NO | Donor | down |
| ABXC-285 | 570 | 1941 | 2051 | Donor 3 | 73% |
| ABXC-286 | 570 | 1942 | 2052 | Donor 3 | 73% |
| ABXC-287 | 570 | 1944 | 2054 | Donor 3 | 82% |
| ABXC-285 | 570 | 1941 | 2051 | Donor 4 | 57% |
| ABXC-286 | 570 | 1942 | 2052 | Donor 4 | 59% |
| ABXC-287 | 570 | 1944 | 2054 | Donor 4 | 71% |
| ABXC-326 | 570 | 1890 | 2290 | Donor 5 | 48% |
| ABXC-328 | 570 | 1893 | 2293 | Donor 5 | 50% |
| ABXC-326 | 570 | 1890 | 2290 | Donor 6 | 51% |
| ABXC-328 | 570 | 1893 | 2293 | Donor 6 | 66% |

These results demonstrate the functional activity of CD71-binding FN3 domains and CD40-targeting siRNA conjugates in human dendritic cells from multiple donors in vitro.

General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St.

Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present embodiments are not to be limited in scope by the specific embodiments described herein. Indeed, various modifications of the embodiments in addition to those described herein will become apparent to those skilled in the art from the foregoing description. Such modifications are intended to fall within the scope of the appended claims.

The foregoing written specification is considered to be sufficient to enable one skilled in the art to practice the embodiments. Various modifications of the embodiments in addition to those shown and described herein will become apparent to those skilled in the art from the foregoing description and fall within the scope of the appended claims.

1. A composition comprising an siRNA molecule comprising a sense strand and antisense strand that targets CD40 gene, wherein:
the sense strand comprises the nucleic acid sequence of SEQ ID NO: 1890 and the antisense strand comprises the nucleic acid sequence of SEQ ID NO: 2290;
the sense strand comprises the nucleic acid sequence of SEQ ID NO: 1893 and the antisense strand comprises the nucleic acid sequence of SEQ ID NO: 2293;
the sense strand comprises the nucleic acid sequence of SEQ ID NO: 1941 and the antisense strand comprises the nucleic acid sequence of SEQ ID NO: 2051;
the sense strand comprises the nucleic acid sequence of SEQ ID NO: 1942 and the antisense strand comprises the nucleic acid sequence of SEQ ID NO: 2052;
the sense strand comprises the nucleic acid sequence of SEQ ID NO: 1944 and the antisense strand comprises the nucleic acid sequence of SEQ ID NO: 2054;
the sense strand comprises the nucleic acid sequence of SEQ ID NO: 2110, or a modified version thereof, and the antisense strand comprises the nucleic acid sequence of SEQ ID NO: 2220, or a modified version thereof;
the sense strand comprises the nucleic acid sequence of SEQ ID NO: 2113, or a modified version thereof, and the antisense strand comprises the nucleic acid sequence of SEQ ID NO: 2223, or a modified version thereof;
the sense strand comprises the nucleic acid sequence of SEQ ID NO: 2111, or a modified version thereof, and the antisense strand comprises the nucleic acid sequence of SEQ ID NO: 2221, or a modified version thereof; or
the sense strand comprises a nucleic acid sequence selected from any one of SEQ ID NOs: 46-178, 312-331, 352-356, 673-805, 939-958, 1850, 1851, 1891, 1892, 1894-1928, 1932-1940, 1943, 1945-1959, 2070, 2071, 2110-2148, 2152-2179, 2298, 2300, 2302, 2304, 2306, and 2308, or a modified version thereof, and the antisense strand comprises a nucleic acid sequence selected from any one of SEQ ID NOs: 179-311, 332-351, 356-359, 806-938, 959-978, 1960, 1961, 2000-2038, 2042-2069, 2180, 2181, 2220-2258, 2262-2297, 2299, 2301, 2303, 2305, 2307, and 2309, or a modified version thereof.

2. The composition of claim 1, wherein the siRNA molecule further comprises a linker covalently attached to the sense strand or the antisense strand.

3. The composition of claim 2, wherein the linker is attached to a 5′ end or a 3′ end of the sense strand or the antisense strand.

4. The composition of claim 1, wherein the siRNA molecule further comprises a vinyl phosphonate modification on the sense strand or the antisense strand.

5. The composition of claim 4, wherein the vinyl phosphonate modification is attached to a 5′ end or a 3′ end of the sense strand or the antisense strand.

6.-9. (canceled)

10. The composition of claim 1, wherein the siRNA molecule has the formula as illustrated in Formula III:
(III)
N₁N₂N₃N₄N₅N₆N₇N₈N₉N₁₀N₁₁N₁₂N₁₃
N₁₄N₁₅N₁₆N₁₇N₁₈N₁₉
Sense-strand (SS)
N₂₁N₂₀N₁₉N₁₈N₁₇N₁₆N₁₅N₁₄N₁₃N₁₂
N₁₁N₁₀N₉N₈N₇N₆N₅N₄N₃N₂N₁
Antisense-strand (AS),
wherein each nucleotide represented by N, is as set forth in SEQ ID NO: 2110 and SEQ ID NO: 2220, SEQ ID NO: 2113 and SEQ ID NO: 2223, SEQ ID NO: 2111 and SEQ ID NO: 2221, or comprising a modified nucleotide base thereof.

11. The composition of claim 10, wherein the sense strand comprises a 2′-fluoro modified nucleotide at N3, N7, N8, N9, N12, and N17, and a 2′O-methyl modified nucleotide at N1, N2, N4, N5, N6, N10, N11, N13, N14, N15, N16, N18, and N19.

12. The composition of claim 10, wherein the antisense strand comprises a vinyl phosphonate moiety with a phosphorothioate (PS) modified backbone attached at N1, a 2′fluoro-modified nucleotide with a PS-modified backbone at N2, a 2′O-methyl modified nucleotide at N3, N4, N5, N6, N7, N8, N9, N10, N11, N12, N13, N15, N16, N17, N18, and N19, a 2′fluoro-modified nucleotide at N14, and a 2′O-methyl modified nucleotide with a PS-modified backbone at N20 and N21.

13. The composition of claim 10, wherein the antisense strand comprises a vinyl phosphonate moiety attached to N1.

14. The composition of claim 10, wherein the siRNA molecule is conjugated to a linker as shown in the following formula:

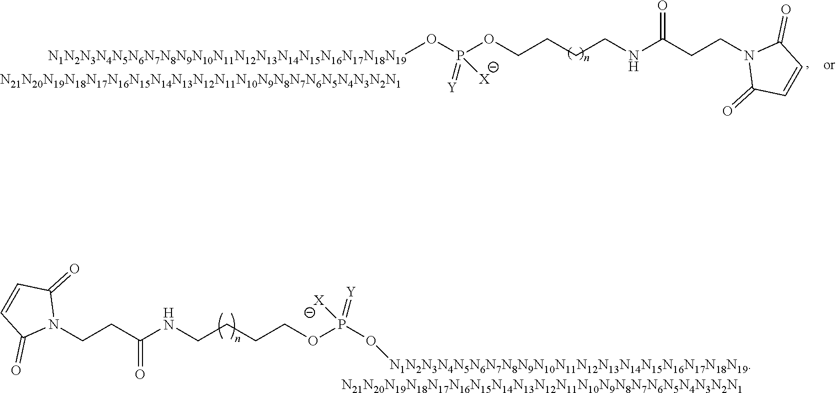

15. The composition of claim 1, wherein the siRNA molecule has a formula as shown in the following formulas: 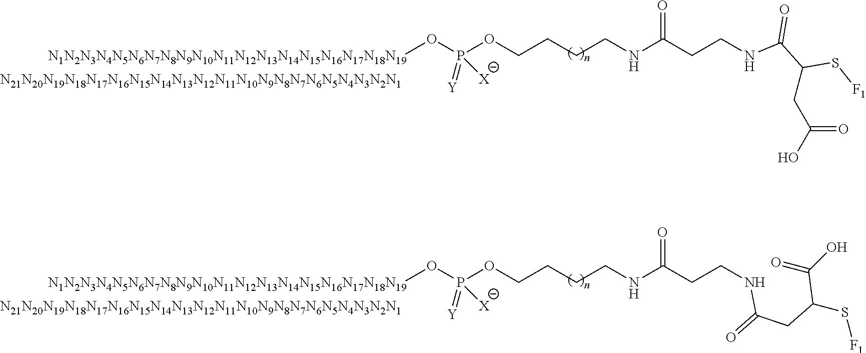
wherein F1 is a polypeptide comprising at least one FN3 domain.

16. The composition of claim 1, further comprising one or more FN3 domains conjugated to the siRNA molecule.

17. The composition of claim 16, wherein the one or more FN3 domains comprises an FN3 domain conjugated to the siRNA molecule through a cysteine in the FN3 domain.

18. The composition of claim 16, wherein the one or more FN3 domains comprises an FN3 domain that binds to CD71.

19. The composition of claim 18, wherein the FN3 domain that binds to CD71 comprises an amino acid sequence that is at least 87%, 88%, 89%, 90%, 91%, 92%, 93%, 94%, 95%, 96%, 97%, 98%, 99% identical to, or is identical to, a sequence selected from any one of SEQ ID NOs: 570, 672, 1848, 1773, 1849, 1767, 360-569, 571-644, 663-671, 1395-1766, 1768-1772, 1774-1847, and 2310.

20. The composition of claim 18, wherein the FN3 domain comprises an amino acid sequence that is at least 95% identical to the amino acid sequence of SEQ ID NO: 570, or SEQ ID NO: 672, SEQ ID NO: 1848, SEQ ID NO: 1773, SEQ ID NO: 1849, or SEQ ID NO: 1767.

21. The composition of claim 18, wherein the FN3 domain comprises an amino acid sequence that comprises the amino acid sequence of SEQ ID NO: 570, SEQ ID NO: 672, SEQ ID NO: 1848, SEQ ID NO: 1773, SEQ ID NO: 1849, or SEQ ID NO: 1767.

22. (canceled)

23. A pharmaceutical composition comprising the composition of claim 1.

24. A kit comprising the composition of claim 1.

25. A method of treating an immunological disease in a subject in need thereof, the method comprising administering to the subject the composition of claim 1.

26.-41. (canceled)
